(19) United States
(12) Patent Application Publication
(10) Pub. No.: US 2007/0021434 A1
(43) Pub. Date: Jan. 25, 2007

(54) NON-NUCLEOSIDE ANTI-HEPACIVIRUS AGENTS AND USES THEREOF

(75) Inventors: Vincent A. Boyd, New Braunfels (CA); Dale R. Cameron, Richmond (CA); Qi Jia, Burnaby (CA); Paulo W.M. Sgarbi, Richmond (CA); Shirley A. Wacowich-Sgarbi, Richmond (CA)

Correspondence Address: DARBY & DARBY P.C., P. O. BOX 5257, NEW YORK, NY, 10150-5257, US

(73) Assignee: Migenix Inc., Vancouver (CA)

(21) Appl. No.: 11/426,580

(22) Filed: Jun. 26, 2006

Related U.S. Application Data
(60) Provisional application No. 60/693,569, filed on Jun. 24, 2005.

(51) Int. Cl. A61K 31/495; A61K 31/4172; A61K 31/16

(52) U.S. Cl. 514/252.12; 514/400; 514/621; 544/399; 548/335.5; 564/193

(57) ABSTRACT

The present dislcosure provides amide-based, non-nucleoside compounds having antiviral activity against Hepacivirus, such as hepatitis C virus (HCV), methods and intermediates for synthesizing such compounds, and methods of using the compounds in a variety of contexts, including in the treatment and prevention of viral infections. The present dislcosure also provides methods for identifying amide-based, non-nucleoside compounds having antiviral activity.

### CROSS-REFERENCE TO RELATED APPLICATION

This application claims the benefit of U.S. Provisional Patent Application No. 60/693,569, filed Jun. 24, 2005, which provisional application is incorporated herein by reference in its entirety.

### TECHNICAL FIELD

The present disclosure relates generally to agents for treating or preventing viral infections and, more specifically, to amide-based compounds for therapeutic use against Hepacivirus infections, such as infections caused by or associated with hepatitis C virus (HCV) infections, and to methods for identifying amide-based, non-nucleoside compounds having antiviral activity.

### BACKGROUND

Several types of viral infections can eventually lead to other related afflictions (e.g., other viral infections or bacterial infections) or diseases (e.g., cancer). For example, infections caused by or associated with hepatitis C virus (HCV) can progress to cirrhosis and hepatocellular carcinoma (HCC) (Hoofnagle, Hepatology 26:15S, 1997). An estimated 30,000 new cases of HCV infection occur every year in the United States (U.S.) alone (Kolykhalov et al., J. Virol. 74:2046, 2000), and of these, up to 85% may progress to chronic infection and more serious diseases (such as, cirrhosis and HCC). Up to 10,000 people die each year from HCV related disease in the U.S., and over 170 million HCV carriers are estimated to exist worldwide. Existing treatments include interferon and ribavirin, but have only a 50% response rate in treated patients (Lindsay, Hepatology 26:71S, 1997; Reichard et al., Hepatology 26:108S, 1997).

The HCV encoded RNA-dependent RNA polymerase (HCV RdRp), also known as non-structural protein 5B (NS 5B), has been vigorously investigated as a target for antiviral therapies because it does not exist in mammalian cells and is essential for viral replication (Kolykhalov et al, J. Virol. 74:2046, 2000). Several publications describe attempts to generate therapeutics that are specific to HCV RdRp, both nucleoside-based (see, e.g., PCT Application Publication Nos. WO 01/90121, WO 02/57425, WO 03/026589) and non-nucleoside-based (see, e.g., PCT Application Publication Nos. WO 00/50424, WO 00/06529, WO 00/10573, WO 00/13708, WO 00/18231, WO 01/60315, WO 02/100851, WO 2004/002944, WO 2004/002977; European Patent Application No. 1162196; U.S. Application Nos. 2003/0236251, 2003/0176433, 2003/0050320, 2003/0229053; U.S. Pat. Nos. 6448281, 6479508; Wang et al., J. Biol. Chem. 278(11):9489, 2003). However, these agents are not approved to date for clinical use, and display limited efficacy, potential toxicity and/or eventually cause the emergence of viral resistance (Migliaccio et al., 16^{th} ICAR Meeting, Savannah, Ga., April, 2003 and Tomei et al., J. Virol. 77(24):13225, 2003).

### SUMMARY

Briefly, the present disclosure provides non-nucleoside compounds that can be used as antiviral agents for treating or preventing Hepacivirus infections, such as infections caused by or associated with hepatitis C virus (HCV).

In one aspect, the present disclosure provides antiviral agents having a structure of formula (IV): 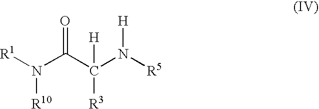
or a stereoisomer, prodrug or pharmaceutically acceptable salt thereof, wherein:

R¹, R³ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;

R⁵ is selected from —C(═O)NR⁹, —C(═O)(NR¹⁰)SO₂R⁹, —C(═S)NR¹⁰R⁹, or —C(═NR¹⁰)NR¹⁰R⁹; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl.

In a further embodiment, provided is a compound having a structure of formula (IV) as defined herein, wherein R³ is not hydrogen, or wherein R³ has an ionizable nitrogen. In still further embodiments, provided is a compound having a structure of formula (IV) as defined herein, wherein the compound is compound 2, 297, 137, 146, 172, 199, 228, 272, 121, 142, 26, 94, 117, 119, 120, 125, 127, 145, 166, 173, 206, 207, 214, 237, 240, 268, 270, or 306.

In a further aspect, the instant disclosure provides compounds having a structure of formula (V): 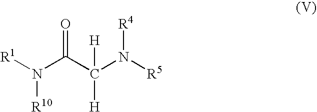
or a stereoisomer, prodrug or pharmaceutically acceptable salt thereof, wherein:

R¹, R⁴ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ and R⁴ are not hydrogen;

R⁵is selected from —C(═O)NR⁹, —C(═O)(NR¹⁰)SO₂R⁹, —C(═S)NR¹⁰R⁹, or —C(═NR¹⁰)NR¹⁰R⁹; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl.

In a further embodiment, provided is a compound having a structure of formula (V) as defined herein, wherein R⁴ has an ionizable nitrogen. In still further embodiments, provided is a compound having a structure of formula (V) as defined herein, wherein the compound is compound 234, 262, 279, 281, 282, 294, 295, or 324.

In a further aspect, provided are compounds having a structure of formula (VI): 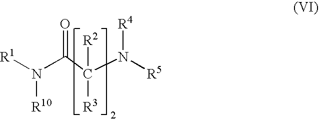
or a stereoisomer, prodrug or pharmaceutically acceptable salt thereof, wherein:

R¹ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;

(i) R², R³ and R⁴ are each independently the same or different substituent as defined for R⁹; or (ii) R³ and R⁴ taken together with the carbon atom and N atom to which they are bonded, respectively, form a five—to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iii) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, or —SO₂R⁹; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl;

and wherein at least one but not more than three of R² , R³, R⁴ and R⁵ is hydrogen, provided that

R¹ is not an amino acid when R⁴ and R⁵ are both H.

In a further embodiment, provided is a compound having a structure of formula (VI) as defined herein, wherein the compounds have a structure of formula (VII): 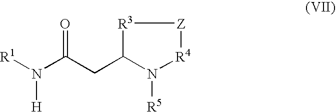
wherein R³ and R⁴ are each independently selected from —CH₂— or —CH₂)₂—; Z is —N(R⁹)—; and R¹, R⁵, R⁹, and R¹⁰ are as defined herein for structure (VI). In one embodiment, there is provided a compound of structure (VII), wherein the R⁹ has an ionizable nitrogen. In another embodiment, there is provided a compound of structure (VII), wherein the R⁹ has an ionizable nitrogen, R³ is —CH₂— and R⁴ is —(CH₂)₂—. In still further embodiments, provided is a compound having a structure of formula (VI) as defined herein, wherein the compound is compound 155, 158, 159, 160, 161, 162, 163, 183, 184, 186, 187, or 197.

In a further aspect, the instant disclosure provides compounds having a structure of formula (VIII): 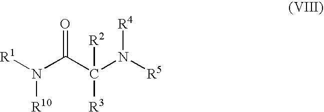
or a stereoisomer, prodrug or pharmaceutically acceptable salt thereof, wherein:
R¹ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂ C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;

(i) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (ii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iii) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, or —SO₂R⁹; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl;

and wherein at least one but not more than three of R², R³, R⁴ and R⁵ is hydrogen, provided that R¹ is not an amino acid when R⁴ and R⁵ are both H.

In a further embodiment, the instant disclosure provides a compound of structure (VIII) as defined herein, wherein at least one of R², R³ or R⁴ has an ionizable nitrogen. In another embodiment, provided is a compound of structure (VIII) as defined herein, wherein R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent. In certain embodiments, the compound of formula (VIII) is compound 85, 86, 87, 122, 123, 130, 131, 132, or 156 as shown in FIG. 5. In still another embodiment, provided is a compound of structure (VIII) as defined herein, wherein R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent. In certain embodiments, the compound of formula (VIII) is compound 109 or 138.

In a further aspect, the instant disclosure provides compounds having a structure of formula (IX): 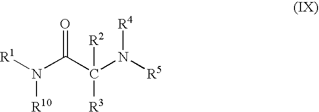
or a stereoisomer, prodrug or pharmaceutically acceptable salt thereof, wherein:

R¹ is the same as R⁹ provided an ionizable nitrogen is present;

(i) R², R³ and R⁴ are each independently the same or different substituent as defined for R⁹; or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R is selected from R⁹; or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR⁹, —C(═O)(NR¹⁰)SO₂R⁹, —C(═S)NR¹⁰R⁹, —C(═NR¹⁰)NR¹⁰ R⁹, —OR⁹, —SR⁹, —NR¹⁰R⁹, —S(═O)R⁹, —SO₂R⁹;

R⁹ is selected from H, (C₁C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl; and wherein at least one but not more than three of R², R³, R⁴ and R⁵ is hydrogen, provided that R¹ is not an amino acid when R⁴ and R⁵ are both H.

In certain embodiments, the compound of formula (IX) is compound 314, 315, 316, 319, or 320 as shown in FIG. 5.

In a further aspect, the instant disclosure provides compounds having a structure of formula (X): 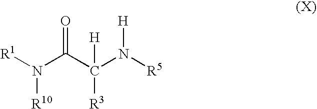
or a stereoisomer, prodrug or pharmaceutically acceptable salt thereof, wherein:

R¹, R³ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;

R⁵ is selected from H, —C(═O)R⁹, —C(═S)R⁹, —C(═NR¹⁰)R⁹, or —CO₂R⁹; R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl.

In a further embodiment, the instant disclosure provides a compound having a structure of formula (X) as defined herein, wherein R³ is not hydrogen or wherein R³ has an ionizable nitrogen. In still further embodiments, provided is a compound having a structure of formula (X) as defined herein, wherein the compound is compound 334 to 369, and in certain embodiments is compound 358, 360, 366, 367 or 368 as shown in FIG. 5.

In a further aspect, any of the antiviral compounds of this disclosure can be used alone or in combination with an adjunctive therapy to treat or prevent Hepacivirus infections, such as HCV infections. In certain embodiments, the compounds or combinations thereof are administered parenterally.

In another aspect, the present disclosure provides a method for identifying an inhibitor of RNA-dependent RNA polymerase (RdRp) activity, comprising (a) contacting an RdRp with a template-primer and non-radioactively labelled nucleotide triphosphate molecules, in the presence or absence of a target antiviral compound, (b) detecting incorporation of the non-radioactively labelled nucleotides into a nucleic acid molecule product, and (c) comparing the amount of labelled nucleic acid molecule product produced in the presence and absence of the target antiviral compound, wherein a decrease in labelled nucleic acid molecule product is indicative of an inhibitor of RdRp activity.

### BRIEF DESCRIPTION OF THE DRAWINGS

FIG. 1 is a schematic representation of the RNA-dependent RNA polymerase (RdRp) assay with ELISA detection. The assay involves sequential steps including compound preparation, polymerase reaction, binding to the streptavidin plates and detection.

FIG. 2 shows a graph comparing optimal Mn²⁺ concentration for recombinantly prepared and Replizyme® HCV RdRp enzymes. Percent of maximum absorbance is plotted against concentration of MnCl₂.

FIGS. 3A-3D are graphs showing the determination of Kₘ for RdRp enzymes. (A) Replizyme(t enzyme reaction velocity, measured as ΔAbs/min, as plotted against the concentration of UTP (μM). (B) The Kₘ was determined to be 9.3 μM (Scatchard plot). (C) Recombinant NS5B enzyme reaction velocity (ΔAbs/min) as plotted against the concentration of template/primer (nM). (D) The Kₘ was determined to be 5 nM (Scatchard plot).

FIGS. 4A and 4B show (A) optimized and non-optimized assay conditions for prepared and Replizyme® HCV RdRp enzymes, with absorbance at 450 nm plotted against time, and (B) a dose-response inhibitory curve for anti-HCV compound E-HCV-5.

FIG. 5 shows exemplary structures of antiviral compounds of structure (I)-(X). See Table 1 for antiviral activity of these compounds.

### DETAILED DESCRIPTION

As set forth herein, the present disclosure provides non-nucleoside compounds that can be used as antiviral agents for treating or preventing hepacivirus infections, such as hepatitis C virus (HCV) infections. The compounds disclosed have an amide-based core structure and an unusually high inhibitory activity against HCV replication, which may be effected by directly or indirectly altering polymerase activity (i.e., RdRp or NS5B activity). In one embodiment of the present disclosure, compounds are provided having a structure of formula (I): 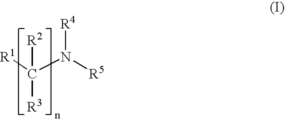
or a stereoisomer, prodrug or pharmaceutically acceptable salt thereof, wherein:

n is 1-5;

R¹ is selected from —C(═O)R⁹, —C(═S)R⁹, —C(═NR¹⁰)R⁹, —C(═O)NR⁹, —C(═O)(NR¹⁰)SO₂R⁹, —C(═S)NR¹⁰R⁹, —C(═NR¹⁰)NR¹⁰R⁹, —S(═O)R⁹, —SO₂R⁹, provided that R⁹ is not H, and that R⁹ does not form an ester with the carbonyl group to which it is bonded when R¹ is —C(═O)R⁹;

(i) R², R³ and R⁴ are each independently the same or different substituent as defined for R⁹; or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, —SO₂R⁹;

R⁹ is selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl; and wherein at least one but not more than three of R², R³, R⁴ and R⁵ is hydrogen, provided that R¹ is not an amino acid when R⁴ and R⁵ are both H.

Prior to setting forth this disclosure in more detail, it may be helpful to an understanding thereof to provide definitions of certain terms to be used herein.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” or “consisting essentially of” mean ±15% of the range or value. The use of the alternative (e.g., “or”) should be understood to mean either one, both or any combination thereof of the alternatives. In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present invention.

“Alkyl” refers to a saturated or unsaturated, branched, straight-chain or cyclic monovalent hydrocarbon group derived by the removal of one hydrogen atom from a single carbon atom of a parent alkane, alkene or alkyne. Representative alkyl groups include methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl , prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl , but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, and the like.

The term “Alkyl” is specifically intended to include straight- or branched-hydrocarbons having from 1 to 12, or 1 to 8, or 1 to 6, or 1 to 4 carbon atoms. The alkyls may have any degree or level of saturation, such as groups having exclusively single carbon-carbon bonds, groups having one or more double carbon-carbon bonds, groups having one or more triple carbon-carbon bonds and groups having mixtures of single, double and triple carbon-carbon bonds. The expressions “alkanyl,” “alkenyl,” and “alkynyl” are used when a specific level of saturation is intended. The expression “lower alkyl” refers to alkyl groups comprising from 1 to 8 carbon atoms. The alkyl group may be substituted or unsubstituted.

“Alkanyl” refers to a saturated branched, straight-chain or cyclic alkyl group. Representative alkanyl groups include methanyl; ethanyl; propanyls such as propan-1-yl, propan-2-yl (isopropyl), cyclopropan-1-yl, etc.; butyanyls such as butan-1-yl, butan-2-yl (sec-butyl), 2-methyl-propan-1-yl (isobutyl), 2-methyl-propan-2-yl (t-butyl), cyclobutan-1-yl, and the like. The alkanyl group may be substituted or unsubstituted.

“Alkenyl” refers to an unsaturated branched, straight-chain or cyclic alkyl group having at least one carbon-carbon double bond derived by the removal of one hydrogen atom from a single carbon atom of a parent alkene. The group may be in either the cis or trans conformation about the double bond(s). Representative alkenyl groups include ethenyl; propenyls such as prop-1-en-1-yl , prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, and the like. The alkenyl group may be substituted or unsubstituted.

“Alkynyl” refers to an unsaturated branched, straight-chain or cyclic alkyl group having at least one carbon-carbon triple bond derived by the removal of one hydrogen atom from a single carbon atom of a parent alkyne. Representative alkynyl groups include ethynyl; propynyls such as prop-1-yn-1-yl, prop-2-yn-1-yl, etc.; butynyls such as but-1-yn-1-yl, but-1-yn-3-yl, but-3-yn-1-yl, and the like. The alkynyl group may be substituted or unsubstituted.

“Alkyldiyl” refers to a saturated or unsaturated, branched, straight-chain or cyclic divalent hydrocarbon group derived by the removal of one hydrogen atom from each of two different carbon atoms of a parent alkane, heteroalkane, alkene, heteroalkene, alkyne or heteroalkyne, or by the removal of two hydrogen atoms from a single carbon atom of a parent alkane, heteroalkane, alkene, heteroalkene, alkyne or heteroalkyne. The two monovalent radical centers or each valency of the divalent radical center can form bonds with the same or different atoms. Representative alkyldiyl groups include methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. The nomenclature of alkanyldiyl, alkenyldiyl and/or alkynyldiyl, as well as heterocompounds thereof, is used when specific levels of saturation are intended. In certain embodiments, the alkyldiyl group is (C₁-C₄) alkyldiyl. In further embodiments, the alkyldiyl group is a saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra). The alkyldiyl group may be substituted or unsubstituted.

“Alkyleno” refers to a straight-chain alkyldiyl group having two terminal monovalent radical centers derived by the removal of one hydrogen atom from each of the two terminal carbon atoms of straight-chain parent alkane, heteroalkane, alkene, heteroalkene, alkyne or heteroalkyne. Representative alkyleno groups include methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, but[1,3]diyno, etc. When specific levels of saturation are intended, the nomenclature alkano, alkeno and/or alkyno is used. In certain embodiments, the alkyleno group is (C₁-C₆) or (C₁-C₄) alkyleno. In further embodiments, the alkyleno group is a straight-chain saturated alkano groups, e.g., methano, ethano, propano, butano, and the like. The alkynyl group may be substituted or unsubstituted.

“Heteroalkyl, Heteroalkanyl, Heteroalkenyl, Heteroalkanyl, Heteroalkyldiyl and Heteroalkyleno” refer to alkyl, alkanyl, alkenyl, alkynyl, alkyldiyl and alkyleno groups, respectively, in which one or more of the carbon atoms (and any associated hydrogen atoms) are each independently replaced with the same or different heteroatoms or heteroatomic groups. Representative heteroatoms or heteroatomic groups include —O—, —S—, —Se—, —O—O—, —S—S—, —O—S—, —O—S—O—, —O—NR⁶—, —NR⁶—, —NR⁶—NR⁶—, ═N—NR═, —NR═N—, —NR═N—NR⁶—, —PH—, —P(O)₂—, —O—P(O)₂—, —SH₂—, —S(O)₂—, —SnH₂— and the like, and combinations thereof, such as —NR⁶—S(O)₂—; wherein each R⁶ is independently selected from the group consisting of hydrogen, alkyl, alkanyl, alkenyl, alkynyl, aryl, arylalkyl, heteroaryl and heteroarylalkyl, as described herein.

“Aryl” refers to a monovalent aromatic hydrocarbon group derived by the removal of one hydrogen atom from a single carbon atom of a parent aromatic ring system. Representative aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, the aryl group is (C₅-C₁₈) or (C₅-C₁₂) aryl. Other representative aryls are cyclopentadienyl, phenyl, biphenyl, and naphthyl. The aryl group may be substituted or unsubstituted.

“Arylalkyl” refers to an acyclic alkyl group in which one of the hydrogen atoms bonded to a carbon atom, such as a terminal or sp³ carbon atom, is replaced with an aryl group.

Representative arylalkyl groups include benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The nomenclature of arylalkanyl, arylakenyl and/or arylalkynyl is used when specific alkyl moieties are intended. In certain embodiments, the arylalkyl group is (C₆-C₂₀) arylalkyl, e.g., the alkanyl, alkenyl or alkynyl moiety of the arylalkyl group is (C₁-C₆) or (C₂-C₆), and the aryl moiety is (C₅-C₁₄). In further embodiments, the arylalkyl group is (C₆-C₁₄), e.g., the alkanyl, alkenyl or alkynyl moiety of the arylalkyl group is (C₁-C₄) or (C₂-C₄), and the aryl moiety is (C₅-C₁₀). The arylalkyl group may be substituted or unsubstituted.

“Heteroaryl” refers to a monovalent heteroaromatic group derived by the removal of one hydrogen atom from a single atom of a parent heteroaromatic ring system, which may be monocyclic or fused ring (i.e., rings that share an adjacent pair of atoms). Representative heteroaryl groups include groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, the heteroaryl group is a 5-14 membered or a 5-10 membered heteroaryl. In further embodiments, heteroaryl groups are those derived from thiophene, pyrrole, furan, benzothiophene, benzofuran, indole, pyridine, pyrimidine, quinoline, imidazole, oxazole and pyrazine. The heteroaryl group may be substituted or unsubstituted.

“Heteroalicyclic” refers to a monocyclic or fused ring group having in the ring(s) one or more atoms selected from, for example, nitrogen, oxygen and sulfur. The rings may also have one or more double bonds. However, the rings do not necessarily have a completely conjugated π-electron system. The heteroalicyclic ring may be substituted or unsubstituted. When substituted, the substituted group(s) may be selected independently from alkyl, aryl, haloalkyl, halo, hydroxy, alkoxy, mercapto, cyano, sulfonamidyl, aminosulfonyl, acyl, acyloxy, nitro, and substituted amino.

“Heteroarylalkyl” refers to an acyclic alkyl group (including heteroalkyl groups, substituted or not substituted) in which one of the hydrogen atoms bonded to a carbon atom, such as a terminal or sp³ carbon atom, is replaced with an aryl or a heteroaryl group. The “heteroarylalkyl” can encompass any combination of “aryl”, “heteroaryl,” “alkyl” and “heteroalkyl,” such as heteroarylalkyl, heteroalkylaryl, heteroarylheteroalkyl, and the like. A “heteroarylalkyl” can be substituted or not substituted. The nomenclature heteroarylalkanyl, heteroarylakenyl and/or heterorylalkynyl are used when specific alkyl moieties are intended. In certain embodiments, the heteroarylalkyl group is a 5-20 membered heteroarylalkyl, e.g., the alkanyl, alkenyl or alkynyl moiety of the heteroarylalkyl is 1-6 membered and the heteroaryl moiety is a 5-14-membered heteroaryl. In further embodiments, the heteroarylalkyl is a 6-13 membered heteroarylalkyl, e.g., the alkanyl, alkenyl or alkynyl moiety is 1-3 membered and the heteroaryl moiety is a 5-10 membered heteroaryl.

The various heteroaryls, such as indole-, naphthalene-, pyridine-, thiophene- and furan-groups, can include the various position isomers when in the form of a heteroarylalkyl. For example, if the alkyl includes a carbonyl group, the heteroarylalkyls can be indole-3-carbonyl, indole-5-carbonyl, naphthalene-1-carbonyl, naphthalene-2-carbonyl, nicotinoyl, isonicotinoyl, N-methyl-dihydro-pyridine-3-carbonyl, thiophene-2-carbonyl, thiophene-3-carbonyl, furan-2-carbonyl and furan-3-carbonyl. The indole, naphthalene, pyridine, thiophene and furan groups can be optionally further substituted, as indicated herein.

“Acyl” group refers to the C(═O)—R″ group, where R″ is selected preferably from hydrogen, hydroxy, alkyl, haloalkyl, cycloalkyl, aryl optionally substituted with one or more alkyl, haloalkyl, alkoxy, halo and substituted amino groups, heteroaryl (bonded through a ring carbon) optionally substituted with one or more alkyl, haloalkyl, alkoxy, halo and substituted amino groups and heteroalicyclic (bonded through a ring carbon) optionally substituted with one or more alkyl, haloalkyl, alkoxy, halo and substituted amino groups. Acyl groups include aldehydes, ketones, acids, acid halides, esters and amides. Preferred acyl groups are carboxy groups, e.g., acids and esters. Esters include amino acid ester derivatives. The acyl group may be attached to a compound's backbone at either end of the acyl group, i.e., via the C or the R″.

Where the acyl group is attached via the R″, then C will bear another substituent, such as hydrogen, alkyl, and the like.

“Halogen” or “halo” refers to fluoro (F), chloro (Cl), bromo (Br), iodo (I). As used herein, —X refers to independently any halogen.

Sulphur (S) atom may be present in several compounds of this disclosure, and when present, the S atom can be at any oxidation state (e.g., S, SO, SO₂).

As used herein, “ionizable nitrogen” refers to a nitrogen containing substiuent wherein the nitrogen is capable of taking on a positive charge within a pH range of about 4 to about 9.

As used herein, “amino acid” refers to a natural (those occurring in nature) amino acid, a substituted natural amino acid, a non-natural amino acid, a substituted non-natural amino acid, or any combination thereof. The designations for natural amino acids are herein set forth as either the standard one- or three-letter code. Natural polar amino acids include asparagine (Asp or N) and glutamine (Gln or Q); as well as basic amino acids such as arginine (Arg or R), lysine (Lys or K), histidine (His or H), and derivatives thereof; and acidic amino acids such as aspartic acid (Asp or D) and glutamic acid (Glu or E), and derivatives thereof. Natural hydrophobic amino acids include tryptophan (Trp or W), phenylalanine (Phe or F), isoleucine (Ile or I), leucine (Leu or L), methionine (Met or M), valine (Val or V), and derivatives thereof; as well as other non-polar amino acids such as glycine (Gly or G), alanine (Ala or A), proline (Pro or P), and derivatives thereof. Natural amino acids of intermediate polarity include serine (Ser or S), threonine (Thr or T), tyrosine (Tyr or Y), cysteine (Cys or C), and derivatives thereof. Unless specified otherwise, any amino acid described herein may be in either the D- or L-configuration.

A capital letter indicates an L-enantiomer amino acid; a small letter indicates a D-enantiomer amino acid.

Other exemplary amino acids include cinnamic acids (such as aminocinnamic acids, amino-trans-cinnamic acids, amino-cis-cinnamic acids, o-amino-cinnamic acids, m-amino-cinnamic acids, p-amino-cinnamic acids, o-amino-trans-cinnamic acid, m-amino-trans-cinnamic acid, p-amino-trans-cinnamic acid, o-amino-cis-cinnamic acid, m-amino-cis-cinnamic acid, p-amino-cis-cinnamic acid), phenylglycine (Phg), 2,3-diaminobutyric acid (Dab), 2,4-diaminobutyric acid (gDab), 2,3-diaminopropionic acid (Dap), β-methylaspartate (MeAsp), cyclohexylalanine (β-Cha), norleucine (Nle), norvaline (Nvl), isonipecotic acid (Ina), pipecolic acid (homoproline) (Pip or hPro), phenylacetic acids (such as aminophenylacetic acids, diaminophenylacetic acids, triaminophenylacetic acids, o-amino-phenylacetic acid, m-amino-phenylacetic acid, p-amino-phenylacetic acid (Apa), o,o-diamino-phenylacetic acid, o,m-diamino-phenylacetic acid, o,p-diamino-phenylacetic acid, m,m-diamino-phenylacetic acid, m,p-diamino-phenylacetic acid, o,o,m-triamino-phenylacetic acid, o,o,p-triamino-phenylacetic acid, o,m,p-triamino-phenylacetic acid, m,m,p-triamino-phenylacetic acid, o,m,m-triamino-phenylacetic acid, o,o,m-triamino-phenylacetic acid), phenylpropanoic acids (such as aminophenylpropanoic acids, diaminophenylpropanoic acids, triaminophenylpropanoic acids, o-amino-phenylpropanoic acid, m-amino-phenylpropanoic acid, p-amino-phenylpropanoic acid, o,o-diamino-phenylpropanoic acid, o,m-diamino-phenylpropanoic acid, o,p-diamino-phenylpropanoic acid, m,m-diamino-phenylpropanoic acid, m,p-diamino-phenylpropanoic acid, o,o,m-triamino-phenylpropanoic acid, o,o,p-triamino-phenylpropanoic acid, o,m,p-triamino-phenylpropanoic acid, m,m,p-triamino-phenylpropanoic acid, o,m,m-triamino-phenylpropanoic acid, o,o,m-triamino-phenylpropanoic acid), 2-aminobutyric acid (Abu), sarcosine (Sar or N-methyl glycine), 6-aminohexanoic acid (Ahx), para-fluoro-Phenylalanine (p-F-Phe), γ-amino-butyric acid (GABA), benzoic acids (such as aminobenzoic acids, diaminobenzoic acids, triaminobenzoic acids, o-amino-benzoic acid, m-amino-benzoic acid, p-aminobenzoic acid (PABA), o,o-diamino-benzoic acid, o,m-diamino-benzoic acid, o,p-diamino-benzoic acid, m,m-diamino-benzoic acid, m,p-diamino-benzoic acid, o,o,m-triamino-benzoic acid, o,o,p-triamino-benzoic acid, o,m,p-triamino-benzoic acid, m,m,p-triamino-benzoic acid, o,m,m-triamino-benzoic acid, o,o,m-triamino-benzoic acid), hydrazinobenzoic acids (such as dihydrazinobenzoic acids, trihydrazinobenzoic acids, o-hydrazino-benzoic acid, m-hydrazino-benzoic acid, p-hydrazino-benzoic acid, o,o-dihydrazino-benzoic acid, o,m-dihydrazino-benzoic acid, o,p-dihydrazino-benzoic acid, m,m-dihydrazino-benzoic acid, m,p-dihydrazino-benzoic acid, o,o,m-trihydrazino-benzoic acid, o,o,p-trihydrazino-benzoic acid, o,m,p-trihydrazino-benzoic acid, m,m,p-trihydrazino-benzoic acid, o,m,m-trihydrazino-benzoic acid, o,o,m-trihydrazino-benzoic acid), homophenylalanine (homoPhe or hPhe), β-cyanoAlanine (β-cyano-Ala), methyl or ethyl aryl ethers of tyrosine (Tyr(Me) or Tyr(Et), respectively), aminoisobutyric acid (Aib, which is also known as α,α-dimethylglycine), S-methylcysteine (MeCys), N,N′-dimethyl-arginine ((Me)₂Arg), hydroxyProline (Hyp), citruline (Cit), N,N,N-trimethyllysine or N,N,N,—(CH₃)₃-lysine or γ,γ,γ-trimethyllysine ((Me)₃Lys), homolysine (homoLys or hLys), 5-aminopentanoic acid or aminovaleric acid (5-Ava), (S)-3-Benzo[b]thiophen-3-yl-aminopropanoic acid (L-BBTA), pyroglutamic acid (pGlu), aminothiazole acetic acids, 2-amino-thiazol-4-yl acetic acid, aminoheptanoic acids, aminooctanoic acids, aminononanoic acids, aminodecanoic acids, aminoundecanoic acids, aminododecanoic acids, 7-aminoheptanoic acid, 8-aminooctanoic acid, 9-aminononanoic acid, 10-aminodecanoic acid, 11-aminoundecanoic acid, 12-aminododecanoic acid, 3- or 4-mercaptoproline derivatives, N⁵-acetyl-NR⁵-hydroxy-L-ornithine, α-NR-hydroxyamino acids, and the like. An antiviral compound disclosed herein may include any one or a combination of the above-noted amino acids or any one or a combination of the above-noted amino acids optionally substituted.

“Substituted” refers to a group in which one or more hydrogen atoms are each independently replaced with the same or different substituent(s). Representative substituents include —X, —R⁶, —O—, ═O, —OR, —SR⁶, —S—, ═S, —NR⁶R⁶, ═NR⁶, CX₃, —CF₃, —CN, —OCN, —SCN, —NO, —NO₂, ═N₂, —N₃, —S(═O)₂O—, —S(═O)₂OH, —S(═O)₂R⁶, —OS(═O)₂O—, —OS(═O)₂OH, —OS(═O)₂R⁶, —P(═O)(O⁻)₂, —P(═O)(OH)(O⁻), —OP(═O)₂(O⁻), —C(—O)R⁶, —C(═S)R⁶, —C(═O)OR⁶, —C(═O)O⁻, —C(═S)OR⁶, —NR⁶—C(═O)—N(R⁶)₂, —NR⁶—C(═S)—N(R⁶)₂, and —C(═NR⁶)NR⁶R⁶, wherein each X is independently a halogen; and each R⁶ is independently hydrogen, halogen, alkyl, aryl, arylalkyl, arylaryl, arylheteroalkyl, heteroaryl, heteroarylalkyl, NR⁷R⁷, —C(═O)R⁷, and —S(═O)₂R⁷; and each R⁷ is independently hydrogen, alkyl, alkanyl, alkynyl, aryl, arylalkyl, arylheteralkyl, arylaryl, heteroaryl or heteroarylalkyl. Aryl containing substituents, whether or not having one or more sustitutions, may be attached in a para (p-), meta (m-) or ortho (o-) conformation, or any combination thereof.

The term “independently” means that a substituent can be the same or different for each item described.

In a further embodiment of formula (I), a carbonyl group can be a bridge between R¹ and the core structure—these compounds have a structure of formula (II): 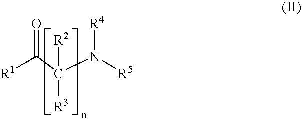
or a stereoisomer, prodrug or pharmaceutically acceptable salt thereof, wherein:

n is 1-5;

R¹ is selected from (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups, provided that R¹ does not form an ester with the carbonyl group to which it is bonded;

(i) R², R³ and R⁴ are each independently the same or different substituent as defined for R⁹; or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, —SO₂R⁹;

R⁹ is selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄ C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl; and wherein at least one but not more than three of R², R³, R⁴ and R⁵is hydrogen, provided that R¹ is not an amino acid when R⁴ and R⁵ are both H.

In another embodiment of formula (I), R¹ is bonded to the core structure via a —NR¹⁰C(═O), and these compounds have a structure of formula (III): 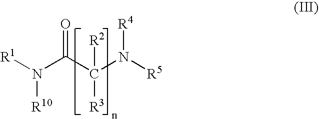
or a stereoisomer, prodrug or pharmaceutically acceptable salt thereof, wherein:

n is 1-5;

R¹ is selected from (C₁-C₁₀) alkyl optionally substituted with one or more of the same or different R groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

(i) R², R³ and R⁴ are each independently the same or different substituent as defined for R⁹; or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, —SO₂R⁹;

R⁹ is selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl; and wherein at least one but not more than three of R², R³, R⁴ and R⁵is hydrogen, provided that R¹ is not an amino acid when R⁴ and R⁵ are both H.

In certain aspects, the instant disclosure provides compounds having a structure of formula (IV): 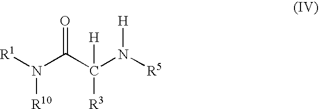
or a stereoisomer, prodrug or pharmaceutically acceptable salt thereof, wherein:

R¹, R³ and R⁹ are each independently selected from H, (C₁-C₁₀) alkyl optionally substituted with one or more of the same or different R groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;

R⁵ is selected from —C(═O)NR⁹, —C(═O)(NR¹⁰)SO₂R⁹, —C(═S)NR¹⁰R⁹, or —C(═NR¹⁰)NR¹⁰R⁹; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl.

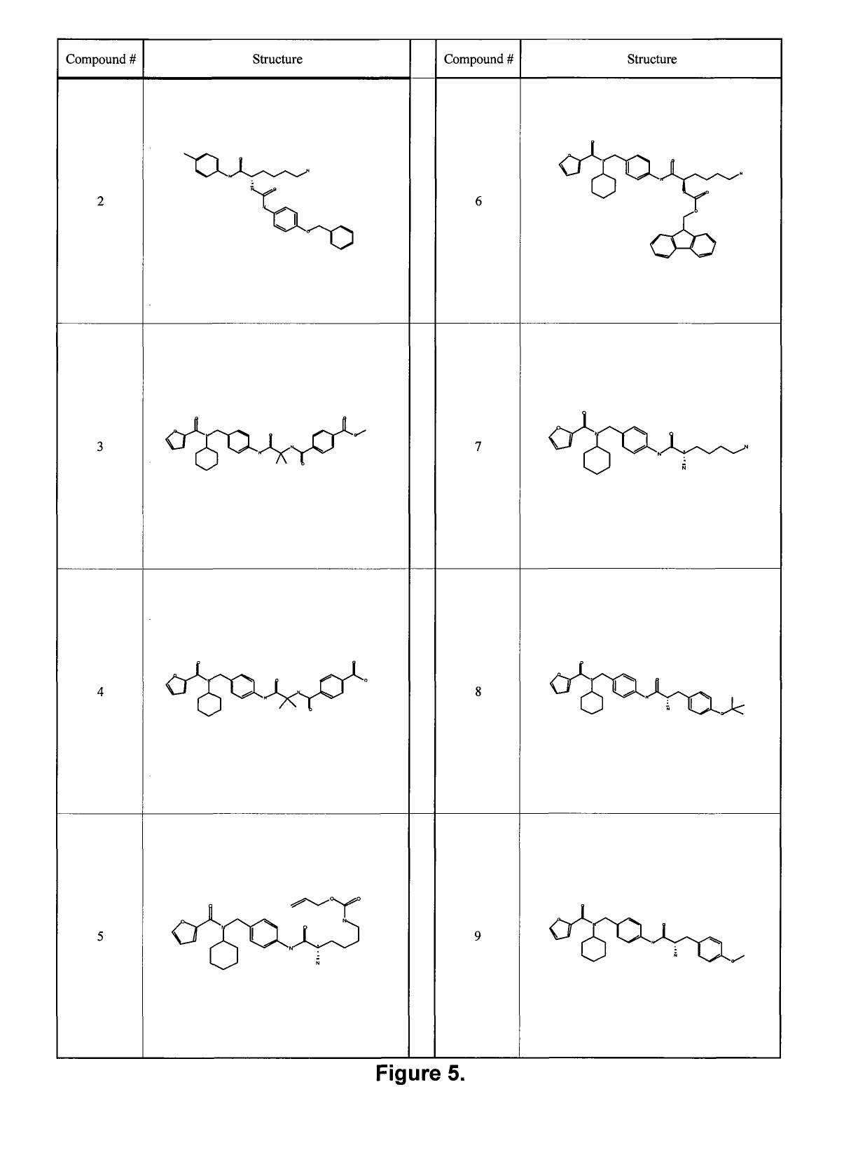

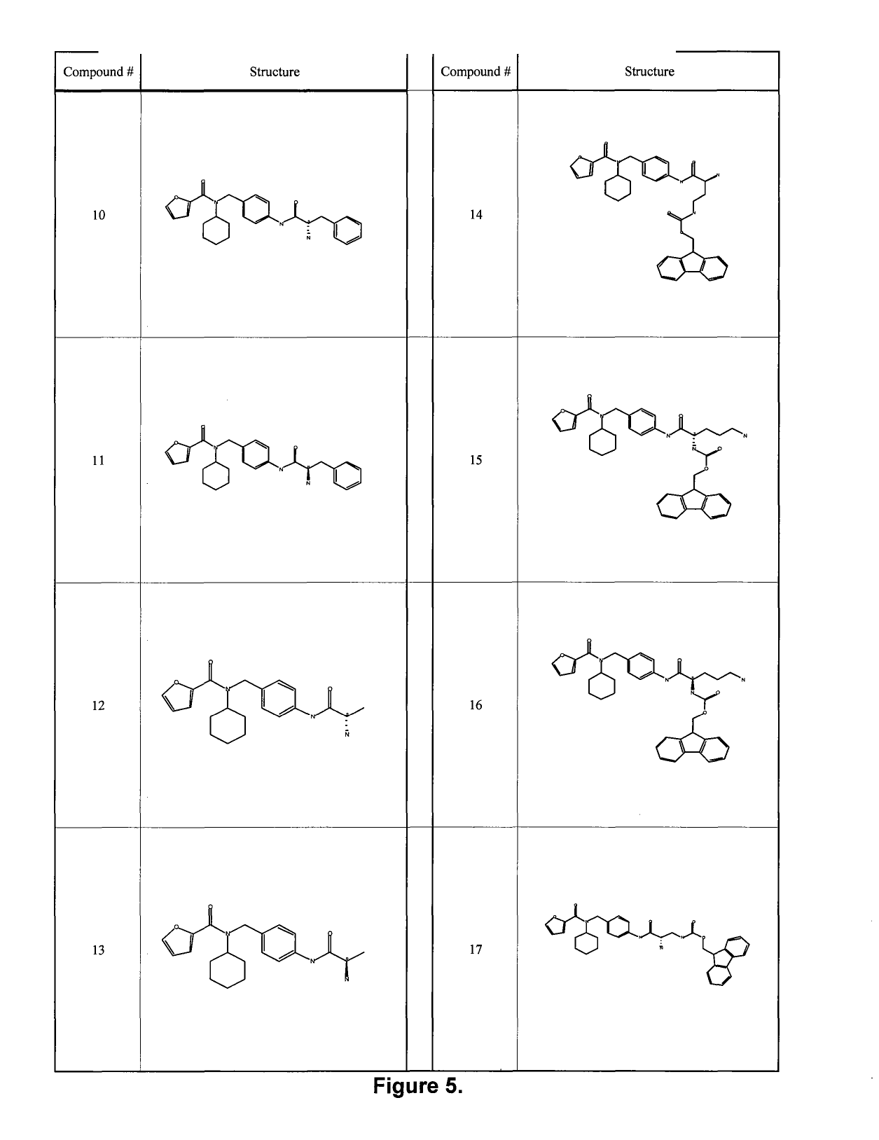

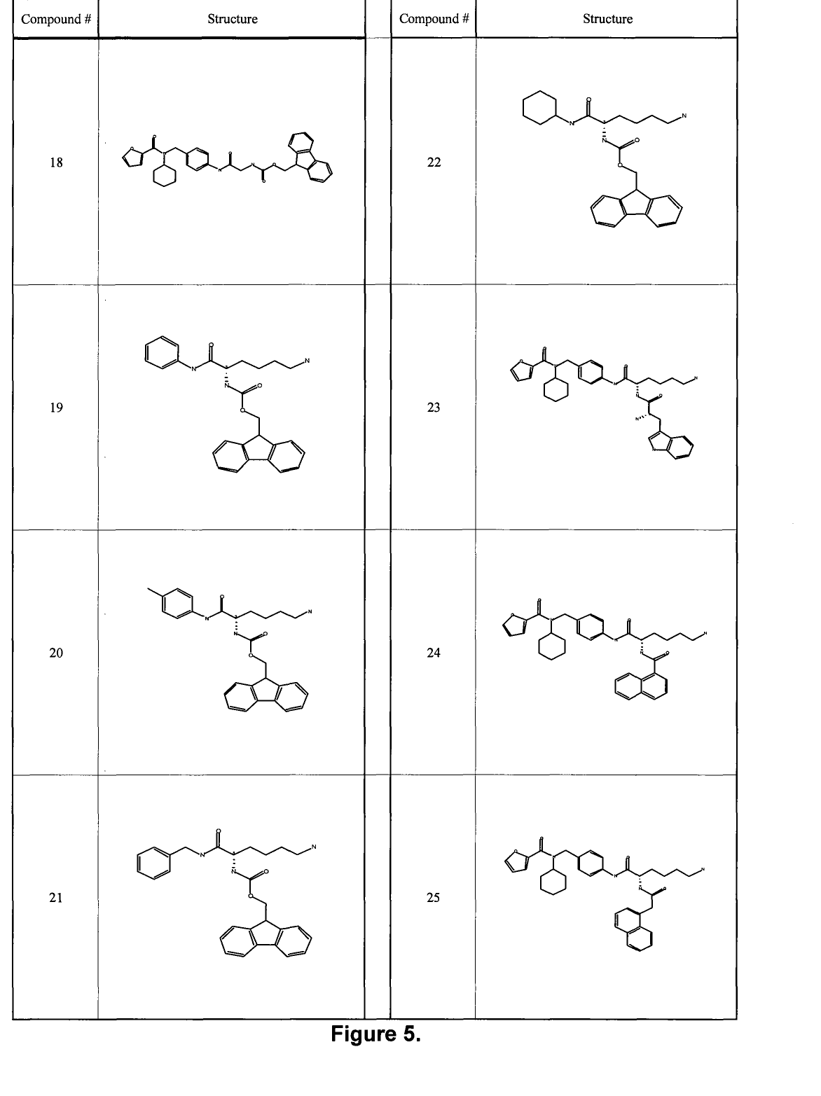

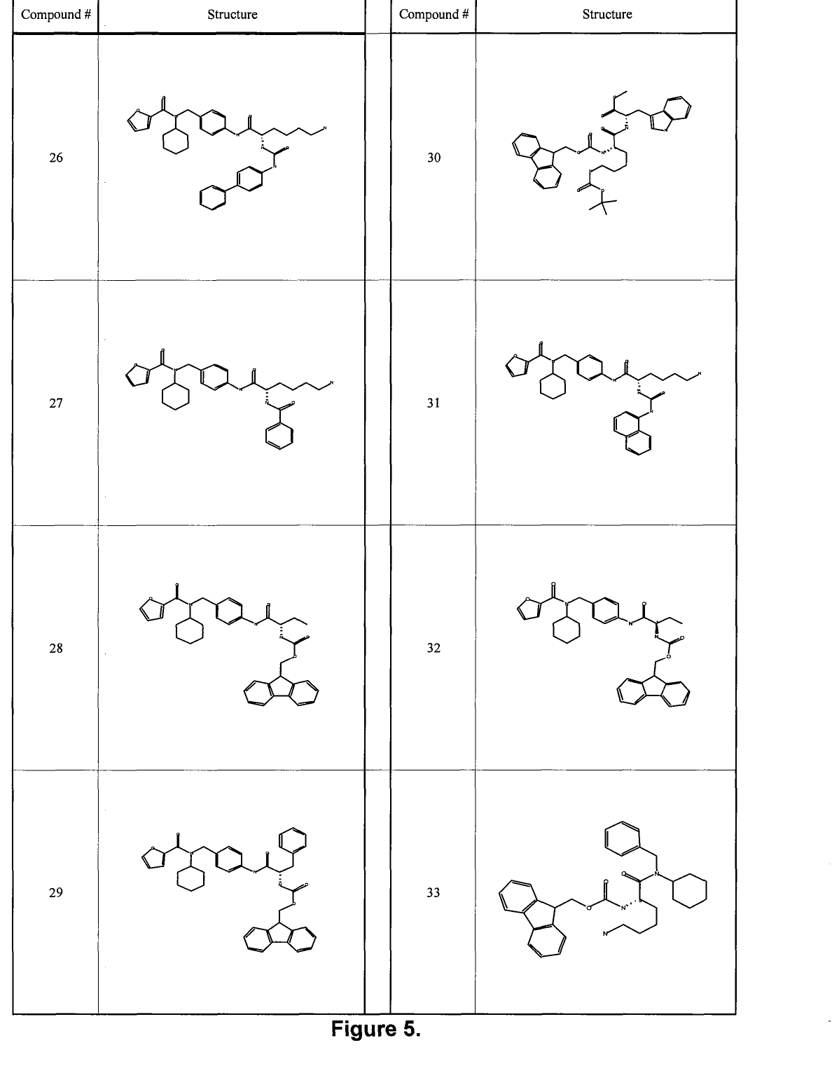

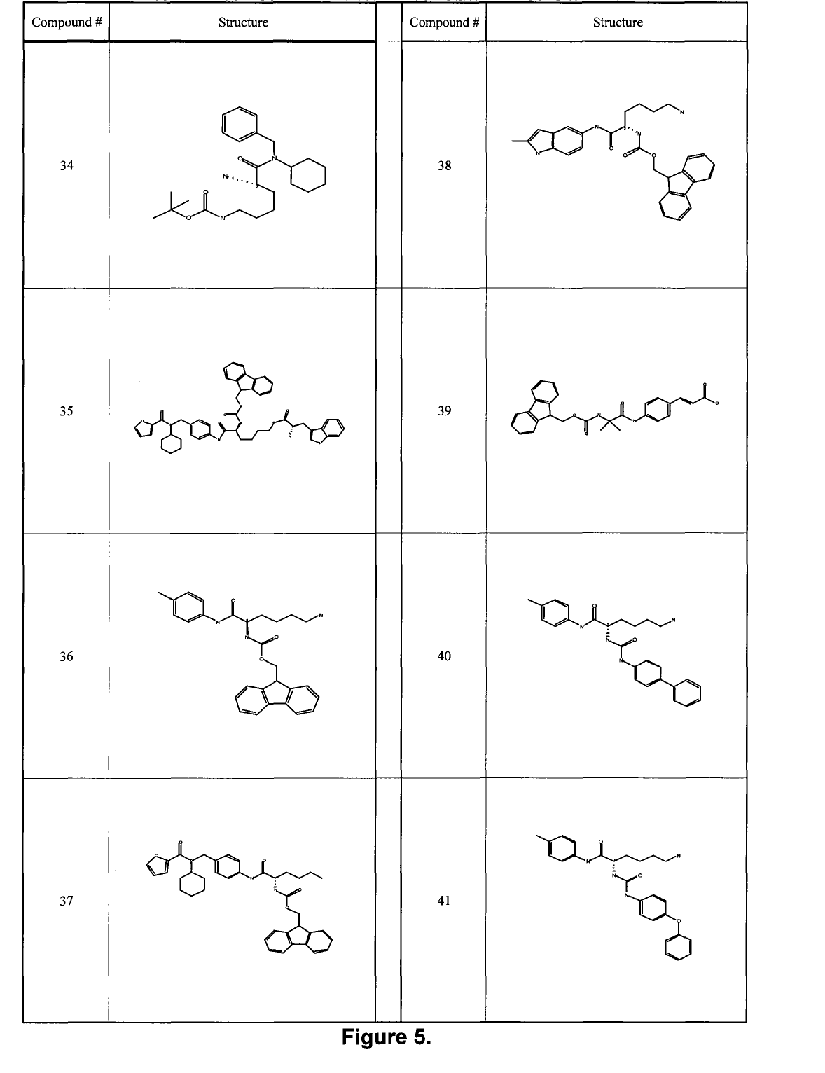

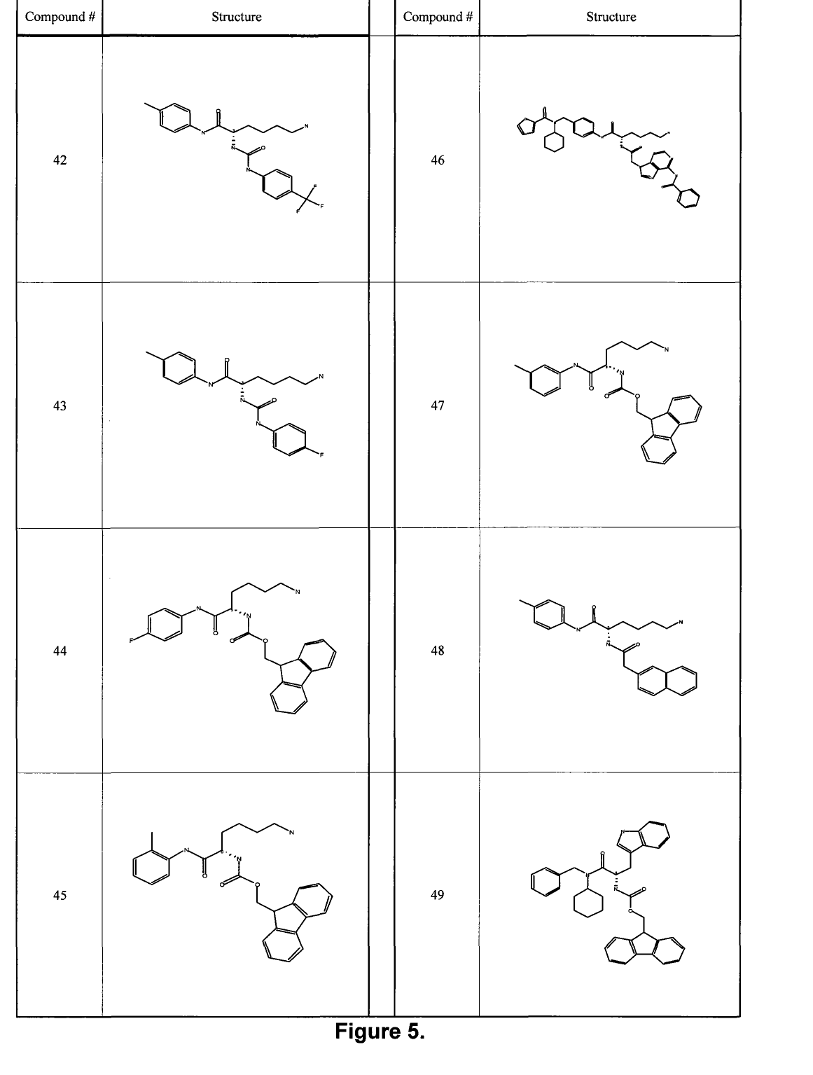

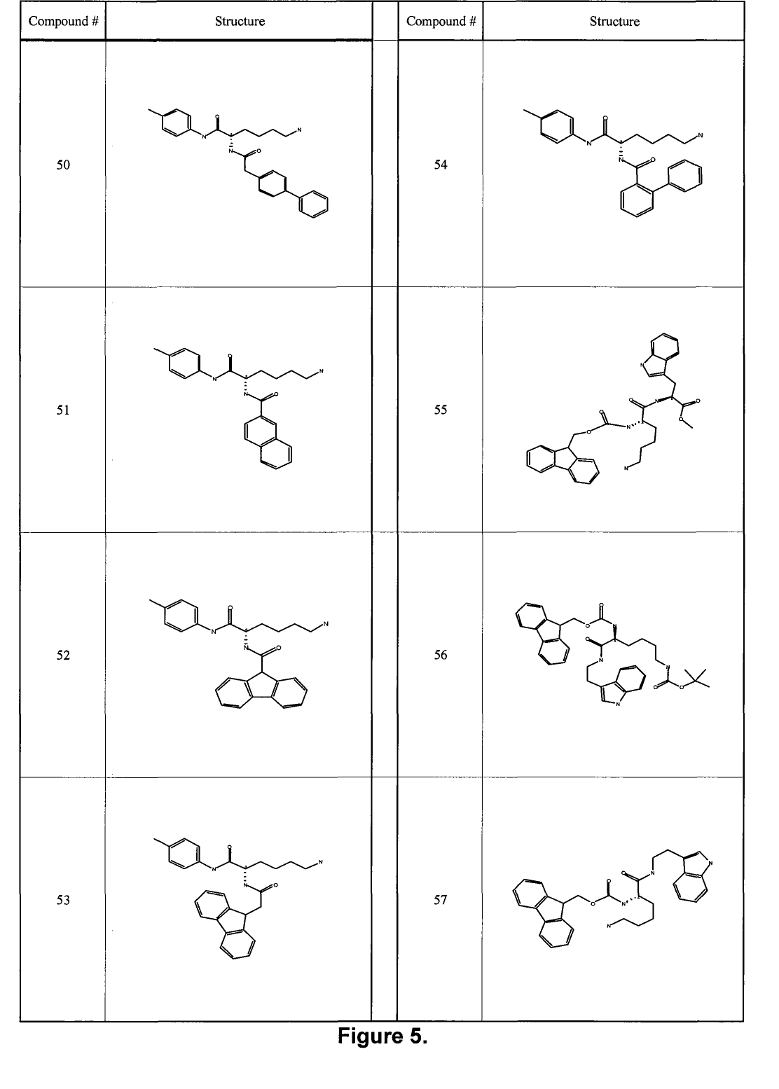

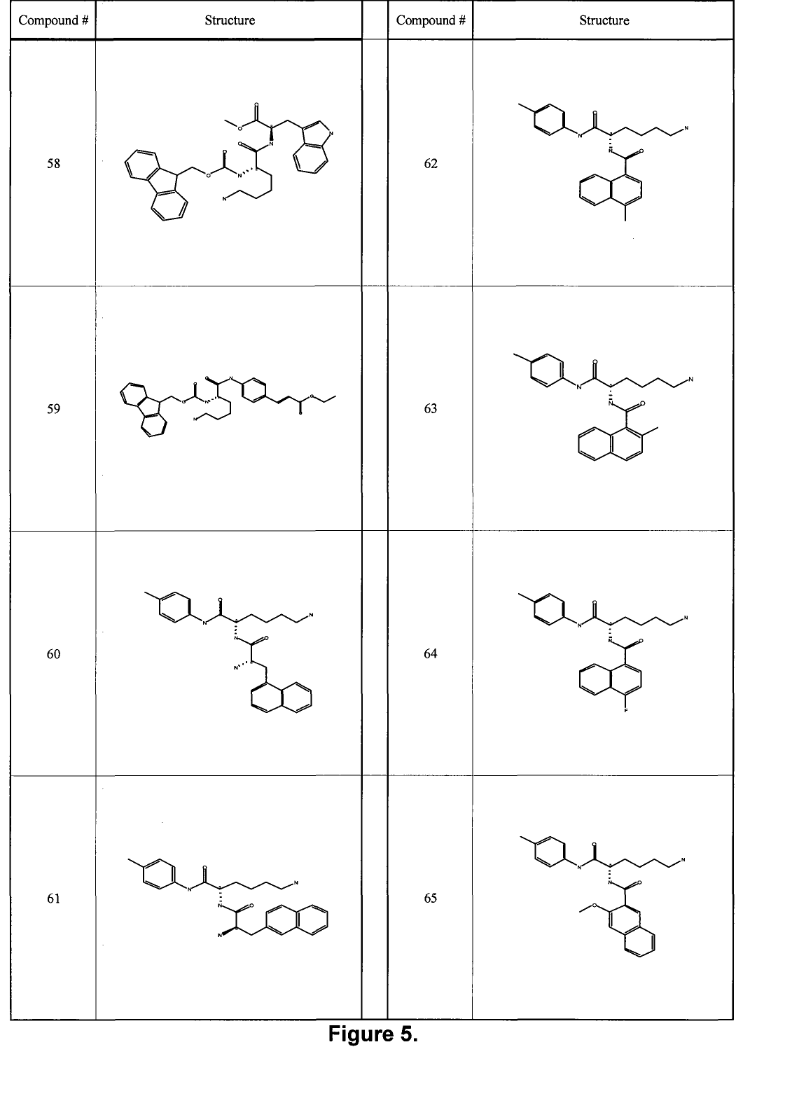

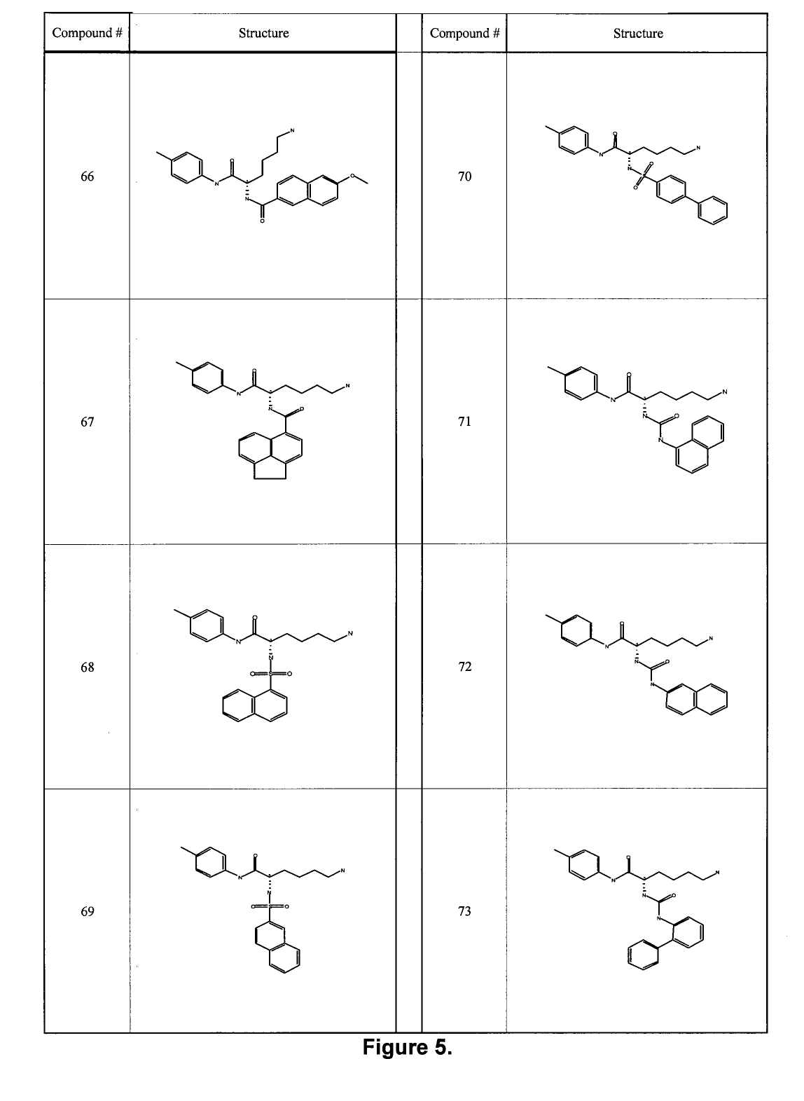

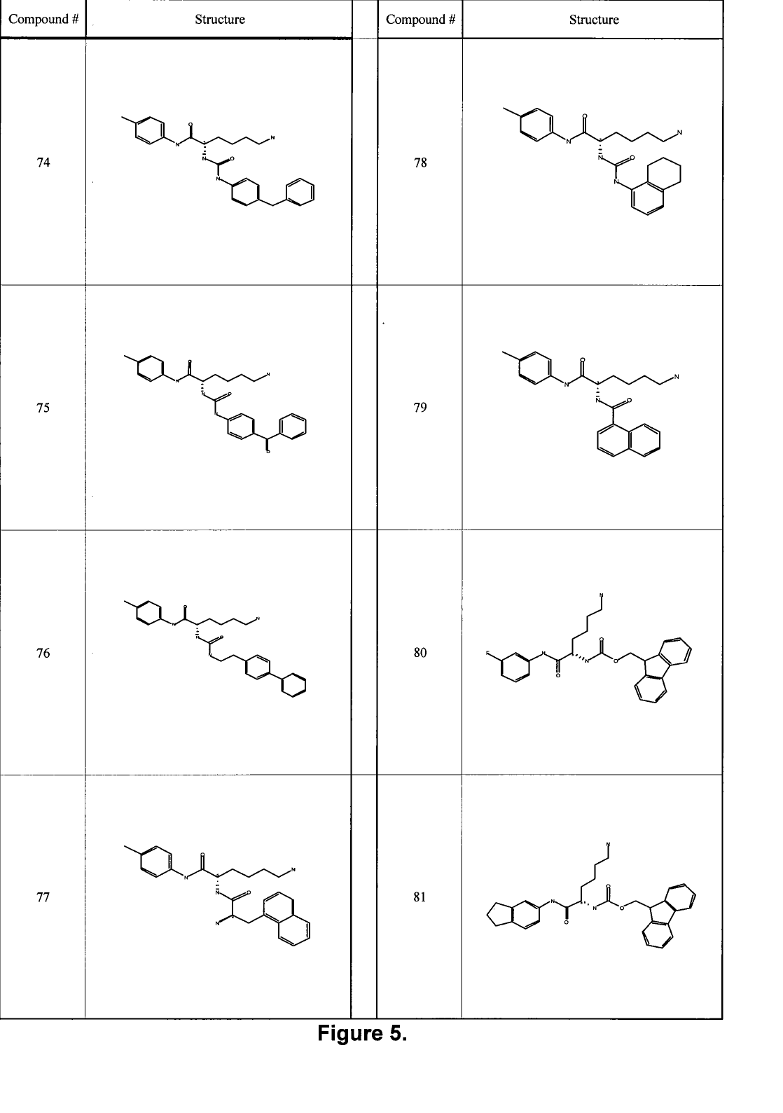

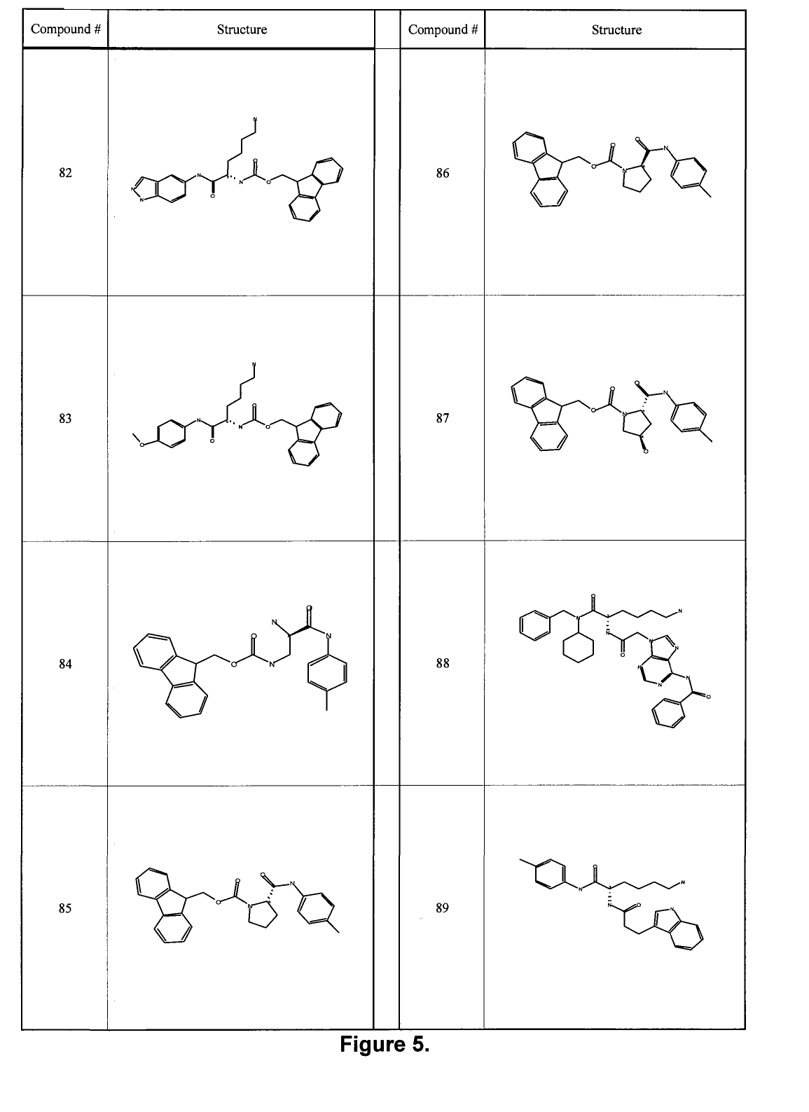

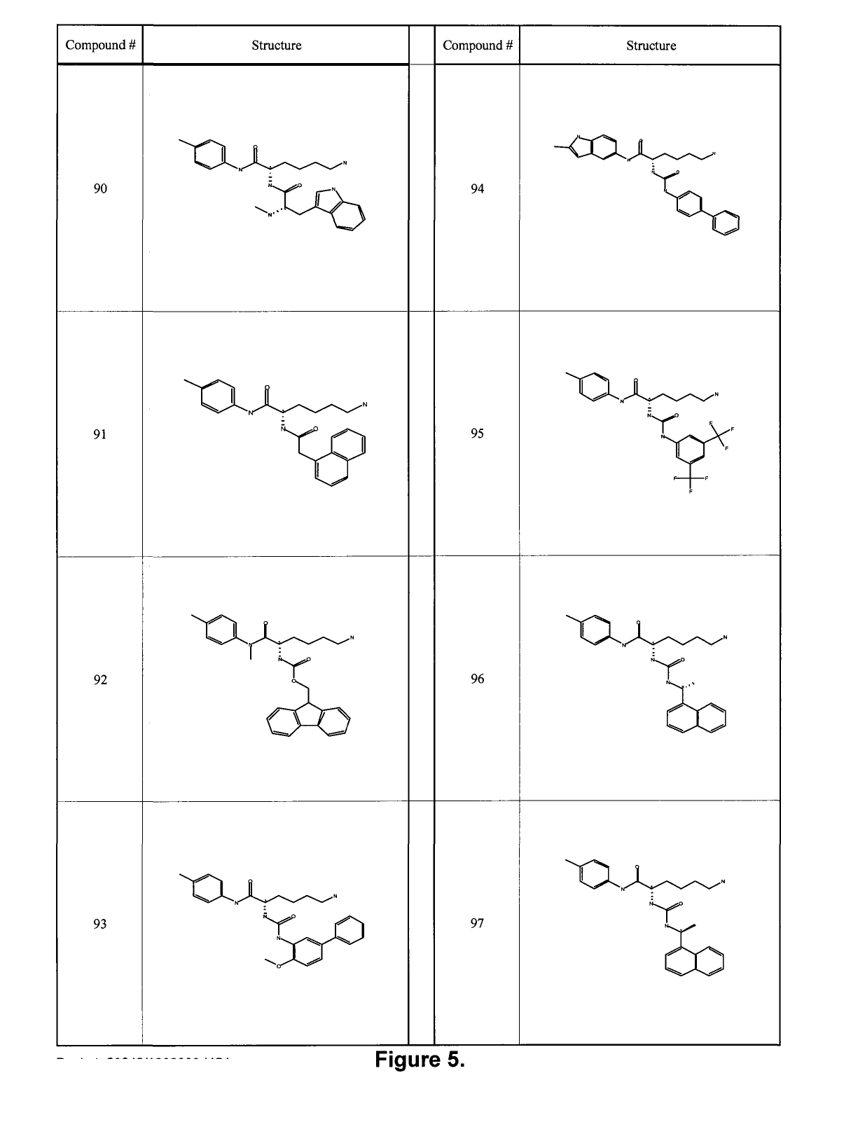

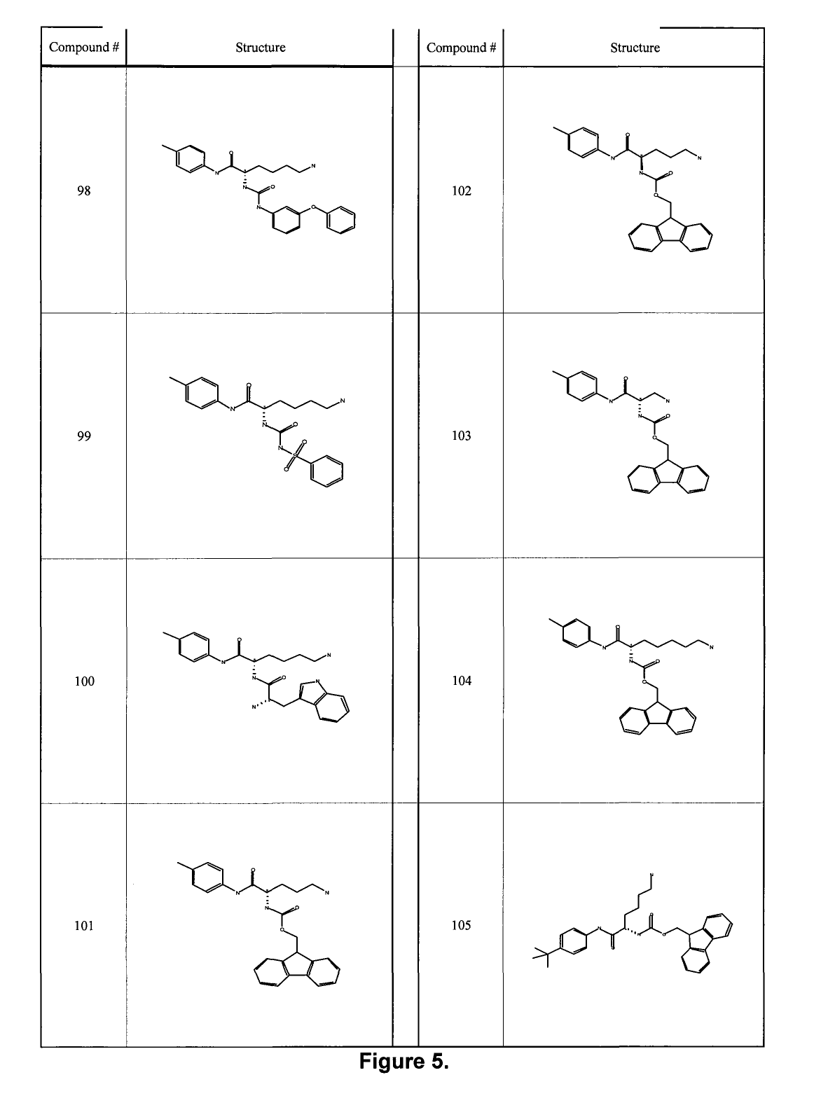

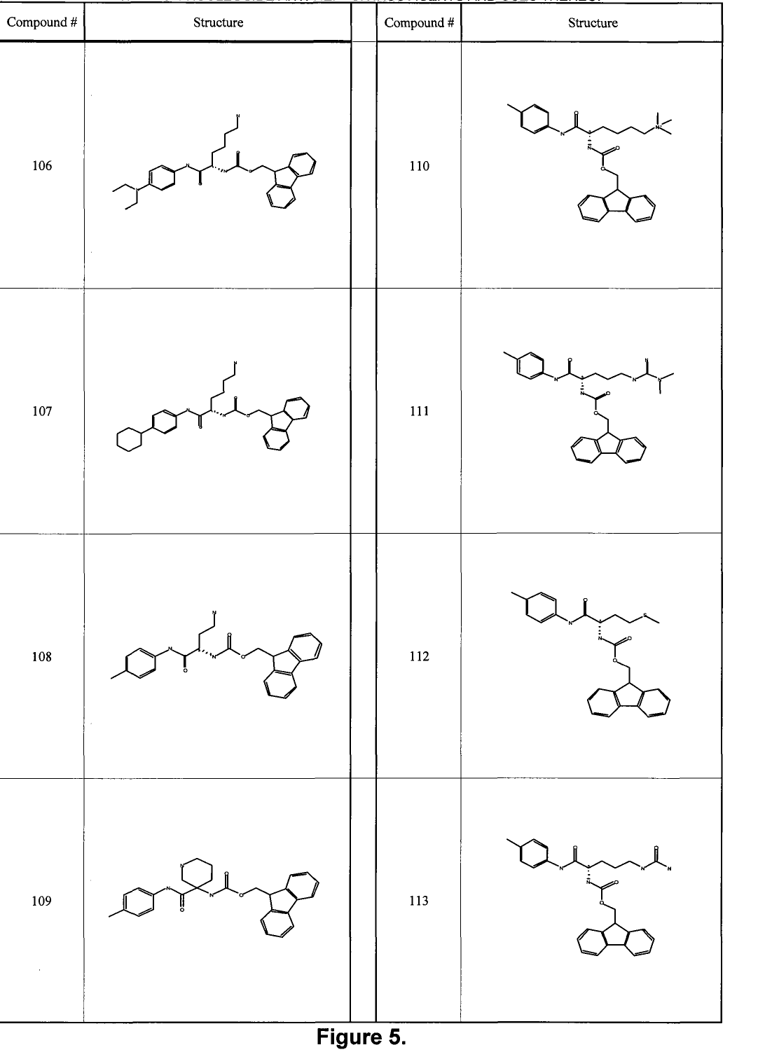

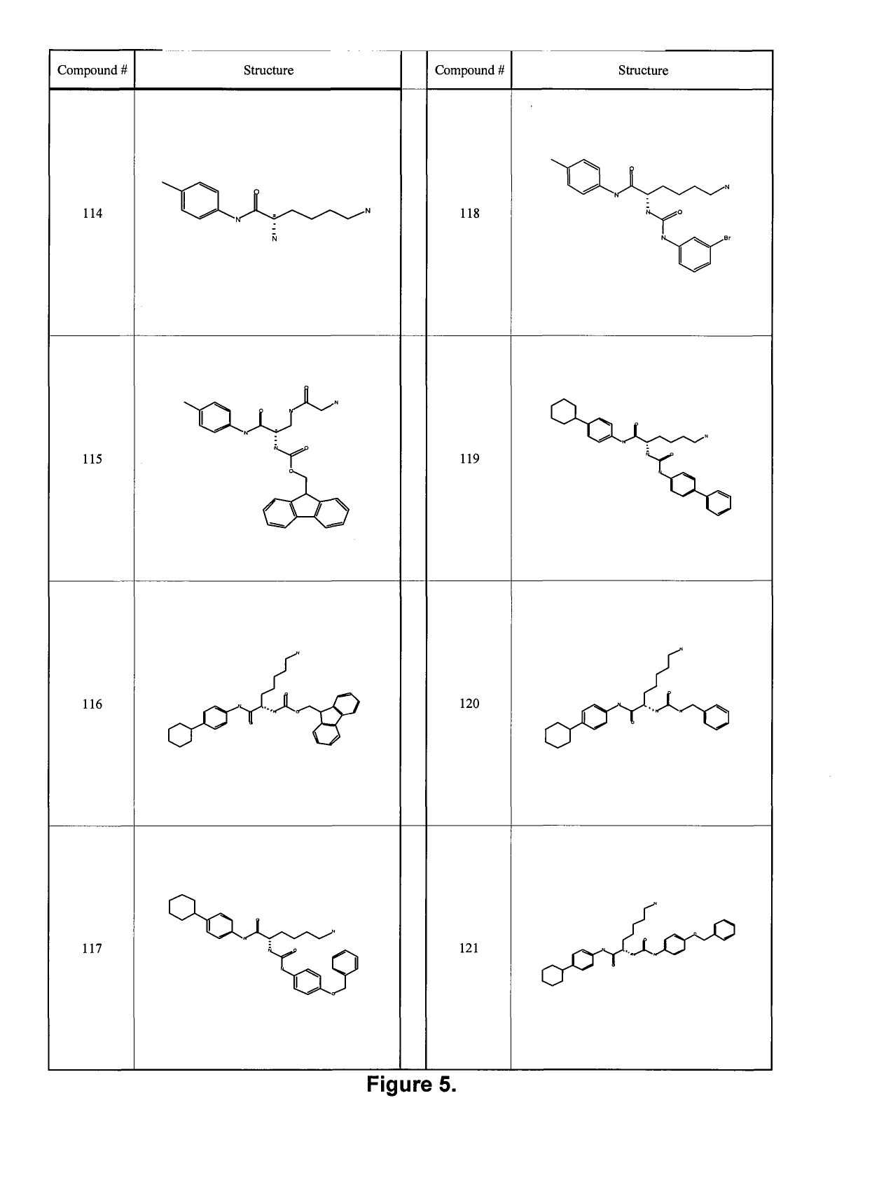

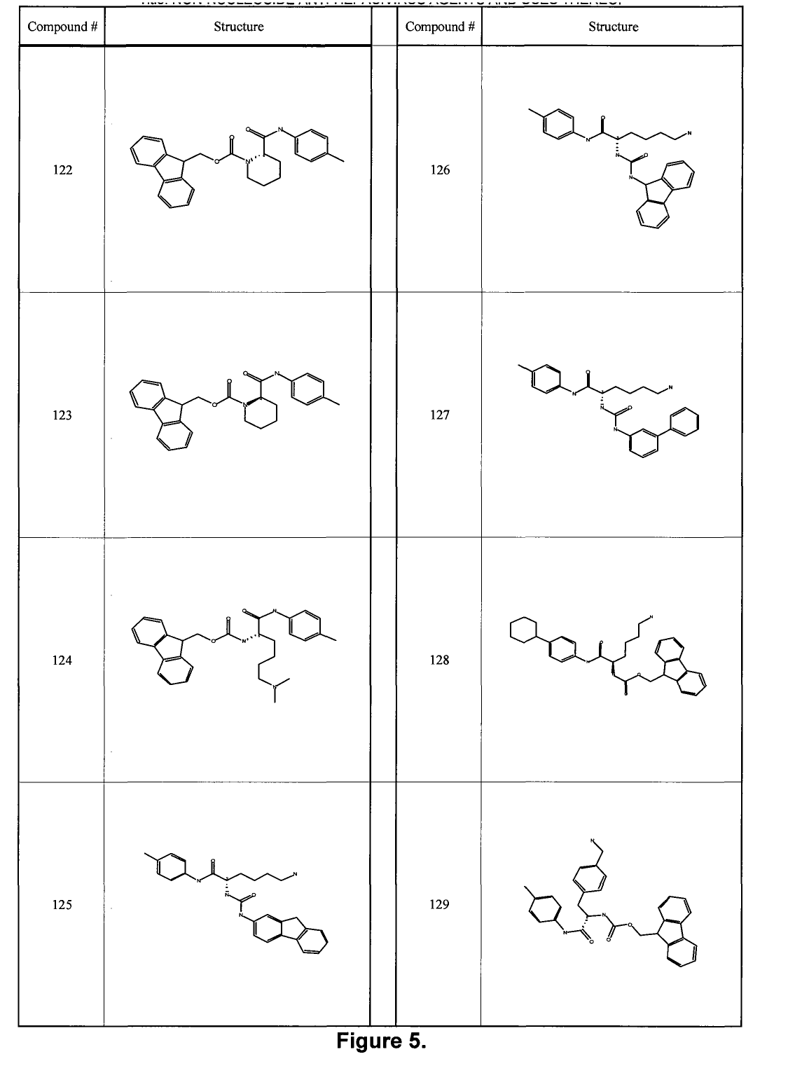

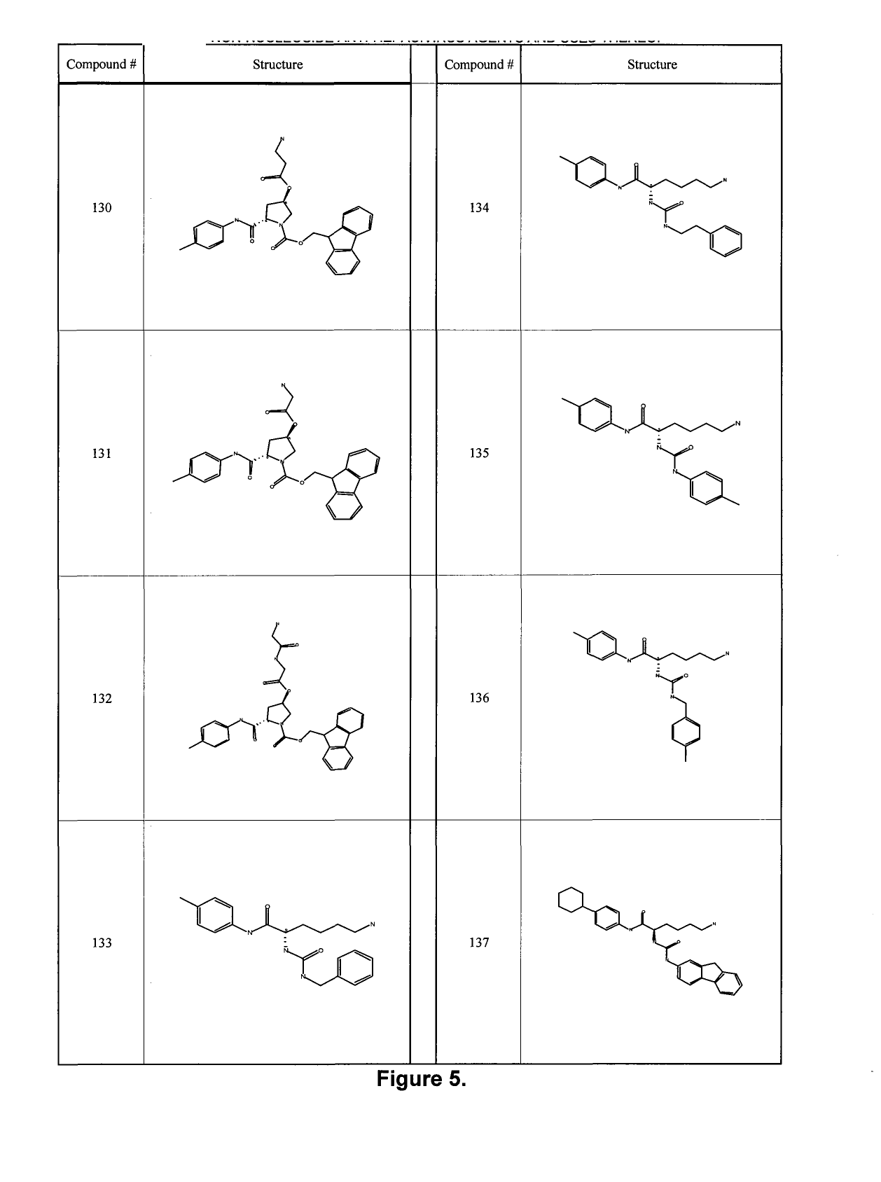

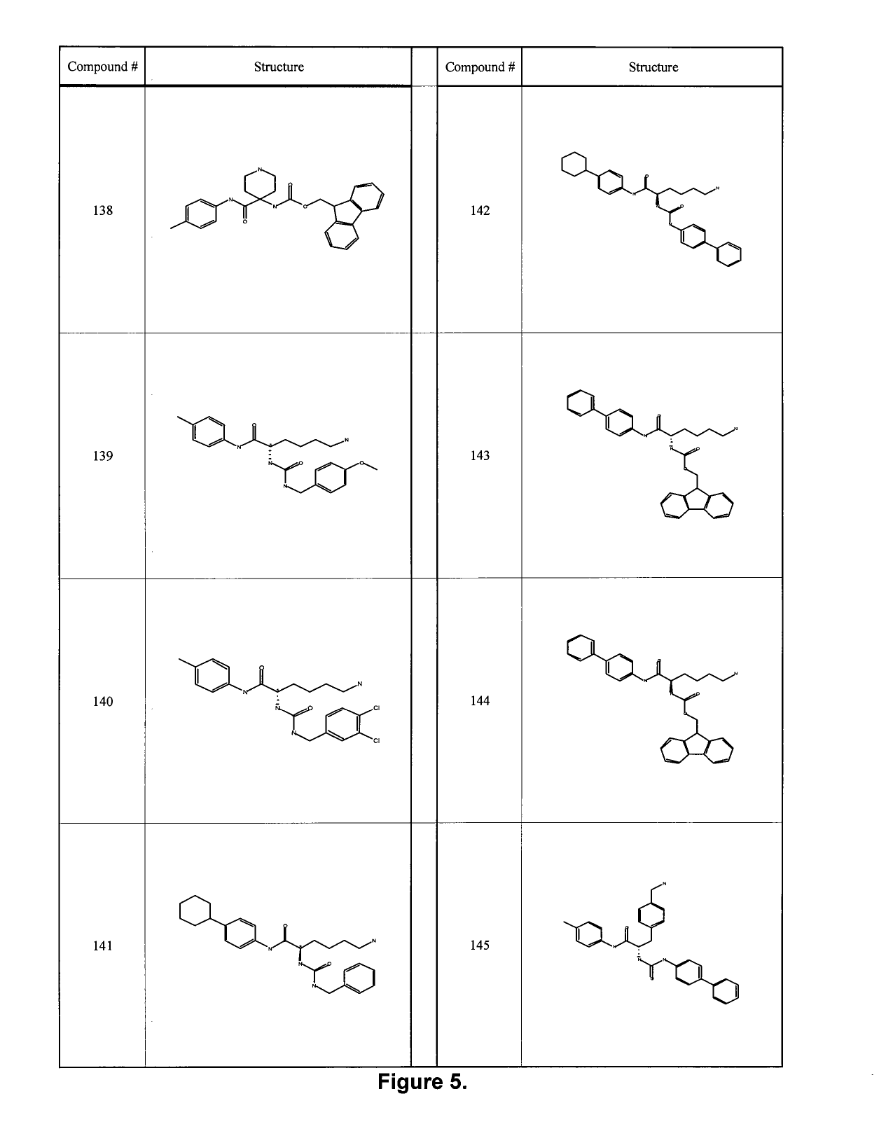

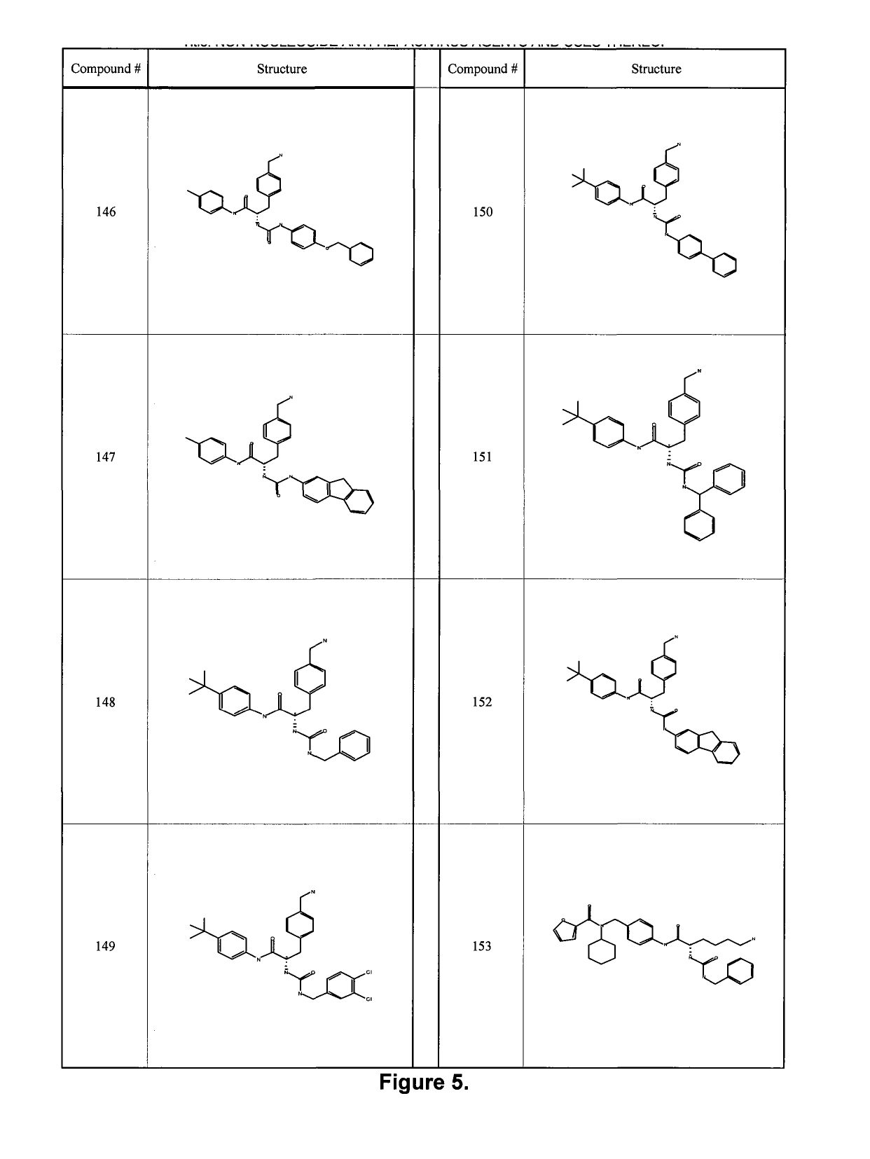

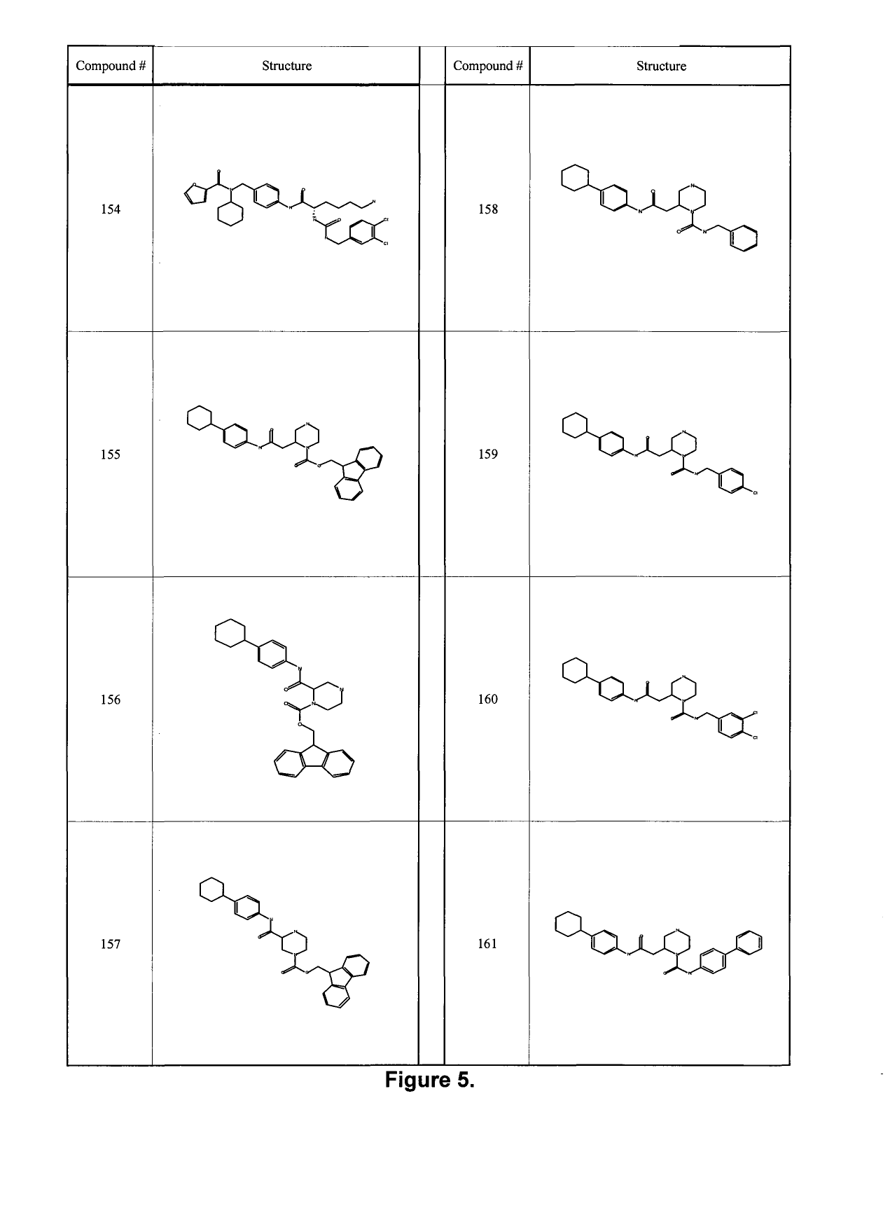

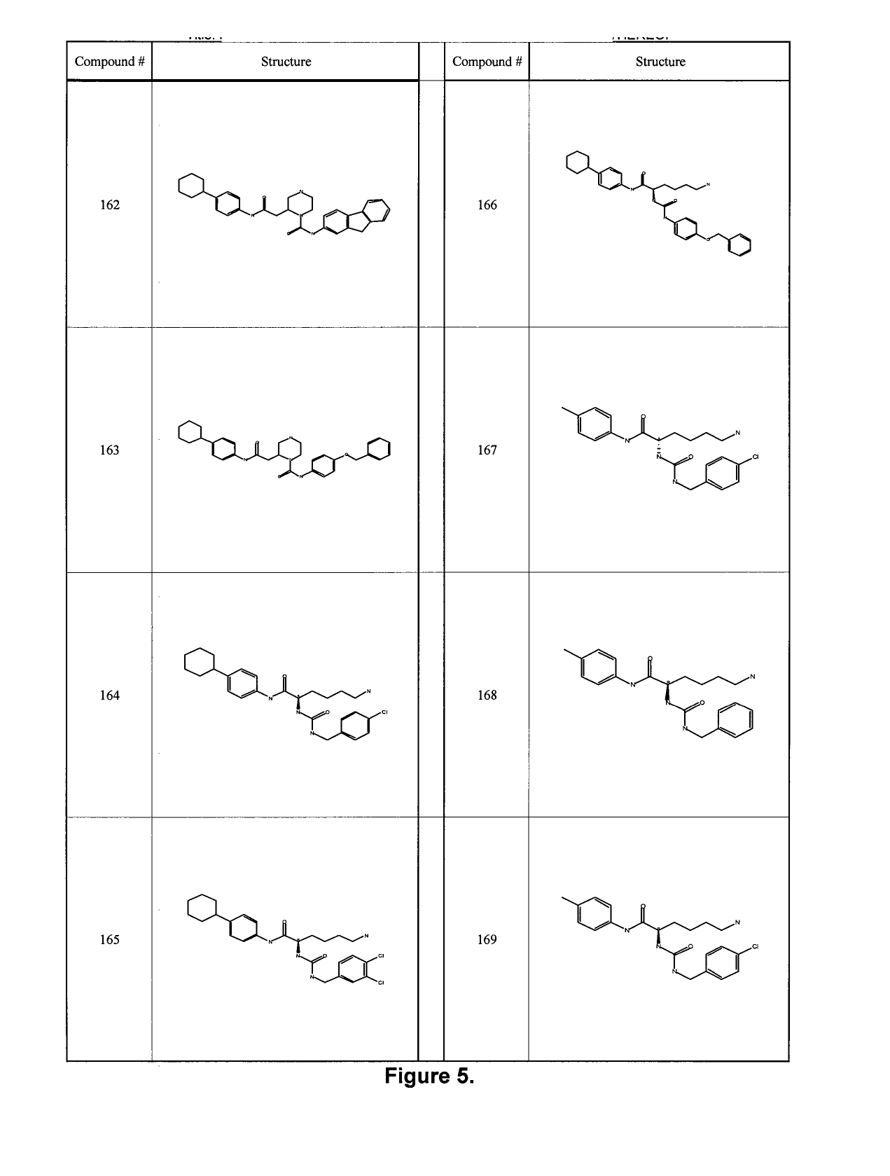

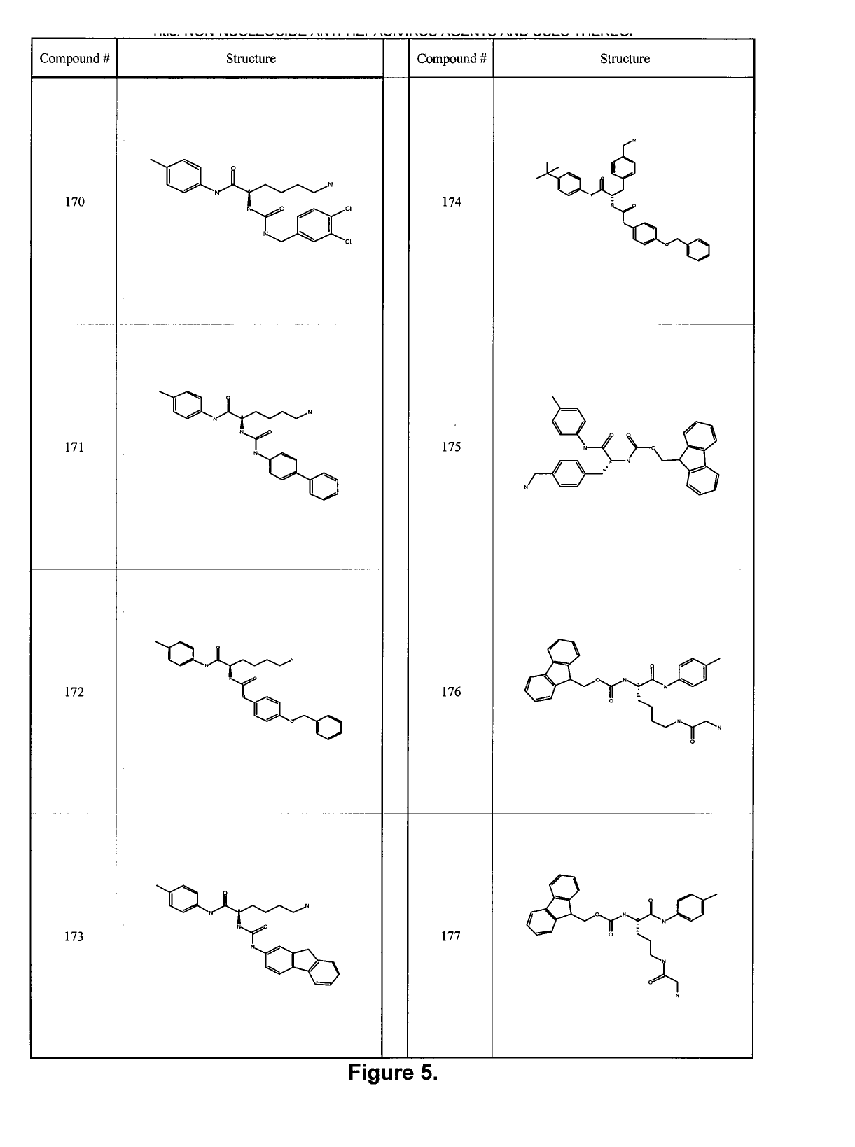

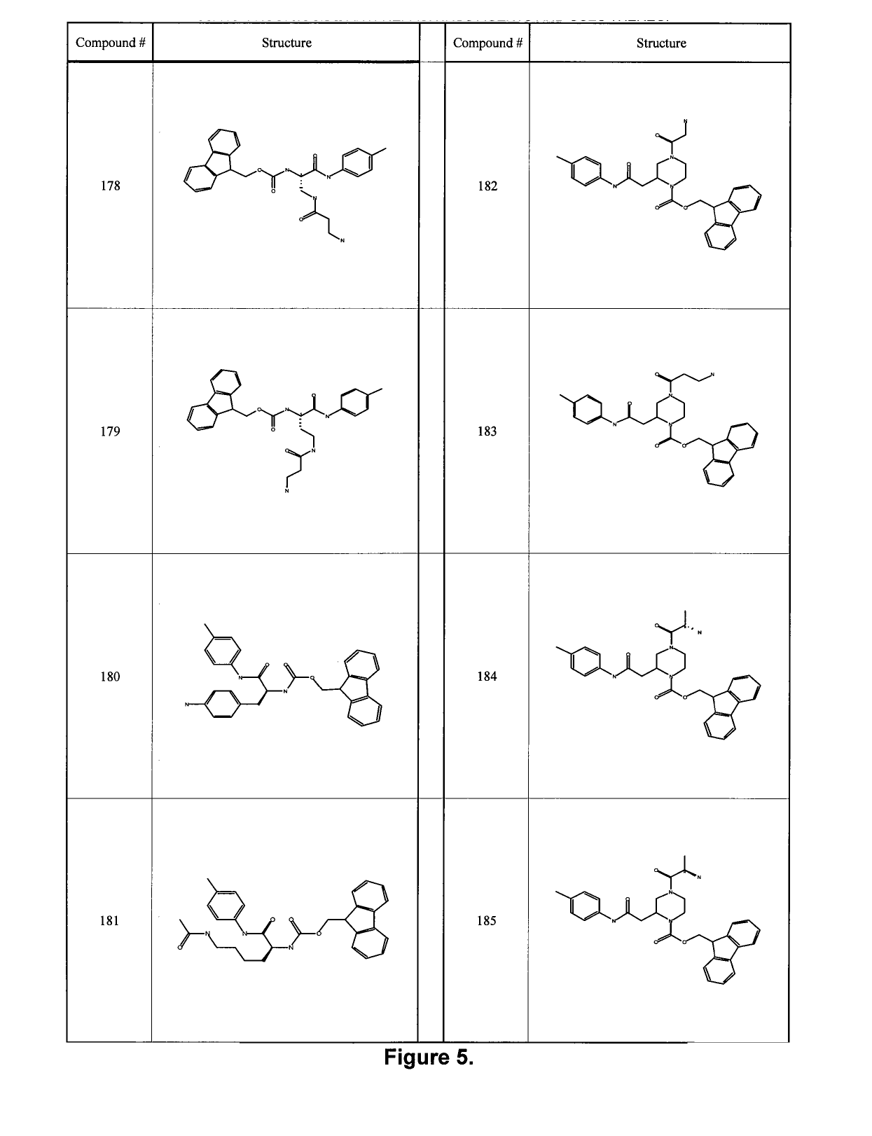

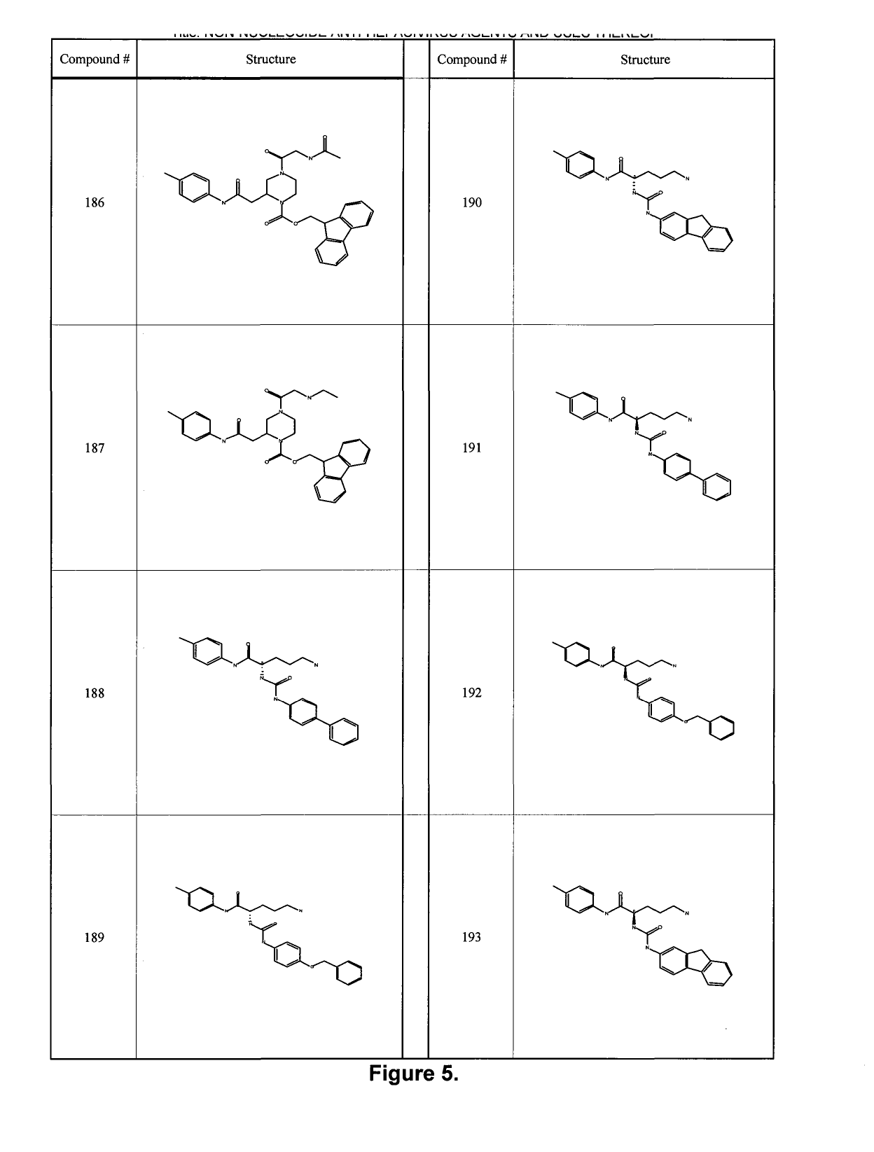

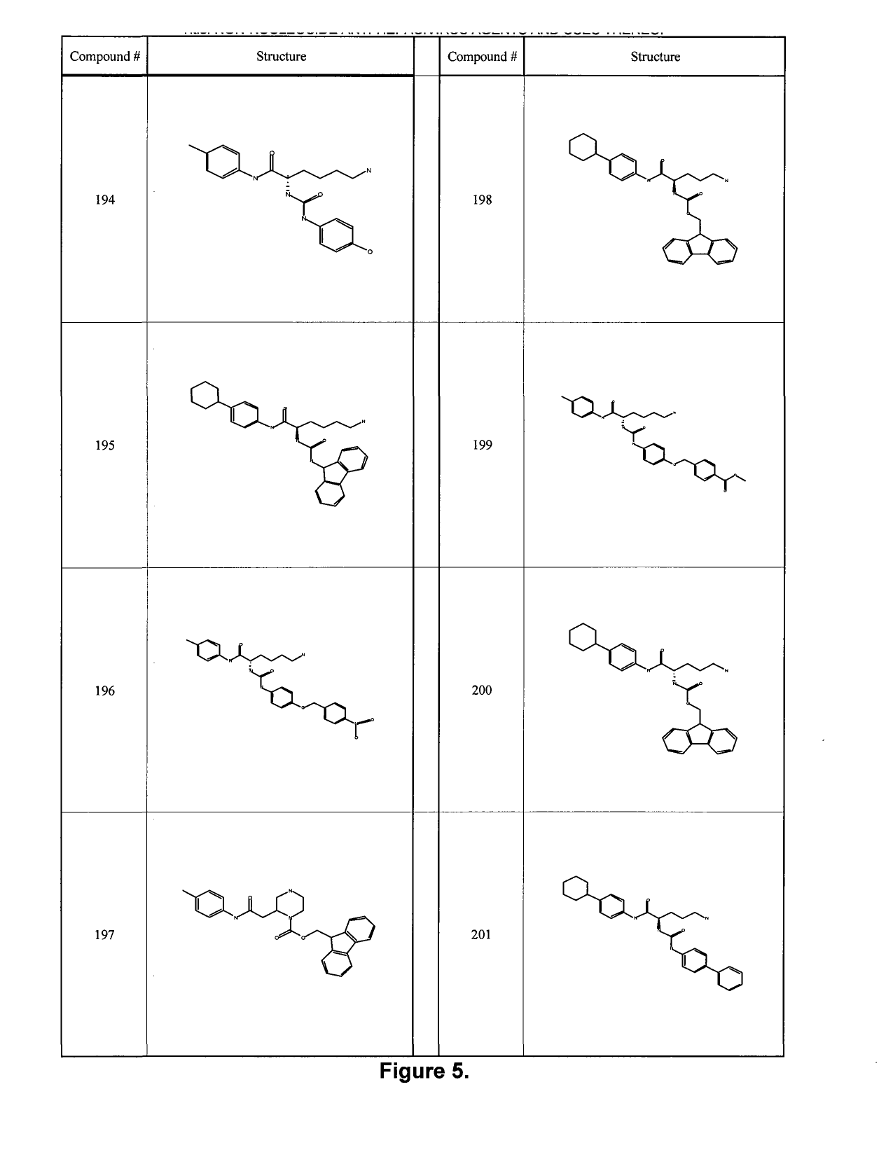

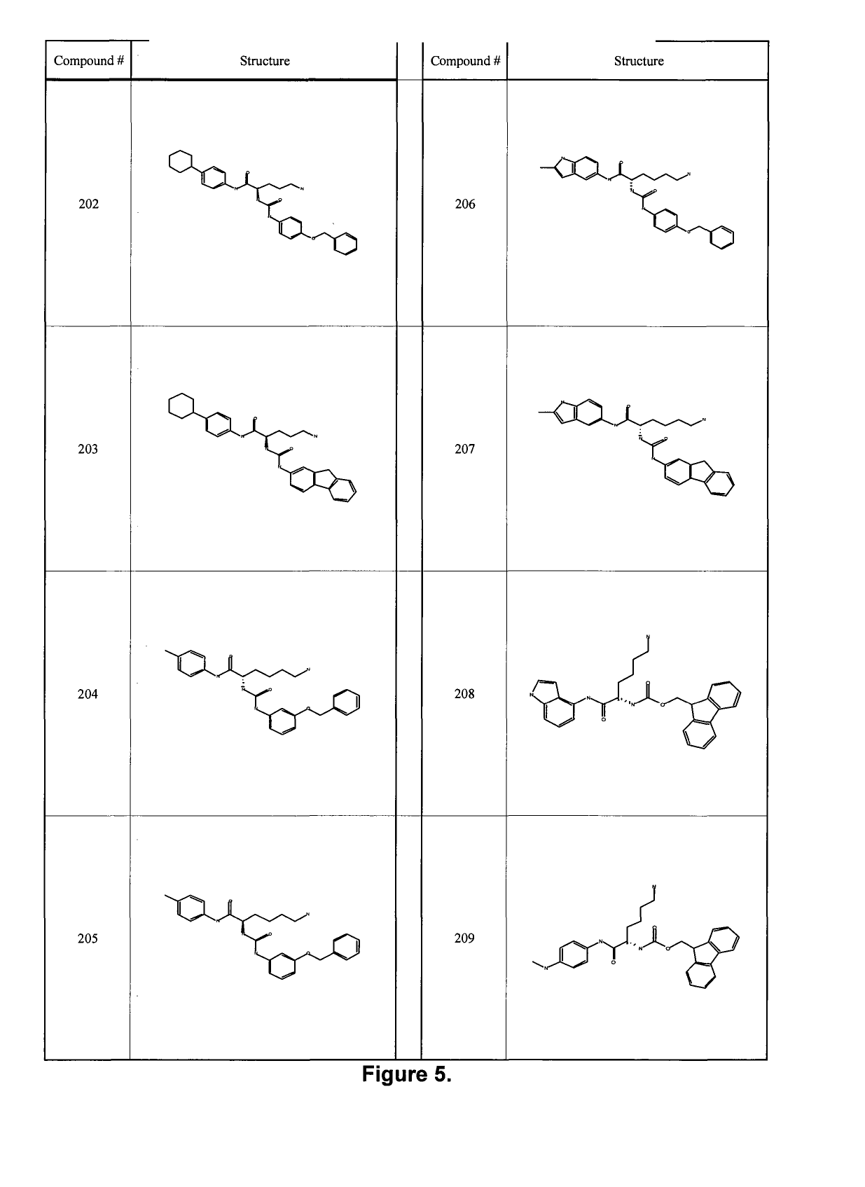

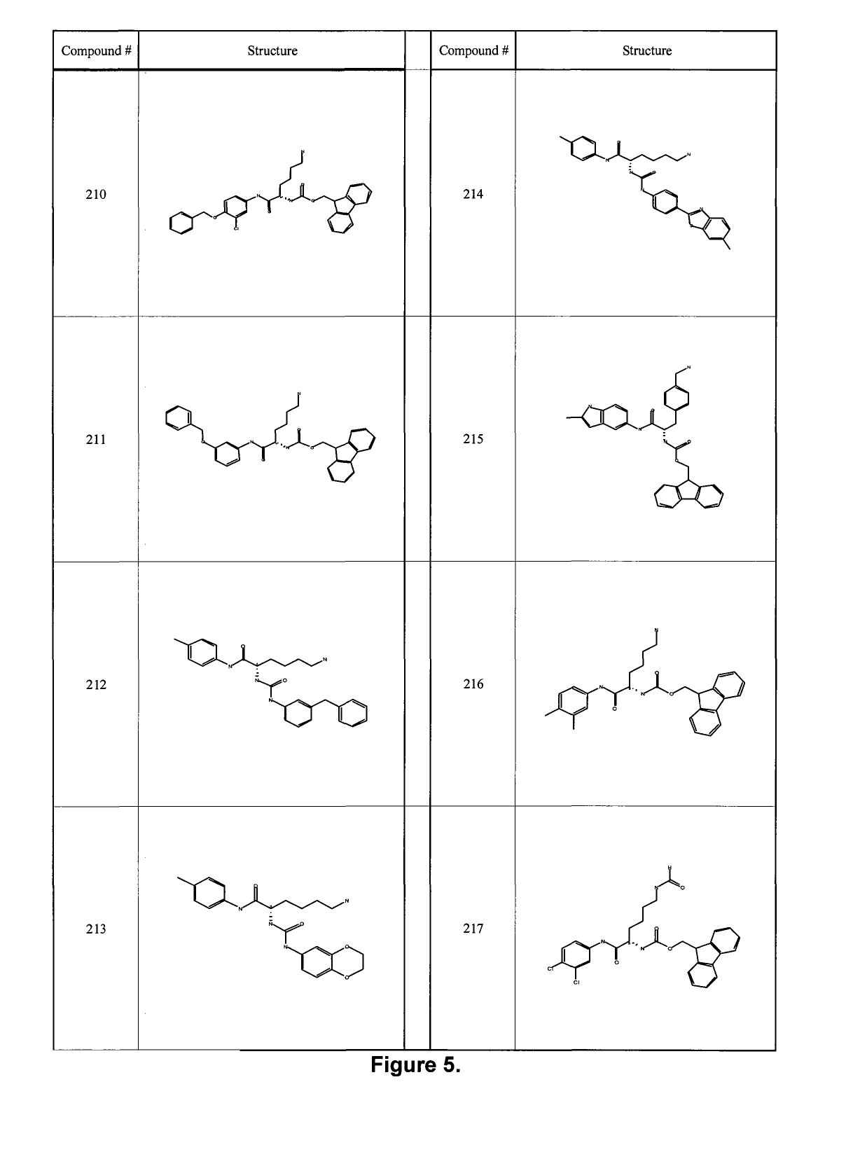

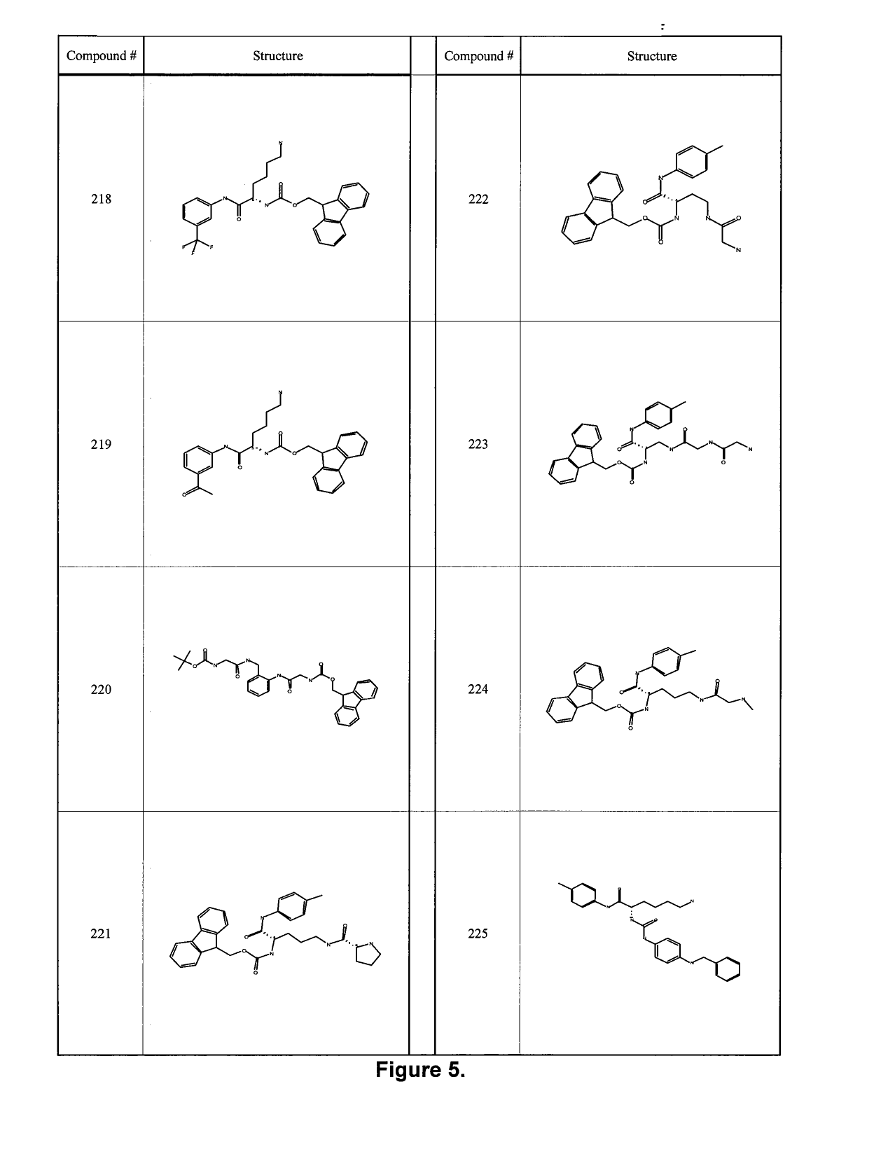

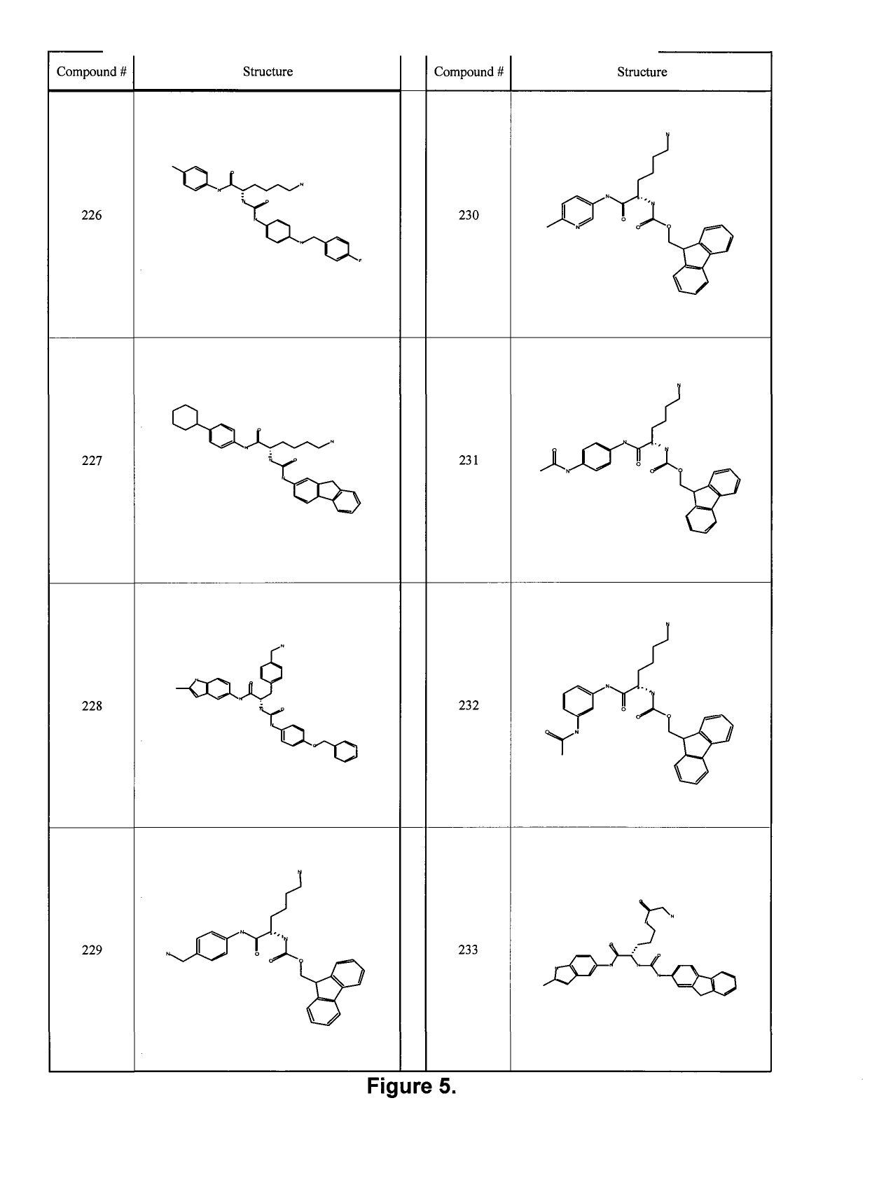

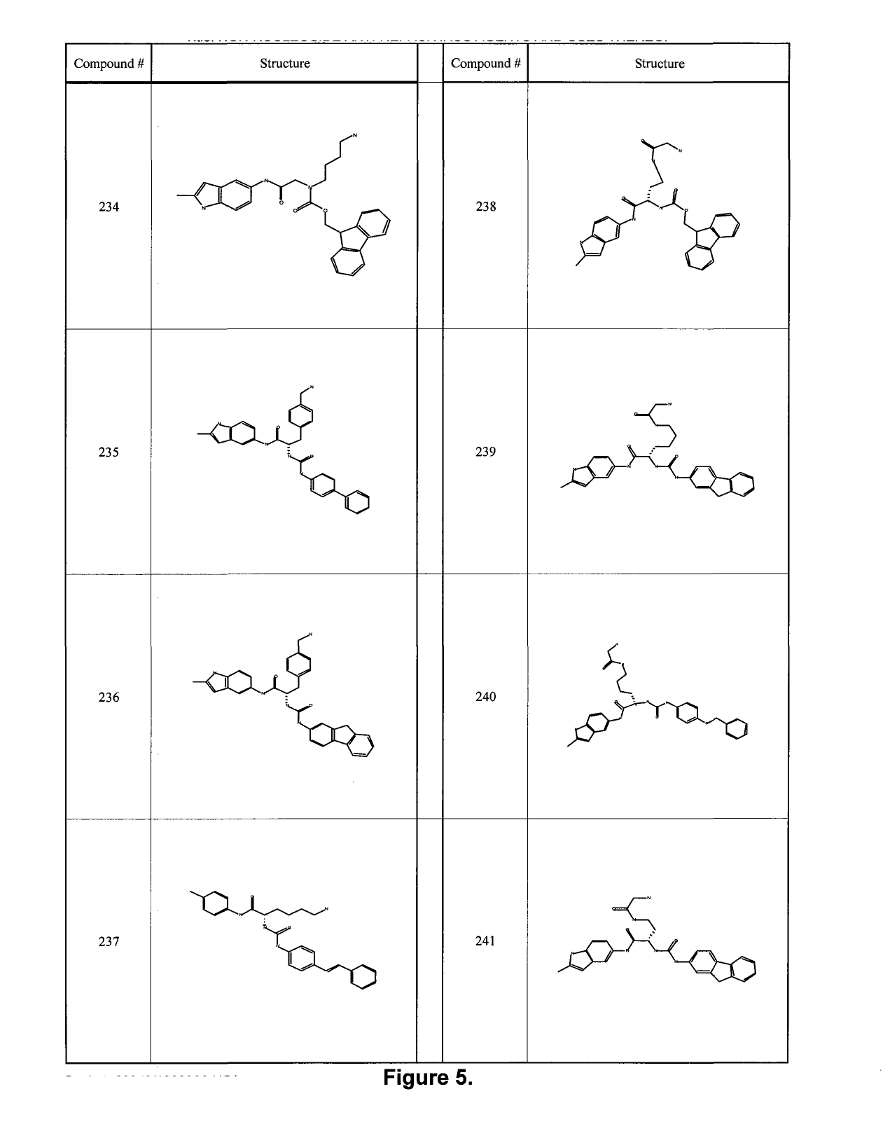

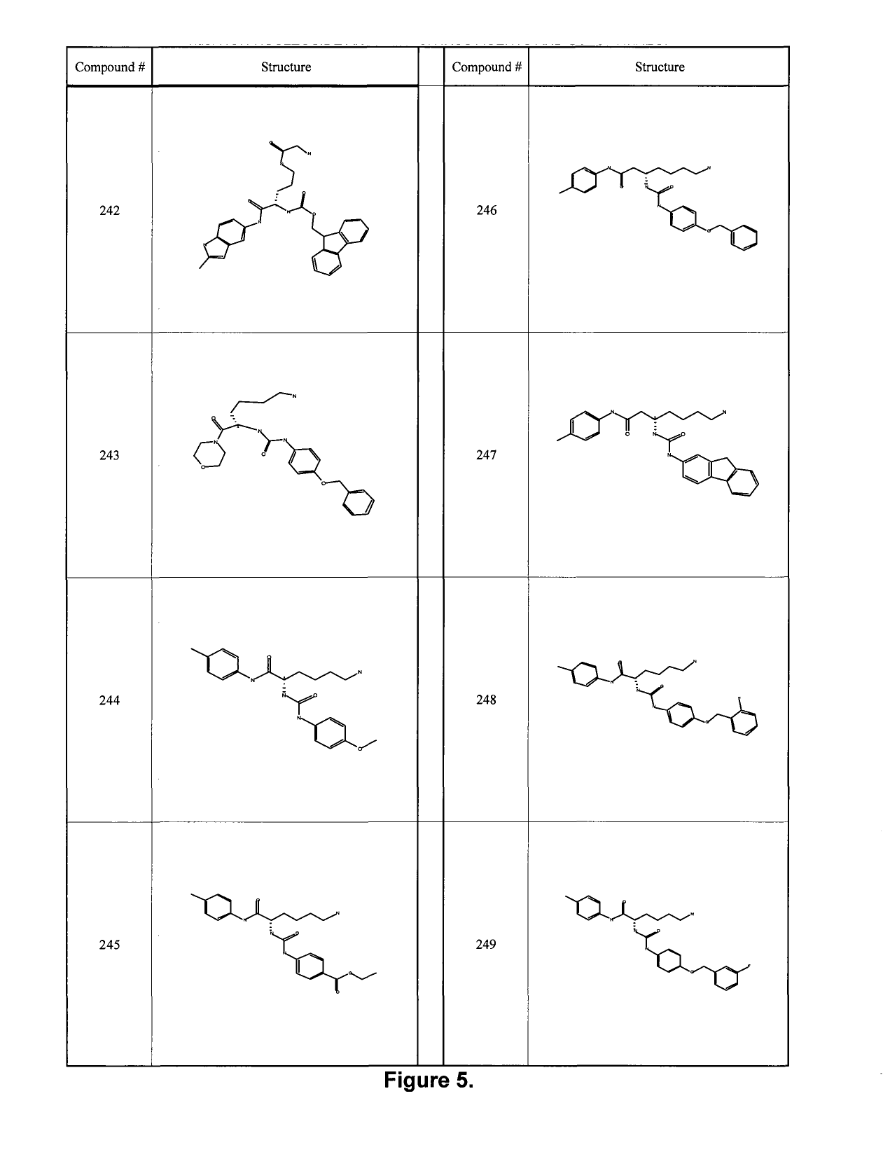

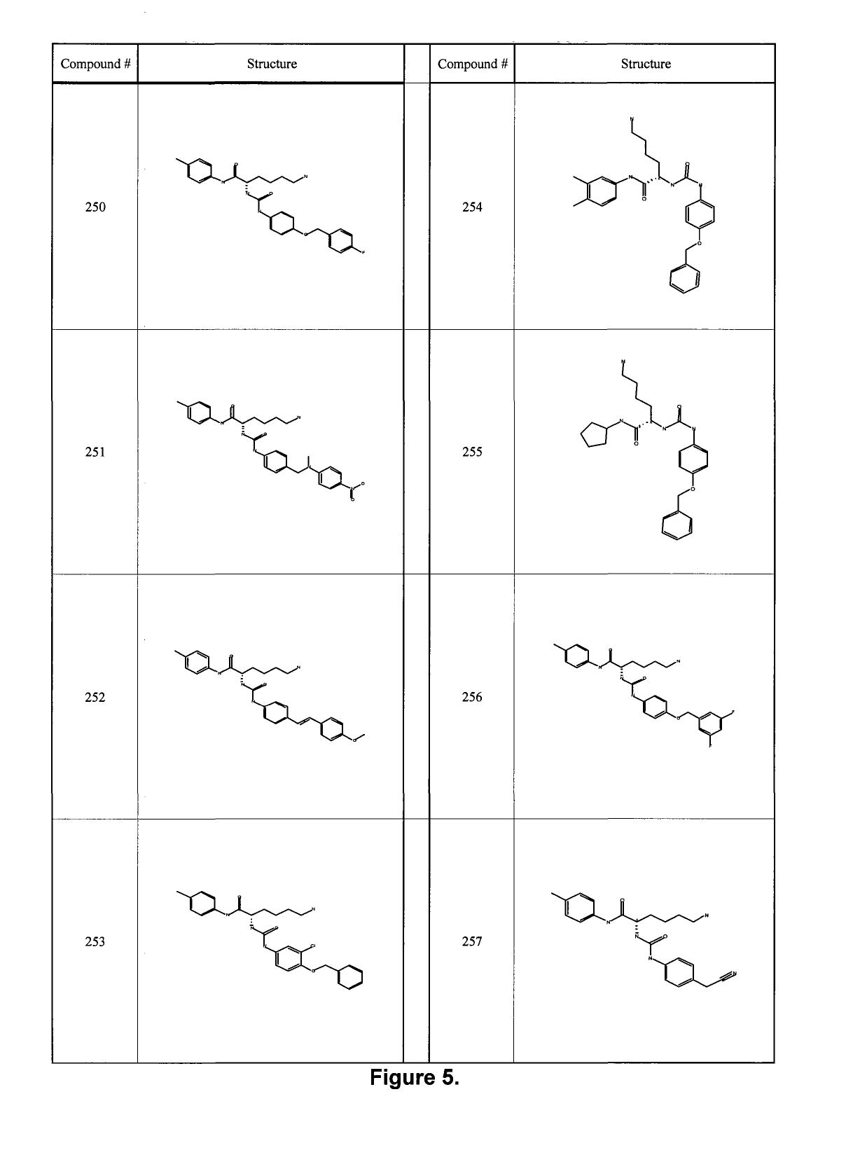

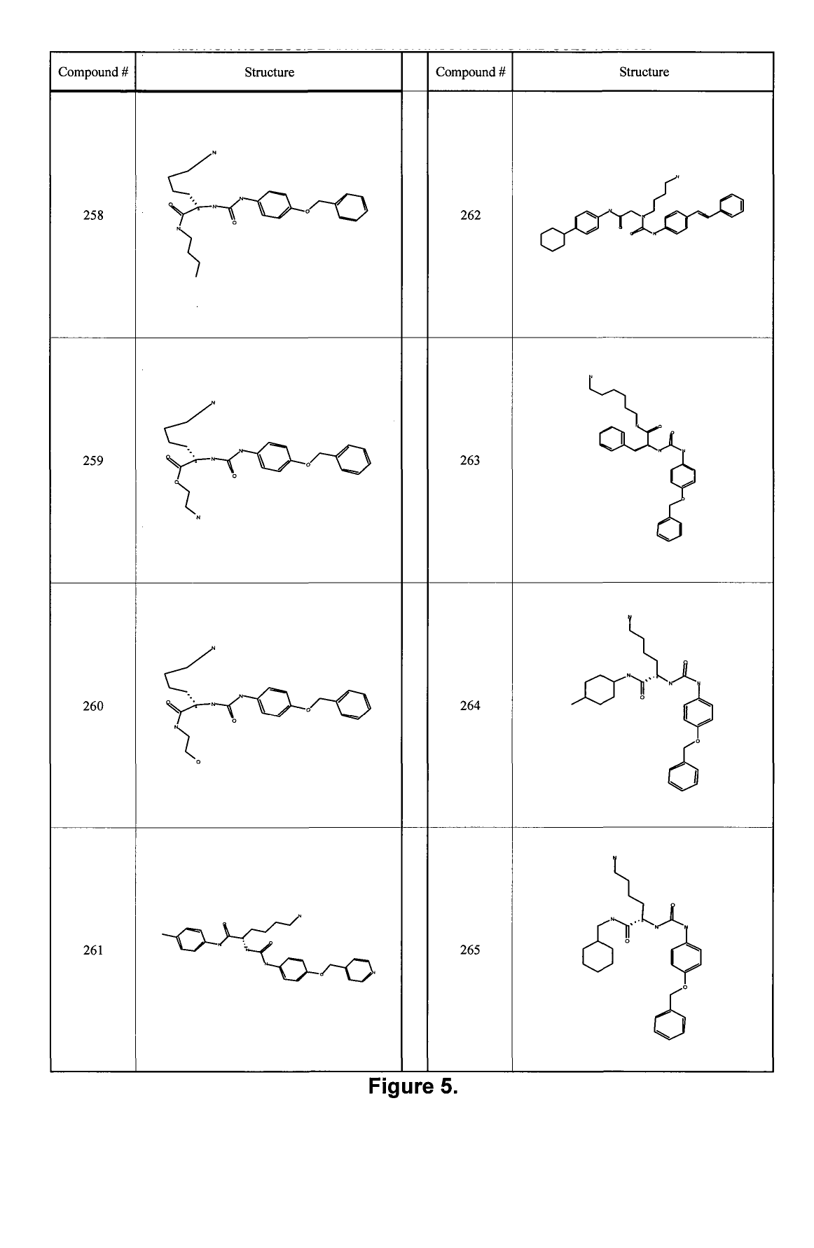

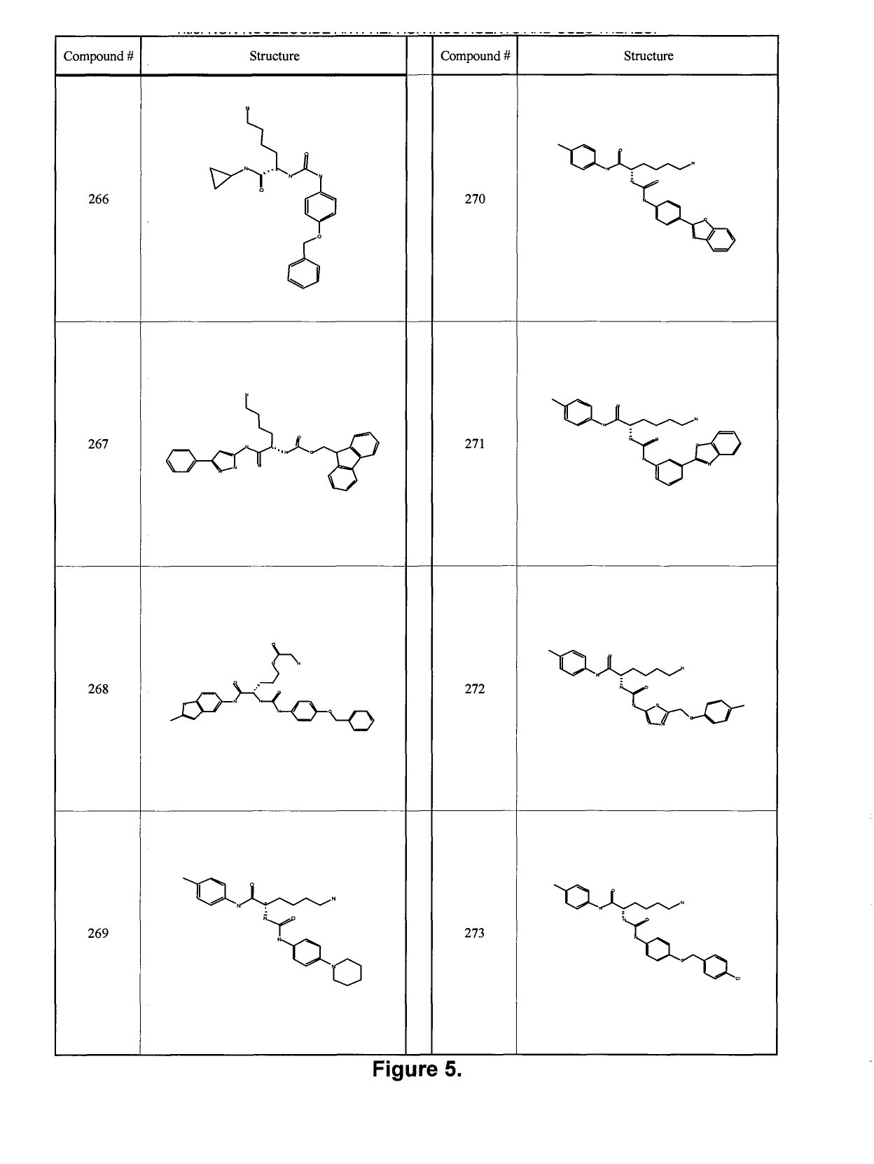

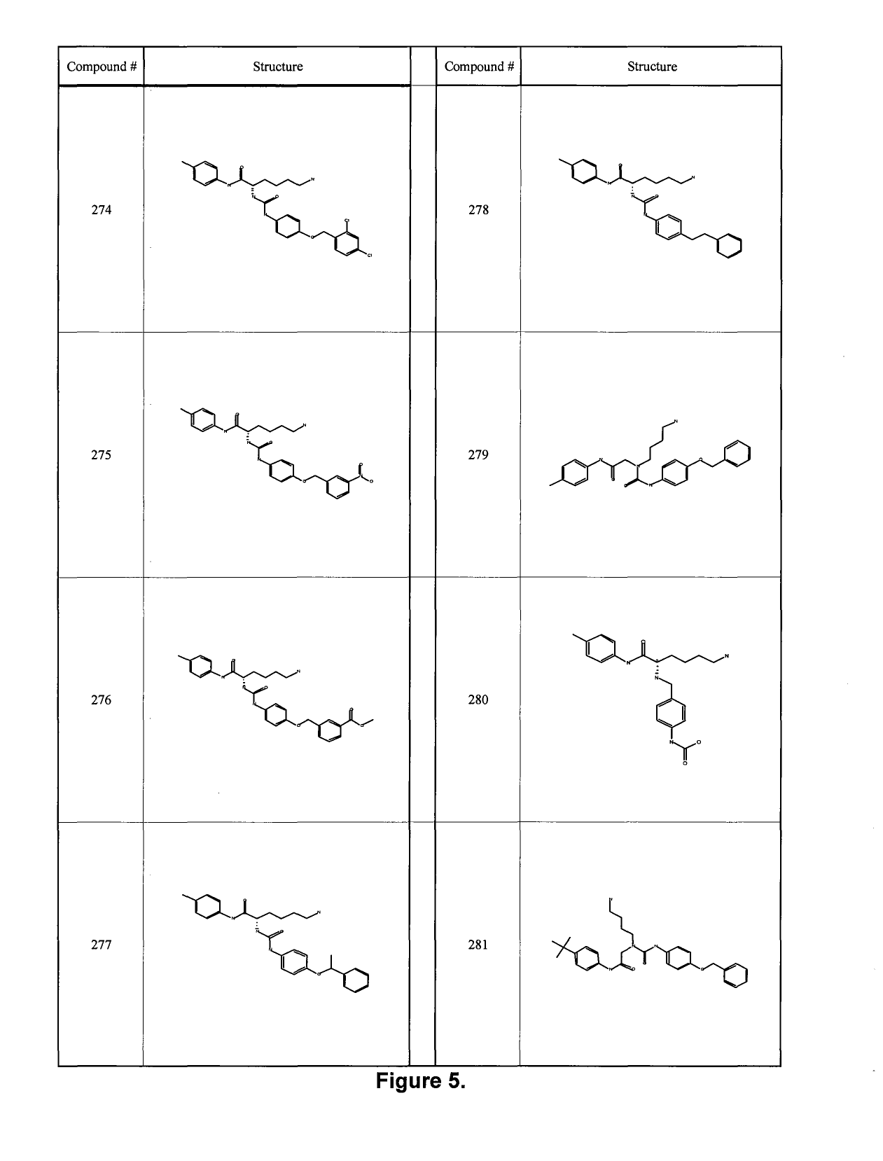

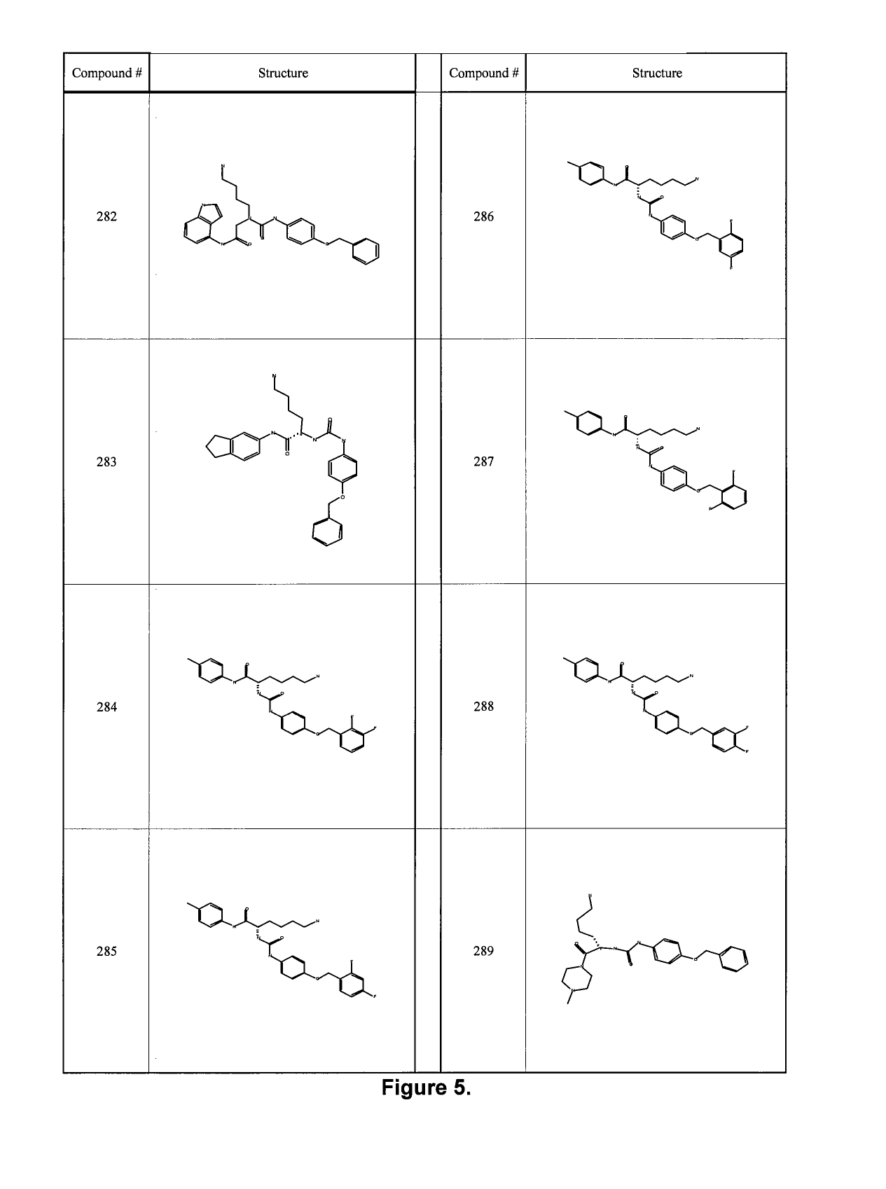

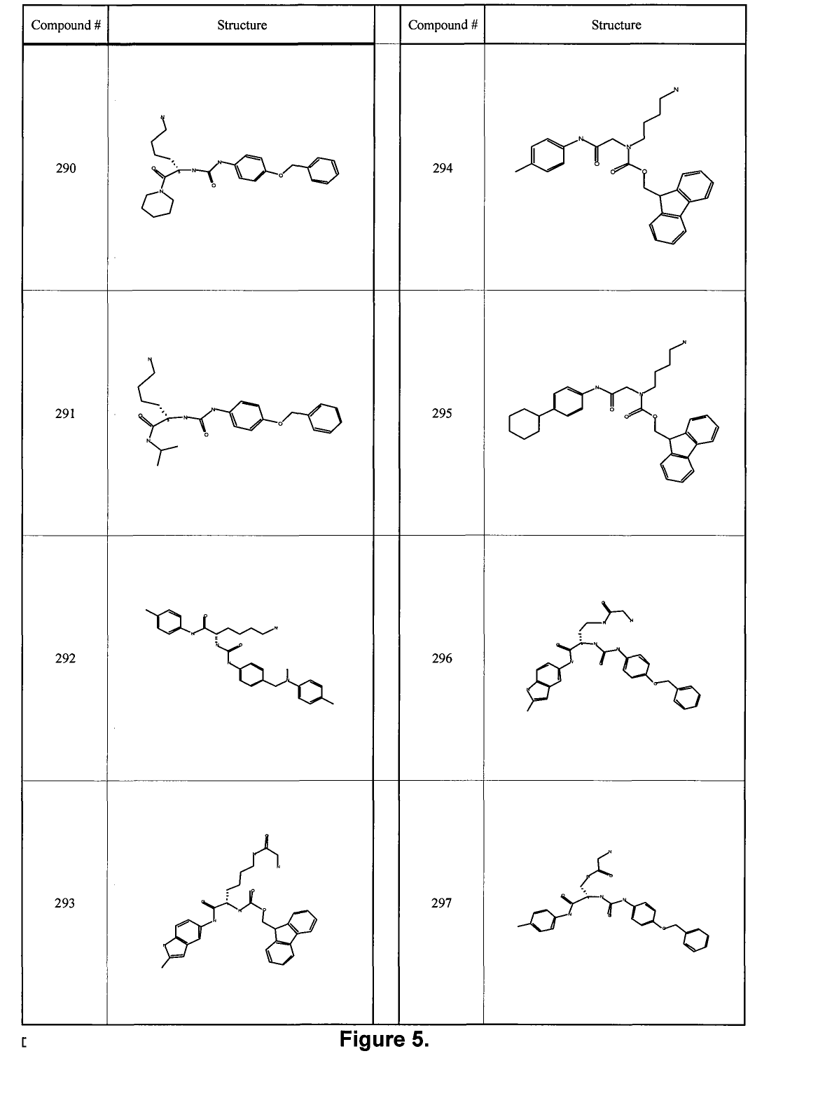

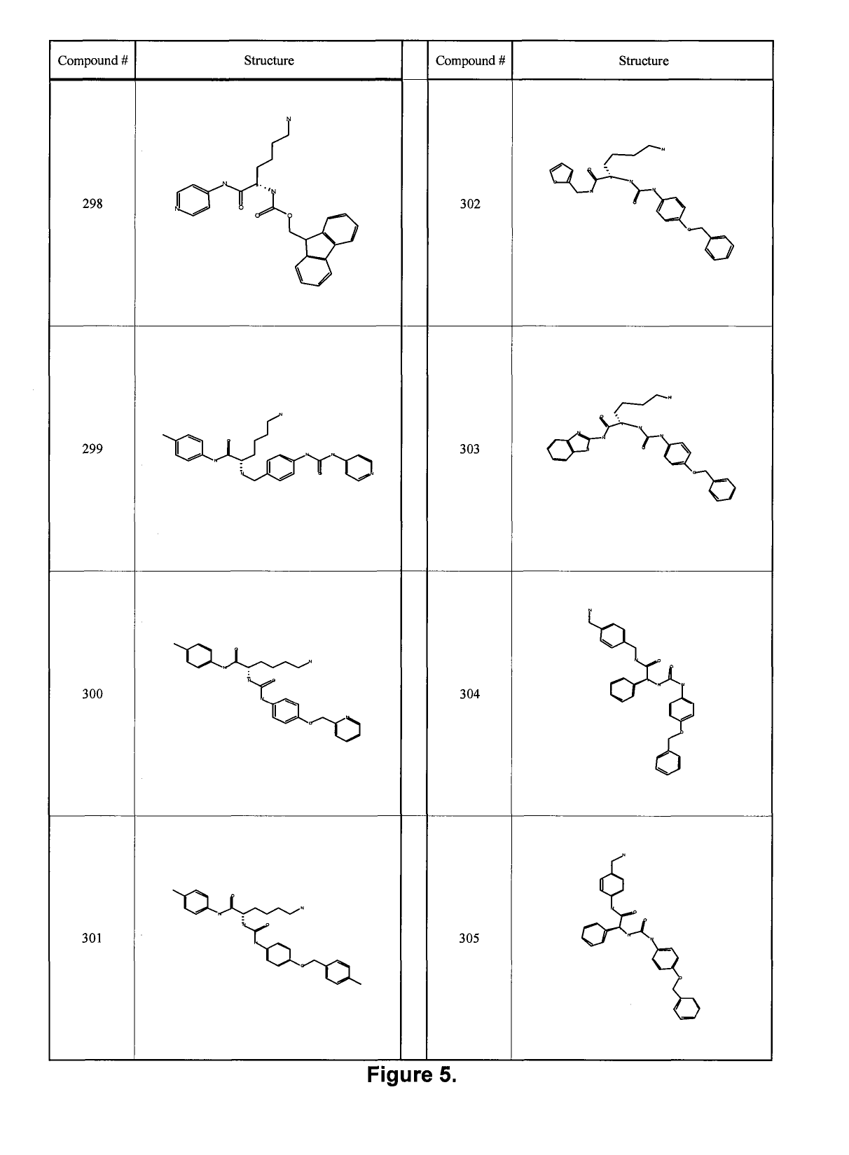

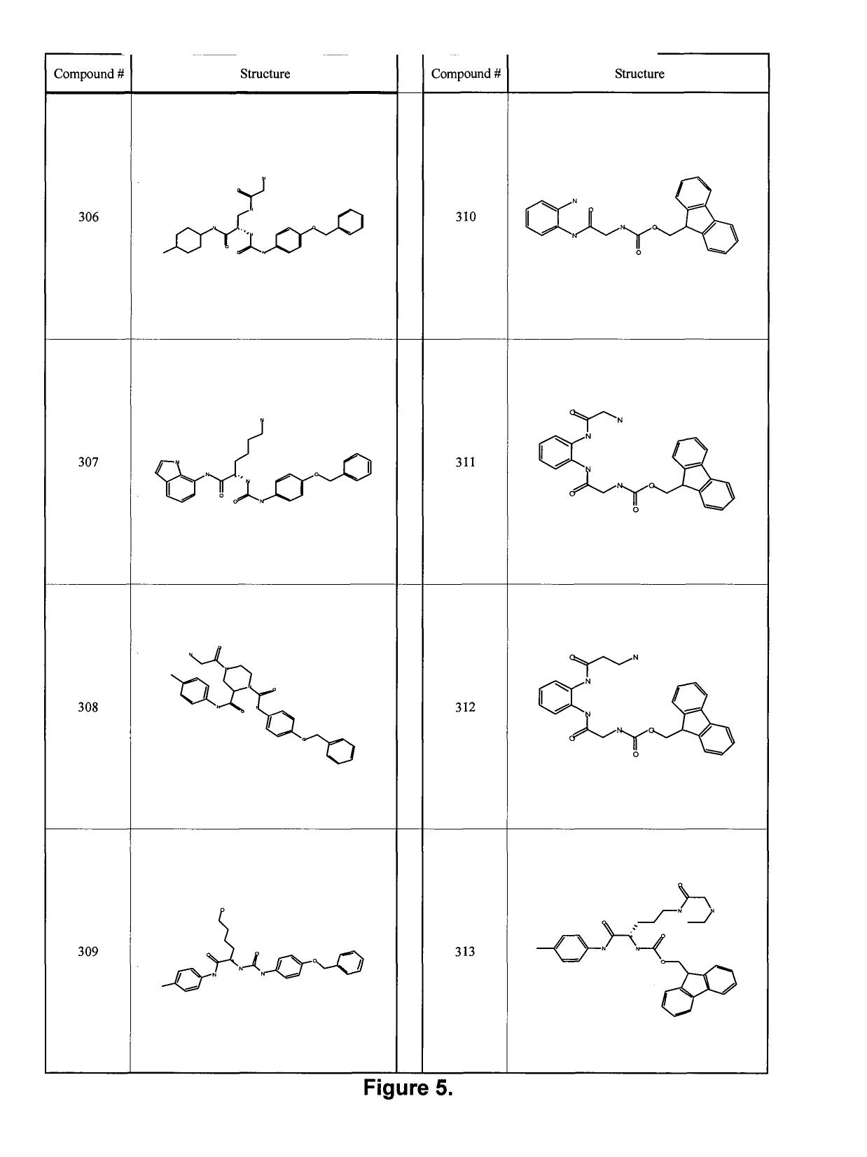

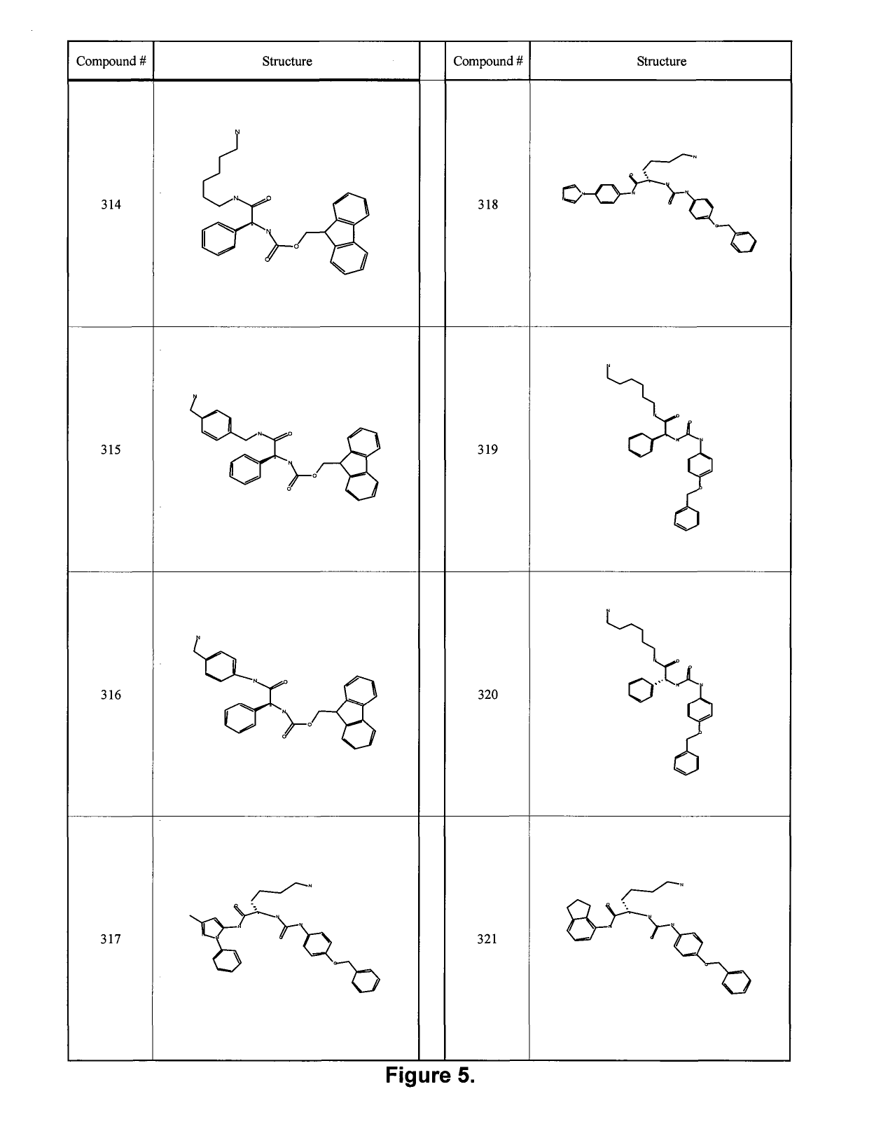

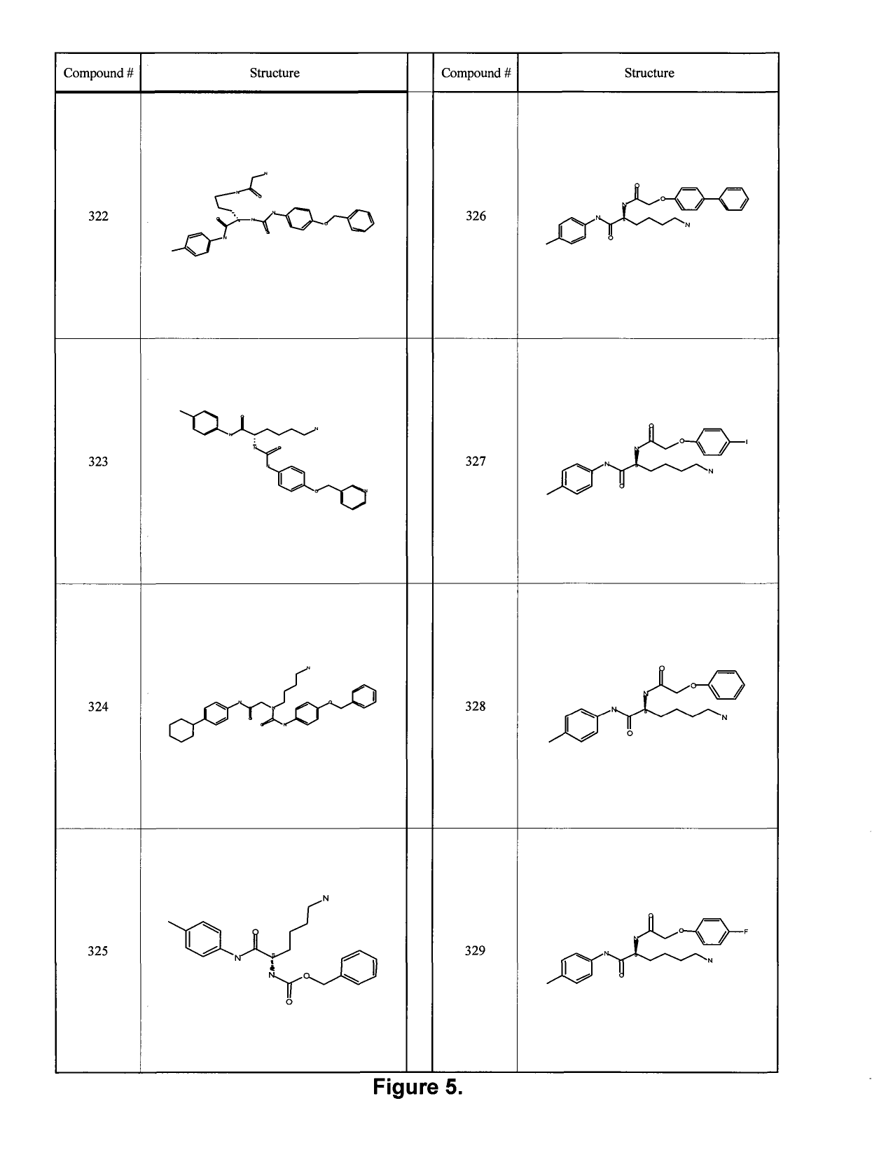

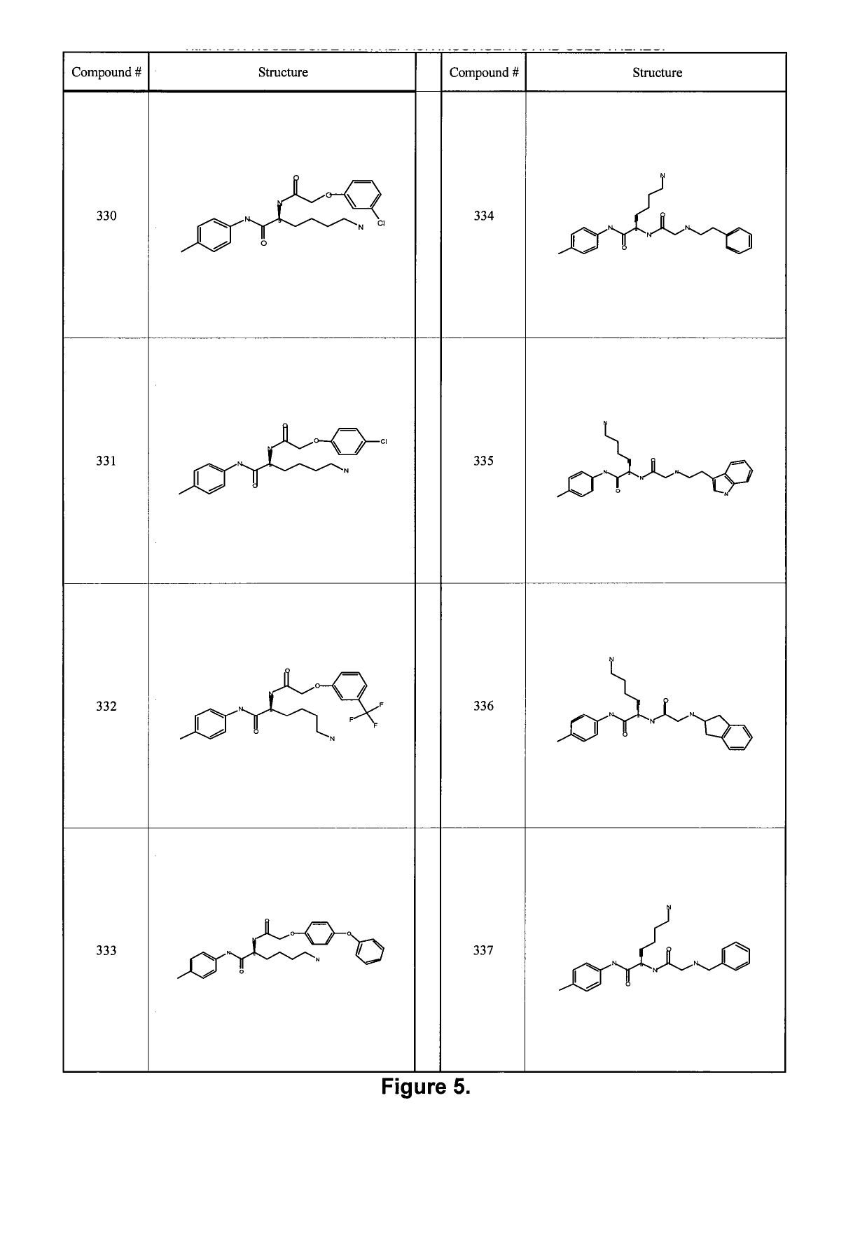

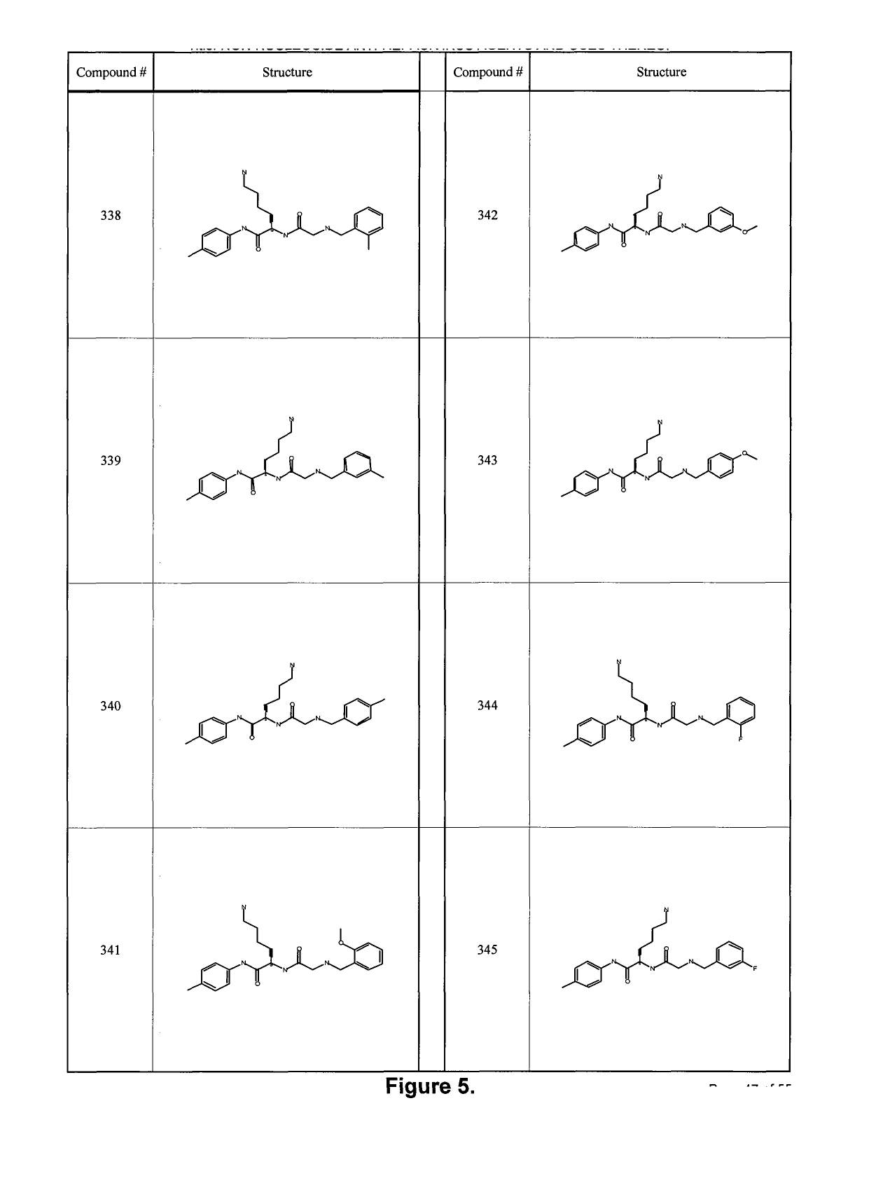

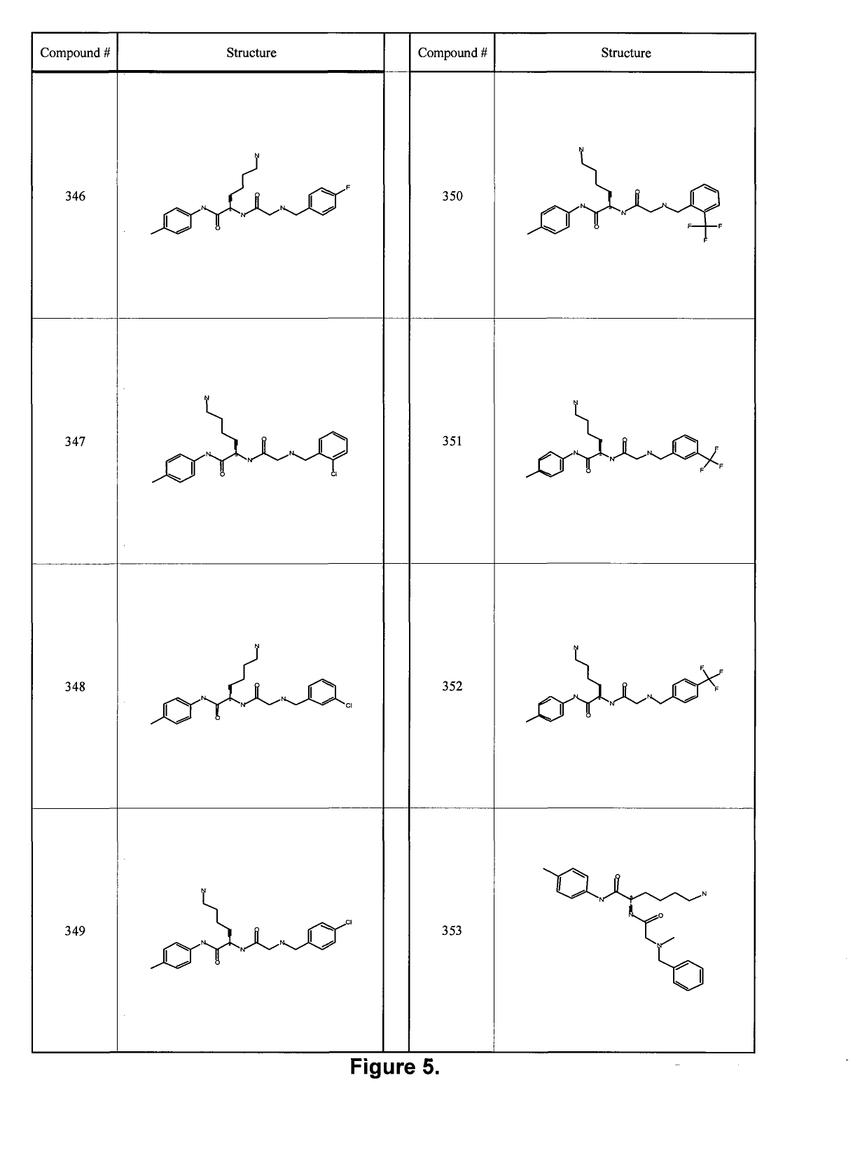

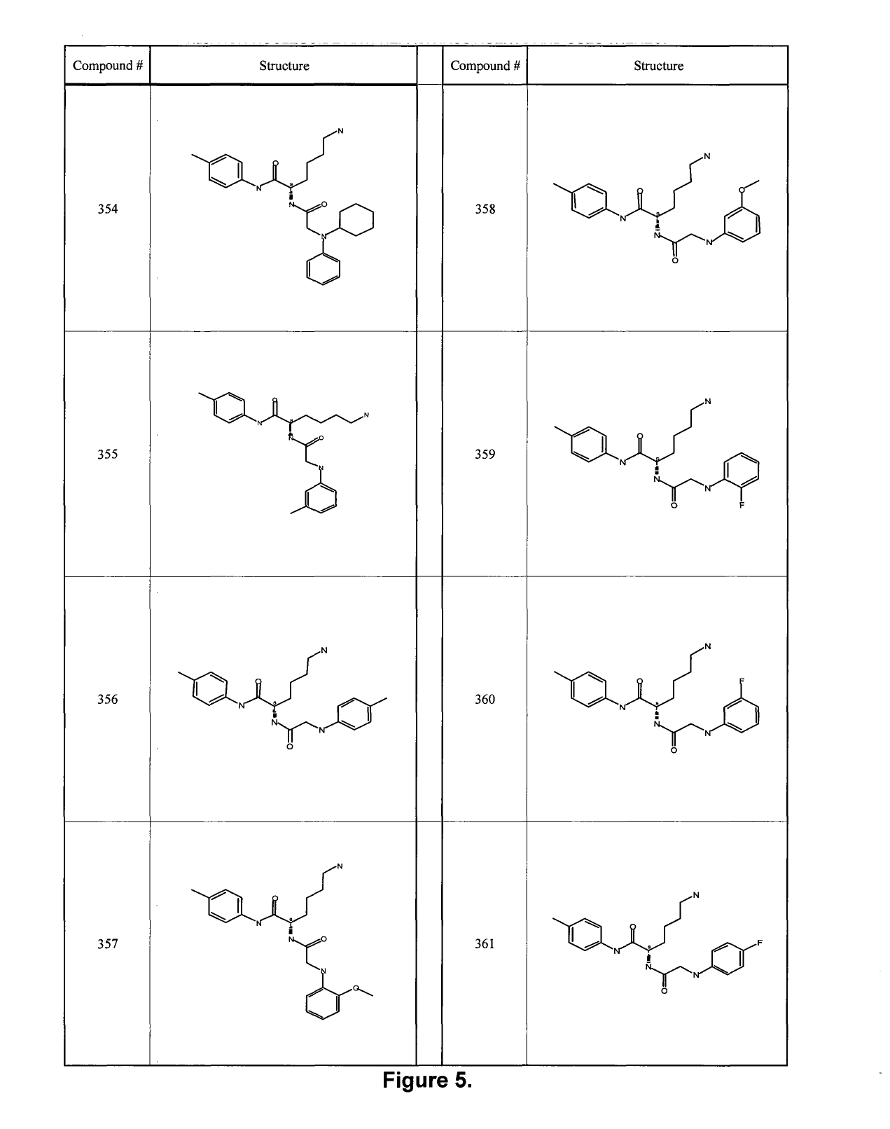

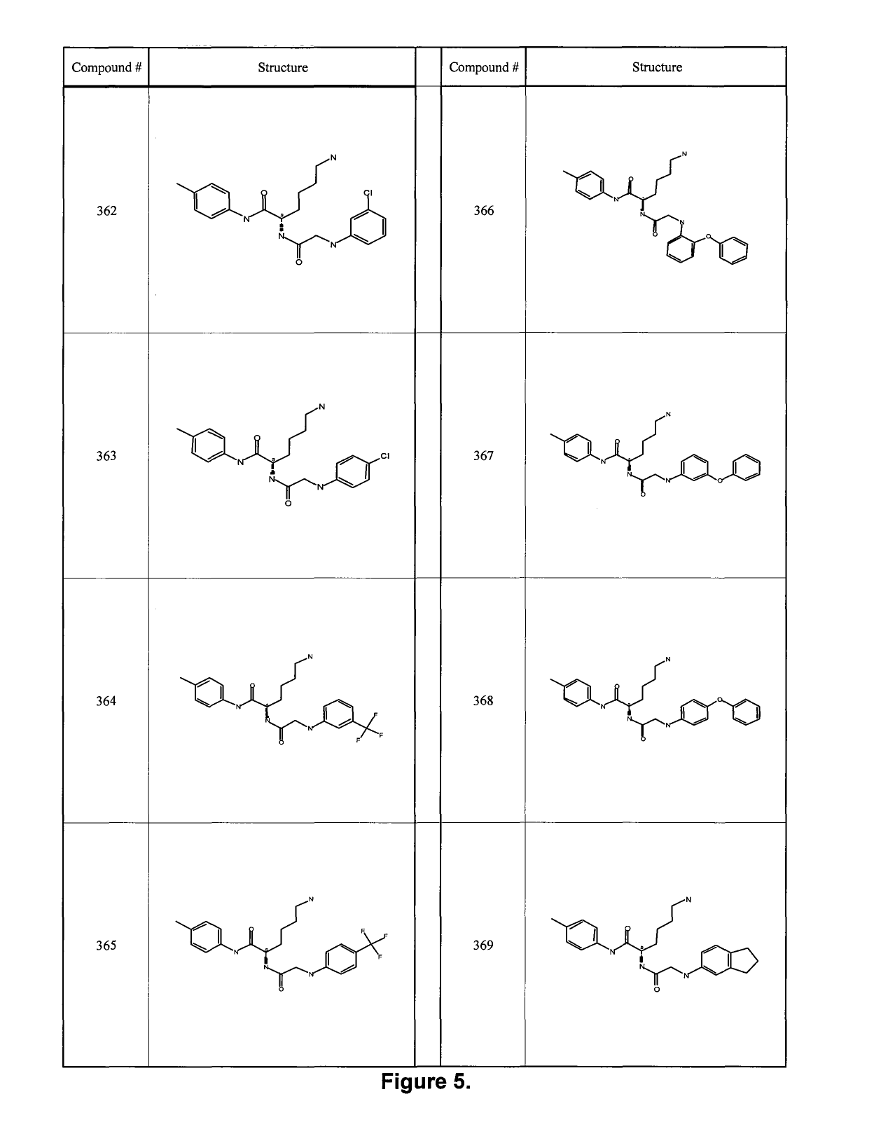

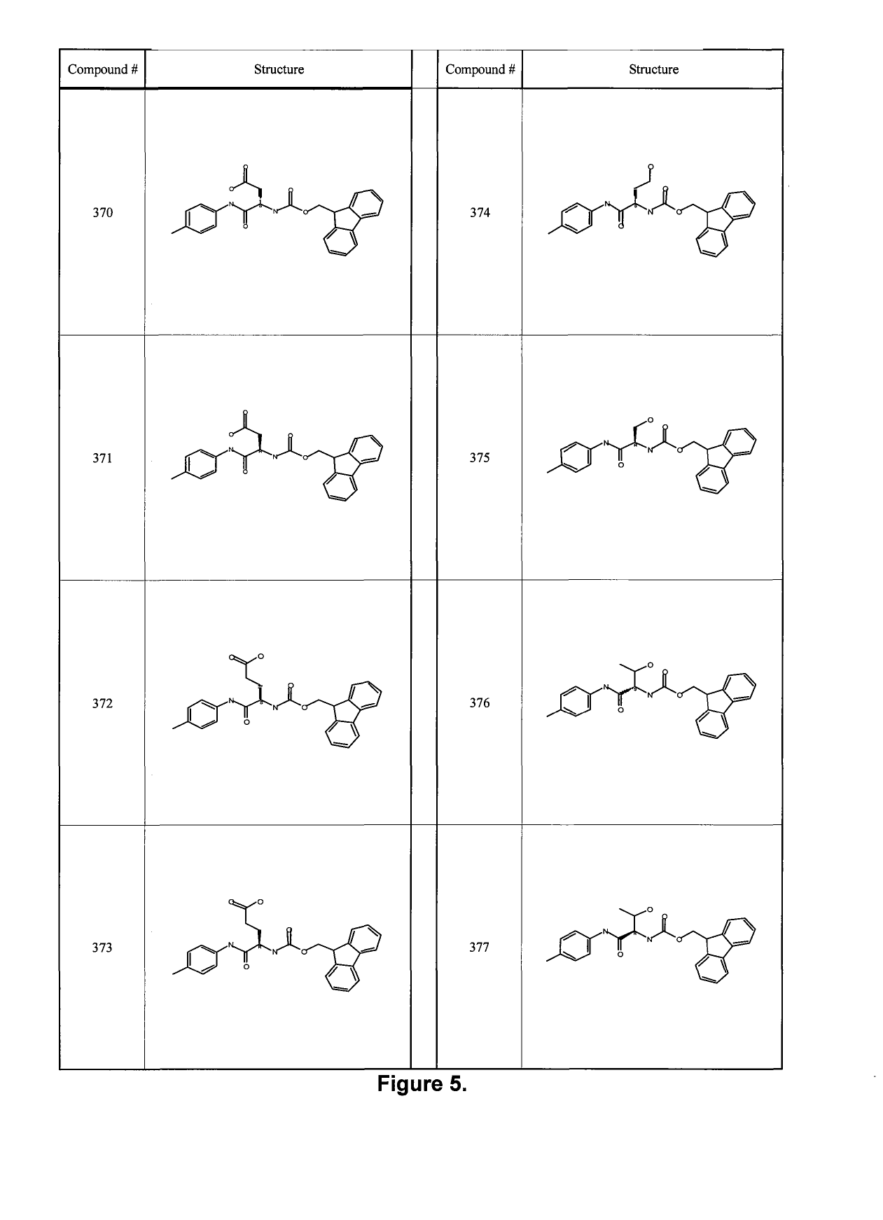

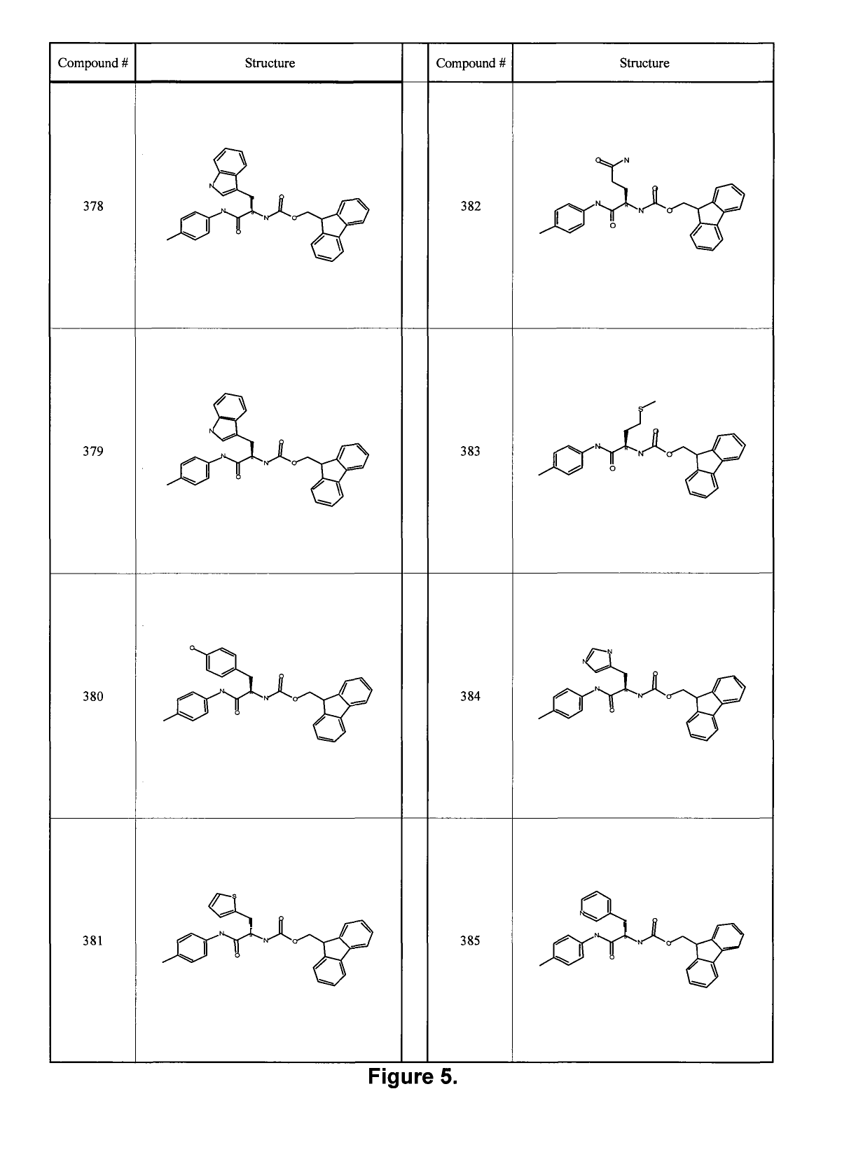

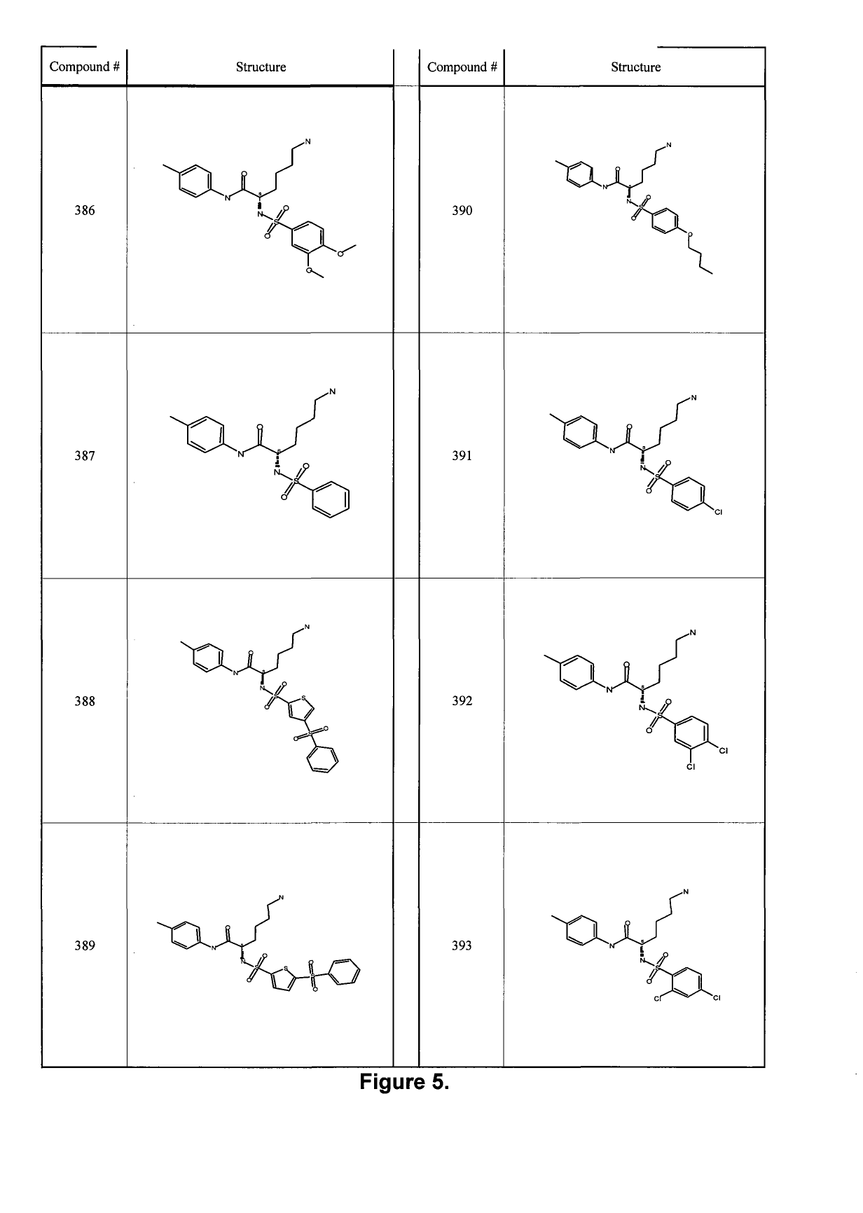

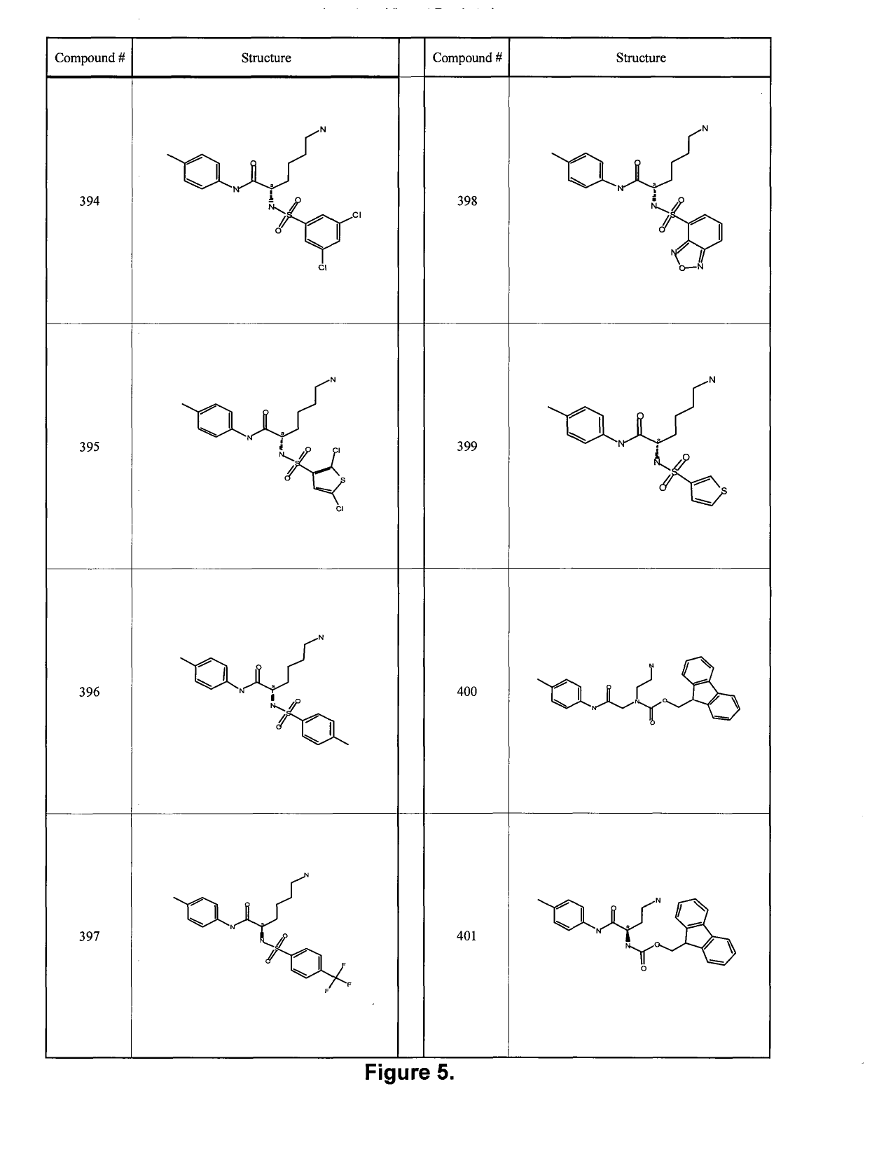

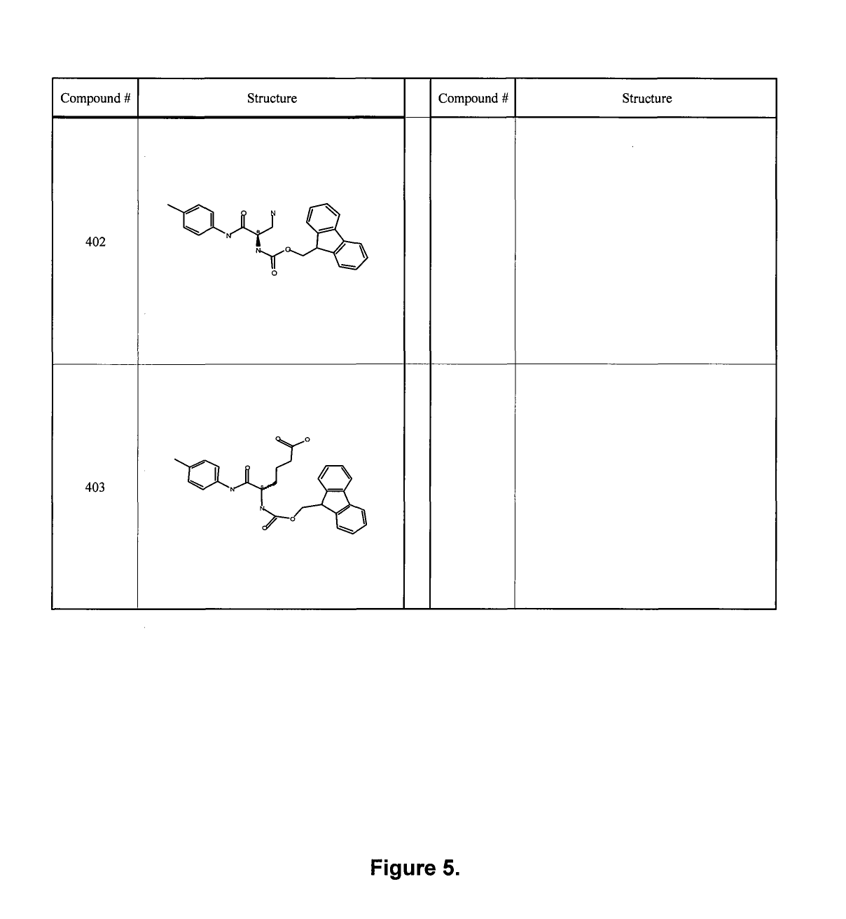

In a further embodiment, provided is a compound having a structure of formula (IV) as defined herein, wherein R³is not hydrogen, or wherein R³ has an ionizable nitrogen. In still further embodiments, provided is a compound having a structure of formula (IV) as defined herein, wherein the compound is compound 2, 297, 137, 146, 172, 199, 228, 272, 121, 142, 26, 94, 117, 119, 120, 125, 127, 145, 166, 173, 206, 207, 214, 237, 240, 268, 270, or 306 (see FIG. 5).

In a further aspect, the instant disclosure provides compounds having a structure of formula (V): 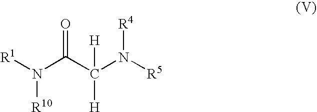
or a stereoisomer, prodrug or pharmaceutically acceptable salt thereof, wherein:

R¹, R⁴ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ and R⁴ are not hydrogen;

R⁵ is selected from —C(═O)NR⁹, —C(═O)(NR¹⁰)SO₂R⁹, —C(═S)NR¹⁰R⁹, or —C(═NR¹⁰)NR¹⁰R⁹; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl.

In a further embodiment, provided is a compound having a structure of formula (V) as defined herein, wherein R⁴ has an ionizable nitrogen. In still further embodiments, provided is a compound having a structure of formula (V) as defined herein, wherein the compound is compound 234, 262, 279, 281, 282, 294, 295, or 324 (see FIG. 5).

In a further aspect, provided are compounds having a structure of formula (VI): 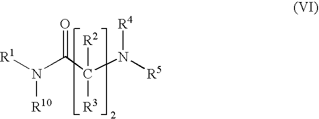
or a stereoisomer, prodrug or pharmaceutically acceptable salt thereof, wherein:

R¹ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀o) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂ C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀)) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;

(i) R², R³ and R⁴ are each independently the same or different substituent as defined for R⁹; or (ii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iii) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, or —SO₂R⁹; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl;

and wherein at least one but not more than three of R², R³, R⁴ and R⁵ is hydrogen, provided that R¹ is not an amino acid when R⁴ and R⁵ are both H.

In a further embodiment, provided is a compound having a structure of formula (VI) as defined herein, wherein the compounds have a structure of formula (VII): 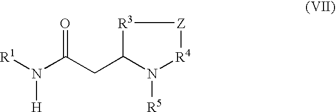
wherein R³ and R⁴ are each independently selected from —CH₂— or —(CH₂)₂—; Z is —NR(R⁹)—; and R¹, R⁵, R⁹, and R¹⁰ are as defined herein for structure (VI). In one embodiment, there is provided a compound of structure (VII), wherein the R⁹ has an ionizable nitrogen. In another embodiment, there is provided a compound of structure (VII), wherein the R⁹ has an ionizable nitrogen, R³is —CH₂— and R⁴ is —CH₂)₂—. In still further embodiments, provided is a compound having a structure of formula (VI) as defined herein, wherein the compound is compound 155, 158, 159, 160, 161, 162, 163, 183, 184, 186, 187, or 197 (see FIG. 5).

In a further aspect, the instant disclosure provides compounds having a structure of formula (VIII): 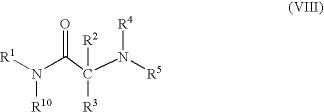
or a stereoisomer, prodrug or pharmaceutically acceptable salt thereof, wherein:

R¹ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;

(i) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (ii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iii) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, or —SO₂R⁹; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl;

and wherein at least one but not more than three of R², R³, R⁴ and R⁵ is hydrogen, provided that R¹ is not an amino acid when R⁴ and R⁵ are both H.

In a further embodiment, the instant disclosure provides a compound of structure (VIII) as defined herein, wherein at least one of R², R³ or R⁴ has an ionizable nitrogen. In another embodiment, provided is a compound of structure (VIII) as defined herein, wherein R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent. In certain embodiments, the compound of formula (VIII) is compound 85, 86, 87, 122, 123, 130, 131, 132, or 156 as shown in FIG. 5. In still another embodiment, provided is a compound of structure (VIII) as defined herein, wherein R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent. In certain embodiments, the compound of formula (VIII) is compound 109 or 138 as shown in FIG. 5.

In a further aspect, the instant disclosure provides compounds having a structure of formula (IX): 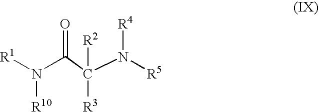
or a stereoisomer, prodrug or pharmaceutically acceptable salt thereof, wherein:

R¹ is the same as R⁹ provided an ionizable nitrogen is present;

(i) R², R³ and R⁴ are each independently the same or different substituent as defined for R⁹; or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, —SO₂R⁹;

R⁹ is selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl;

and wherein at least one but not more than three of R², R³, R⁴ and R⁵ is hydrogen, provided that R¹ is not an amino acid when R⁴ and R⁵ are both H.

In certain embodiments, the compound of formula (IX) is compound 314, 315, 316, 319, or 320 as shown in FIG. 5.

In a further aspect, the instant disclosure provides compounds having a structure of formula (X): 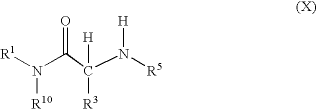
or a stereoisomer, prodrug or pharmaceutically acceptable salt thereof, wherein:

R¹, R³ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁2) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;

R⁵ is selected from H, —C(═O)R⁹, —C(═S)R⁹, —C(═NR¹⁰)R⁹, or —CO₂R⁹; R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl.

In a further embodiment, the instant disclosure provides a compound having a structure of formula (X) as defined herein, wherein R³ is not hydrogen or wherein R³ has an ionizable nitrogen. In still further embodiments, provided is a compound having a structure of formula (X) as defined herein, wherein the compound is compound 334 to 369, and in certain embodiments is compound 358, 360, 366, 367 or 368 as shown in FIG. 5.

The antiviral compounds of the instant disclosure can be utilized as a free acid, free base, or in the form of acid or base addition salts (e.g., pharmaceutically acceptable salts). “Pharmaceutically acceptable salt” refers to a salt of a compound of this disclosure that is pharmaceutically acceptable and that possesses the desired pharmacological activity of the parent compound. Such salts may include the following: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

In another aspect, the antiviral compounds of the instant disclosure may be in the form of a prodrug. “Prodrug” as used herein refers to a compound that can be converted into the parent compound in vivo. Prodrugs often are useful because, in some situations, they may be easier to administer than the parent compound. For example, the prodrug may be more bioavailable by oral administration or for cellular uptake than a parent compound. The prodrug may also have improved solubility in pharmaceutical compositions over the parent compound or an extended half-life in vivo. An example of a prodrug can be a compound as described herein that is administered as an ester (a “prodrug”) to, for example, facilitate transmittal across a cell membrane (when water solubility is detrimental to mobility across such as membrane). Once in the cell, the prodrug may then be metabolically hydrolyzed to a more water soluble form where water solubility is beneficial. Alternatively, a prodrug compound may be converted into its metabolite before entry into a cell. Other representative examples of prodrugs include acetate, formate, and benzoate derivatives of alchohol and amine functional groups that would be converted into hydroxy or amine groups. In certain embodiments, such a prodrug compound may be inactive (or less active) until converted into a metabolite (i.e., parent compound or derivative thereof). In other embodiments, a prodrug compound may be remain active (or have substantially similar activity to the parent compound) before being converted into a metabolite. “Structurally pure” refers to a compound composition in which a substantial percentage, e.g., on the order of 95% to 100% and preferably ranging from about 95%, 96%, 97%, 98%, 99% or more, of the individual molecules comprising the composition each contain the same number and types of atoms attached to each other in the same order and with the same bonds. As used herein, “structurally pure” is not intended to distinguish different geometric isomers or different optical isomers from one another. For example, as used herein a mixture of cis- and trans-but-2,3-ene is considered structurally pure, as is a racemic mixture. When compositions are intended to include a substantial percentage of a single geometric isomer and/or optical isomer, the nomenclature “geometrically pure” and “optically or enantiomerically pure,” respectively, are used.

The phrase “structurally pure” is also not intended to discriminate between different tautomeric forms or ionization states of a molecule, or other forms of a molecule that result as a consequence of equilibrium phenomena or other reversible interconversions. Thus, a composition of, for example, an organic acid is structurally pure even though some of the carboxyl groups may be in a protonated state (—CO₂H) and others may be in a deprotonated state (—CO₂⁻). Likewise, a composition comprising a mixture of keto and enol tantomers, unless specifically noted otherwise, is considered structurally pure.

The antiviral compounds of this disclosure may contain a chiral center on any of the substituents and these can exist in the form of two optical isomers (the (+) and (−) isomers, also referred to as the (R) and (S) isomers). All such enantiomers and mixtures thereof, including racemic mixtures, are included within the scope of this disclosure. A single optical isomer (or enantiomer) can be obtained by methods known in the art, such as by chiral HPLC or other chiral chromatography, enzymatic resolutions, use of chiral auxillaries, selective crystallization, or any combination thereof. In certain embodiments, some of the crystalline forms of the antiviral compounds of this disclosure may exist as polymorphs, which are included within the scope of this disclosure. In further embodiments, some of the antiviral compounds of this disclosure may form solvates with solvents (e.g., water, organic solvents), which are included within the scope of this disclosure.

In certain embodiments, the present disclosure provides compounds in the form of a single enantiomer that is at least 90%, 95%, 97% or at least 99% free of a corresponding enantiomer. In one embodiment, the single enantiomer is in the (+) form and is at least 90%, at least 95%, at least 97% or at least 99% free of a corresponding (−) enantiomer. In one embodiment, the single enantiomer is in the (−) form and is at least 90%, at least 95%, at least 97% or at least 99%, free of a corresponding (+) enantiomer.

Methods of Synthesis

The compounds of the invention may be synthesized via several different synthetic routes using commercially available starting materials or starting materials prepared by conventional synthetic or biosynthetic methods. For example, the synthesis may be carried out in solution or in solid phase. An exemplary synthetic approach in solution is illustrated in Scheme (I) (see Examples 2-325), as follows:

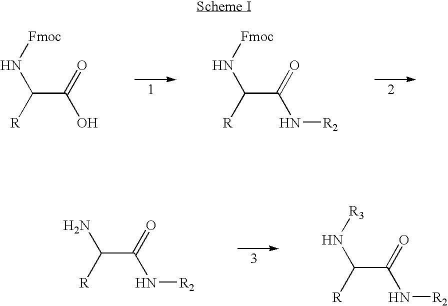

Prior to using Scheme I, a compound of interest is coupled with an appropriate protective group, such as t-Boc or Fmoc, generate a protected reactant. While Scheme I illustrates the use of an Fmoc protecting group, a person having ordinary skill in the art will recognize that other protecting groups may be employed. In addition, in some instances, a parent compound may include other or additional functionalities that may require protection. Groups suitable for protecting a wide variety of different functionalities, as well as conditions for their removal, are well known and will be apparent to those of ordinary skill in the art. Specific guidance for selectively protecting a wide variety of functionalities may be found, for example, in Greene & Wuts, Protective Groups in Organic Synthesis, 3^{rd} edition, 1999 (“Greene & Wuts”). Preferred protecting groups are those that may be easily removed. Exemplary groups for protecting primary amines are tert-butyloxycarbonyl (“t-Boc”), 9-fluorenylmethoxycarbonyl (“Fmoc”) benzyloxycarbonyl (“Z”), and allyloxycarbonyl (Alloc).

In a first step of Scheme I, a protected reactant is subjected to an amidation reaction, or other nucleophilic substitution, to provide a protected intermediate (or mixture of intermediates) in reasonable crude yields. Purification of this protected intermediate by chromatography is optional. Reaction conditions for coupling amines with carboxylic acids to yield amide linkages are known to those of ordinary skill in the art and may be found in any compendium of standard synthetic methods or literature related to the synthesis of peptides and proteins. See e.g., March, J., Advanced Organic Chemistry, Reactions, Mechanisms and Structure, 4^{th} ed., 1992; Larock, Comprehensive Organic Transformations, VCH, New York, 1999; Bodanzsky, Principles of Peptide Synthesis, Springer Verlag, 1984; Bodanzsky, Practice of Peptide Synthesis, Springer Verlag, 1984; Lloyd-Williams et al., Chemical Approaches to the Synthesis of Peptides and Proteins, CRC Press, 1997 (see especially pp. 105-114); and Atherton & Sheppard, Solid Phase Peptide Synthesis: A Practical Approach, IRL Press, 1989). Alternative reactive groups can be utilized, such as isocyanate (which would yield a urea) and others exemplified herein, in methods known in the art. In a second step, deprotection provides a free amine use in a third step of coupling with an appropriate electrophile, such as an isocyanate or acyl chloride, to provide a crude final product.

The derivative compounds of the invention may be isolated and purified using standard techniques, such as high-pressure liquid chromatography (HPLC), fast protein liquid chromatography (FPLC), counter current extraction, centrifugation, filtration, precipitation, ion exchange chromatography, gel electrophoresis, affinity chromatography, flash chromatography, and the like. Specific methods of isolation are provided in the Examples section below. Standard characterization and purity analysis known in the art can be used to verify final products and intermediates.

An exemplary solid-phase synthetic approach is illustrated in Scheme (II) (see Examples 326-403), as follows: 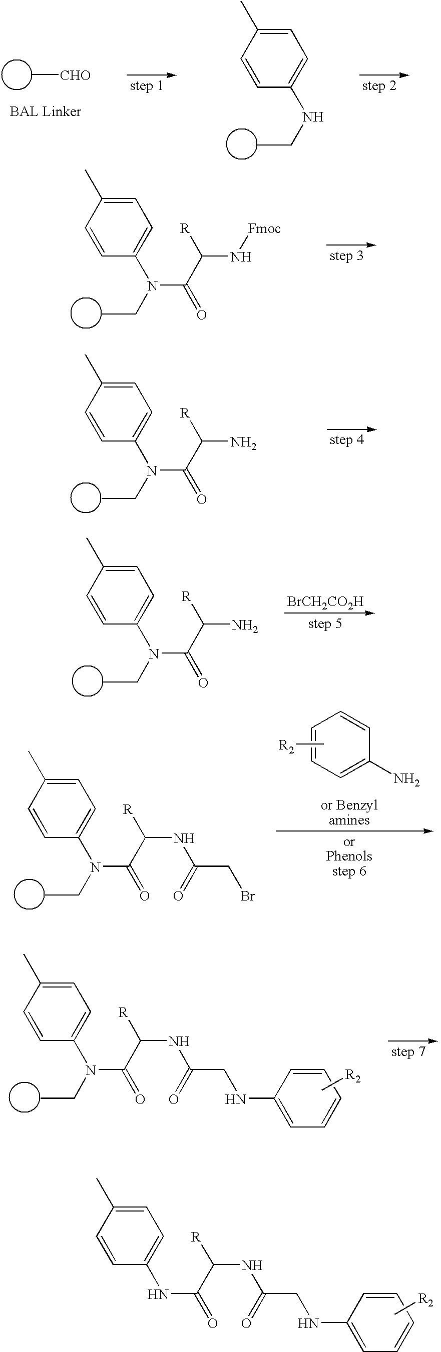
Step 1. Reductive Amination

Used in these procedures were SynPhase™ polystyrene D-series Lanterns derivatized with backbone amide linker (BAL) with a nominal loading capacity if 35 μmol available from Mimotopes (code MIL1018). The working volume of solvent per Lantern was 0.7 mL.

A stock solution was prepared of sodium cyanoborohydride (0.1 M) in acetic acid/dimethylformamide (DMF) (1:99). The appropriate amines (1.0 M) were dispensed into glass vials or Schott bottles, to which the sodium cyanoborohydride stock solution was added. The Lanterns were added and the mixture heated for 17 h at 60° C. For certain types of amines, a gelatinous substance was formed. Lanterns were pushed into the jelly using tweezers prior to heating.

The reaction solution was removed and the Lanterns washed with methanol (3×5 min), DMF (3×5 min), methanol (3×5 min) and dichloromethane (DCM) (3×5 min). Lanterns were air-dried in a fumehood for 48 h, which were then ready for the next reaction.

Step 2. Acylation of p-Toluidine mounted on BAL Lanterns with α-Fmoc-Lys(NBoc)

Fmoc-Lys(NBoc) acid (5.6 g, 12 mmol) was dissolved in DCM (60 mL) as a 0.2 M solution in a Schott bottle. Diisopropylcarbodiimide (DIC) (1.88g, 12mmol) was added, the mixture was shaken, and then let to stand for 15min at room temperature. Diisopropyl urea precipitated as a fine colorless solid over this time.

BAL double Lanterns (100) from step 1 were added to solution, shaken to make sure all Lanterns were immersed in solution, and then left to stand overnight at room temperature. The supernatant phase was removed from the Lanterns by aspiration followed by a washing procedure to remove excess reagents. The Lanterns were washed in DCM (2× rapid). Further, longer washing with DCM (3×15 min) followed, after which the Lanterns were air-dried. An Fmoc loading test on two lanterns gave an average loading of 32.5 μmole/Lantern. Proceeding to step 7 retains the Fmoc protection for the isolation of some of the final compounds below (see, e.g., Example 403).

Step 3. Acylation of BAL-p-Toluidine-Lys(Boc) Lanterns with Bromoacetic Acid

The double Lanterns from Step 2 (98) were immersed in freshly prepared piperidine/DMF (1:4) for 30 min at room temperature to effect Fmoc deprotection. After aspiration of the supernatant phase, the Lanterns were washed with DMF (2× rapid) and then further washed with DMF for a longer period (3×10 min). The still wet Lanterns after the final aspiration were used below.

Bromoacetic acid (3.8 g, 27.5 mmol) was dissolved in DMF (55 mL) to make a 0.5 M solution in a Schott bottle. Diisopropyl carbodiimide (4.39 mL, 28.05 mmol) was transferred by pipette and the mixture was shaken and then left to stand for 15 min at room temperature. The deprotected Lanterns (98) were added with some shaking to ensure complete immersion and the mixture was left standing overnight at room temperature.

The supernatant reaction mixture was removed by aspiration and the Lanterns were washed with N,N-dimethylacetamide (DMA) (2× rapid) followed by DMA (2×10 min), DCM (1×10 min), and finally DMA (1×10 min). The reactive derivatized Lanterns were used in the following steps 4-6 without further drying, and protected from the atmosphere as rapidly as possible.

Step 4. Reaction of BAL-p-Toluidine-Lys(BOC) Bromoacetyl Lanterns with Phenols

Phenol solutions (0.25 M) in DMA (105 mL) with anhydrous potassium carbonate (0.25 M) were prepared in screw-capped glass vials. After capping, the mixtures were heated to 40° C. for 30 min, and then cooled to room temperature before placing three lanterns from step 3 into the phenol solutions ensuring complete immersion of the Lanterns. The mixtures were shaken gently at room temperature for 19 h.

A small section of one of the Lanterns from each reaction was excised with a razor blade or scalpel and each section was washed separately in a labeled vial with DMA (2×10 min) followed by DCM (2×10 min). The still wet sections were cleaved as described in Step 7, samples were removed from the TFA/DCM solutions, and diluted in acetonitrile for LC and liquid chromatography/mass spectroscopy (LC/MS) analysis. Phenols that had not completely reacted by substitution with the solid-phase bound bromoacetyl group were heated either at 40° C. or 60° C., depending on the rate of substitution observed. Sampling of the solid phase reaction was repeated as described and as necessary.

At the completion of the reactions, they were aspirated to remove reagents and the Lanterns were then washed with DMA (2×10 min), water (1×10 min), DMA (2×10 min), and finally DCM (4×10 min) while still in their original reaction tubes. The still wet Lanterns were then subjected to cleavage as described in Step 7. Samples were prepared for final LC and LC/MS analysis from the intermediate water/acetonitrile solutions.

Step 5. Reaction of BAL-p-Toluidine-Lys(Boc) Bromoacetyl Lanterns with Primary Amines.

Amine solutions (2 M) in DMA (1.5 mL) were prepared in screw-capped glass vials. Three lanterns from step 3 were placed into the solutions ensuring complete immersion of the Lanterns. The mixtures were shaken gently at room temperature for 19 h.

A small section of one of the Lanterns from each reaction was excised with a razor blade or scalpel and each section was washed separately in a labeled vial with DMA (2×10 min) and then DCM (2×10 min). The still wet sections were cleaved as described in Step 7. Samples were removed from the TFA/DCM solutions and diluted in acetonitrile for LC and LC/MS analysis. All of the primary amines were found to have reacted to completion.

At the completion of the reactions, they were aspirated to remove reagents and the Lanterns were then washed with DMA (2×10 min) and then DCM (3×10 min) while still in their original reaction tubes. The still wet Lanterns were then subjected to cleavage as in step 7. Samples were prepared for final LC and LC/MS analysis from the intermediate water/acetonitrile solutions.

Step 6. Reaction of BAL-p-Toluidine-Lys(Boc) Bromoacetyl Lanterns with Secondary Amines and Anilines

Solutions of aniline or secondary amine (2 M) in DMA (1.5 mL) were prepared in screw-capped glass vials. Three lanterns from Step 3 were placed into the solutions ensuring complete immersion of the Lanterns. The mixtures were heated at room temperature, 60° C. or 80° C., until complete.

The progress of the reaction was determined by analyzing a small sample of the cleaved residue by LC/MS. A small section of one of the Lanterns from each reaction was excised with a razor blade or scalpel and each section was washed separately in a labeled vial with DMA (2×10 min) and then DCM (2×10 min). The still wet sections were cleaved as described in Step 7. Samples were removed from the TFA/DCM solutions and diluted in acetonitrile for LC and LC/MS analysis. When reactions were determined to be incomplete, the temperature of the reaction and/or the reaction time was increased.

Lanterns were allowed to soak in DCM, and the DCM was analyzed by LC/MS. A 150 μL aliquot of the DCM wash solution was placed in an MS vial and allowed to stand in a fumehood so that the DCM would evaporate. The solvent was replaced with neat acetonitrile. Samples were injected (10 μL loop) and if the Lanterns were washed properly, the UV trace of the chromatogram should show no peaks of significance. The presence of a sharp peak was an indication that some non-solid, bound impurities may remain on the surface of the Lantern. Further washing with DMF and DMSO was used to remove the impurities.

Step 7. BAL Cleavage Reaction

The BAL linker amide products are cleaved with freshly prepared mixed TFA and DCM (1:4) cleavage reagent solution. The Lanterns to be cleaved were placed in glass vials and sufficient cleavage solution was added to cover the Lanterns. The vials were capped and allowed to stand for 1 h at room temperature. The Lanterns were removed, washed with methanol, and discarded. The reaction solutions were evaporated under a stream of nitrogen or placed in a centrifugal evaporator certified to handle TFA vapor. The residues were dissolved in neat acetonitrile and samples were then transferred into pre-weighed 10 mL plastic tubes or equivalent and frozen in liquid nitrogen. The samples were lyophilized by freeze-drying.

These and other methods known in the art can be used to synthesize the compounds of the instant disclosure. Those of ordinary skill in the art will appreciate that many of the amide-based antiviral compounds of the instant disclosure may exhibit the phenomena of tautomerism, conformational isomerism, geometric isomerism or optical isomerism. As the formula drawings within the specification and claims can represent only one of the possible tautomeric, conformational isomeric, optical isomeric or geometric isomeric forms, it should be understood that the invention encompasses any tautomeric, conformational isomeric, optical isomeric or geometric isomeric forms of the compounds having one or more of the utilities described herein, as well as mixtures of these various different forms. In addition, although the exact optical configurations of the chiral centers of the various illustrated amide-based antiviral compounds are not all specified, it is to be understood that the structural illustrations are intended to be a short-hand for describing these compounds, and are not intended to be limiting.

Antiviral Activity

The non-nucleoside, amide-based compounds of structure (I)-(X) can be tested for antiviral activity in various assays, including, for example, enzyme- and cell-based assays. An exemplary assay involves measuring antiviral activity against hepatitis C virus (HCV) in an RNA-dependent RNA polymerase (RdRp) assay, as described herein. The IC₅₀ range of the antiviral compounds of this disclosure varies from 1-300 μM (see Table 1). In certain embodiments, the antiviral compounds of structure (I)-(X) inhibit or functionally alter a viral polymerase. In one embodiment, the viral polymerase inhibited or functionally altered is an RdRp. In another embodiment, the viral polymerase inhibited or functionally altered is a Hepacivirus polymerase, such as an HCV polymerase.

The antiviral compounds of structure (I)-(X) were analyzed using an RdRp assay as described herein, and activities were verified using in vitro screening assays as described herein or as known in the art. In certain embodiments, the present disclosure provides methods for identifying amide-based antiviral compounds and methods for diagnosing the presence of a viral infection, such as an HCV infection.

Replication of hepatitis C virus (HCV) is catalyzed by the NS5B RNA-dependent RNA polymerase (RdRp). This enzyme is a common target for drug discovery in non-cell based assays due to its essential role in the viral life cycle. A current challenge for screening potential RdRp inhibitors is the difficulty in implementing a medium to high throughput assay because most of the widely used methods are based on the incorporation of radio-labelled nucleotides and use of radioisotopes requires specialized facilities. Although several non-cell based methods using alternative non-radioactive isotopic detection have been published (Lahser and Malcolm, Analytical Biochem. 325: 247, 2004; Park et al., J. Virological Methods 101(1-2): 211, 2002), there is a lack of described protocols that are optimized and standardized for evaluation of RdRp inhibition. The method of altering RdRp activity disclosed herein has the advantage of using non-radioactive detection, being optimized as a simple end-point assay, and providing flexibility to modify assay conditions because the reaction occurs in solution. This flexibility is particularly useful for the determination of kinetic parameters and for mechanism of action studies.

In certain embodiments, the instant disclosure provides a method for identifying an inhibitor of RNA-dependent RNA polymerase (RdRp) activity comprising (1) contacting an RdRp with a template-primer and non-radioactively labelled nucleotide triphosphate substrate(s), in the presence or absence of a target antiviral compound, (2) detecting incorporation of the non-radioactively labelled nucleotides into a nucleic acid molecule product, and (3) comparing the amount of labelled nucleic acid molecule product produced in the presence and absence of the target antiviral compound, wherein a decrease in labelled nucleic acid molecule product is indicative of an inhibitor of RdRp activity (see Example 404).

For example, test (target) antiviral compounds of structure (I)-(X) can be dissolved in DMSO or another appropriate solvent, diluted to the desired concentration and transferred to a microtiter plate already having a reaction mix. A reaction mix can be comprised of a template-primer substrate, such as a Poly A-Oligo d(T)20-biotin, and nucleotide triphosphate substrates, such as DIG-11-UTP and UTP, at a desired concentration. The reaction mix may also contain buffer components and RNase inhibitors. The RdRp can be a recombinant HCV polymerase, which is used to start the reaction. After allowing the reaction to proceed (e.g., incubate at 30° C.) for a pre-determined time, the reaction is stopped by addition of a chelating agent, such as EDTA. The reaction mix can then be transferred to streptavidin coated well of a microtiter plate containing buffer for binding of the biotinylated product to the plates.

To quantify the DIG-11-UTP incorporated in the product, a labelled anti-DIG antibody or antibody fragment, such as anti-DIG POD (peroxidase) Fab fragments, and an appropriate colorimetric substrate, such as BM Blue POD substrate, can be used. After development, the colorimetric reaction is stopped by adding acid and then measuring absorbance at 450 nm. As an alternative to DIG-11-UTP, other labelled nucleotide triphosphates can be used, such as Dinitrophenyl-11-dUTP (DNP-11-dUTP) with a corresponding antibody and development system. Modifications of the assay may include replacing DIG-11-UTP with a fluorescently labelled nucleotide, such as UTP conjugated with Oregon Green 488, Rhodamine Green, Texas Red, Coumarin, Cyanine or Fluorescein. Use of a fluorescently labeled nucleotide allows for real-time and continuous measuring of substrate incorporation, which facilitates performing kinetic and mechanism of action studies.

HCV is difficult to propagate efficiently in cell culture, thus rendering analysis and identification of potential anti-HCV agents difficult. In the absence of a suitable cell culture system capable of supporting replication of human HCV and re-infection of cells in vitro, use of another member of the Flaviviridae family, bovine viral diarrhea virus (BVDV), as an art-accepted surrogate virus for use in cell culture models (Buckwold et al., Antiviral Res. 60: 1, 2003; Stuyver et al., Antimicrob. Agents Chemother. 47:244, 2003; Whitby et al., supra) can be used to identify anti-HCV compounds (see Example 405). HCV and BVDV share a significant degree of local protein homology, a common replication strategy, and probably the same subcellular location for viral envelopment. Both HCV and BVDV have single-stranded genomes (approximately 9,600 and 12,600 nucleotides, respectively) that encode nine functionally analogous gene products, including the E1 and E2 envelope glycoproteins (see, e.g., Rice, Flaviviridae: The Viruses and Their Replication, in Fields Virology, 3rd Ed. Philadelphia, Lippincott, 931, 1996). Other assays well-known in the art include HCV pseudoparticles (see, e.g., Bartosch et al., J. Exp. Med. 197:633, 2003; Hsu et al., Proc. Nat'l Acad. Sci. USA 100:7271, 2003) and HCV replicons of any type, such as fall length replicons, expressing E1 and E2, and also resistant to IFN-α or ribavirin (see, e.g., U.S. Pat. Nos. 5,372,928; 5,698,446; 5,874,565; 6,750,009).

The compounds described herein may be useful research tools for in vitro and cell-based assays to study the biological mechanisms of viral infection, growth, and replication, and to identify other antiviral compounds. In one embodiment, a method is provided for identifying anti-viral compounds, comprising contacting a host cell infected with a virus with a candidate antiviral compound, such as an antiviral compound of structure (I)-(X), for a time sufficient to inhibit viral replication, and identifying a candidate antiviral compound that inhibits (prevents, slows, abrogates, interferes with) infection, viral replication, and/or viral assembly. In certain embodiments, the methods described herein may be used to identify a test compound that acts synergistically when combined with another antiviral agent (e.g., interferon, ribavirin, castanospermine, celgosivir or any combination thereof). In another embodiment, a method is provided for identifying cells suspected of having a viral infection, comprising contacting a host cell suspected of being infected with a virus with an antiviral compound of structure (I)-(X) under conditions and for a time sufficient to inhibit infection, viral replication, or viral assembly, and identifying cells infected with a virus. In certain embodiments, the viral infection may be caused by or associated with a Hepacivirus, such as HCV. The assays described herein may be used to determine the therapeutic value of a candidate compound or combination, may be used for diagnostic purposes (e.g., detect the presence of a viral infection), and may be useful for determining dosage parameters that would be useful in treating a subject in need thereof.

Compositions and Therapeutic Uses

The present disclosure provides amide-based antiviral compounds and compositions thereof. In addition, the present disclosure provides methods for using such compounds or compositions in reducing or inhibiting the activity of a viral polymerase in a host. The reduction or inhibition of viral polymerase activity may be accomplished by administering a therapeutically effective amount of an amide-based compound having any of the structural forms described herein, or composition thereof, such that a viral infection is treated or prevented.

Pharmaceutical compositions comprising antiviral compounds of structure (I)-(X) may be manufactured by means of conventional mixing, dissolving, granulating, dragee making, levigating, emulsifying, encapsulating, entrapping or lyophilizing processes. Pharmaceutical compositions may be formulated in conventional manner using one or more physiologically acceptable carriers, diluents, excipients or auxiliaries that facilitate formulating active antiviral compounds of structure (I)-(X) into preparations that can be used pharmaceutically. A single antiviral compound of structure (I)-(X), a plurality of antiviral compounds of structure (I)-(X), or antiviral compounds of structure (I)-(X) in combination with one or more biologically active agents (e.g., other antivirals, antibacterials, antifingals, etc.) may be formulated with a pharmaceutically acceptable carrier, diluent or excipient to generate pharmaceutical compositions of the instant disclosure. The combination therapies may be conveniently formulated together or separately in pharmaceutical formulations comprising a combination as defined herein together with a pharmaceutically acceptable carrier or carriers. The individual components of the combinations above may be administered either simultaneously or sequentially, either in separate or combined pharmaceutical formulations, each in similar or different dosage forms, each by similar or different dosage schedules as appropriately determined by those skilled in the art.

For example, an antiviral compound of structure (I)-(X) may be used in combination with one or more other adjunctive therapies, such as other antiviral treatments. In one aspect of the disclosure, the antiviral compounds of structure (I)-(X) may be utilized with one or more of a polymerase inhibitor, a helicase inhibitor, a protease inhibitor, an α-glucosidase inhibitor, an inhibitor of the IRES, an inhibitor of any other non-structural HCV protein activity, a compound that binds to a structural or non-structural protein abrogating, complementing or affecting its activity; a compound that alters immune function such as interferon (including α-interferon, (β-interferon, γ-interferon, and derivatives thereof), and a nucleoside analog (such as ribavirin or derivatives thereof).

Exemplary glucosidase inhibitors include castanospermine and derivatives thereof (e.g., esters of castanospermine such as celgosivir) and certain imino sugars, such as deoxynojirimycin (DNJ), which are inhibitors of ER α-glucosidases that potently inhibit the early stages of glycoprotein processing (see, e.g., Ruprecht et al., J. Acquir. Immune Defic. Syndr. 2:149, 1989; see also, e.g., Whitby et al., Antiviral Chem. Chemother. 15:141, 2004; Branzα-Nichita et al., J. Virol. 75:3527, 2001; Courageot et al., J. Virol. 75:564, 2000; Choukhi et al., J. Virol. 72:3851, 1998; WO 99/29321; WO 02/089780).

Another exemplary adjunctive agent or compound is one that inhibits the binding to or infection of cells by a Hepacivirus, such as HCV. Examples of such compounds include antibodies that specifically bind to one or more HCV gene products (e.g., E1 or E2 proteins) or to a cell receptor to which the HCV binds. The antibody may be a monoclonal or polyclonal antibody, or antigen binding fragments thereof, including genetically engineered chimeric, humanized, sFv, or other such immunoglobulins. Other compounds that prevent binding or infection of cells by a virus include glucosaminoglycans (such as heparan sulfate and suramin).

Another exemplary adjunctive agent or compound is one that inhibits the release of viral RNA from the viral capsid or inhibits the function of HCV gene products, including inhibitors of the internal ribosome entry site (IRES), protease inhibitors (e.g., serine protease inhibitors), helicase inhibitors, and inhibitors of the viral polymerase/replicase (see, e.g., Olsen et al., Antimicrob. Agents Chemother. 48:3944, 2004; Stansfield et al., Bioorg. Med. Chem. Lett. 14:5085, 2004). Inhibitors of IRES include, for example, nucleotide sequence specific antisense (see, e.g., McCaffrey et al., Hepatology 38:503, 2003); small yeast RNA (see, e.g., Liang et al., World J. Gastroenterol. 9:1008, 2003); or short interfering RNA molecules (siRNA) that inhibit translation of mRNA; and cyanocobalamin (CNCbl, vitamin B12) (Takyar et al., J. Mol. Biol. 319:1, 2002). NS3 serine protease (helicase) inhibitors include peptides that are derived from NS3 substrates and act to block enzyme activity. Exemplary serine protease inhibitors include BILN 2061 (see, e.g., Lamarre et al., Nature 426:186, 2003) (Boehringer Ingelheim (Canada) Ltd., Quebec), HCV-796 (Wyeth/Viropharma), SCH-503034 (Schering-Plough), ITMN-A (or ITMN-B) (Intermune), and VX-950 (Vertex Pharmaceuticals, Inc. Cambridge, Mass.).

Still another exemplary adjunctive agent or compound is one that perturbs cellular functions involved in or that influence viral replication, including inhibitors of RNA-dependent RNA polymerase (RdRp) and nucleoside analogs. Exemplary nucleoside inhibitors may be inhibitors of inosine monophosphate dehydrogenase (e.g., ribavirin, mycophenolic acid, and VX497 (merimepodib, Vertex Pharmaceuticals)), or nucleoside analogues may be 2′-C-methyl cytidine (NM107, Idenix Pharmaceuticals), valopicitabine (NM283, the valine ester prodrug of NM107; Idenix Pharmaceuticals) or the like. NM107 is an active species in cell-based assays and can be delivered to a subject (e.g., humans) as the prodrug NM283. NM107 may be active as is or may be active as a further activated metabolite. Other antiviral compounds can be used as well, such as broad spectrum compounds including amantadine, (Symmetrel®, Endo Pharamceuticals), rimantadine (Flumadine®, Forest Pharmaceuticals, Inc.). In certain embodiments, the antiviral compounds of structure (I)-(X) are combined with ribavirin, 2′-C-methyl cytidine, or valopicitabine.

Antiviral compounds of structure (I)-(X) may be further optionally combined with an adjunctive agent or compound that modulates (preferably decreases or reduces the severity or intensity of, reduces the number of, or abrogates) the symptoms and effects of Hepacivirus infection, such as an HCV infection. Exemplary compounds that modulate symptoms of Hepacivirus infection include antioxidants such as the flavinoids.

An adjunctive therapeutic may comprise another antiviral compound, for example, an anti-viral compound or drug that is used for treatment of an infectious agent frequently identified as co-infecting a subject who is infected with a flavivirus, such as HCV. Such a co-infection may be by HBV, a human retrovirus such as HIV 1 and 2, or human T-cell lymphotrophic virus (HTLV) type 1 or type 2. Examples of anti-viral compounds include nucleotide reverse transcriptase (RT) inhibitors (e.g., Lamivudine (3TC), zidovudine, stavudine, didanosine, adefovir dipivoxil, and abacavir); non-nucleoside RT inhibitors (e.g., nevirapine); and protease inhibitors (e.g., saquinavir, indinavir, and ritonavir).

Yet another adjunctive agent or compound is one that acts to alter immune function (increase or decrease in a statistically significant, clinically significant, or biologically significant manner), preferably to enhance or stimulate an immune function or an immune response against a Hepacivirus infection, such as an HCV infection. For example, a compound may stimulate a T cell response or enhance a specific immune response (e.g., thymosin-α, and interferons such as α-interferons and β-interferons), or may stimulate or enhance a humoral response. Examples of compounds that alter an immune function include type I interferons, such as interferon-α (see, e.g., Nagata et al., Nature 287:401, 1980), interferon-β (see, e.g., Tanigushi et al., Nature 285:547, 1980), and interferon-ω (Adolf, J. Gen. Virol. 68:1669, 1987).

The combination of an interferon-α with ribavirin for treating an HCV infection has been superior to either treatment alone, and the combination is the current standard of care. The effectiveness, doses, and frequency of administration were studied in three large double-blind, placebo-controlled clinical trials (Reichard et al., Lancet 351:83, 1998; Poynard et al., Lancet 352:1426, 1998; McHutchison et al., New Engl. J. Med. 339:1485, 1998; see also Buckwold et al., Antimicrob. Agents Chemother. 47:2293, 2003; Buckhold, J. Antimicrob. Chemother. 53:412, 2004), although adverse effects are associated with this treatment regime. In certain embodiments, the antiviral compounds of structure (I)-(X) are combined with interferon and ribavirin, or interferon and 2′-C-methyl cytidine, or interferon and valopicitabine.

In certain embodiments, an antiviral compound of structure (I)-(X) is administered in combination with an interferon, such as interferon-α. Interferon-α has been used in the treatment of a variety of viral infections, either as a monotherapy or as a combination therapy (see, e.g., Liang, New Engl. J. Med. 339:1549, 1998; Hulton et al., J. Acquir. Immune Defic. Syndr. 5:1084, 1992; Johnson et al., J. Infect. Dis. 161:1059, 1990). Interferon-α binds to cell surface receptors and stimulates signal transduction pathways that lead to activation of cellular enzymes (e.g., double-stranded RNA-activated protein kinase and RNase L that inhibit translation initiation and degrade viral RNA, respectively) that repress virus replication (see, e.g., Samuel, Clin. Microbiol. Rev. 14:778, 2001; Kaufman, Proc. Natl. Acad. Sci. USA 96:11693, 1999). HCV E2 glycoprotein and NS5a may block RNA-activated protein kinase activity such that some HCV strains are more resistant to interferon-α; thus, combination therapies of interferon-α and one or more other compounds may be necessary for treatment of persistent viral infection (see, e.g., Ouzounov et al., supra, and references cited therein). In some embodiments, a polyethylene glycol moiety is linked to interferon-α (known as pegylated interferon-α; peginterferon α-2b (Peg-Intron; Schering-Plough) and peginterferon α-2a (Pegasys®; Hoffmann-La Roche)), which may have an improved pharmacokinetic profile and may also manifest fewer undesirable side effects (see, e.g., Zeuzem et al., New Engl. J. Med. 343:1666, 2000; Heathcote et al., New Engl. J. Med. 343:1673, 2000; Matthews et al., Clin. Ther. 26:991, 2004).

Interferon-α-2a (Roferon®-A; Hoffman-La Roche), Interferon-α-2b (Intron-A; Schering-Plough), and interferon-α-con-1 (Infergen®; Intermune) are approved for use as single agents in the U.S. for treatment of adults with chronic hepatitis C. The recommended dose of interferons-α-2b and -α-2a for the treatment of chronic hepatitis C is 3,000,000 units three times a week, administered by subcutaneous or intramuscular injection. Treatment is administered for six months to two years. For interferon-α-con-1, the recommended dose is 9 μg three times a week for first time treatment and 15 μg three times a week for another six months for patients who do not respond or relapse. During the treatment periods with any of these recombinant interferons, the patient must be monitored for side effects, which include flu-like symptoms, depression, rashes, and abnormal blood counts. Treatment with interferon alone leads to a sustained response in less than 15% of subjects. Due to this low response rate, these interferons are rarely used as a monotherapy for the treatment of patients with chronic hepatitis C.

Pharmaceutically acceptable carriers, diluents or excipients for therapeutic use are well known in the pharmaceutical art, and are described herein and, for example, in Remington's Pharmaceutical Sciences, Mack Publishing Co. (A. R. Gennaro, ed., 18^{th} Edition, 1990) and in CRC Handbook of Food, Drug, and Cosmetic Excipients, CRC Press LLC (S. C. Smolinski, ed., 1992). In certain embodiments, antiviral compounds of structure (I) may be formulated with a pharmaceutically or physiologically acceptable carrier, diluent or excipient is aqueous, such as water or a mannitol solution (e.g., about 1% to about 20%), hydrophobic carrier (e.g., oil or lipid), or a combination thereof (e.g., oil and water emulsions). In certain embodiments, any of the pharmaceutical compositions described herein are sterile.

The formulations of the present invention, having an amount of one or more antiviral compounds of structure (I), with or without other adjunctive therapies, sufficient to treat or prevent an infection are, for example, suitable for topical (e.g., creams, ointments, skin patches, eye drops, ear drops, shampoos) application or administration. Other exemplary routes of administration include oral, parenteral, sublingual, bladder wash-out, vaginal, rectal, enteric, suppository, nasal, or inhalation. The term parenteral, as used herein, includes subcutaneous, intravenous, intramuscular, intraarterial, intraabdominal, intraperitoneal, intraarticular, intraocular or retrobulbar, intraaural, intrathecal, intracavitary, intracelial, intraspinal, intrapulmonary or transpulmonary, intrasynovial, and intraurethral injection or infusion techniques. The pharmaceutical compositions of the present disclosure are formulated so as to allow the antiviral compounds of structure (I) contained therein to be bioavailable upon administration of the composition to a subject. The level of antiviral compound in serum and other tissues after administration can be monitored by various well-established techniques, such as chromatographic- or antibody-based (e.g., ELISA) assays. In certain embodiments, antiviral compounds of structure (I) are formulated for topical application to a target site on a subject in need thereof, such as an animal or a human. In other embodiments, antimicrobial lipopeptides derivatives are formulated for parenteral administration to a subject in need thereof (e.g., having a Hepacivirus infection, such as an HCV infection), such as an animal or a human.

Proper formulation is generally dependent upon the route of administration chosen, as is known in the art. For example, in exemplary embodiments for topical administration, the antiviral compounds of structure (I) may be formulated as solutions, gels, ointments, creams, suspensions, pastes, and the like. Systemic formulations are another embodiment, which includes those designed for administration by injection, e.g. subcutaneous, intravenous, intramuscular, intrathecal or intraperitoneal injection, as well as those designed for transdermal, transmucosal, oral, intranasal, or pulmonary administration. In one embodiment, the systemic formulation is sterile. In embodiments for injection, the antiviral compounds of structure (I) may be formulated in aqueous solutions, preferably in physiologically compatible solutions or buffers such as Hank's solution, Ringer's solution, mannitol solutions or physiological saline buffer. In certain embodiments, any of the compositions described herein may contain formulatory agents, such as suspending, stabilizing or dispersing agents. Alternatively, the antiviral compounds of structure (I) may be in solid (e.g., powder) form for constitution with a suitable vehicle (e.g., sterile pyrogen-free water) before use. In embodiments for transmucosal administration, penetrants, solubilizers or emollients appropriate to the barrier to be permeated may be used in the formulation. For example, 1-dodecylhexahydro-2H-azepin-2-one (Azone®), oleic acid, propylene glycol, menthol, diethyleneglycol ethoxyglycol monoethyl ether (Transcutol®), polysorbate polyethylenesorbitan monolaurate (Tween®-20), and the drug 7-chloro-1-methyl-5-phenyl-3H-1,4-benzodiazepin-2-one (Diazepam), isopropyl myristate, and other such penetrants, solubilizers or emollients generally known in the art may be used in any of the compositions of the instant disclosure.

In other embodiments, the antiviral compounds of structure (I) can be formulated with a pharmaceutically acceptable carrier in the form of tablets, pills, dragees, capsules, liquids, gels, syrups, slurries, suspensions and the like, for oral ingestion by a subject or patient to be treated. In certain embodiments for oral solid formulations, such as powders, capsules or tablets, suitable excipients include fillers, such as sugars (e.g., lactose, sucrose, mannitol, sorbitol); cellulose preparations such as maize starch, wheat starch, rice starch, potato starch, gelatin, gum tragacanth, methyl cellulose, hydroxypropylmethyl-cellulose, sodium carboxymethylcellulose, or polyvinylpyrrolidone (PVP); granulating agents; or binding agents. Optionally, disintegrating agents may be added, such as cross-linked polyvinylpyrrolidone, agar, or alginic acid (or a salt thereof, such as sodium alginate). If desired, solid dosage forms may be sugar-coated or enteric-coated using standard techniques. In some embodiments for oral liquid preparations, such as suspensions, elixirs or solutions, suitable carriers, excipients or diluents include water, glycols, oils, alcohols, or combinations thereof. Additionally, flavoring agents, preservatives, viscosity-increasing agents, humectants, coloring agents, or the like, may be added. In embodiments for oral or buccal administration, the compositions may take the form of, for example, tablets or lozenges, formulated as is known in the art and described herein.

In embodiments for administration by inhalation, the compounds for use according to the present disclosure may be formulated for convenient delivery in the form of drops for intranasal administration, or in the form of an aerosol spray from pressurized packs or nebulizer having a suitable propellant (e.g., dichlorodifluoromethane, trichlorofluoromethane, dichlorotetrafluoroethane, carbon dioxide or other suitable gas). In certain embodiments, the drops or aerosol composition is sterile. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base, such as lactose or starch.

In other embodiments, the antiviral compounds of structure (I) may be formulated into rectal or vaginal compositions such as suppositories or retention enemas, e.g., containing conventional suppository bases, such as cocoa butter or other glycerides.

In addition to the formulations described herein, the antiviral compounds may also be formulated as a depot preparation. For example, antiviral compounds of structure (I) can be in the form of the slow-release formulation such that they can provide activity over time. Such long-acting formulations may be administered by implantation (for example, subcutaneously or intramuscularly) or by intramuscular injection. In certain embodiments, the compounds may be formulated with suitable a polymer (including poly(lactides), poly(glycolides), poly(caprolactones), and blends thereof), a hydrophobic material, (including a physiologically acceptable oil, which can be in the form of an emulsion), an ion exchange resin, or as sparingly soluble derivatives (such as a sparingly soluble salt).

Alternatively, other pharmaceutical delivery systems may be employed. In certain embodiments, the compounds are formulated with liposomes or emulsions as delivery vehicles. Certain organic solvents, such as dimethylsulfoxide (DMSO), may also be employed. Additionally, the antiviral compounds of structure (I) may be delivered using a sustained-release system, such as semipermeable matrices of solid or semi-solid polymers (e.g., thermopaste) containing the therapeutic agent. Sustained-release capsules may, depending on their chemical nature, release the compounds for a few hours, a few days, a few weeks, or for up to about 100 days.

As certain of the carboxyl groups of the antiviral compounds of structure (I) are acidic, or the substituents R¹, R², R³, R⁴, and R⁵ may include acidic or basic substituents, the antiviral compounds of structure (I) may be included in any of the above-described formulations as a free acid, a free base, or as a pharmaceutically acceptable salt. Pharmaceutically acceptable salts are those salts that substantially retain the antiviral activity of the free acid or base, and which are prepared by reaction with a base or acid, respectively. Suitable acids and bases are well known to those of ordinary skill in the art and are described herein. Exemplary pharmaceutical salts may tend to be more soluble in aqueous and other protic solvents than is the corresponding free base or acid form.

Antiviral compounds of structure (I) can be provided in dosage amounts and intervals, which can be adjusted on a case-by-case basis, to provide plasma levels of one or more of the antiviral compounds sufficient to maintain a therapeutic effect. Exemplary clinical dosages for administration by injection may range from about 0.1 to about 200 mg/kg/day, or range from about 1.5 to about 15 mg/kg/day. In certain embodiments, therapeutically effective serum levels may be achieved by administering a single dose or as a single daily dose or multiple doses each day over a specified time period. That is, the desired dose may be conveniently provided in divided doses administered at appropriate intervals, for example, two, three, four or more doses per day, or one dose per day, one dose per two days, etc. In other embodiments, therapeutically effective serum levels may also be achieved by administering at less frequent dosing schedules such as, for example, once every two days, twice a week, once a week or at longer intervals between dosing, or any combination thereof. For example, combination administration schedules may be utilized to reach therapeutically effective does, such as multiple does on one or more days followed by less frequent dosing such as, for example, once every two days, twice a week or once a week, or longer.

The antiviral compositions of this disclosure may be administered to a subject as a single dosage unit form (e.g., a tablet, capsule, injection or gel), or the compositions may be administered, as described herein, as a plurality of dosage unit forms (e.g., in aerosol or injectable form, tablet, capsule), or in any combination thereof For example, the antiviral formulations may be sterilized and packaged in single-use, plastic laminated pouches or plastic tubes of dimensions selected to provide for routine, measured dispensing. In one example, the container may have dimensions anticipated to dispense 0.5 mL of the antiviral composition (e.g., a drop, gel or injection form) to a subject, or to a limited area of a target surface on or in a subject, to treat or prevent an infection. A target surface, for example, may be in the immediate vicinity of a skin infection or an organ (e.g., liver), where the target surface area will depend on the extent of an infection.

In cases of local administration or selective uptake, the effective local concentration of antimicrobial lipopeptide derivatives may not be related to plasma concentration. A person having ordinary skill in the art will be able to optimize therapeutically effective local dosages without undue experimentation. The amount of an active antiviral compound of structure (I)-(X) administered will be dependent upon, among other factors, the subject being treated, the subject's weight, the severity of the affliction, the manner of administration and the judgment of the prescribing physician.

The antiviral compositions may be provided in various forms, depending on the amount and number of different pharmaceutically acceptable excipients present. For example, the lipopeptide compositions may be in the form of a solid, a semi-solid, a liquid, a lotion, a cream, an ointment, a cement, a paste, a gel, or an aerosol. In one embodiment, the antiviral formulation is in the form of a liquid or a gel. The pharmaceutically acceptable excipients suitable for use in the antiviral formulation compositions as described herein may optionally include, for example, a viscosity-increasing agent, a buffering agent, a solvent, a humectant, a preservative, a chelating agent (e.g., EDTA or EGTA), an oleaginous compound, an emollient, an antioxidant, an adjuvant, or the like. Exemplary buffering agents suitable for use with the antiviral compounds of structure (I) or compositions thereof include monocarboxylate or dicarboxylate compounds (such as acetate, fumarate, lactate, malonate, succinate, or tartrate). Exemplary preservatives include benzoic acid, benzyl alcohol, phenoxyethanol, methylparaben, propylparaben, and the like. The function of each of these excipients is not mutually exclusive within the context of the present invention. For example, glycerin may be used as a solvent or as a humectant or as a viscosity-increasing agent.

The present disclosure provides a method for treating or preventing a viral infection, such as a Hepacivirus infection, in a host comprising administering a therapeutically effective amount of an antiviral compound of structure (I)-(X). In one embodiment, the Hepacivirus infection being treated or prevented is an HCV infection. The antiviral therapy may be repeated intermittently while infections are detectable or even when they are not detectable.

Treatment, as provided by the present disclosure, encompasses prophylaxis or preventative administration of any combination described herein. For example, effective treatment of a Hepacivirus infection may include a cure of the infection (i.e., eradication of the virus from the host or host tissue); a sustained response in which viral RNA is no longer detectable in the blood of the subject six months after completing a therapeutic regimen (such a sustained response may be equated with a favorable prognosis and may be equivalent to a cure); slowing or reducing any associated tissue damage (e.g., HCV have associated liver scarring (fibrosis)); slowing or reducing production of virus; reducing, alleviating, or abrogating symptoms in a subject; or preventing symptoms or infection from worsening or progressing. For example, if the infections is caused by or associated with HCV, the compositions described herein may be used for accomplishing at least one of the following goals: (1) elimination of infectivity and potential transmission of a an HCV infection to another subject; (2) arresting the progression of liver disease and improving clinical prognosis; (3) preventing development of cirrhosis and HCC; (4) improving the clinical benefit of currently used therapeutic molecules or modalities; and (5) improving the host immune response to HCV infection. To date, a therapeutic agent that adequately treats or prevents an HCV infection and any associated disease without severe side-effects has remained elusive.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. The invention having been described, the following examples are intended to illustrate, and not limit, the invention.

### EXAMPLES

A number of non-nucleoside derivatives were prepared according to Scheme (I) (in solution; see, e.g., Examples 2-325) or Scheme (II) (in solid phase; see, e.g., Examples 325-403) from commercially available starting materials or starting materials prepared by conventional synthetic or biosynthetic methods. Where needed, alternative methods of preparation are further described in specific examples.

Sample preparation for purification by high-pressure liquid chromatography (HPLC) involved diluting a sample with 2 ml of 0.2% trifluoroacetic acid (TFA) in acetonitrile and 2 mL H₂O, and then filtering with Pall GHP Acrodisc® GF 25 mm Syringe Filter with a GF/0.45um GHP Membrane. The HPLC system used was a BioCAD® Sprint™ Perfusion Chromatography®, with UV wavelengths of 220 nm and 280 nm used and a flow rate of 15mL/min. Solvent A is 0.1 % TFA in mQ water, and Solvent B is 0.1 % TFA in Acetonitrile (HPLC Grade). The column used was a Waters C18 (or C8) Symmetry Prep 7 um, 19×150 mm. The general liquid chromatography (LC) method used was a gradient protocol: 80% solvent A to 30% solvent A over 9 column volumes. Fractions containing a desired compound were combined, organic solvents removed in vacuo, with the remaining aqueous layer being frozen and lyophilized to obtain an amphorous solid that was generally expected to be the TFA salt of a desired product.

### Example 1

### General Compound Synthesis in Solution

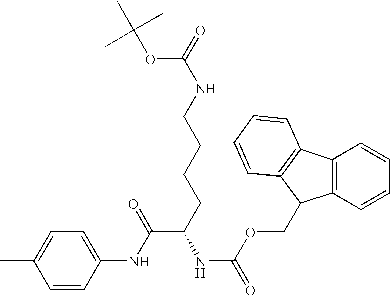 (a) Synthesis of (S)-(5-tert-Butoxycarbonylamino-1-p-tolylcarbamoyl-pentyl)-carbamic acid 9H-fluoren-9-ylmethyl esterr (Intermediate #1): A solution of (S)-6-tert-Butoxycarbonylamino-2-(9H-fluoren-9-ylmethoxycarbonylamino)-hexanoic acid (3.0 g, 6.4 mmol), p-tolylamine (0.75 g, 7.1 mmol), and 2.7 g (8.3 mmol) of 2-(1H-benzotriazole-1-yl)-1,1,3,3-tetramethyluronium tetrafluoroborate in 150 mL of dimethylformamide was stirred at room temperature under inert atmosphere (nitrogen). Diisopropylethylamine (4.5 mL, 25.6 mmol) was added to the mixture and the reaction was stirred until deemed complete by thin layer chromatography (approximately 3 h). 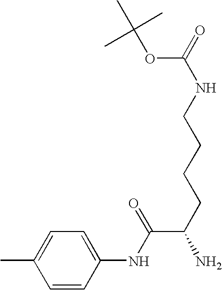
(b) Deprotection of 9H-fluoren-9-ylmethyl ester (Fmoc) protecting group to produce (S)-(5-Amino-5-p-tolylcarbamoyl-pentyl)-carbamic acid tert-butyl ester (Intermediate #2):

Fmoc deprotection of Intermediate #1 was accomplished by charging the crude reaction mixture with excess piperdine (20 mL, typically 5 equivalents or more), then allowing the mixture to stir for 30 minutes. Upon completion, the mixture was concentrated in vacuo then purified by flash chromatography using a gradient solvent system of 25%, 50%, then 75% ethyl acetate in hexanes to afford the pure free amine in 93% yield for the two steps: [M+H]+ calcd for C₁₈H₃₀N₃O₃, 336; found 336. 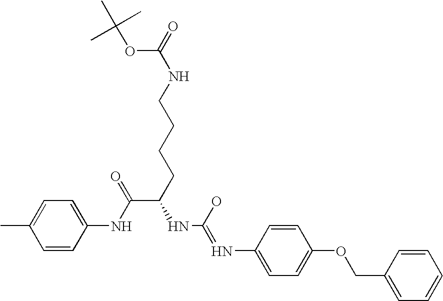
(c) Synthesis of (S)-{5-[3-(4-Benzyloxy-phenyl)-ureido]-5-p-tolylcarbamoyl-pentyl}-carbamic acid tert-butyl ester (Intermediate #3): To a solution of Intermediate #2 (40 mg, 0.12 mmol) in 2 mL of dichloromethane was added to 1-benzyloxy-4-isocyanato-benzene (32 mg, 0.14 mmol), and then stirred at room temperature under inert atmosphere (nitrogen) until deemed complete by TLC. Concentration in vacuo produced the crude Intermediate #3.
Scheme (I) Compounds

### Example 2

### (S)-6-AMINO-2-[3-(4-BENZYLOXY-PHENYL)-UREIDO]-HEXANOIC ACID P-TOLYLAMIDE

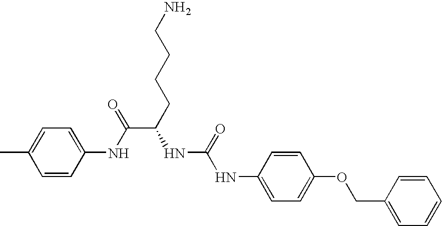

A solution of Intermediate #3 of Example 1 (0.12 mmol) in tetrahydrofuran (0.1M solution) was charged with trifluoroacetic acid (0.05M), and the mixture was stirred for 30 minutes. Upon completion, based on TLC, the mixture was reconcentrated in vacuo to produce a crude oily substance that was purified by HPLC to produce the title compound (16 mg, 28% yield for the two steps): 1H NMR (400 MHz, DMSO-d₆) δ 10.07 (s, 1H), 8.55 (s, 1H), 7.67 (br s, 3H), 7.49 (d, J=8.4 Hz, 2H), 7.42 (d, J=7.2 Hz, 2H), 7.38 (t, J=7.5 Hz, 2H), 7.31 (t, J=7.2 Hz, 1H), 7.28 (d, J=9.0 Hz, 2H), 7.11 (d, J=8.5 Hz, 2H), 6.89 (d, J=9.0 Hz, 2H), 6.44 (d, J=8.3 Hz, 1H), 5.03 (s, 2H), 4.37 (dt, J=5.7, 8.1 Hz, 1H), 2.81-2.75 (m, 2H), 2.25 (s, 3H), 1.76-1.69 (m, 1H), 1.63-1.52 (m, 3H), 1.44-1.3 (m, 2H); Low resolution mass spectrum (ES) m/e 461 [(M+1)+, calcd for C₂₇H₃₃N₄O₃: 461]; 99.7% purity based on HPLC.

### Example 3

### N-[1-(4-{[CYCLOHEXYL-(FURAN-2-CARBONYL)-AMINO]-METHYL}-PHENYLCARBAMOYL)-1-METHYL-ETHYL]-TEREPHTHALAMIC ACID METHYL ESTER

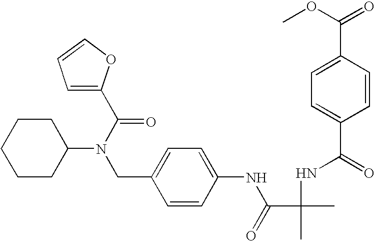

Furan-2-carboxylic acid (4-amino-benzyl)-cyclohexyl-amide, and 2-(9H-Fluoren-9-ylmethoxycarbonylamino)-2-methyl-propionic acid were coupled using the method described for Intermediates #1 and #2 of Example 1. Coupling with terephthalic acid monomethyl ester was achieved as described for Intermediate #1 of Example 1. Purification by HPLC produced the title compound. 1H NMR (400 MHz, DMSO-d₆) δ 9.42 (s, 1H), 8.55 (s, 1H), 8.43 (s, 4H), 7.77 (br s, 1H), 7.49 (d, J=8 Hz, 2H), 7.14 (d, J=9 Hz, 2H), 6.86 (br s, 1H), 6.56 (br s, 1H), 4.62 (br s, 2H), 4.10 (br s, 1H), 3.87 (s, 3H), 2.68 (s, 4H), 1.69-1.42 (m, 13H), 1.24-0.96 (m, 3H); Low resolution mass spectrum (ES) m/e 546 [(M+H)+, calcd for C₃₁H₃₆N₃O₆: 546]; 98% purity based on HPLC.

### Example 4

### N-[1-(4-{[CYCLOHEXYL-(FURAN-2-CARBONYL)-AMINO]-METHYL}-PHENYLCARBAMOYL)-1-METHYL-ETHYL]-TEREPHTHALAMIC ACID

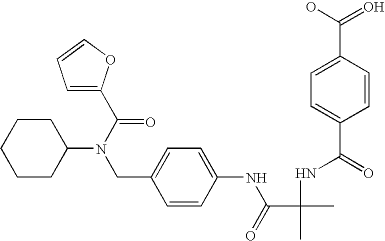

Standard hydrolysis (0.1 N sodium hydroxide in methanol for 12 hours) of the compound from Example 3, and purification by HPLC produced the title compound. 1H NMR (400 MHz, DMSO-d₆) δ 13.18 (s, 1H), 9.41 (s, 1H), 8.51 (s, 1H), 8.03-7.98 (m, 4H), 7.77 (br s, 1H), 7.49 (d, J=8 Hz, 2H), 7.14 (d, J=8 Hz, 2H), 6.86 (br s, 1H), 6.55 (br s, 1H), 4.62 (s, 2H), 4.10 (br s, 1H), 1.7-1.42 (m, 7H), 1.52 (m, 6H), 1.27-0.96 (m, 3H); Low resolution mass spectrum (ES) m/e 532 [(M+H)+, calcd for C₃₀H₃₄N₃O₆: 532]; 90.4% purity based on HPLC.

### Example 5

### (S)-[5-AMINO-5-(4-{[CYCLOHEXYL-(FURAN-2-CARBONYL)-AMINO]-METHYL}-PHENYLCARBAMOYL)-PENTYL]-CARBAMIC ACID ALLYL ESTER

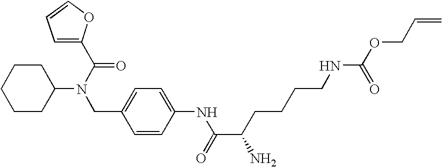

Furan-2-carboxylic acid (4-amino-benzyl)-cyclohexyl-amide was coupled to (S)-6-Allyloxycarbonylamino-2-(9H-fluoren-9-ylmethoxycarbonylamino)-hexanoic acid as described in the method for Intermediates #1 and #2 of Example 1. Purification by HPLC produced the title compound: 1H NMR (400 MHz, DMSO-d₆) δ 10.4 (s, 1H), 8.2 (d, J=3 Hz, 1H), 7.79 (br s, 1H), 7.51 (d, J=8 Hz, 2H), 7.24 (d, J=8 Hz, 2H), 7.15 (t, J=5 Hz, 1H), 6.89 (br s, 1H), 6.58 (br s, 1H), 5.86 (ddd, J=5, 11, 22 Hz, 1H), 5.23 (dd, J=2, 17 Hz, 1H), 5.13 (dd, J=1.4, 10 Hz, 1H), 4.64 (br s, 2H), 4.41 (d, J=5 Hz, 2H), 4.11 (t, J=11Hz, 1H), 3.87 (m, 2H), 2.96 (q, J=6 Hz, 2H), 1.81-1.63 (m, 6H), 1.55-1.17 (m, 9H), 1.08-0.98 (m, 1H); Low resolution mass spectrum (ES) m/e 511 [(M+H)+, calcd for C₂₈H₃₉N₄O₅: 511]; 100% purity based on HPLC.

### Example 6

### (S)-[5-AMINO-1-(4-{[CYCLOHEXYL-(FURAN-2-CARBONYL)-AMINO]-METHYL}-PHENYLCARBAMOYL)-PENTYL]-CARBAMIC ACID 9H-FLUOREN-9-YL METHYL ESTER

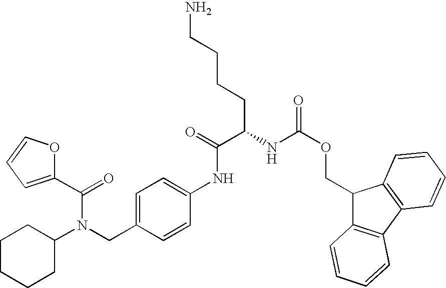

Furan-2-carboxylic acid (4-amino-benzyl)-cyclohexyl-amide was coupled to (S)-6-tert-butoxycarbonylamino-2-(9H-fluoren-9-ylmethoxycarbonylamino)-hexanoic acid as described in the method for Intermediate #1 of Example 1. Deprotection using the method described for Example 2 and purification by HPLC produced the title compound. 1H NMR (400 MHz, DMSO-d₆) δ 9.99 (s, 1H), 7.88 (d, J=8 Hz, 2H), 7.78 (br s, 1H), 7.72 (t, J=8 Hz, 2H), 7.64-7.59 (m, 4H), 7.52 (d, J=8 Hz, 2H), 7.40 (dt, J=3, 8 Hz, 2H), 7.31 (dd, J=7, 13 Hz, 2H), 7.19 (d, J=8 Hz, 2H), 6.88 (br s, 1H), 6.57 (br s, 1H), 4.63 (br s, 2H), 4.33-4.19 (m, 3H), 4.11 (dd, J=8, 14 Hz, 2H), 2.77 (qd, J=6, 12 Hz, 2H), 1.70-1.16 (m, 15H), 1.07-0.98 (m, 1H); Low resolution mass spectrum (ES) m/e 649[(M+H)+, calcd for C₃₉H₄₅N₄O₅: 649]; 98.8% purity based on HPLC.

### Example 7

### (S)-FURAN-2-CARBOXYLIC ACID CYCLOHEXYL-[4-(2,6-DIAMINO-HEXANOYLAMINO)-BENZYL]-AMIDE

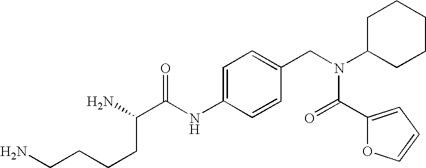

(S)-[5-Amino-1-(4-{[cyclohexyl-(furan-2-carbonyl)-amino]-methyl}-phenylcarbamoyl)-pentyl]-carbamic acid 9H-fluoren-9-ylmethyl ester from Example 6 was deprotected as described in the method for Intermediate #2 of Example 1. Purification by HPLC produced the title compound. 1H NMR (400 MHz, DMSO-d₆) δ 10.45 (s, 1H), 8.27 (d, J=3 Hz, 3H), 7.79 (s, 1H), 7.74 (s, 3H), 7.53 (d, J=8.5 Hz, 2H), 7.24 (d, J=8.5 Hz, 2H), 6.89 (s, 1H), 6.58 (s, 1H), 4.64 (s, 2H), 4.11 (br s, 1H), 3.91 (m, 1H), 2.76 (dd, J=6.5, 14 Hz, 1H), 1.81-1.76 (m, 2H), 1.71-1.63 (m, 4H), 1.58-1.45 (m, 5H), 1.40-1.33 (m, 2H), 1.27-1.17 (m, 2H), 1.08-0.98 (m, 1H); Low resolution mass spectrum (ES) m/e 427[(M+H)+, calcd for C₂₄H₃₅N₄O₃: 427]; 99.1% purity based on HPLC.

### Example 8

### (S)-FURAN-2-CARBOXYLIC ACID {4-[2-AMINO-3-(4-TERT-BUTOXY-PHENYL)-PROPIONYLAMINO]-BENZYL}-CYCLOHEXYL-AMIDE

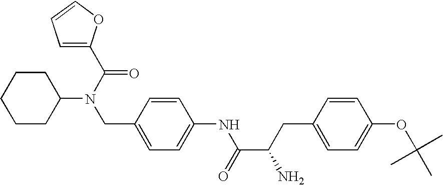

Furan-2-carboxylic acid (4-amino-benzyl)-cyclohexyl-amide was coupled to (S)-3-(4-tert-Butoxy-phenyl)-2-(9H-fluoren-9-ylmethoxycarbonylamino)-propionic acid as described in the method of Example 5. Purification by HPLC produced the title compound. 1H NMR (400 MHz, DMSO-d₆) δ 10.22 (s, 1H), 8.28 (br s, 3H), 7.79 (br s, 2H), 7.38 (d, J=8 Hz, 2H), 7.20 (d, J=8 Hz, 2H), 7.14 (d, J=8 Hz, 2H), 6.88 (d, J=8 Hz, 3H), 6.58 (br s, 1H), 4.63 (br s, 2H), 4.13-4.06 (m, 2H), 3.10-3.00 (m, 2H), 1.71-1.62 (m, 4H), 1.55-1.44 (m, 3H), 1.27-1.17 (m, 2H), 1.22 (s, 9H), 1.09-0.98 (m, 1H); Low resolution mass spectrum (ES) m/e 518[(M+H)+, calcd for C₃₁H₄₀N₃O₄: 518]; 98.6% purity based on HPLC.

### Example 9

### (S)-FURAN-2-CARBOXYLIC ACID {4-[2-AMINO-3-(4-METHOXY-PHENYL)-PROPIONYLAMINO]-BENZYL}-CYCLOHEXYL-AMIDE

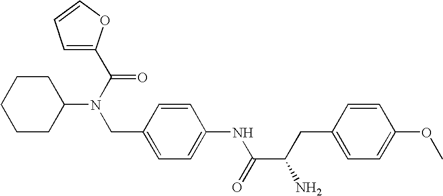

Furan-2-carboxylic acid (4-amino-benzyl)-cyclohexyl-amide was coupled to (S)-3-(4-methoxy-phenyl)-2-(9H-fluoren-9-ylmethoxycarbonylamino)-propionic acid as described in the method of Example 5. Purification by HPLC produced the title compound. 1H NMR (400 MHz, DMSO-d₆) δ 10.34 (s, 1H), 8.23 (br s, 3H), 7.79 (m, 1H), 7.45 (d, J=8 Hz, 2H), 7.23 (d, J=9 Hz, 2H), 7.16 (d, J=9 Hz, 2H), 6.88 (d, J=9 Hz, 3H), 6.58 (m, 1H), 4.64 (br s, 2H), 4.14-4.06 (m, 2H), 3.71 (s, 3H), 3.10 (dd, J=6, 14 Hz, 1H), 2.99 (dd, J=7, 14 Hz, 1H), 1.71-1.63 (m, 4H), 1.55-1.44 (m, 3H), 1.29-1.17 (m, 2H), 1.08-0.98 (m, 1H); Low resolution mass spectrum (ES) m/e 476[(M+H)+, calcd for C₂₈H₃₄N₃O₄: 476]; 95.4% purity based on HPLC.

### Example 10

### (S)-FURAN-2-CARBOXYLIC ACID [4-(2-AMINO-3-PHENYL-PROPIONYLAMINO) -BENZYL]-CYCLOHEXYL-AMIDE

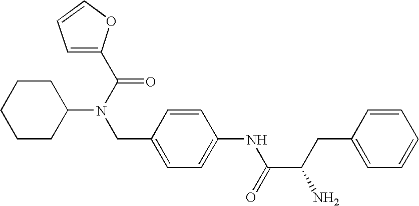

Furan-2-carboxylic acid (4-amino-benzyl)-cyclohexyl-amide was coupled to (S)-3-phenyl-2-(9H-fluoren-9-ylmethoxycarbonylamino)-propionic acid as described in the method of Example 5. Purification by HPLC produced the title compound. 1H NMR (400 MHz, DMSO-d₆) δ 10.32 (s, 1H), 8.29 (br s, 3H), 7.79 (br s, 1H), 7.42 (d, J=8 Hz, 2H), 7.34-7.21 (m, 7H), 6.89 (br s, 1H), 6.58 (br s, 1H), 4.63 (br s, 2H), 4.11 (m, 2H), 3.15 (dd, J=7, 14 Hz, 1H), 3.06 (dd, J=7, 14 Hz, 1H), 1.71-1.63 (m, 4H), 1.55-1.44 (m, 3H), 1.27-1.17 (m, 2H), 1.08-0.98 (m, 1H); Low resolution mass spectrum (ES) m/e 446[(M+H)+, calcd for C₂₇H₃₂N₃O₃: 446]; 100% purity based on HPLC.

### Example 11

### (R)-FURAN-2-CARBOXYLIC ACID [4-(2-AMINO-3-PHENYL-PROPIONYLAMINO)-BENZYL]-CYCLOHEXYL-AMIDE

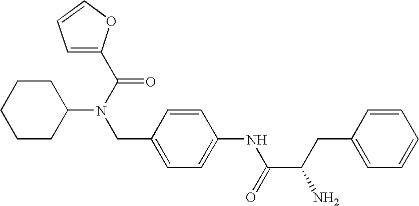

Furan-2-carboxylic acid (4-amino-benzyl)-cyclohexyl-amide was coupled to (R)-3-phenyl-2-(9H-fluoren-9-ylmethoxycarbonylamino)-propionic acid as described in the method of Example 5. Purification by HPLC produced the title compound. 1H NMR (400 MHz, DMSO-d₆) δ 10.33 (s, 1H), 8.30 (br s, 3H), 7.79 (br s, 1H), 7.43 (d, J=8 Hz, 2H), 7.34-7.21 (m, 7H), 6.89 (br s, 1H), 6.58 (br s, 1H), 4.63 (br s, 2H), 4.11 (br m, 2H), 3.15 (dd, J=7, 14 Hz, 1H), 3.06 (dd, J=7, 14 Hz, 1H), 1.71-1.63 (m, 4H), 1.55-1.44 (m, 3H), 1.27-1.17 (m, 2H), 1.09-0.98 (m, 1H); Low resolution mass spectrum (ES) m/e 446[(M+H)+, calcd for C₂₇H₃₂N₃O₃: 446]; 100% purity based on HPLC.

### Example 12

### (S)-FURAN-2-CARBOXYLIC ACID [4-(2-AMINO-PROPIONYLAMINO)-BENZYL]-CYCLOHEXYL-AMIDE

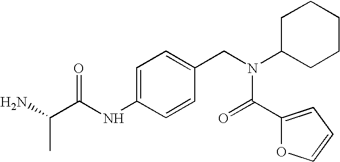

Furan-2-carboxylic acid (4-amino-benzyl)-cyclohexyl-amide coupled to (S)-2-(9H-Fluoren-9-ylmethoxycarbonylamino)-propionic acid as described in the method of Example 5. Purification by HPLC produced the title compound. 1H NMR (400 MHz, DMSO-d₆) δ 10.38 (s, 1H), 8.17 (br s, 3H), 7.79 (br s, 1H), 7.52 (d, J=8 Hz, 2H), 7.23 (d, J=8 Hz, 2H), 6.89 (br s, 1H), 6.58 (br s, 1H), 4.64 (br s, 2H), 4.11 (t, J=11 Hz, 1H), 3.97 (br s, 1H), 1.71-1.63 (m, 4H), 1.55-1.47 (m, 3H), 1.43 (d, J=7 Hz, 3H), 1.26-1.17 (m, 2H), 1.08-0.98 (m, 1H); Low resolution mass spectrum (ES) m/e 370[(M+H)+, calcd for C₂₁H₂₈N₃O₃: 370]; 99% purity based on HPLC.

### Example 13

### (R)-FURAN-2-CARBOXYLIC ACID [4-(2-AMINO-PROPIONYLAMINO)-BENZYL]-CYCLOHEXYL-AMIDE

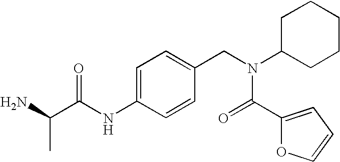

Furan-2-carboxylic acid (4-amino-benzyl)-cyclohexyl-amide was coupled to (R)-2-(9H-Fluoren-9-ylmethoxycarbonylamino)-propionic acid as described in the method of Example 5. Purification by HPLC produced the title compound. 1H NMR (400 MHz, DMSO-d₆) δ 10.36 (s, 1H), 8.16 (br s, 3H), 7.79 (br s, 1H), 7.52 (d, J=9 Hz, 2H), 7.23 (d, J=8 Hz, 2H), 6.89 (br s, 1H), 6.58 (br s, 1H), 4.64 (br s, 2H), 4.11 (t, J=11 Hz, 1H), 3.97 (br s, 1H), 1.71-1.63 (m, 4H), 1.55-1.47 (m, 3H), 1.43 (d, J=7 Hz, 3H), 1.26-1.17 (m, 2H), 1.08-0.98 (m, 1H); Low resolution mass spectrum (ES) m/e 370[(M+H)+, calcd for C₂₁H₂₈N₃O₃: 370]; 99.3% purity based on HPLC.

### Example 14

### (S)-[3-AMINO-3-(4-{[CYCLOHEXYL-(FURAN-2-CARBONYL)-AMINO]-METHYL}-PHENYLCARBAMOYL)-PROPYL]-CARBAMIC ACID 9H-FLUOREN-9-YLMETHYL ESTER

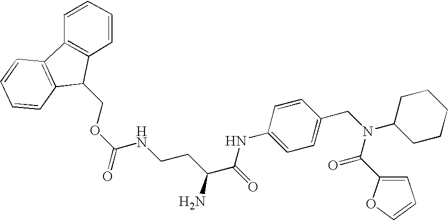

Furan-2-carboxylic acid (4-amino-benzyl)-cyclohexyl-amide was coupled to (S)-2-tert-Butoxycarbonylamino-6-(9H-fluoren-9-ylmethoxycarbonylamino)-hexanoic acid as described in the method of Example 6. Purification by HPLC produced the title compound. 1H NMR (400 MHz, DMSO-d₆) δ 10.40 (s, 1H), 8.23 (d, J=2 Hz, 3H), 7.88 (d, J=8 Hz, 2H), 7.78 (br s, 1H), 7.65 (d, J=7 Hz, 2H), 7.51 (d, J=8 Hz, 2H), 7.42-7.29 (m, 5H), 7.23 (d, J=8 Hz, 2H), 6.88 (br s, 1H), 6.58 (br s, 1H), 4.63 (br s, 2H), 4.32 (m, 2H), 4.19 (t, J=7 Hz, 1H), 4.10 (br s, 1H), 3.88 (m, 1H), 3.10 (dd, J=7, 13Hz, 2H), 1.93 (d, J=7 Hz, 2H), 1.69-1.62 (m, 4H), 1.54-1.43 (m, 3H), 1.25-1.16 (m, 2H), 1.06-0.97 (m, 1H); Low resolution mass spectrum (ES) m/e 621 [(M+H)+, calcd for C₃₇H₄₁N₄O₅: 621]; 96.6% purity based on HPLC.

### Example 15

### (S)-[4-AMINO-1-(4-{[CYCLOHEXYL-(FURAN-2-CARBONYL)-AMINO]-METHYL}-PHENYLCARBAMOYL)-BUTYL]-CARBAMIC ACID 9H-FLUOREN-9-YLMETHYL ESTER

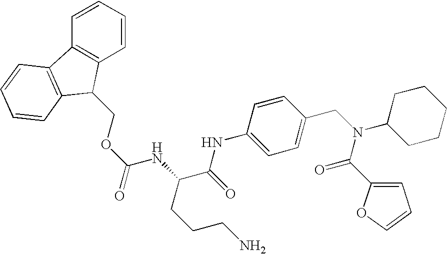

Furan-2-carboxylic acid (4-amino-benzyl)-cyclohexyl-amide was coupled to (S)-5-tert-Butoxycarbonylamino-2-(9H-fluoren-9-ylmethoxycarbonylamino)-pentanoic acid as described in the method of Example 6. Purification by HPLC produced the title compound. 1H NMR (400 MHz, DMSO-d₆) δ 10.06 (s, 1H), 7.89 (d, J=7.5 Hz, 2H), 7.79 (m, 1H), 7.74-7.65 (m, 6H), 7.53 (d, J=8.4 Hz, 2H), 7.40 (dt, J=3, 7.4 Hz, 2H), 7.31 (dd, J=6.7, 13.5 Hz, 2H), 7.20 (d, J=8.4 Hz, 2H), 6.88 (br s, 1H), 6.58 (br s, 1H), 4.63 (br s, 2H), 4.35-4.12 (m, 5H), 2.81-2.71 (m, 2H), 1.71-1.41 (m, 11H), 1.26-1.17 (m, 2H); Low resolution mass spectrum (ES) m/e 635[(M+H)+, calcd for C₃₈H₄₃N₄O₅: 635]; 97.7% purity based on HPLC.

### Example 16

### (R)-[4-AMINO-1-(4-{[CYCLOHEXYL-(FURAN-2-CARBONYL)-AMINO]-METHYL}-PHENYLCARBAMOYL)-BUTYL]-CARBAMIC ACID 9H-FLUOREN-9-YLMETHYL ESTER

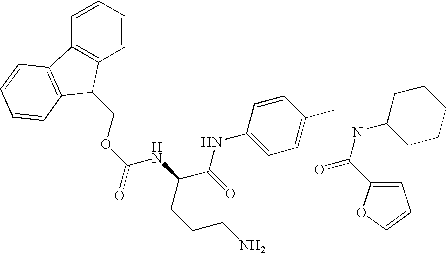

Furan-2-carboxylic acid (4-amino-benzyl)-cyclohexyl-amide was coupled to (R)-5-tert-Butoxycarbonylamino-2-(9H-fluoren-9-ylmethoxycarbonylamino)-pentanoic acid as described in the method of Example 6. Purification by HPLC produced the title compound. 1H NMR (400 MHz, DMSO-d₆) δ 10.06 (s, 1H), 7.89 (d, J=7.5 Hz, 2H), 7.79 (m, 1H), 7.74-7.66 (m, 6H), 7.53 (d, J=8.4 Hz, 2H), 7.40 (dt, J=2.5, 7.3 Hz, 2H), 7.31 (dd, J=6.7, 13.5 Hz, 2H), 7.20 (d, J=8.4 Hz, 2H), 6.88 (br s, 1H), 6.57 (br s, 1H), 4.63 (br s, 2H), 4.35-4.12 (m, 5H), 2.81-2.77 (m, 2H), 1.70-1.44 (m, 11H), 1.26-1.17 (m, 2H); Low resolution mass spectrum (ES) m/e 635[(M+H)+, calcd for C₃₈H₄₃N₄O₅: 635]; 97.7% purity based on HPLC.

### Example 17

### (S)-[2-AMINO-2-(4-{[CYCLOHEXYL-(FURAN-2-CARBONYL)-AMINO]-METHYL}-PHENYLCARBAMOYL)-ETHYL]-CARBAMIC ACID 9H-FLUOREN-9-YLMETHYL ESTER

Furan-2-carboxylic acid (4-amino-benzyl)-cyclohexyl-amide was coupled to (S)-2-tert- 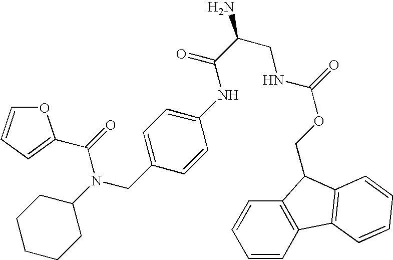
Butoxycarbonylamino-3-(9H-fluoren-9-ylmethoxycarbonylamino)-propionic acid as described in the method of Example 6. Purification by HPLC produced the title compound. 1H NMR (400 MHz, DMSO-d₆) δ 10.42 (s, 1H), 8.26 (br s, 3H), 7.87 (d, J=7.5 Hz, 2H), 7.79 (br s, 1H), 7.63 (d, J-=7.5 Hz, 2H), 7.58 (t, J=6 Hz, 1H), 7.48 (d, J=8.4 Hz, 2H), 7.39 (t, J=7.5 Hz, 2H), 7.27 (t, J=7.5 Hz, 2H), 7.20 (d, J=8.4 Hz, 2H), 6.89 (br s, 1H), 6.58 (br s, 1H), 4.63 (br s, 2H), 4.32-4.23 (m, 2H), 4.18 (t, J=6.7 Hz, 2H), 4.10 (m, 1H), 3.96 (m, 1H), 3.58-3.41 (m, 2H), 1.68-1.42 (m, 7H), 1.24-1.16 (m, 2H), 1.06-0.96 (m, 1H); Low resolution mass spectrum (ES) m/e 607[(M+H)+, calcd for C₃₆H₃₉N₄O₅: 607]; 97.1% purity based on HPLC.

### Example 18

### [(4-{[CYCLOHEXYL-(FURAN-2-CARBONYL)-AMINO]-METHYL}-PHENYLCARBAMOYL)-METHYL]-CARBAMIC ACID 9H-FLUOREN-9-YLMETHYL ESTER

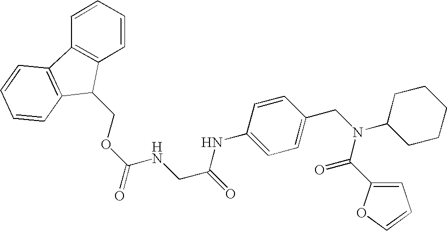

Furan-2-carboxylic acid (4-amino-benzyl)-cyclohexyl-amide was coupled to (9H-Fluoren-9-ylmethoxycarbonylamino)-acetic acid as described in the method for Intermediate #1 of Example 1. Purification by HPLC produced the title compound. 1H NMR (400 MHz, DMSO-d₆) δ 9.93 (s, 1H), 7.89 (d, J=7.5 Hz, 2H), 7.82 (m, 1H), 7.72 (d, J=7.4 Hz, 2H), 7.63-7.58 (m, 1H), 7.50 (t, J=8.4 Hz, 2H), 7.41 (t, J=7.4 Hz, 2H), 7.32 (t, J=7.4 Hz, 2H), 7.18 (d, J=8.4 Hz, 2H), 6.88 (br s, 1H), 6.57 (br s, 1H), 4.63 (br s, 2H), 4.30-4.18 (m, 3H), 4.10 (m, 1H), 3.77 (d, J=6 Hz, 2H), 1.70-1.62 (m, 4H), 1.51-1.44 (m, 3H), 1.25 (m, 2H), 1.07-0.96 (m, 1H); Low resolution mass spectrum (ES) m/e 578[(M+H)+, calcd for C₃₅H₃₆N₃O₅: 578]; 98.4% purity based on HPLC.

### Example 19

### (S)-(5-AMINO-1-PHENYLCARBAMOYL-PENTYL)-CARBAMIC ACID 9H-FLUOREN-9-YLMETHYL ESTER

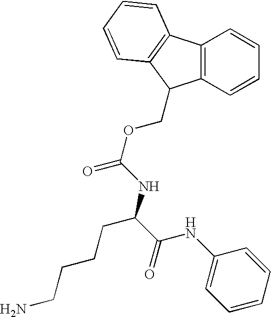

Aniline was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 10.03 (s, 1H), 7.89 (d, J=7.5 Hz, 2H), 7.73 (t, J=7.6 Hz, 2H), 7.64 (m, 4H), 7.60 (d, J=7.9 Hz, 2H), 7.41 (t, J=7.2 Hz, 2H), 7.34-7.28 (m, 4H), 7.04 (t, J=7.4 Hz, 1H), 4.33-4.20 (m, 3H), 4.15-4.10 (m, 1H), 2.77 (m, 2H), 1.74-1.27 (m, 6H); Low resolution mass spectrum (ES) m/e 444[(M+H)+, calcd for C₂₇H₃₀N₃O₃: 444]; 95% purity based on HPLC.

### Example 20

### (S)-(5-AMINO-1-P-TOLYLCARBAMOYL-PENTYL)-CARBAMIC ACID 9H-FLUOREN-9-YLMETHYL ESTER

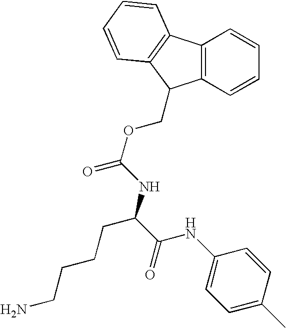

p-Tolylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 9.93 (s, 1H), 7.89 (d, J=7.5 Hz, 2H), 7.72 (t, J=7.6 Hz, 2H), 7.61 (m, 4H), 7.47 (d, J=8.3 Hz, 2H), 7.41 (t, J=7.3 Hz, 2H), 7.34-7.29 (m, 2H), 7.10 (d, J=8.3 Hz, 2H), 4.33-4.20 (m, 3H), 4.13-4.08 (m, 1H), 2.76 (m, 2H), 2.24 (s, 3H), 1.73-1.28 (m, 6H); Low resolution mass spectrum (ES) m/e 458[(M+H)+, calcd for C₂₈H₃₂N₃O₃: 458]; 95% purity based on HPLC.

### Example 21

### (S)-(5-AMINO-1-BENZYLCARBAMOYL-PENTYL)-CARBAMIC ACID 9H-FLUOREN-9-YLMETHYL ESTER

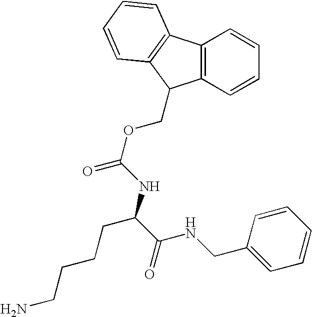

Benzylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 8.41 (t, J=5.9 Hz, 1H), 7.89 (d, J=7.5 Hz, 2H), 7.72 (t, J=6.7 Hz, 2H), 7.65 (br s, 3H), 7.51 (d, J=8.2 Hz, 1H), 7.41 (d, J=7.4 Hz, 2H), 7.33-7.20 (m, 7H), 4.32-4.19 (m, 5H), 4.02-3.97 (m, 1H), 2.75 (m, 2H), 1.70-1.22 (m, 6H); Low resolution mass spectrum (ES) m/e 458[(M+H)+, calcd for C₂₈H₃₂N₃O₃: 458]; 95% purity based on HPLC.

### Example 22

### (S)-(5-AMINO-1-CYCLOHEXYLCARBAMOYL-PENTYL)-CARBAMIC ACID 9H-FLUOREN-9-YLMETHYL ESTER

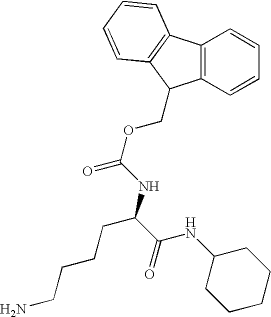

Cyclohexylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 7.89 (d, J=7.5 Hz, 2H), 7.73-7.65 (m, 6H), 7.40 (dd, J=8.1, 15.7 Hz, 3H), 7.31 (t, J=7 Hz, 2H), 4.30-4.18 (m, 3H), 3.92 (dd, J=8.5, 14.1 Hz, 1H), 3.53-3.46 (m, 1H), 2.75 (m, 2H), 1.70-1.46 (m, 9H), 1.35-1.07 (m, 7H); Low resolution mass spectrum (ES) m/e 450[(M+H)+, calcd for C₂₇H₃₆N₃O₃: 450]; 96% purity based on HPLC.

### Example 23

### (S)-FURAN-2-CARBOXYLIC ACID (4-{6-AMINO-2-[2-(S)-AMINO-3-(1H-INDOL-3-YL)-PROPIONYLAMINO]-HEXANOYLAMINO}-BENZYL)-CYCLOHEXYL-AMIDE

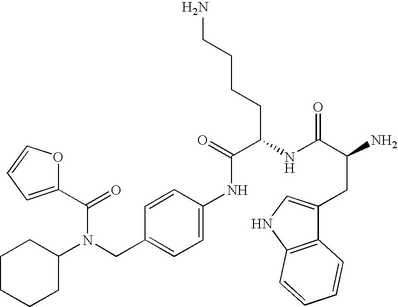

Furan-2-carboxylic acid (4-amino-benzyl)-cyclohexyl-amide was coupled to (S)-6-tert-butoxycarbonylamino-2-(9H-fluoren-9-ylmethoxycarbonylamino)-hexanoic acid as described in the method of Example 5, followed by coupling to (S)-2-tert-Butoxycarbonylamino-3-(1H-indol-3-yl)-propionic acid as described in the method of Example 6. Purification by HPLC produced the title compound. 1H NMR (400 MHz, DMSO-d₆) δ 11.00 (d, J=1.8 Hz, 1H), 10.19 (s, 1H), 8.91 (d, J=7.9 Hz, 1H), 8.02 (m, 3H), 7.80 (m, 1H), 7.71 (br s, 2.4H), 7.66 (d, J=7.9 Hz, 1H), 7.55 (d, J=8.5 Hz, 2H), 7.32 (d, J=8.14 Hz, 1H), 7.23 (m, 3H), 7.02 (t, J=7.5 Hz, 1H), 6.89 (t, J=7.4 Hz, 2H), 6.56 (m, 1H), 4.65 (br s, 2H), 4.48 (dd, J=7.8, 13.8 Hz, 1H), 4.09 (m, 2H), 3.25 (dd, J=4.9, 15 Hz, 1H), 3.06 (dd, J=8.2, 15 Hz, 1H), 2.76 (m, 2H), 1.76-1.18 (m, 15H), 1.09-0.99 (m, 1H); Low resolution mass spectrum (ES) m/e 612[(M+H)+, calcd for C₃₅H₄₅N₆O₄: 612]; 96% purity based on HPLC.

### Example 24

### (S)-FURAN-2-CARBOXYLIC ACID (4-{6-AMINO-2-[(NAPHTHALENE-1-CARBONYL)-AMINO]-HEXANOYLAMINO}-BENZYL)-CYCLOHEXYL-AMIDE

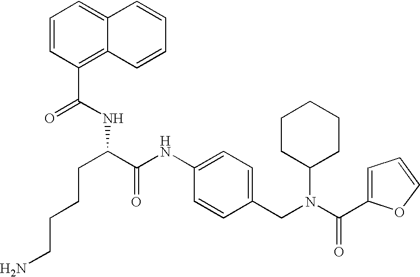

The title compound was prepared by coupling furan-2-carboxylic acid (4-amino-benzyl)-cyclohexyl-amide to (S)-6-tert-Butoxycarbonylamino-2-(9H-fluoren-9-ylmethoxycarbonylamino)-hexanoic acid, and then to naphthalene-1-carboxylic acid as described for the method of Example 23. Purification by HPLC produced the title compound. 1H NMR (400 MHz, DMSO-d₆) δ 10.17 (s, 1H), 8.74 (d, J=7.5 Hz, 1H), 8.23 (dd, J=3.5, 6.3 Hz, 1H), 8.02 (d, J=8.2 Hz, 1H), 7.97 (dd, J=3.3, 6.2 Hz, 1H), 7.79 (br s, 1H), 7.66 (m, 1H), 7.60-7.53 (m, 5H), 7.22 (d, J=8.5 Hz, 2H), 6.89 (br s, 1H), 6.58 (br s, 1H), 4.65-4.59 (m, 3H), 4.12 (m, 11H), 2.80 (p, J=6.1 Hz, 2H), 1.83-1.44 (m, 13H), 1.27-1.17 (m, 2H), 1.08-0.99 (m, 1H); Low resolution mass spectrum (ES) m/e 581 [(M+H)+, calcd for C₃₅H₄₁N₄O₄: 581]; 96.7% purity based on HPLC.

### Example 25

### (S)-FURAN-2-CARBOXYLIC ACID {4-[6-AMINO-2-(2-NAPHTHALEN-1-YL-ACETYLAMINO)-HEXANOYLAMINO]-BENZYL}-CYCLOHEXYL-AMIDE

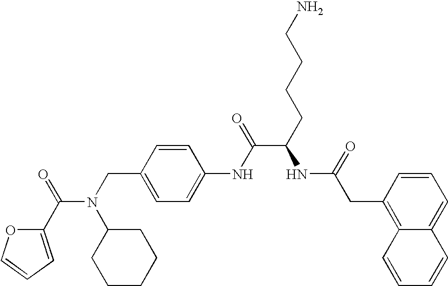

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid, and then with naphthalen-1-yl-acetic acid as described in the method of Example 23. Purification by HPLC produced the title compound. 1H NMR (400 MHz, DMSO-d₆) δ 10.04 (s, 1H), 8.52 (d, J=8 Hz, 1H), 8.09 (m, 1H), 7.90 (m, 1H), 7.81 (m, 2H), 7.65 (m, 3H), 7.52-7.42 (m, 6H), 7.18 (d, J=8.5 Hz, 2H), 6.88 (br s, 1H), 6.57 (br s, 1H), 4.63 (m, 2H), 4.40 (m, 1H), 4.10 (m, 1H), 4.01 (d, J=15 Hz, 1H), 3.95 (d, J=15 Hz, 1H), 2.72 (m, 2H), 1.76-1.16 (m, 15H), 1.07-0.99 (m, 1H); Low resolution mass spectrum (ES) m/e 595[(M+H)+, calcd for C₃₆H₄₃N₄O₄: 595]; 93.0% purity based on HPLC.

### Example 26

### (S)-FURAN-2-CARBOXYLIC ACID {4-[6-AMINO-2-(3-BIPHENYL-4-YL-UREIDO)-HEXANOYLAMINO]-BENZYL}-CYCLOHEXYL-AMIDE

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid as described in the method of Example 5. Coupling to 4-isocyanato-biphenyl was done as described in the method for Intermediate #3 of Example 1, and then final deprotection as described in the method Example 2. Purification by HPLC produced the title compound. 1H NMR (400 MHz, DMSO-d₆) δ 10.17 (s, 1H), 8.85 (s, 1H), 7.78 (br s, 1H), 7.66 (br s, 3H), 7.60 (d, J=7.3 Hz, 2H), 7.54 (d, J=8.8 Hz, 4H), 7.48 (d, J=8.8 Hz, 2H), 7.41 (t, J=7.7 Hz, 2H), 7.28 (t, J=7.3 Hz, 1H), 7.20 (d, J=8.5 Hz, 2H), 6.88 (br s, 1H), 6.60 (m, 2H), 4.63 (br s, 2H), 4.40 (dd, J=7.9, 13.4 Hz, 1H), 4.11 (m, 1H), 2.82-2.74 (m, 2H), 1.79-1.31 (m, 13H), 1.26-1.16 (m, 2H), 1.07-0.98 (m, 1H); Low resolution mass spectrum (ES) m/e 622[(M+H)+, calcd for C₃₇H₄₄N₅O₄: 622]; 98.0% purity based on HPLC.

### Example 27

### (S)-FURAN-2-CARBOXYLIC ACID [4-(6-AMINO-2-BENZOYLAMINO-HEXANOYLAMINO)-BENZYL]-CYCLOHEXYL-AMIDE

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid, and then benzoic acid as described in the method of Example 23, and purification by HPLC produced the title compound. 1H NMR (400 MHz, DMSO-d₆) δ 10.09 (s, 1H), 8.57 (d, J=7.7 Hz, 1H), 7.90 (d, J=8.6 Hz, 2H), 7.78 (br s, 1H), 7.64 (br s, 3H), 7.55 (m, 3H), 7.47 (t, J=7.4 Hz, 2H), 7.19 (d, J=8.5 Hz, 2H), 6.88 (br s, 1H), 6.57 (br s, 1H), 4.63 (br s, 2H), 4.55 (dd, J=7.4, 14.8 Hz, 1H), 4.10 (m, 1H), 2.81-2.76 (m, 2H), 1.81 (dd, J=7.6, 14.8 Hz, 2H), 1.0-1.35 (m, 11H), 1.26-1.16 (m, 2H), 1.07-0.97 (m, 1H); Low resolution mass spectrum (ES) m/e 531 [(M+H)+, calcd for C₃₁H₃₉N₄O₄: 531]; 99.0% purity based on HPLC.

### Example 28

### (S)-[1-(4-{[CYCLOHEXYL-(FURAN-2-CARBONYL)-AMINO]-METHYL}-PHENYLCARBAMOYL)-PROPYL]-CARBAMIC ACID 9H-FLUOREN-9-YLMETHYL ESTER

Furan-2-carboxylic acid (4-amino-benzyl)-cyclohexyl-amide was coupled to (S)-2-(9H-Fluoren-9-ylmethoxycarbonylamino)-butyric acid as described in the method for Intermediate #1 of Example 1. Purification by HPLC produced the title compound. ¹H NMR (400 MHz, DMSO-d₆) δ 9.99 (s, 1H), 7.88 (d, J=7.5 Hz, 2H), 7.78 (br m, 1H), 7.73 (t, J=7.3 Hz, 2H), 7.62 (d, J=7.9 Hz, 1H), 7.52 (d, J=8.4 Hz, 2H), 7.40 (dt, J=2.8, 7.4 Hz, 2H), 7.31 (dt, J=4.3, 7.3 Hz, 2H), 6.87 (br s, 1H), 6.57 (br s, 1H), 4.3 (br s, 2H), 4.27-4.02 (m, 5H), 1.75-1.43 (m, 9H), 1.25-1.16 (m, 2H), 1.07-0.95 (m, 1H); Low resolution mass spectrum (ES- m/e 606[(M+H)+, calcd for C₃₇H₄₀N₃O₅: 606]; 97.7% purity based on HPLC.

### Example 29

### (S)-[1-(4-{[CYCLOHEXYL-(FURAN-2-CARBONYL)-AMINO]-METHYL}-PHENYL CARBAMOYL)-2-PHENYL-ETHYL]-CARBAMIC ACID 9H-FLUOREN-9-YLMETHYL ESTER

Furan-2-carboxylic acid (4-amino-benzyl)-cyclohexyl-amide was coupled to (S)-2-(9H-fluoren-9-ylmethoxycarbonylamino)-3-phenyl-propionic acid as described in the method for Intermediate #1 of Example 1. Purification by HPLC produced the title compound. 1H NMR (400 MHz, DMSO-d₆) δ 10.10 (s, 1H), 7.86 (d, J=7.5 Hz, 2H), 7.80 (m, 2H), 7.65 (t, J=7.7 Hz, 2H), 7.52 (d, J=8.4 Hz, 2H), 7.41-7.17 (m, 11H), 6.88 (br s, 1H), 6.57 (br s, 1H), 4.64 (br s, 2H), 4.42-4.36 (m, 1H), 4.18-4.09 (m, 4H), 3.02 (dd, J=4.4, 13.6 Hz, 1H), 2.88 (dd, J=10.4, 13.5 Hz, 1H), 1.70-1.44 (m, 7H), 1.25-1.16 (m, 2H), 1.07-0.98 (m, 1H); Low resolution mass spectrum (ES) m/e 668[(M+H)+, calcd for C₄₂H₄₂N₃O₅: 668]; 96.7% purity based on HPLC.

### Example 30

### (S)-2-[6-TERT-BUTOXYCARBONYLAMINO-2-(S)-((9H-FLUOREN-9-YLMETHOXYCARBONYL)AMINO)-HEXANOYL-AMINO]-3-(1H-INDOL-3-YL)-PROPIONIC ACID METHYL ESTER

(S)-2-amino-3-(1H-indol-3-yl)-propionic acid methyl ester was coupled to (S)-6-tert-Butoxycarbonylamino-2-(9H-fluoren-9-ylmethoxycarbonylamino)-hexanoic acid as described in the method for Intermediate #1 of Example 1. Purification by HPLC produced the title compound. 1H NMR (400 MHz, DMSO-d₆) δ 10.84 (s, 0.5H), 8.27 (d, J=7.2 Hz, 1H), 7.86 (d, J=7.5 Hz, 2H), 7.69 (dd, J=5.7, 6.8 Hz, 2H), 7.45 (d, J=7.8 Hz, 1H), 7.39 (t, J=7.4 Hz, 2H), 7.34-7.26 (m, 2H), 7.14 (s, 1H), 7.04 (t, J=7.4 Hz, 1H), 6.96 (t, J=7.5 Hz, 1H), 4.48 (dd, J=7.3, 13.7 Hz, 1H), 4.31-4.12 (m, 3H), 4.04-3.94 (m, 1H), 3.52 (s, 1H), 3.54-3.44 (m, 2H); Low resolution mass spectrum (ES) m/e 669[(M+H)+, calcd for C₃₈H₄₅N₄O₇: 669]; 90% purity based on NMR.

### Example 31

### (S)-FURAN-2-CARBOXYLIC ACID {4-[6-AMINO-2-(3-NAPHTHALEN-1-YL-UREIDO)-HEXANOYLAMINO]-BENZYL}-CYCLOHEXYL-AMIDE

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid then to 1-isocyanato-naphthalene as described in the method of Example 26. Purification by HPLC produced the title compound. 1H NMR (400 MHz, DMSO-d₆) δ 10.20 (s, 1H), 8.76 (s, 1H), 8.51 (s, 1H), 8.12 (d, J=8.2 Hz, 1H), 8.03 (d, J=6.9 Hz, 1H), 7.89 (m, 2H), 7.78 (br s, 1H), 7.70 (d, J=8.1 Hz, 1H), 7.65 (br s, 2.5H), 7.57-7.37 (m, 9H), 7.20 (d, J=8.5 Hz, 2H), 7.09 (d, J =8.2 Hz, 1H), 6.88 (br s, 1H), 6.58 (br s, 1H), 4.64 (br s, 2H), 4.46 (dd, J=7.8, 13.6 Hz, 1H), 4.11 (m, 1H), 3.47 (m, 2H), 2.83-2.75 (m, 2H), 1.81-1.40 (m, 15H), 1.26-1.17 (m, 2H), 1.07-0.98 (m, 1H); Low resolution mass spectrum (ES) m/e 596[(M+H)+, calcd for C₃₅H₄₂N₅O₄: 596]; 83% purity based on HPLC.

### Example 32

### (R)-FURAN-2-CARBOXYLIC ACID {4-[6-AMINO-2-(3-NAPHTHALEN-1-YL-UREIDO)-HEXANOYLAMINO]-BENZYL}-CYCLOHEXYL-AMIDE

Furan-2-carboxylic acid (4-amino-benzyl)-cyclohexyl-amide was coupled to (R)-2-(9H-Fluoren-9-ylmethoxycarbonylamino)-butyric acid, and then to 1-isocyanato-naphthalene as described in the method of Example 26. Purification by HPLC produced the title compound. 1H NMR (400 MHz, DMSO-d₆) δ 9.99 (s, 1H), 7.88 (d, J=8.2 Hz, 1H), 8.03 (d, J=7.59 Hz, 2H), 7.78 (m, 1H), 7.73 (t, J=7.3 Hz, 2H), 7.62 (d, J=7.9 Hz, 1H), 7.52 (d, J=8.4 Hz, 2H), 7.40 (dt, J=7.4, 7.8 Hz, 2H), 7.31 (dt, J=4.3, 7.3 Hz, 2H), 7.18 (d, J=8.4 Hz, 2H), 6.87 (br s, 1H), 6.57 (br s, 1H), 4.63 (br s, 2H), 4.27-4.02 (m, 5H), 1.75-1.59 (m, 6H), 1.53-1.43 (m, 3H), 1.25-1.16 (m, 2H), 1.07-0.98 (m, 1H), 0.91 (t, J=7.3 Hz, 3H); Low resolution mass spectrum (ES) m/e 606[(M+H)+, calcd for C₃₇H₄₀N₃O₅: 606]; 95% purity based on HPLC.

### Example 33

### (S)-[5-AMINO-1-(BENZYL-CYCLOHEXYL-CARBAMOYL)-PENTYL]-CARBAMIC ACID 9H-FLUOREN-9-YLMETHYL ESTER

Benzyl-cyclohexyl-amine was coupled to (S)-6-tert-Butoxycarbonylamino-2-(9H-fluoren-9-ylmethoxycarbonylamino)-hexanoic acid as described in the method of Example 23. Purification by HPLC produced the title compound. 1H NMR (400 MHz, DMSO-d₆) δ 7.88 (d, J=7.6 Hz, 2H), 7.7 (d, J=7.4 Hz, 2H), 7.4 (t, J=7.4 Hz, 2H), 7.35-7.18 (m, 5H), 7.18-7.10 (m, 2H), 4.66-4.36 (m, 3H), 4.33-4.05 (m, 4H), 3.8-3.67 (m, 1H), 2.76 (t, J=7.5 Hz, 1H), 2.62 (t, J =7.5 Hz, 1H), 1.72-0.89 (m, 16H); Low resolution mass spectrum (ES) m/e 540[(M+H)+, calcd for C₃₄H₄₂N₃O₃: 540]; 95% purity based on HPLC.

### Example 34

### (S)-[5-AMINO-5-(BENZYL-CYCLOHEXYL-CARBAMOYL)-PENTYL]-CARBAMIC ACID TERT-BUTYL ESTER

Benzyl-cyclohexyl-amine was coupled to (S)-6-tert-Butoxycarbonylamino-2-(9H-fluoren-9-ylmethoxycarbonylamino)-hexanoic acid as described in the method of Example 5. Purification by HPLC produced the title compound. 1H NMR (400 MHz, DMSO-d₆) δ 7.39-7.23 (m, 3H), 7.22-7.14 (m, 2H), 4.66-4.39 (m, 3H), 4.15-4.01 (m, 0.5H), 3.96-3.84 (m, 0.5H), 3.67-3.46 (m, 2H), 2.94-2.64 (m, 2H), 1.82-0.91 (m, 25H); Low resolution mass spectrum (ES) m/e 418[(M+H)+, calcd for C₂₄H₄₀N₃O₃: 418]; 90% purity based on HPLC.

### Example 35

### (S)-[5-[2-(S)-AMINO-3-(1H-INDOL-3-YL)-PROPIONYLAMINO]-1-(4-{[CYCLOHEXYL-(FURAN-2-CARBONYL)-AMINO]-METHYL}-PHENYLCARBAMOYL)-PENTYL]-CARBAMIC ACID 9H-FLUOREN-9-YLMETHYL ESTER

(S)-[4-Amino-1-(4-{[cyclohexyl-(furan-2-carbonyl)-amino]-methyl}-phenylcarbamoyl)-butyl]-carbamic acid 9H-fluoren-9-ylmethyl ester from Example 15 was coupled to (S)-2-tert-Butoxycarbonylamino-3-(1H-indol-3-yl)-propionic acid as described in the method of Example 6. Purification by HPLC produced the title compound. 1H NMR (400 MHz, DMSO-d₆) δ 11 (s, 1H), 10 (s, 1H), 8.43 (t, J=5.2 Hz, 1H), 8.03 (app d, J=3.5 Hz, 2H), 7.88 (app d, J=7.5 Hz, 2H), 7.78 (br s, 1H), 7.70 (dd, J=5.2, 6.8 Hz, 2H), 7.62 (d, J=7.7 Hz, 2H), 7.52 (d, J=8.3 Hz, 2H), 7.44-7.25 (m, 5H), 7.23-7.13 (m, 3H), 7.07 (t, J=7.4 Hz, 1H), 6.99 (t, J=7.4 Hz, 1H), 6.87 (br s, 1H), 6.57 (br s, 1H), 4.62 (br s, 2H), 4.3-3.7 (m, 4H), 3.23-3 (m, 5H), 1.78-0.98 (m, 16H); Low resolution mass spectrum (ES) m/e 835[(M)+, calcd for C₅₀H₅₄N₆O₆: 835]; 95% purity based on HPLC.

### Example 36

### (R)-(5-AMINO-1-P-TOLYLCARBAMOYL-PENTYL)-CARBAMIC ACID 9H-FLUOREN-9-YLMETHYL ESTER

p-Tolylamine was coupled to (R)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 9.94 (s, 1H), 7.90 (d, J=7.6 Hz, 2H), 7.74 (t, J=7.6 Hz, 2H), 7.66 (br s, 4H), 7.49 (d, J=8.4 Hz, 2H), 7.42 (t, J=7.4 Hz, 2H), 7.33 (m, 2H), 7.11 (d, J=8.3 Hz, 2H), 4.34-4.09 (m, 4H), 2.78 (m, 2H), 2.25 (s, 3H), 1.74-1.49 (m, 4H), 1.43-1.29 (m, 2H); Low resolution mass spectrum (ES) m/e 458[(M)+, calcd for C₂₈H₃₁N₃O₃: 458]; 90% purity based on HPLC.

### Example 37

### (S)-[1-(4-{[CYCLOHEXYL-(FURAN-2-CARBONYL)-AMINO]-METHYL}-PHENYLCARBAMOYL)-PENTYL]-CARBAMIC ACID 9H-FLUOREN-9-YLMETHYL ESTER

Furan-2-carboxylic acid (4-amino-benzyl)-cyclohexyl-amide was coupled to (S)-2-(9H-fluoren-9-ylmethoxycarbonylamino)-hexanoic acid as described in the method for Intermediate #1 of Example 1. Purification by HPLC produced the title compound. 1H NMR (400 MHz, DMSO-d₆) δ 10.0 (s, 1H), 7.89 (d, J=7.5 Hz, 2H), 7.79 (br s, 1H), 6.74 (t, J=7.0 Hz, 2H), 7.64 (d, J=8.0 Hz, 1H), 7.54 (d, J=8.4 Hz, 2H), 7.41 (dt, J=3.4, 7.4 Hz, 2H), 7.31 (dt, J=3.5, 7.4 Hz, 2H), 7.19 (d, J=8.4 Hz, 2H), 6.88 (br s, 1H), 6.58 (br s, 1H), 4.64 (br s, 2H), 4.3-4.08 (m, 5H), 1.71-1.44 (m, 9H), 1.39-1.17 (m, 6H), 1.08-0.98 (m, 1H), 0.87 (t, J=6.8 Hz, 3H); Low resolution mass spectrum (ES) m/e 634[(M+1)+, calcd for C₃₉H₄₄N₃O₅: 634]; 95% purity based on HPLC.

### Example 38

### (S)-[5-AMINO-1-(2-METHYL-1H-INDOL-5-YLCARBAMOYL)-PENTYL]-CARBAMIC ACID 9H-FLUOREN-9-YL METHYL ESTER

2-Methyl-1H-indol-5-ylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 10.6 (s, 1H), 9.72 (s, 1H), 7.89 (d, J=7.5 Hz, 2H), 7.79-7.53 (m, 6H), 7.41 (ddd, J=2.6, 7.3, 7.4 Hz, 2H), 7.37-7.27 (m, 2H), 7.22-7.04 (m, 2H), 6.05 (s, 1H), 4.35-4.18 (m, 3H), 4.13 (dt, J=5.4, 8.3 Hz, 1H), 2.78 (dq, J=5.9, 12 Hz, 2H), 2.34 (s, 3H), 1.78-1.27 (m, 6H); Low resolution mass spectrum (ES) m/e 497[(M+1)+, calcd for C₃₀H₃₃N₄O₃: 497]; 96.4% purity based on HPLC.

### Example 39

### 3-{4-[2-((9H-FLUOREN-9-YLMETHOXYCARBONYL)AMINO)-2-METHYL-PROPIONYLAMINO]-PHENYL}-ACRYLIC ACID

3-(4-Amino-phenyl)-acrylic acid methyl ester was coupled to 2-(9H-Fluoren-9-ylmethoxycarbonylamino)-2-methyl-propionic acid as described in the method for Intermediate #1 of Example 1. Ester hydrolysis (0.1 N sodium hydroxide in methanol for 12 hours) was performed, and then purification by HPLC produced the title compound. 1H NMR (400 MHz, DMSO-d₆) δ 9.61 (s, 1H), 7.87 (d, J=7.5 Hz, 2H), 7.75-7.65 (m, 4H), 7.58 (d, J=8.5 Hz, 2H), 7.5 (d, J=16 Hz, 1H), 7.39 (t, J=7.3 Hz, 2H), 7.3 (t, J=6.8 Hz, 2H), 6.39 (d, J=16 Hz, 1H), 4.27 (app q, J=6.9 Hz, 2H), 4.19 (t, J=6.4 Hz, 1H), 1.41 (s, 6H); Low resolution mass spectrum (ES) m/e 471 [(M+1)+, calcd for C₂₈H₂₇N₂O₅: 471]; 86% purity based on HPLC.

### Example 40

### (S)-6-AMINO-2-(3-BIPHENYL-4-YL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 4-isocyanato-biphenyl as described in the method of Example 26. Purification by HPLC produced the title compound. 1H NMR (400 MHz, DMSO-d₆) δ 10.10 (s, 1H), 8.85 (s, 1H), 7.67 (br s, 3H), 7.61 (d, J=7.3 Hz, 2H), 7.56 (d, J=8.7 Hz, 2H), 7.49 (dd, J=8.6, 10.4 Hz, 4H), 7.42 (t, J=7.4 Hz, 2H), 7.30 (t, J=7.3 Hz, 1H), 7.12 (d, J=8.4 Hz, 2H), 6.59 (d, J=8.2 Hz, 1H), 4.42 (dd, J=8, 13.5 Hz, 1H), 2.79 (dd, J=6.8, 12.7 Hz, 2H), 2.25 (s, 3H), 1.8-1.71 (m, 1H), 1.67-1.52 (m, 3H), 1.42-1.32 (m, 2H); Low resolution mass spectrum (ES) m/e 431 [(M+1)+, calcd for C₂₆H₃₁N₄O₂: 431]; 99.9% purity based on HPLC.

### Example 41

### (S)-6-AMINO-2-[3-(4-PHENOXY-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-isocyanato-4-phenoxy-benzene as described in the method of Example 26. Purification by HPLC produced the title compound. 1H NMR (400 MHz, DMSO-d₆) δ 10.08 (s, 1H), 8.76 (s, 1H), 7.67 (br s, 3H), 7.50 (d, J=8.4 Hz, 2H), 7.40 (d, J=9.0 Hz, 2H), 7.33 (dd, J=7.5, 8.5 Hz, 2H), 7.11 (d, J=8.4 Hz, 2H), 7.06 (t, J=7.4 Hz, 1H), 6.92 (t, J=8.3 Hz, 4H), 6.52 (d, J=8.3 Hz, 1H), 4.38 (dd, J=8, 13.5 Hz, 1H), 2.82-2.74 (m, 2H), 2.24 (s, 3H), 1.77-1.69 (m, 1H), 1.64-1.52 (m, 3H), 1.45-1.30 (m, 2H); Low resolution mass spectrum (ES) m/e 447[(M+1)+, calcd for C₂₆H₃₁N₄O₃: 447]; 99.7% purity based on HPLC.

### Example 42

### (S)-6-AMINO-2-[3-(4-TRIFLUOROMETHYL-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid,and then to 1-isocyanato-4-trifluoromethyl-benzene as described in the method of Example 26. Purification by HPLC produced the title compound. 1H NMR (400 MHz, DMSO-d₆) δ 10.10 (s, 1H), 9.20 (s, 1H), 7.67 (br s, 3H), 7.58 (s, 4H), 7.49 (d, J=8.4 Hz, 2H), 7.12 (dd, J=8.4 Hz, 2H), 6.74 (d, J=8.1 Hz, 1H), 4.40 (dd, J=7.9, 13.4 Hz, 1H), 2.78 (br s, 2H), 2.25 (s, 3H), 1.8-1.71 (m, 1H), 1.67-1.52 (m, 3H), 1.46-1.32 (m, 2H); Low resolution mass spectrum (ES) m/e 423[(M+1)+, calcd for C₂₁H₂₆F₃N₄O₂: 423]; 99.2% purity based on HPLC.

### Example 43

### (S)-6-AMINO-2-[3-(4-FLUORO-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-fluoro-4-isocyanato-benzene as described in the method of Example 26. Purification by HPLC produced the title compound. 1H NMR (400 MHz, DMSO-d₆) δ 10.08 (s, 1H), 8.76 (s, 1H), 7.65 (br s, 3H), 7.49 (d, J=8.4 Hz, 2H), 7.39 (m, 2H), 7.12 (d, J=8.4 Hz, 2H), 7.06 (t, J=8.9 Hz, 2H), 6.51 (d, J=8.3 Hz, 1H), 4.38 (dd, J=8.0, 13.5 Hz, 1H), 2.81-2.75 (m, 2H), 2.25 (s, 3H), 1.78-1.72 (m, 7H); Low resolution mass spectrum (ES) m/e 373[(M+1)+, calcd for C₂₀H₂₆FN₄O₂: 373 ]; 90% purity based on HPLC.

### Example 44

### (S)-[5-AMINO-1-(4-FLUORO-PHENYLCARBAMOYL)-PENTYL]-CARBAMIC ACID 9H-FLUOREN-9-YLMETHYL ESTER

p-Fluorophenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d6) δ 10.1 (s, 1H); 7.89 (d, J=7.55, 2H); 7.72 (t, J=7.61 Hz, 2H); 7.69-7.58 (m, 5H); 7.41 (t, J=7.40Hz, 2H); 7.31 (ddd, J=4.31,7.29,7.32 Hz, 2H); 7.14 (t, J=8.87 Hz, 2H); 4.35-4.18 (m, 3H); 4.10 (ddd, J=5.93, 8.46, 8.38, 1H); 2.77 (dddd, J=5.95, 5.95, 6.10, 12.56 Hz, 2H); 1.75-1.47 (m, 4H); 1.47-1.24 (m, 2H); Low resolution mass spectrum (ES) m/e 462 [(M+H)+, calcd for C₂₇H₂₉FN₃O₃: 462 ]; 100% purity based on HPLC.

### Example 45

### (S)-(5-AMINO-1-O-TOLYLCARBAMOYL-PENTYL)-CARBAMIC ACID 9H-FLUOREN-9-YLMETHYL ESTER

o-Tolylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d6) δ 9.32 (s, 1H); 7.89 (d, J=7.52 Hz, 2H); 7.73 (t, J=6.67 Hz, 2H); 7.69-7.57 (m, 4H); 7.41 (t, J=7.42 Hz, 2H); 7.36 (d, J=7.70 Hz, 1H); 7.31 (ddd, J=1.88,7.39,7.40 Hz, 2H); 7.20 (d, J=7.39 Hz, 1H); 7.16 (t, J=7.54 Hz, 1H); 7.08 (ddd, J=0.82, 7.51, 7.66 Hz, 1H); 4.37-4.13 (m, 4H); 2.79 (dddd, J=7.51, 7.51, 6.76, 12.82 Hz, 2H); 2.16 (s, 3H); 1.84-1.50 (m, 4H); 1.50-1.31 (m, 2H); Low resolution mass spectrum (ES) m/e 458 [(M+H)+, calcd for C₂₈H₃₂N₃O₃: 458]; 99% purity based on HPLC.

### Example 46

### (S)-FURAN-2-CARBOXYLIC ACID (4-{6-AMINO-2-[2-(6-BENZOYLAMINO-PURIN-9-YL)-ACETYLAMINO]-HEXANOYLAMINO}-BENZYL)-CYCLOHEXYL-AMIDE

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid, and then to (6-Benzoylamino-purin-9-yl)-acetic acid as described in the method of Example 23. Purification by HPLC produced the title compound. 1H NMR (400 MHz, DMSO-d₆) δ 11.16 (br s, 1H), 10.14 (s, 1H), 8.82 (d, J=8.0 Hz, 1H), 8.7 (s, 1H), 8.43 (s, 1H), 8.05 (d, J=7.3 Hz, 2H), 7.79 (br s, 1H), 7.65 (m, 4H), 7.51 (m, 4H), 7.21 (d, J=8.5 Hz, 2H), 6.89 (br s, 1H), 6.59 (br s, 1H), 5.59 (s, 2H), 4.64 (br s, 2H), 4.47 (dd, J=8.1, 13.8 Hz, 1H), 4.12 (m, 1H), 2.79 (m, 2H), 1.80-1.31 (m, 13H), 1.27-1.17 (m, 2H), 1.02 (m, 1H); Low resolution mass spectrum (ES) m/e 706[(M+1)+, calcd for C₃₈H₄₄N₉O₅: 706]; 98.7% purity based on HPLC.

### Example 47

### (S)-(5-AMINO-1-M-TOLYLCARBAMOYL-PENTYL)-CARBAMIC ACID 9H-FLUOREN-9-YLMETHYL ESTER

m-Tolylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 9.94 (s, 1H), 7.89 (d, J=7.5 Hz, 2H), 7.86 (d, J=7.5 Hz, 1H), 7.73 (app t, J=7.3 Hz, 2H), 7.68-7.57 (m, 3H), 7.46-7.28 (m, 6H), 7.17 (t, J=7.8 Hz, 1H), 4.34-4.18 (m, 3H), 4.11 (ddd, J=5.7, 8.4, 8.5 Hz, 1H), 2.77 (dddd, J=6.4, 6.4, 6.2, 12.5 Hz, 2H), 2.26 (s, 3H), 1.77-1.23 (m, 6H); Low resolution mass spectrum (ES) m/e 458[(M+1)+, calcd for C₂₈H₃₂N₃O₃: 458]; 100% purity based on HPLC.

### Example 48

### (S)-6-AMINO-2-(2-NAPHTHALEN-2-YL-ACETYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to naphthalen-2-yl-acetic acid as described in the method of Example 23. Purification by HPLC produced the title compound. 1H NMR (400 MHz, DMSO-d₆) δ 9.97 (s, 1H), 8.46 (d, J=8.0 Hz, 1H), 7.87-7.82 (m, 3H), 7.76 (s, 1H), 7.65 (br s, 3H), 7.5-7.43 (m, 5H), 7.09 (d, J=8.3 Hz, 2H), 4.4 (dt, J=5.8, 8.3 Hz, 1H), 3.67 (s, 2H), 2.77-2.69 (m, 2H), 2.23 (s, 3H), 1.77-1.48 (m, 4H), 1.42-1.22 (m, 2H); Low resolution mass spectrum (ES) m/e 404[(M+1)+, calcd for C₂₅H₃₀N₃O₂: 404]; 95.9% purity based on HPLC.

### Example 49

### (S)-[1-(BENZYL-CYCLOHEXYL-CARBAMOYL)-2-(1H-INDOL-3-YL)-ETHYL]-CARBAMIC ACID 9H-FLUOREN-9-YLMETHYL ESTER

Benzyl-cyclohexyl-amine was coupled to (S)-2-(9H-Fluoren-9-ylmethoxycarbonylamino)-3-(1H-indol-3-yl)-propionic acid as described in the method for Intermediate #1 of Example 1. Purification by HPLC produced the title compound. 1H NMR (400 MHz, DMSO-d₆) δ 7.87 (d, J=7.6 Hz, 2H), 7.71 (d, J=7.4 Hz, 1H), 7.61 (d, J=7.7 Hz, 1H), 7.44-6.94 (m, 14H), 4.93 (dd, J=7.2, 7.7 Hz, 1H), 4.46 (d, J=16.2 Hz, 1H), 4.53-4.07 (m, 5H), 3.17 (dd, J=8.5, 14.0 Hz, 1H), 3.05 (dd, J=6.3, 14.1 Hz, 1H), 1.7-0.67 (m, 10H); Low resolution mass spectrum (ES) m/e 598[(M+1)+, calcd for C₃₉H₄₀N₃O₃: 598 ]; 85% purity based on HPLC.

### Example 50

### (S)-6-AMINO-2-(2-BIPHENYL-4-YL-ACETYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to biphenyl-4-yl-acetic acid as described in the method of Example 23. Purification by HPLC produced the title compound. 1H NMR (400 MHz, DMSO-d₆) δ 9.98 (s, 1H), 8.43 (d, J=8.0 Hz, 1H), 7.671 (br s, 3H), 7.64 (d, J=7.2 Hz, 2H), 7.59 (d, J=8.2 Hz, 2H), 7.49-7.43 (m, 4H), 7.37-7.33 (m, 3H), 7.10 (d, J=8.3 Hz, 2H), 4.41 (dd, J=8.3, 13.8 Hz, 1H), 3.55 (s, 2H), 2.78-2.73 (m, 2H), 2.24 (s, 3H), 1.78-1.5 (m, 4H), 1.43-1.23 (m, 2H); Low resolution mass spectrum (ES) m/e 430[(M+1)+, calcd for C₂₇H₃₂N₃O₂: 430]; 98.6% purity based on HPLC.

### Example 51

### (S)-NAPHTHALENE-2-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to naphthalene-2-carboxylic acid as described in the method of Example 23. Purification by HPLC produced the title compound. 1H NMR (400 MHz, DMSO-d₆) δ 10.07 (s, 1H), 8.75 (d, J=7.8 Hz, 1H), 8.55 (s, 1H), 8.05-7.98 (m, 4H), 7.68 (br s, 3H), 7.65-7.59 (m, 2H), 7.52 (d, J=8.4 Hz, 2H), 7.12 (d, J=8.4 Hz, 2H), 4.64 (q, J=7.5 Hz, 1H), 2.81 (m 2H), 2.25 (s, 3H), 1.87 (dd, J=7.6, 14.8 Hz, 2H), 1.65-1.39 (m, 4H); Low resolution mass spectrum (ES) m/e 390[(M+1)+, calcd for C₂₄H₂₈N₃O₂: 390]; 96.9% purity based on HPLC.

### Example 52

### (S)-9H-FLUORENE-9-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to 9H-fluorene-9-carboxylic acid as described in the method of Example 23. Purification by HPLC produced the title compound. 1H NMR (400 MHz, DMSO-d₆) δ 10.04 (s, 1H), 8.84 (d, J=8.1 Hz, 1H), 7.88 (dd, J=5.0, 7.4 Hz, 2H), 7.71 (br s, 3H), 7.53 (t, J=7.4 Hz, 2H), 7.49 (d, J=8.4 Hz, 2H), 7.41 (dd, J=7.8, 16.6 Hz, 2H), 7.32 (dtd, J=0.9, 7.4, 11.3 Hz, 2H), 7.12 (d, J=8.4 Hz, 2H), 4.99 (s, 1H), 4.48 (dt, J=5.7, 8.4 Hz, 1H), 2.81 (m 2H), 2.25 (s, 3H); Low resolution mass spectrum (ES) m/e 429[(M+1)+, calcd for C₂₇H₃₀N₃O₂: 429]; 98.6% purity based on HPLC.

### Example 53

### (S)-6-AMINO-2-(2-9H-FLUOREN-9-YL-ACETYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (9H-Fluoren-9-yl)-acetic acid as described in the method of Example 23. Purification by HPLC produced the title compound. 1H NMR (400 MHz, DMSO-d₆) δ 10.09 (s, 1H), 8.32 (d, J=7.8 Hz, 1H), 7.87 (dd, J=6.0, 6.9 Hz, 2H), 7.70 (br s, 3H), 7.55 (m, 4H), 7.37 (dt, J=7.5, 10.1 Hz, 2H), 7.29 (dt, J=1.0, 7.5 Hz, 1H), 7.20 (dt, J=0.9, 7.5 Hz, 1H), 7.14 (d, J=8.3 Hz, 2H), 4.56 (dt, J=5.8, 8.2 Hz, 1H), 4.36 (t, J=7.5 Hz, 1H), 2.78 (m 2H), 2.62 (d, J=7.6 Hz, 2H); Low resolution mass spectrum (ES) m/e 443[(M+1)+, calcd for C₂₈H₃₂N₃O₂: 443]; 97.4% purity based on HPLC.

### Example 54

### (S)-BIPHENYL-2-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to biphenyl-2-carboxylic acid as described in the method of Example 23. Purification by HPLC produced the title compound. 1H NMR (400 MHz, DMSO-d₆) δ 9.89 (s, 1H), 8.39 (d, J=7.7 Hz, 1H), 7.66 (br s, 3H), 7.53-7.4 (m, 8H), 7.33-7.24 (m, 3H), 7.11 (d, J=8.3 Hz, 2H), 4.32 (dt, J=5.5, 8.4 Hz, 1H), 2.71 (m, 2H), 2.25 (s, 3H), 1.65-1.39 (m, 4H), 1.20-1.08 (m, 2H); Low resolution mass spectrum (ES) m/e 417[(M+1)+, calcd for C₂₆H₃₀N₃O₂: 417]; 100% purity based on HPLC.

### Example 55

### (+/−)-2-[6-AMINO-2-(S)-((9H-FLUOREN-9-YLMETHOXYCARBONYL)AMINO)-HEXANOYL-AMINO]-3-(1H-INDOL-3-YL)-PROPIONIC ACID METHYL ESTER

(S)-2-amino-3-(1H-indol-3-yl)-propionic acid methyl ester was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 8.26 (d, J=7.2 Hz, 0.6H), 7.86 (d, J=7.6 Hz, 2H), 7.67 (dd, J=7.9, 8.27 Hz, 2H), 7.44 (d, J=7.9 Hz, 1H), 7.39 (t, J=7.4 Hz, 2H), 7.34-7.25 (m, 3H), 7.12 (s, 1H), 7.05 (dd, J=7.5, 7.7 Hz, 1H), 6.96 (dd, J=7.1, 7.4 Hz, 1H), 4.53-4.43 (m, 1H), 4.26 (d, J=6.9 Hz, 2H), 4.21-4.14 (m, 1H), 3.99 (dd, J=5.1, 9.1 Hz, 1H), 3.52 (s, 3H), 3.12 (dd, J=6.0, 14.7 Hz, 1H), 3.05 (dd, J=7.6, 14.7 Hz, 1H), 2.70 (t, J=7.1 Hz, 2H), 1.63-1.40 (m, 4H), 1.33-1.14 (m, 2H); Low resolution mass spectrum (ES) m/e 570[(M+1)+, calcd for C₃₃H₃₇N₄O₅: 570]; 100% purity based on HPLC.

### Example 56

### (S)-{5-((9H-FLUOREN-9-YLMETHOXYCARBONYL)AMINO)-5-[2-(1H-INDOL-3-YL)-ETHYLCARBAMOYL]-PENTYL}-CARBAMIC ACID TERT-BUTYL ESTER

2-(1H-Indol-3-yl)-ethylamine was coupled to (S)-6-tert-Butoxycarbonylamino-2-(9H-fluoren-9-ylmethoxycarbonylamino)-hexanoic acid as described in the method for Intermediate #1 of Example 1. Purification by HPLC produced the title compound. 1H NMR (400 MHz, DMSO-d₆) δ 7.87 (d, J=7.5 Hz, 2H), 7.71 (d, J=7.1 Hz, 2H), 7.51 (d, J=8.1 Hz, 1H), 7.4 (t, J=7.4 Hz, 2H), 7.34-7.27 (m, 3H), 7.10 (s, 1H), 7.04 (dd, J=7.1, 7.5 Hz, 1H), 6.95 (dd, J=7.2, 7.5 Hz, 1H), 4.31-4.16 (m, 3H), 3.88 (dd, J=5.0, 9.0 Hz, 1H), 3.39-3.22 (m, 2H), 2.91-2.81 (m, 2H), 2.78 (t, J=7.4 Hz, 2H), 1.60-1.39 (m, 3H), 1.33 (s, 9H), 1.31-1.09 (m, 3H); Low resolution mass spectrum (ES) m/e 611[(M+1)+, calcd for C₃₆H₄₃N₄O₅: 611]; 92% purity based on HPLC.

### Example 57

### (S)-{5-AMINO-1-[2-(1H-INDOL-3-YL)-ETHYLCARBAMOYL]-PENTYL}-CARBAMIC ACID 9H-FLUOREN-9-YLMETHYL ESTER

(S)-{5-((9H-Fluoren-9-ylmethoxycarbonyl)amino)-5-[2-( 1H-indol-3-yl)-ethylcarbamoyl]-pentyl}-carbamic acid tert-butyl ester from Example 56 was deprotected as described in the method for Intermediate #2 of Example 1. Purification by HPLC produced the title compound. 1H NMR (400 MHz, DMSO-d₆) δ 7.88 (d, J=7.6 Hz, 2H), 7.71 (t, J=6.5 Hz, 2H), 7.52(d, J=7.9 Hz, 1H), 7.41 (t, J=7.3 Hz, 2H), 7.35-7.27 (m, 3H), 7.11 (s, 1H), 7.05 (dd, J=7.3, 7.6 Hz, 1H), 6.96 (dd, J=7.1, 7.7 Hz, 1H), 4.28 (d, J=7.1 Hz, 2H), 4.24-4.16 (m, 1H), 3.90 (dd, J=5.1, 9.0 Hz, 1H), 3.37-3.24 (m, 2H), 2.79 (dd, J=7.3, 7.5 Hz, 2H), 2.75-2.65 (m, 2H), 1.65-1.39 (m, 4H), 1.37-1.12 (m, 2H); Low resolution mass spectrum (ES) m/e 512[(M+1)+, calcd for C₃₁H₃₅N₄O₃: 512]; 100% purity based on HPLC.

### Example 58

### (S)-2-[6-AMINO-2-((9H-FLUOREN-9-YLMETHOXYCARBONYL)AMINO)-HEXANOYL-(R)-AMINO]-3-(1H-INDOL-3-YL)-PROPIONIC ACID METHYL ESTER

(R)-2-amino-3-(1H-indol-3-yl)-propionic acid methyl ester was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 8.28 (d, J=7.9 Hz, 0.5H), 7.87 (d, J=7.5 Hz, 2H), 7.70 (d, J=7.4 Hz, 2H), 7.47 (d, J=8.2 Hz, 1H), 7.40 (t, J=7.3 Hz, 2H), 7.34-7.26 (m, 3H), 7.10 (s, 1H), 7.05 (dd, J=7.4, 7.5 Hz, 1H), 6.97 (dd, J=7.3, 7.5 Hz, 1H), 4.52 (dd, J=5.7, 8.3 Hz, 1H), 4.26 (d, J=7.1 Hz, 2H), 4.22-4.14 (m, 1H), 4.00 (t, J=5.0 Hz, 1H), 3.58 (d, J=8.9 Hz, 3H), 3.15 (m, 1H), 3.02 (dd, J=5.5, 14.4 Hz, 1H), 2.71-2.57 (m, 2H), 1.49-1.25 (m, 4H), 1.19-1.10 (m, 2H); Low resolution mass spectrum (ES) m/e 570[(M+1)+, calcd for C₃₃H₃₇N₄O₅: 570]; 100% purity based on HPLC.

### Example 59

### (S)-3-{4-[6-AMINO-2-((9H-FLUOREN-9-YLMETHOXYCARBONYL)AMINO)-HEXANOYLAMINO]PHENYL}-ACRYLIC ACID ETHYL ESTER

3-(4-Amino-phenyl)-acrylic acid methyl ester was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 7.87 (d, J=7.5 Hz, 2H), 7.83-7.51 (m, 7H), 7.40(dd, J=6.3, 7.3 Hz, 2H), 7.31 (dd, J=7.3, 12.0 Hz, 2H), 6.49 (d, J=15.8 Hz, 1H), 4.34-4.04 (m, 6H), 2.75 (t, J=7.4 Hz, 2H), 1.75-1.25 (m, 6H), 1.22 (t, J=7.0 Hz, 3H); Low resolution mass spectrum (ES) m/e 543[(M+1)+, calcd for C₃₂H₃₆N₃O₅: 543]; 100% purity based on HPLC.

### Example 60

### (S,S)-6-AMINO-2-(2-AMINO-3-NAPHTHALEN-1-YL-PROPIONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (S)-2-tert-Butoxycarbonylamino-3-naphthalen-1-yl-propionic acid as described in the method of Example 23. Purification by HPLC produced the title compound. ¹H NMR (400 MHz, DMSO-d₆) δ 10.06 (s, 1H), 8.75 (d, J=8.0 Hz, 1H), 8.27 (br s, 3H), 8.20 (d, J=8.3 Hz, 1H), 7.92 (d, J=8.0 Hz, 1H), 7.78 (d, J=8.1 Hz, 1H), 7.84-7.69 (br s, 3H), 7.55 (m, 2H), 7.47 (d, J=8.5 Hz, 2H), 7.36 (d, J=6.9 Hz, 1H), 7.28 (app t, J=7.5 Hz, 1H), 7.154 (d, J=8.2 Hz, 2H), 4.55 (dd, J=7.7, 14.2 Hz, 1H), 4.19 (t, J=7.1 Hz, 1H), 3.50 (dd, J=7.3, 14.0 Hz, 1H), 3.41 (dd, J=7.1, 14.1 Hz, 1H), 2.75 (t, J=7.5 Hz, 2H), 2.28 (s, 3H), 1.74-1.65 (m, 1H), 1.61-1.5 (m, 3H), 1.42-1.24 (m, 2H); Low resolution mass spectrum (ES) m/e 433[(M+1)+, calcd for C₂₈H₃₃N₄O₂: 433]; 98.8% purity based on HPLC.

### Example 61

### (S,R)-6-AMINO-2-(2-AMINO-3-NAPHTHALEN-1-YL-PROPIONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (R)-2-tert-Butoxycarbonylamino-3-naphthalen-2-yl-propionic acid as described in the method of Example 23. Purification by HPLC produced the title compound. 1H NMR (400 MHz, DMSO-d₆) δ 10.08 (s, 1H), 8.79 (d, J=8.0 Hz, 1H), 8.26 (br s, 3H), 7.92-7.85 (m, 2H), 7.77 (s, 1H), 7.70 (br s, 3H), 7.55-7.45 (m, 5H), 7.11 (d, J=8.4 Hz, 2H), 4.38 (dd, J=7.9, 13.9 Hz, 1H), 4.24 (br m, 1H), 3.24 (d, J=6.8, 13.7 Hz, 1H), 3.17 (dd, J=7.7, 13.7 Hz, 1H), 2.55 (m, 2H), 2.24 (s, 1H), 1.56-1.29 (m, 4H), 1.08-0.91 (m, 2H); Low resolution mass spectrum (ES) m/e 433[(M+1)+, calcd for C₂₆H₃₃N₄O₂: 433]; 95.6% purity based on HPLC.

### Example 62

### (S)-4-METHYL-NAPHTHALENE-1-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to 4-methyl-naphthalene-1-carboxylic acid as described in the method of Example 23. Purification by HPLC produced the title compound. ¹H NMR (400 MHz, DMSO-d₆) δ 10.09 (s, 1H), 8.66 (d, J=7.6 Hz, 1H), 8.28 (d, J=7.9 Hz, 1H), 8.08 (d, J=8.3 Hz, 1H), 7.69 (br s, 3H), 7.62-7.53 (m, 5H), 7.41 (d, J=7.2 Hz, 1H), 7.14 (d, J=8.4 Hz, 2H), 4.62 (dd, J=8.1, 13.8 Hz, 1H), 2.85-2.77 (m, 2H), 2.69 (s, 3H), 2.27 (s, 3H), 1.87-1.74 (m, 2H), 1.67-1.41 (m, 4H); Low resolution mass spectrum (ES) m/e 404[(M+1)+, calcd for C₂₅H₃₀N₃O₂: 404]; 100% purity based on HPLC.

### Example 63

### (S)-2-METHYL-NAPHTHALENE-1-CARBOXYLIC ACID (5-AMINO-1-P-TOLYLCARBAMOYL-PENTYL)-AMIDE

The title compound was prepared by coupling p-Tolylamine to (S)-6-tert-Butoxycarbonylamino-2-(9H-fluoren-9-ylmethoxycarbonylamino)-hexanoic acid, and then to 2-methyl-naphthalene-1-carboxylic acid as described in the method of Example 23. Purification by HPLC produced the title compound. ¹H NMR (400 MHz, DMSO-d₆) δ 10.10 (s, 1H), 8.78 (d, J=7.1 Hz, 1H), 7.92-7.86 (m, 3H), 7.69 (br s, 3H), 7.55 (d, J=8.4 Hz, 2H), 7.53-7.45 (m, 2H), 7.40 (d, J=8.5 Hz, 1H), 7.15 (d, J=8.3 Hz, 2H), 4.64 (dd, J=7.9, 13.5 Hz, 1H), 2.83-2.75 (m, 2H), 2.43 (s, 3H), 2.27 (s, 3H), 1.85-1.69 (m, 2H), 1.66-1.41 (m, 4H); Low resolution mass spectrum (ES) m/e 404[(M+1)+, calcd for C₂₅H₃₀N₃O₂: 404]; 98.9% purity based on HPLC.

### Example 64

### (S)-4-FLUORO-NAPHTHALENE-1-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to 4-fluoro-naphthalene-1-carboxylic acid as described in the method of Example 23. Purification by HPLC produced the title compound. 1H NMR (400 MHz, DMSO-d₆) δ 10.10 (s, 1H), 8.77 (d, J=7.5 Hz, 1H), 8.32 (m, 1H), 8.11 (m, 1H), 7.71-7.65 (m, 6H), 7.53 (d, J=8.4 Hz, 2H), 7.40 (dd, J=8.0, 10.6 Hz, 1H), 7.13 (d, J=8.3 Hz, 2H), 4.61 (dd, J=8.4, 13.5 Hz, 1H), 2.84-2.76 (m, 2H), 2.26 (s, 3H), 1.85-1.72 (m, 2H), 1.66-1.38 (m, 4H); Low resolution mass spectrum (ES) m/e 408[(M+1)+, calcd for C₂₄H₂₇FN₃O₂: 408]; 96.3% purity based on HPLC.

### Example 65

### (S)-3-METHOXY-NAPHTHALENE-2-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to 3-methoxy-naphthalene-2-carboxylic acid as described in the method of Example 23. Purification by HPLC produced the title compound. ¹H NMR (400 MHz, DMSO-d₆) δ 10.10 (s, 1H), 8.67 (d, J=7.6 Hz, 1H), 8.38 (s, 1H), 7.97 (d, J=8.1 Hz, 1H), 7.89 (d, J=8.2 Hz, 1H), 7.64 (br s, 3H), 7.58-7.5 (m, 4H), 7.42 (ddd, J=0.9, 6.8, 8.0 Hz, 1H), 7.14 (d, J=8.4 Hz, 2H), 4.73 (dt, J=5.4, 7.8 Hz, 1H), 4.03 (s, 3H), 2.84-2.76 (m, 2H), 2.26 (s, 3H), 1.93-1.74 (m, 2H), 1.62-1.55 (m, 2H), 1.49-1.38 (m, 2H); Low resolution mass spectrum (ES) m/e 420[(M+1)+, calcd for C₂₅H₃₀N₃O₃: 420]; 98.8% purity based on HPLC.

### Example 66

### (S)-6-METHOXY-NAPHTHALENE-2-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to 6-methoxy-naphthalene-2-carboxylic acid as described in the method of Example 23. Purification by HPLC produced the title compound. 1H NMR (400 MHz, DMSO-d₆) δ 10.05 (s, 1H), 8.64 (d, J=7.7 Hz, 1H), 8.48 (s, 1H), 7.94 (m, 2H), 7.889 (d, J=8.6 Hz, 1H), 7.67 (br s, 3H), 7.50 (d, J=8.4 Hz, 2H), 7.41 (d, J=8.4 Hz, 2H), 7.38 (d, J=2.3 Hz, 1H), 7.22 (dd, J=2.3, 9.0 Hz, 1H), 4.62 (dd, J=7.6, 14.8 Hz, 1H), 3.90 (s, 3H), 2.83-2.78 (m, 2H), 2.24 (s, 3H), 1.87-1.82 (m, 2H), 1.63-1.37 (m, 4H); Low resolution mass spectrum (ES) m/e 420[(M+1)+, calcd for C₂₅H₃₀N₃O₃: 420]; 99.3% purity based on HPLC.

### Example 67

### (S)-ACENAPHTHENE-5-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to acenaphthene-5-carboxylic acid as described in the method of Example 23. Purification by HPLC produced the title compound. 1H NMR (400 MHz, DMSO-d₆) δ 10.07 (s, 1H), 8.55 (d, J=7.7 Hz, 1H), 8.10 (d, J=8.4 Hz, 1H), 7.99 (d, J=7.1 Hz, 1H), 7.68 (br s, 3H), 7.54-7.49 (m, 3H), 7.36 (d, J=7.7 Hz, 2H), 7.13 (d, J=8.3 Hz, 2H), 4.64 (dd, J=7.7, 14.4 Hz, 1H), 3.34 (obscured, 4H), 2.84-2.79 (m, 2H), 2.26 (s, 3H), 1.85-1.80 (m, 2H), 1.65-1.41 (m, 4H); Low resolution mass spectrum (ES) m/e 416[(M+1)+, calcd for C₂₆H₃₀N₃O₂: 416]; 99.5% purity based on HPLC.

### Example 68

### (S)-6-AMINO-2-(NAPHTHALENE-1-SULFONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to naphthalene-1-Sulfonyl chloride as described in the method of Example 26. Purification by HPLC produced the title compound. 1H NMR (400 MHz, DMSO-d₆) δ 9.70 (s, 1H), 8.69 (d, J=8.5 Hz, 1H), 8.45 (d, J=9.1 Hz, 1H), 8.14 (dd, J=0.8, 7.3 Hz, 1H), 8.11 (d, J=8.2 Hz, 1H), 8.01 (d, J=8.1 Hz, 1H), 7.69 (ddd, J=1.2, 6.9, 8.5 Hz, 1H), 7.63 (t, J=7.9 Hz, 1H), 7.61 (br s, 3H), 7.53 (t, J=7.8 Hz, 1H), 7.10 (d, J=8.4 Hz, 2H), 7.00 (d, J=8.5 Hz, 2H), 3.83 (dt, J=5.7, 8.8 Hz, 1H), 2.59-2.5 (m, 2H), 2.21 (s, 3H), 1.57-1.45 (m, 2H), 1.37-1.15 (m, 3H), 1.09-0.99 (m, 1H); Low resolution mass spectrum (ES) m/e 426[(M+1)+, calcd for C₂₃H₂₈N₃O₃S: 426]; 100% purity based on HPLC.

### Example 69

### (S)-6-AMINO-2-(NAPHTHALENE-2-SULFONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to naphthalene-2-Sulfonyl chloride as described in the method of Example 26. Purification by HPLC produced the title compound. 1H NMR (400 MHz, DMSO-d₆) δ 9.98 (s, 1H), 8.37 (d, J=1.2 Hz, 1H), 8.21 (d, J=9.0 Hz, 1H), 7.98 (t, J=7.5 Hz, 2H), 7.93 (d, J=8.2 Hz, 1H), 7.79 (dd, J=1.8, 8.7 Hz, 1H), 7.63 (m, 4H), 7.57 (t, J=7.2 Hz, 1H), 7.03 (d, J=8.4 Hz, 2H), 6.89 (d, J=8.4 Hz, 2H), 3.92 (dt, J=6.0, 8.7 Hz, 1H), 2.72-2.62 (m, 2H), 2.17 (s, 3H), 1.64-1.43 (m, 4H), 1.39-1.3 (m, 1H), 1.25-1.06 (m, 1H); Low resolution mass spectrum (ES) m/e 426[(M+1)+, calcd for C₂₃H₂₈N₃O₃S: 426]; 99.7% purity based on HPLC.

### Example 70

### (S)-6-AMINO-2-(BIPHENYL-4-SULFONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to biphenyl-4-Sulfonyl chloride as described in the method of Example 26. Purification by HPLC produced the title compound. 1H NMR (400 MHz, DMSO-d₆) δ 9.82 (s, 1H), 8.13 (d, J=9.3 Hz, 1H), 7.81 (d, J=8.4 Hz, 2H), 7.67 (m, 5H), 7.50-7.39 (m, 5H), 7.19 (d, J=8.4 Hz, 2H), 6.97 (d, J=8.4 Hz, 2H), 3.89 (dt, J=6.1,8.8 Hz, 1H), 2.76-2.72 (m, 2H), 2.18 (s, 3H), 1.65-1.46 (m, 4H), 1.42-1.34 (m, 1H), 1.29-1.20 (m, 1H); Low resolution mass spectrum (ES) m/e 452[(M+1)+, calcd for C₂₅H₃₀N₃O₃S: 452]; 99.7% purity based on HPLC.

### Example 71

### (S)-6-AMINO-2-(3-NAPHTHALEN-1-YL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-isocyanato-naphthalene as described in the method of Example 26. Purification by HPLC produced the title compound. ¹H NMR (400 MHz, DMSO-d₆) δ 10.14 (s, 1H), 8.77 (s, 1H), 8.14 (d, J=8.3 Hz, 1H), 8.04 (d, J=7.6 Hz, 1H), 7.90 (d, J=8.1 Hz, 1H), 7.66 (br s, 3H), 7.56-7.51 (m, 4.3H), 7.41 (t, J=7.9 Hz, 1H), 7.12 (m, 2.7H), 4.47 (dd, J=7.4, 13.8 Hz, 1H), 2.81 (m, 2H), 2.26 (s, 3H), 1.82-1.75 (m, 1H), 1.69-1.56 (m, 3H), 1.45-1.37 (m, 2H); Low resolution mass spectrum (ES) m/e 405[(M+1)⁺, calcd for C₂₄H₂₉N₄O₂: 405]; 99.8% purity based on HPLC.

### Example 72

### (S)-6-AMINO-2-(3-NAPHTHALEN-2-YL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 2-isocyanato-naphthalene as described in the method of Example 26. Purification by HPLC produced the title compound. ¹H NMR (400 MHz, DMSO-d₆) δ 10.12 (s, 1H), 8.97 (s, 1H), 8.03 (s, 1H), 7.78 (dd, J=4.3, 8.4 Hz, 2H), 7.72 (d, J=8.3 Hz, 1H), 7.68 (br s, 3H), 7.51 (d, J=8.3 Hz, 2H), 7.41 (m, 2H), 7.32 (t, J=7.5 Hz, 1H), 7.12 (d, J=8.2 Hz, 2H), 6.66 (d, J=8.4 Hz, 1H), 4.43 (dd, J=7.8, 13.8 Hz, 1H), 2.81 (m, 2H), 2.25 (s, 3H), 1.81-1.74 (m, 1H), 1.68-1.53 (m, 3H), 1.47-1.35 (m, 2H); Low resolution mass spectrum (ES) m/e 405[(M+1)⁺, calcd for C₂₄H₂₉N₄O₂: 405]; 99.0% purity based on HPLC.

### Example 73

### (S)-6-AMINO-2-(3-BIPHENYL-2-YL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 2-Isocyanato-biphenyl as described in the method of Example 26. Purification by HPLC produced the title compound. 1H NMR (400 MHz, DMSO-d₆) δ 10.04 (s, 1H), 7.88 (d, J=8.2 Hz, 1H), 7.67 (br s, 3H), 7.62 (s, 1H), 7.46 (m, 4H), 7.38 (t, J=7.2 Hz, 1H), 7.33 (d, J=7.7 Hz, 2H), 7.24 (t, J=7.7 Hz, 1H), 7.13 (d, J=7.5 Hz, 1H), 7.09 (d, J=8.1 Hz, 2H), 7.03 (m, 2H), 4.43 (dd, J=7.7, 14.0 Hz, 1H), 2.75 (m, 2H), 2.23 (s, 3H), 1.68-1.63 (m, 1H), 1.57-1.47 (m, 3H), 1.37-1.21 (m, 2H); Low resolution mass spectrum (ES) m/e 431[(M+1)+, calcd for C₂₆H₃₁N₄O₂: 431]; 99.6% purity based on HPLC.

### Example 74

### (S)-6-AMINO-2-[3-(4-BENZYL-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-benzyl-4-isocyanato-benzene as described in the method of Example 26. Purification by HPLC produced the title compound. 1H NMR (400 MHz, DMSO-d₆) δ 10.07 (s, 1H), 8.66 (s, 1H), 7.68 (br s, 3H), 7.49 (d, J=8.4 Hz, 2H), 7.27 (m, 4H), 7.17 (m, 3H), 7.11 (d, J=8.5 Hz, 2H), 7.07 (d, J=8.5 Hz, 2H), 6.51 (d, J=8.3 Hz, 1H), 4.37 (dt, J=5.7, 8.1 Hz, 1H), 3.84 (s, 2H), 2.81-2.74 (m, 2H), 2.25 (s, 3H), 1.76-1.69 (m, 1H), 1.63-1.51 (m, 3H), 1.43-1.30 (m, 2H); Low resolution mass spectrum (ES) m/e 445[(M+1)⁺, calcd for C₂₇H₃₃N₄O₂: 445]; 99.8% purity based on HPLC.

### Example 75

### (S)-6-AMINO-2-[3-(4-BENZOYL-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (4-isocyanato-phenyl)-phenyl-methanone as described in the method of Example 26. Purification by HPLC produced the title compound. 1H NMR (400 MHz, DMSO-d₆) δ 10.12 (s, 1H), 9.28 (s, 1H), 7.70-7.63 (m, 7H), 7.55 (m, 4H), 7.50 (d, J=8.4 Hz, 2H), 7.12 (d, J=8.5 Hz, 2H), 6.79 (d, J=8.2 Hz, 1H), 4.41 (dt, J=5.6, 8.0 Hz, 1H), 2.82-2.76 (m, 2H), 2.25 (s, 3H), 1.80-1.73 (m, 1H), 1.67-1.52 (m, 3H), 1.45-1.32 (m, 2H); Low resolution mass spectrum (ES) m/e 459[(M+1)⁺, calcd for C₂₇H₃₁N₄O₃: 449]; 99.7% purity based on HPLC.

### Example 76

### (S)-6-AMINO-2-[3-(2-BIPHENYL-4-YL-ETHYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 4-(2-Isocyanato-ethyl)-biphenyl as described in the method of Example 26. Purification by HPLC produced the title compound. 1H NMR (400 MHz, DMSO-d₆) δ 9.98 (s, 1H), 7.68 (br s, 3H), 7.62 (d, J=7.5 Hz, 2H), 7.51 (d, J=8.1 Hz, 2H), 7.49 (d, J=8.4 Hz, 2H), 7.45 (t, J=7.7 Hz, 2H), 7.34 (t, J=7.4 Hz, 1H), 7.29 (d, J=8.1 Hz, 2H), 7.10 (d, J=8.1 Hz, 2H), 6.27 (d, J=8.5 Hz, 1H), 6.14 (t, J=5.7 Hz, 1H), 4.29 (dt, J=5.6, 8.2 Hz, 1H), 3.34-3.22 (m, 2H), 2.80-2.75 (m, 1H), 2.72 (t, J=7.1 Hz, 2H), 2.25 (s, 3H), 1.68-1.48 (m, 4H), 1.40-1.23 (m, 2H); Low resolution mass spectrum (ES) m/e 459[(M+1)⁺, calcd for C₂₈H₃₅N₄O₂: 459]; 99.6% purity based on HPLC.

### Example 77

### (S)-6-AMINO-2-(2-(S)-AMINO-3-NAPHTHALEN-1-YL-PROPIONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid then to (R)-2-tert-Butoxycarbonylamino-3-naphthalen-1-yl-propionic acid as described in the method of Example 23. Purification by HPLC produced the title compound. ¹H NMR (400 MHz, DMSO-d₆) δ 10.03 (s, 1H), 8.66 (d, J=8.0 Hz, 1H), 8.34 (br s, 3H), 8.20 (d, J=8.3 Hz, 1H), 7.97 (d, J=7.8 Hz, 1H), 7.88 (d, J=8.2 Hz, 1H), 7.68 (br s, 3H), 7.64-7.57 (m, 2H), 7.46 (m, 3H), 7.40 (d, J=6.9 Hz, 1H), 7.10 (d, J=8.4 Hz, 1H), 4.32 (dd, J=7.4, 14.1 Hz, 1H), 4.23 (br s, 1H), 3.50 (dd, J=8.5, 13.7 Hz, 1H), 3.44 (dd, J=6.8, 13.8 Hz, 1H), 2.63 (m, 2H), 2.23 (s, 3H), 1.40-1.33 (m, 3H), 1.26-1.2 (m, 1H), 0.94-0.78 (m, 2H); Low resolution mass spectrum (ES) m/e 433[(M+1)+, calcd for C₂₆H₃₃N₄O₂: 433]; 97.0% purity based on HPLC.

### Example 78

### (S)-6-AMINO-2-[3-(5,6,7,8-TETRAHYDRO-NAPHTHALEN-1-YL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid then to 5-Isocyanato-1,2,3,4-tetrahydro-naphthalene as described in the method of Example 26. Purification by HPLC produced the title compound. 1H NMR (400 MHz, DMSO-d₆) δ 10.08 (s, 1H), 7.75 (s, 1H), 7.66 (br s, 3H), 7.63 (d, J=7.9 Hz, 1H), 7.50 (d, J=8.4 Hz, 2H), 7.12 (d, J=8.4 Hz, 2H), 7.01 (d, J=8.3 Hz, 1H), 6.96 (t, J=7.8 Hz, 1H), 6.69 (d, J=7.5 Hz, 1H), 4.38 (dd, J=7.8, 13.4 Hz, 1H), 2.78 (m, 2H), 2.69 (t, J=5.9 Hz, 2H), 2.25 (s, 3H), 1.79-1.3 (m, 11H); Low resolution mass spectrum (ES) m/e 409[(M+1)+, calcd for C₂₄H₃₃N₄O₂: 409]; 97.8% purity based on HPLC.

### Example 79

### NAPHTHALENE-1-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid then to Naphthalene-1-carboxylic acid as described in the method of Example 23. Purification by HPLC produced the title compound. 1H NMR (400 MHz, DMSO-d₆) δ 10.10 (s, 1H), 8.74 (d, J=7.5 Hz, 1H), 8.24 (dq, J=3.3, 6.8 Hz, 1H), 8.03 (d, J=8.3 Hz, 1H), 7.98 (dq, J=3.4, 6.9 Hz, 1H), 7.70 (br s, 3H), 7.67 (dd, J=1.1, 7.0 Hz, 1H), 7.58-7.54 (m, 5H), 7.14 (d, J=8.4 Hz, 2H), 4.63 (ddd, J=5.4, 7.9, 8.9 Hz, 1H), 2.84-2.78 (m, 2H), 2.27 (s, 3H), 1.86-1.75 (m, 2H), 1.6-1.42 (m, 4H); Low resolution mass spectrum (ES) m/e 390[(M+1)+, calcd for C₂₄H₂₈N₃O₂: 460]; 99.9% purity based on HPLC.

### Example 80

### (S)-[5-AMINO-1-(3-FLUORO-PHENYLCARBAMOYL)-PENTYL]-CARBAMIC ACID 9H-FLUOREN-9-YLMETHYL ESTER

m-Fluoro phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 10.3 (s, 1H), 7.89 (d, J=7.5 Hz, 2H), 7.72 (t, J=7.4 Hz, 2H), 7.69-7.56 (m, 4H), 7.41 (t, J=7.4 Hz, 2H), 7.38-7.27 (m, 4H), 6.88 (dd, J=7.6, 9.3 Hz, 1H), 4.35-4.16 (m, 3H), 4.11 (ddd, J=5.8, 8.4, 8.4 Hz, 1H), 2.77 (dt, J=5.9, 8.1 Hz, 2H), 1.76-1.23 (m, 6H); Low resolution mass spectrum (ES) m/e 462[(M+1)+, calcd for C₂₇H₂₉FN₃O₃: 462]; 100% purity based on HPLC.

### Example 81

### (S)-[5-AMINO-1-(INDAN-5-YLCARBAMOYL)-PENTYL]-CARBAMIC ACID 9H-FLUOREN-9-YLMETHYL ESTER

Indan-5-ylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 9.88 (s, 1H), 7.89 (d, J=7.5 Hz, 2H), 7.73 (t, J=7.5 Hz, 2H), 7.6 (d, J=8.1 Hz, 2H), 7.51 (s, 1H), 7.41 (t, J=7.5 Hz, 1H), 7.33 (dd, J=3.4, 7.4 Hz, 2H), 7.28 (d, J=8.1 Hz, 1H), 7.13 (d, J=8.1 Hz, 1H), 4.36-4.17 (m, 1H), 4.25 (AB q, J=7.32, 21.9 Hz, 2H), 4.11 (dt, J=5.9, 8.4 Hz, 1H), 2.87-2.7 (m, 6H), 1.98 (p, J=7.4 Hz, 2H), 1.74-1.25 (m, 6H); Low resolution mass spectrum (ES) m/e 484[(M+1)+, calcd for C₃₀H₃₄N₃O₃: 484]; 98.5% purity based on HPLC.

### Example 82

### (S)-[5-AMINO-1-(1H-INDAZOL-5-YLCARBAMOYL)-PENTYL]-CARBAMIC ACID 9H-FLUOREN-9-YLMETHYL ESTER

1H-Indazol-5-ylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 8.15 (s, 1H), 7.99-7.84 (m, 2H), 7.79-7.57 (m, 5H), 7.51-7.28 (m, 4H), 4.48-4.08 (m, 4H), 2.78 (d, J=6.2 Hz, 2H), 1.92-1.18 (m, 6H); Low resolution mass spectrum (ES) m/e 484[(M+1)+, calcd for C₂₈H₃₀N₅O₃: 484]; 85.2% purity based on HPLC.

### Example 83

### (S)-[5-AMINO-1-(4-METHOXY-PHENYLCARBAMOYL)-PENTYL]-CARBAMIC ACID 9H-FLUOREN-9-YLMETHYL ESTER

p-Methoxy phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 9.88 (s, 1H), 7.89 (d, J=7.5 Hz, 2H), 7.73 (t, J=7.5 Hz, 2H), 7.6 (br s, 2H), 7.61 (d, J=8.2 Hz, 2H), 7.5 (d, J=9.0 Hz, 2H), 7.41 (t, J=7.4 Hz, 2H), 7.31 (ddd, J=4.1, 7.4, 7.4 Hz, 2H), 6.87 (d, J=6.9 Hz, 2H), 4.35-4.17 (m, 3H), 4.09 (ddd, J=5.9, 8.5, 8.5 Hz, 1H), 3.71 (s, 3H), 2.77 (dd, J=5.7, 10.6 Hz, 2H), 1.79-1.23 (m, 6H); Low resolution mass spectrum (ES) m/e 474[(M+1)+, calcd for C₂₈H₃₂N₃O₄: 484]; 98.3% purity based on HPLC.

### Example 84

### (R)-(2-AMINO-2-P-TOLYLCARBAMOYL-ETHYL)-CARBAMIC ACID 9H-FLUOREN-9-YLMETHYL ESTER

p-Tolylamine was coupled to (R)-2-tert-Butoxycarbonylamino-3-(9H-fluoren-9-ylmethoxycarbonylamino)-propionic acid as described in the method of Example 6. Purification by HPLC produced the title compound. 1H NMR (400 MHz, DMSO-d₆) δ 7.86 (d, J=7.6 Hz, 2H), 7.64-7.57 (m, 2H), 7.4 (t, J=8.2 Hz, 4H), 7.28 (t, J=7.4 Hz, 2H), 7.11 (d, J=8.3 Hz, 2H), 4.32-4.22 (m, 2H), 4.16 (t, J=6.6 Hz, 1H), 3.93 (t, J=5.5 Hz, 1H), 3.53 (m, 1H), 3.44 (dd, J=6.0, 14.4 Hz, 1H), 2.22 (s, 3H); Low resolution mass spectrum (ES) m/e 416[(M+1)+, calcd for C₂₅H₂₆N₃O₃: 416]; 99% purity based on HPLC.

### Example 85

### (S)-2-P-TOLYLCARBAMOYL-PYRROLIDINE-1-CARBOXYLIC ACID 9H-FLUOREN-9-YLMETHYL ESTER

p-Tolylamine was coupled to (S)-Pyrrolidine-1,2-dicarboxylic acid 1-(9H-fluoren-9-ylmethyl) ester as described in the method for intermediate #1 of Example 1. Purification by HPLC produced the title compound. 1H NMR (400 MHz, DMSO-d₆) δ 10.12 (s, 0.3H), 9.94 (s, 0.3H), 7.87 (d, J=7.4 Hz, 1H), 7.79 (d, J=7.6 Hz, 1H), 7.64 (t, J=7.6 Hz, 1H), 7.59-7.47 (m, 2H), 7.45-7.36 (m, 2H), 7.36-7.26 (m, 2H), 7.13-6.98 (m, 3H), 4.46 (dd, J=3.2, 8.7 Hz, 0.5H), 4.29-4.13 (m, 2.5H), 4.09-3.99 (m, 1H), 3.54-3.29 (m, 2H), 2.35-2.09 (m, 4H), 2.00-1.75 (m, 3H); Low resolution mass spectrum (ES) m/e 427[(M+1)+, calcd for C₂₇H₂₇N₂O₃: 427]; 98% purity based on HPLC.

### Example 86

### (R)-2-P-TOLYLCARBAMOYL-PYRROLIDINE-1-CARBOXYLIC ACID 9H-FLUOREN-9-YLMETHYL ESTER

p-Tolylamine was coupled to (R)-Pyrrolidine-1,2-dicarboxylic acid 1-(9H-fluoren-9-ylmethyl) ester as described in the method for Intermediate #1 of Example 1. Purification by HPLC produced the title compound. 1H NMR (400 MHz, DMSO-d₆) δ 10.12 (s, 0.3H), 9.94 (s, 0.3H), 7.87 (d, J=7.4 Hz, 1H), 7.79 (d, J=7.6 Hz, 1H), 7.64 (t, J=7.6 Hz, 1H), 7.59-7.47 (m, 2H), 7.45-7.36 (m, 2H), 7.36-7.26 (m, 2H), 7.13-6.98 (m, 3H), 4.46 (dd, J=3.2, 8.7 Hz, 0.5H), 4.29-4.13 (m, 2.5H), 4.09-3.99 (m, 1H), 3.54-3.29 (m, 2H), 2.35-2.09 (m, 4H), 2.00-1.75 (m, 3H); Low resolution mass spectrum (ES) m/e 427[(M+1)+, calcd for C₂₇H₂₇N₂O₃: 427]; 98% purity based on HPLC.

### Example 87

### (4R,S)-4-HYDROXY-2-P-TOLYLCARBAMOYL-PYRROLIDINE-1-CARBOXYLIC ACID 9H-FLUOREN-9-YLMETHYL ESTER

p-Tolylamine was coupled to (4R,S)-4-Hydroxy-pyrrolidine-1,2-dicarboxylic acid 1-(9H-fluoren-9-ylmethyl) ester as described in the method for Intermediate # 1 of Example 1. Purification by HPLC produced the title compound. 1H NMR (400 MHz, DMSO-d₆) δ 10.17 (s, 0.15H), 10.01 (s, 0.15H), 7.87 (d, J=7.5 Hz, 1H), 7.78 (d, J=7.6 Hz, 1H), 7.64 (dd, J=5.8, 6.7 Hz, 1H), 7.61-7.52 (m, 1H), 7.51 (d, J=8.4 Hz, 1H), 7.46-7.36 (m, 2H), 7.35-7.27 (m, 2H), 7.12-7.02 (m, 3H), 4.56 (t, J=7.9 Hz, 0.5H), 4.41-4.28 (m, 1.5H), 4.27-4.15 (m, 2H), 4.06-3.98 (m, 1H), 3.57-3.34 (m, 2H); Low resolution mass spectrum (ES) m/e 443[(M+1)+, calcd for C₂₇H₂₇N₂O₄: 443]; 98% purity based on HPLC.

### Example 88

### (S)-N-(9-{[5-AMINO-1-(BENZYL-CYCLOHEXYL-CARBAMOYL)-PENTYLCARBAMOYL]-METHYL}-9H-PURIN-6-YL)-BENZAMIDE

Benzyl-cyclohexyl-amine was coupled to (S)-6-tert-Butoxycarbonylamino-2-(9H-fluoren-9-ylmethoxycarbonylamino)-hexanoic acid, and then to (6-Benzoylamino-purin-9-yl)-acetic acid as described in the method of Example 23. Purification by HPLC produced the title compound. ¹H NMR (400 MHz, DMSO-d₆) δ 8.68 (d, J=6.9 Hz, 1H), 8.4 (s, 1H), 8.02 (d, J=7.3 Hz, 2H), 7.64 (dd, J=7.2, 7.5 Hz, 1H), 7.55 (dd, J=7.4, 7.8 Hz, 2H), 7.32-7.10 (m, 5H), 5.05 (s, 0.85H), 4.99 (s, 0.65H), 4.84 (d, J=5.0, 9.0 Hz, 0.5H), 4.6 (d, J=19.5 Hz, 0.7H), 4.51 (d, J=18.1 Hz, 0.7H), 4.46-4.35 (m, 0.9H), 4.24-4.13 (m, 0.8H), 3.8-3.65 (m, 1H), 2.83-2.7 (m, 1H); Low resolution mass spectrum (ES) m/e 597[(M+1)+, calcd for C₃₃H₄₁N₈O₃: 597 ]; 98% purity based on HPLC.

### Example 89

### (S)-6-AMINO-2-(3-1H-INDOL-3-YL-PROPIONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 3-(1H-Indol-3-yl)-propionic acid as described in the method of Example 23. Purification by HPLC produced the title compound. ¹H NMR (400 MHz, DMSO-d6) δ 10.77 (s, 1H), 9.96 (s, 1H), 8.17 (d, J=8.0 Hz, 1H), 7.68 (br s, 3H), 7.53 (d, J=7.9 Hz, 1H), 7.48 (d, J=8.4 Hz, 2H), 7.32 (d, J=8.1 Hz, 1H), 7.11 (m, 3H), 7.05 (t, J=7.7 Hz, 1H), 6.96 (d, J=7.0 Hz, 1H), 4.41 (dt, J=5.6, 8.4 Hz, 1H), 2.92 (t, J=7.7 Hz, 2H), 2.77-2.71 (m, 2H), 2.54 (m, 2H), 2.25 (s, 3H), 1.72-1.65 (m, 1H), 1.61-1.47 (m, 3H), 1.37-1.21 (m, 2H); Low resolution mass spectrum (ES) m/e 407[(M+1)+, calcd for C₂₄H₃₁N₄O₂: 407]; 96.6% purity based on HPLC.

### Example 90

### (S)-6-AMINO-2-[3-(1H-INDOL-3-YL)-2-(S)-METHYLAMINO-PROPIONYLAMINO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (S)-3-(1H-Indol-3-yl)-2-methylamino-propionic acid as described in the method of Example 23. Purification by HPLC produced the title compound. 1H NMR (400 MHz, DMSO-d₆) δ 11.03 (d, J=2.1 Hz, 1H), 10.14 (s, 1H), 9.04 (d, J=8.1 Hz, 1H), 8.88 (br s, 1H), 8.71 (br s, 1H), 7.75 (br s, 3H), 7.58 (d, J=8.0 Hz, 1H), 7.50 (d, J=8.4 Hz, 2H), 7.32 (d, J=8.1 Hz, 1H), 7.21 (d, J=2.4 Hz, 1H), 7.14 (d, J=8.4 Hz, 2H), 7.02 (t, J=7.3 Hz, 1H), 6.88 (t, J=7.5 Hz, 1H), 4.51 (dt, J=5.9, 8.1 Hz, 1H), 4.11 (m, 1H), 3.28 (dd, J=6.0, 15.0 Hz, 1H), 3.2(t, J=6.5, 15.1 Hz, 1H), 2.78-2.74 (m, 2H), 2.47 (t, J=4.9 Hz, 3H), 2.27 (s, 3H), 1.79-1.52 (m, 3H), 1.42-1.23 (m, 3H); Low resolution mass spectrum (ES) m/e 436[(M+1)+, calcd for C₂₅H₃₄N₅O₂: 436]; 96.5% purity based on HPLC.

### Example 91

### (S)-6-AMINO-2-(2-NAPHTHALEN-1-YL-ACETYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to Naphthalen-1-yl-acetic acid as described in the method of Example 23. Purification by HPLC produced the title compound. 1H NMR (400 MHz, DMSO-d₆) δ 9.98 (s, 1H), 8.53 (d, J=8.1 Hz, 1H), 8.11 (m, 1H), 7.92 (m, 1H), 7.81 (m, 1H), 7.68 (br s, 3H), 7.50 (m, 3H), 7.47 (d, J=8.4 Hz, 2H), 7.45 (m, 2H), 7.10 (d, J=8.3 Hz, 2H), 4.41 (dt, J=5.5, 8.4 Hz, 1H), 4.00 (q, J=15.0 Hz, 2H), 2.73 (m, 2H), 2.24 (s, 3H), 1.75 (m, 1H), 1.68-1.60 (m, 1H), 1.54 (m, 2H), 1.42-1.26 (m, 2H); Low resolution mass spectrum (ES) m/e 404[(M+1)+, calcd for C₂₅H₃₀N₃O₂: 404]; 99.9% purity based on HPLC.

### Example 92

### (S)-[5-AMINO-1-(METHYL-P-TOLYL-CARBAMOYL)-PENTYL]-CARBAMIC ACID 9H-FLUOREN-9-YLMETHYL ESTER

p-Tolylmethylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 7.90 (d, J=7.5 Hz, 2H), 7.73 (dd, J=5.0, 7.3 Hz, 2H), 7.58 (br s, 3H), 7.56 (d, J=8.1 Hz, 1H), 7.42 (t, J=7.5 Hz, 2H), 7.33 (dt, J=3.6, 7.3 Hz, 2H), 7.26 (m, 4H), 4.25-4.18 (m, 3H), 4.04 (dd, J=8.3, 13.4 Hz, 1H), 3.13 (s, 3H), 2.63 (m, 2H), 2.32 (s, 3H), 1.49-1.43 (m, 2H), 1.29-1.16 (m, 3H), 1.04-0.96 (m, 1H); Low resolution mass spectrum (ES) m/e 472[(M+1)+, calcd for C₂₉H₃₄N₃O₃: 72 ]; 88% purity based on HPLC.

### Example 93

### (S)-6-AMINO-2-[3-(4-METHOXY-BIPHENYL-3-YL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 3-Isocyanato-4-methoxy-biphenyl as described in the method of Example 26. Purification by HPLC produced the title compound. 1H NMR (400 MHz, DMSO-d₆) δ 10.04 (s, 1H), 8.43 (d, J=2.3 Hz, 1H), 8.27 (s, 1H), 7.66 (br s, 3H), 7.50 (m, 4H), 7.39 (t, J=7.8 Hz, 2H), 7.35 (d, J=8.1 Hz, 1H), 7.27 (t, J=7.3 Hz, 1H), 7.16 (dd, J=2.3, 8.4 Hz, 1H), 7.09 (d, J=8.3 Hz, 2H), 7.04 (d, J=8.5 Hz, 1H), 4.34 (dt, J=5.5, 8.2 Hz, 1H), 3.87 (s, 3H), 2.77 (m, 2H), 2.23 (s, 3H), 1.76-1.69 (m, 1H), 1.62-1.51 (m, 3H), 1.45-1.30 (m, 2H); Low resolution mass spectrum (ES) m/e 461 [(M+1)+, calcd for C₂₇H₃₃N₄O₃: 461]; 99.8% purity based on HPLC.

### Example 94

### (S)-6-AMINO-2-(3-BIPHENYL-4-YL-UREIDO)-HEXANOIC ACID (2-METHYL-1H-INDOL-5-YL)-AMIDE

2-Methyl-1H-indol-5-ylamine was coupled to (S)-6-tert-Butoxycarbonylamino-2-(9H-fluoren-9-ylmethoxycarbonylamino)-hexanoic acid, and then to 4-Isocyanato-biphenyl as described in the method of Example 26. Purification by HPLC produced the title compound. 1H NMR (400 MHz, DMSO-d₆) δ 10.84 (s, 1H), 9.92 (s, 1H), 8.89 (s, 1H), 7.72 (d, J=1.6 Hz, 1H), 7.69 (br s, 3H), 7.61 (d, J=7.3 Hz, 2H), 7.56 (d, J=8.8 Hz, 2H), 7.49 (d, J=8.8 Hz, 2H), 7.42 (t, J=7.7 Hz, 2H), 7.29 (t, J=7.4 Hz, 1H), 7.18(d, J=8.6 Hz, 1H), 7.13 (dd, J=1.9, 8.7 Hz, 1H), 6.59 (d, J=8.2 Hz, 1H), 6.06 (s, 1H), 4.04 (dd, J=7.8, 13.6 Hz, 1H), 2.84-2.76 (m, 2H), 2.35 (s, 3H), 1.82-1.73 (m, 1H), 1.69-1.55 (m, 3H), 1.47-1.34 (m, 2H); Low resolution mass spectrum (ES) m/e 470[(M+1)+, calcd for C₂₈H₃₂N₅O₂: 470]; 99.9% purity based on HPLC.

### Example 95

### (S)-6-AMINO-2-[3-(3,5-BIS-TRIFLUOROMETHYL-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanato-3,5-bis-trifluoromethyl-benzene as described in the method of Example 26. Purification by HPLC produced the title compound. 1H NMR (400 MHz, DMSO-d₆) δ 10.09 (s, 1H), 9.65 (s, 1H), 8.06 (s, 1H), 7.70 (br s, 3H), 7.57 (s, 1H), 7.50 (d, J=8.4 Hz, 2H), 7.12 (d, J=8.4 Hz, 2H), 6.98 (d, J=7.6 Hz, 1H), 4.38 (dd, J=7.9, 13.2 Hz, 1H), 2.82-2.75 (m, 2H), 2.25 (s, 3H), 1.82-1.51 (m, 4H), 1.46-1.3 (m, 2H); Low resolution mass spectrum (ES) m/e 491 [(M+1)+, calcd for C₂₂H₂₅F₆N₄O₂: 491]; 99.4% purity based on HPLC.

### Example 96

### (R,S)-6-AMINO-2-[3-(1-NAPHTHALEN-1-YL-ETHYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (R)-1-(1-Isocyanato-ethyl)-naphthalene as described in the method of Example 26. Purification by HPLC produced the title compound. 1H NMR (400 MHz, DMSO-d₆) δ 9.95 (s, 1H), 8.10 (m, 1H), 7.92 (m, 1H), 7.80 (d, J=7.7 Hz, 1H), 7.67 (br s, 3H), 7.52-7.47 (m, 4H), 7.44 (d, J=8.3 Hz, 2H), 7.09 (d, J=8.4 Hz, 2H), 6.77 (d, J=8.0 Hz, 1H), 6.22 (d, J=8.5 Hz, 1H), 5.53 (p, J=5.9 Hz, 1H), 4.29 (dd, J=7.9, 13.8 Hz, 1H), 2.78 (br s, 2H), 2.24 (s, 3H), 1.71-1.63 (m, 1H), 1.91-1.5 (m, 3H), 1.46 (d, J=6.8 Hz, 3H), 1.40-1.26 (m, 2H); Low resolution mass spectrum (ES) m/e 433[(M+1)+, calcd for C₂₆H₃₃N₄O₂: 433]; 99.2% purity based on HPLC.

### Example 97

### (S,S)-6-AMINO-2-[3-(1-NAPHTHALEN-1-YL-ETHYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (S)-1-(1-Isocyanato-ethyl)-naphthalene as described in the method of Example 26. Purification by HPLC produced the title compound. 1H NMR (400 MHz, DMSO-d₆) δ 10.01 (s, 1H), 8.13 (d, J=8.0 Hz, 1H), 7.95 (m, 1H), 7.82 (dd, J=2.9, 6.0 Hz, 1H), 7.65 (br s, 3H), 7.58-7.48 (m, 6H), 7.12 (d, J=8.3 Hz, 2H), 6.78 (d, J=8.1 Hz, 1H), 6.22 (d, J=8.5 Hz, 1H), 5.55 (p, J=6.9 Hz, 1H), 4.30 (dd, J=8.0,13.9 Hz, 1H), 2.75-2.67 (m, 2H), 2.25 (s, 3H), 1.69-1.60 (m, 1H), 1.54-1.46 (m, 2H), 1.45 (d, J=6.9 Hz, 3H), 1.35-1.21 (m, 2H); Low resolution mass spectrum (ES) m/e 433[(M+1)+, calcd for C₂₆H₃₃N₄O₂: 433]; 99.8% purity based on HPLC.

### Example 98

### (S)-6-AMINO-2-[3-(3-PHENOXY-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanato-3-phenoxy-benzene as described in the method of Example 26. Purification by HPLC produced the title compound. 1H NMR (400 MHz, DMSO-d₆) δ 10.06 (s, 1H), 8.89 (s, 1H), 7.68 (br s, 3H), 7.48 (d, J=8.4 Hz, 2H), 7.38 (dd, J=7.6, 8.3 Hz, 2H), 7.22 (m, 2H), 7.13 (m, 3H), 7.00 (m, 3H), 6.57-6.53 (m, 2H), 4.33 (dd, J=8.0, 13.5 Hz, 1H), 2.8-2.75 (m, 2H), 2.25 (s, 3H), 1.76-1.68 (m, 1H), 1.63-1.51 (m, 3H), 1.43-1.28 (m, 2H); Low resolution mass spectrum (ES) m/e 447[(M+1)+, calcd for C₂₆H₃₁N₄O₃: 447 99.7% purity based on HPLC.

### Example 99

### (S)-6-AMINO-2-[PHENYLSULFONAMIDE-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to Benzenesulfonyl isocyanate as described in the method of Example 23. Purification by HPLC produced the title compound. 1H NMR (400 MHz, DMSO-d₆) δ 10.77 (s, 1H), 10.04 (s, 1H), 7.90 (dd, J=1.2, 7.6 Hz, 2H), 7.7-7.58 (m, 6H), 7.43 (d, J=8.8 Hz, 2H), 7.10 (d, J=8.1 Hz, 2H), 6.92 (d, J=8.0 Hz, 1H), 4.21 (dd, J=7.7, 13.4 Hz, 1H), 2.73-2.68 (m, 2H), 2.24 (s, 3H), 1.7-1.62 (m, 1H), 1.58-1.4 (m, 3H), 1.27-1.16 (m, 2H); Low resolution mass spectrum (ES) m/e 419[(M+1)+, calcd for C₂₀H₂₇N₄O₂S: 419]; 95.2% purity based on HPLC.

### Example 100

### (S,S)-6-AMINO-2-[2-AMINO-3-(1H-INDOL-3-YL)-PROPIONYLAMINO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 2-tert-Butoxycarbonylamino-3-(1H-indol-3-yl)-propionic acid as described in the method of Example 23. Purification by HPLC produced the title compound. 1H NMR (400 MHz, DMSO-d₆) δ 11.02 (d, J=1.3 Hz, 1H), 10.14 (s, 1H), 8.93 (d, J=7.8 Hz, 1H), 8.04 (br s, 3H), 7.79 (br s, 3H), 7.68 (d, J=7.9 Hz, 1H), 7.51 (d, J=8.4 Hz, 2H), 7.34 (d, J=8.1 Hz, 1H), 7.21 (d, J=2.3 Hz, 1H), 7.14 (d, J=8.3 Hz, 2H), 7.05 (m, 1H), 6.92 (t, J=7.4 Hz, 1H), 4.48 (dd, J=8.0, 13.8 Hz, 1H), 4.10 (m, 1H), 3.27 (dd, J=4.9, 14.9 Hz, 1H), 3.08 (dd, J=8.2, 14.9 Hz, 1H), 2.8-2.75 (m, 2H), 2.26 (s, 3H), 1.81-1.53 (m, 4H), 1.47-1.29 (m, 2H); Low resolution mass spectrum (ES) m/e 422[(M+1)+, calcd for C₂₄H₃₂N₅O₂: 422]; 96.1% purity based on HPLC.

### Example 101

### (S)-(4-AMINO-1-P-TOLYLCARBAMOYL-BUTYL)-CARBAMIC ACID 9H-FLUOREN-9-YLMETHYL ESTER

p-Tolylamine was coupled to (S)-5-tert-Butoxycarbonylamino-2-(9H-fluoren-9-ylmethoxycarbonylamino)-pentanoic acid as described in the method of Example 6. Purification by HPLC produced the title compound. 1H NMR (400 MHz, DMSO-d₆) δ 10.00 (s, 1H), 7.9 (d, J=7.5 Hz, 2H), 7.76-7.71 (m, 6H), 7.49 (d, J=8.3 Hz, 2H), 7.42 (t, J=7.4 Hz, 2H), 7.33 (dd, J=7.2, 12.5 Hz, 2H), 7.12 (d, J=8.3 Hz, 2H), 4.34-4.2 (m, 3H), 4.16 (dd, J=8.0, 13.5 Hz, 1H), 2.79 (m, 2H), 2.25 (s, 3H), 1.78-1.55 (m, 4H); Low resolution mass spectrum (ES) m/e 444[(M+1)+, calcd for C₂₇H₃₀N₃O₃: 444]; 99.9% purity based on HPLC.

### Example 102

### (R)-(4-AMINO-1-P-TOLYLCARBAMOYL-BUTYL)-CARBAMIC ACID 9H-FLUOREN-9-YLMETHYL ESTER

p-Tolylamine was coupled to (R)-5-tert-Butoxycarbonylamino-2-(9H-fluoren-9-ylmethoxycarbonylamino)-pentanoic acid as described in the method of Example 6. Purification by HPLC produced the title compound. 1H NMR (400 MHz, DMSO-d₆) δ 10.00 (s, 1H), 7.9 (d, J=7.5 Hz, 2H), 7.76-7.71 (m, 6H), 7.49 (d, J=8.4 Hz, 2H), 7.42 (t, J=7.4 Hz, 2H), 7.33 (dd, J=7.2, 12.5 Hz, 2H), 7.12 (d, J=8.3 Hz, 2H), 4.34-4.2 (m, 3H), 4.16 (dd, J=8.0, 13.4 Hz, 1H), 2.79 (m, 2H), 2.25 (s, 3H), 1.78-1.56 (m, 4H); Low resolution mass spectrum (ES) m/e 444[(M+1)+, calcd for C₂₇H₃₀N₃O₃: 444]; 99.9% purity based on HPLC.

### Example 103

### (S)-(2-AMINO-1-P-TOLYLCARBAMOYL-ETHYL)-CARBAMIC ACID 9H-FLUOREN-9-YLMETHYL ESTER

p-Tolylamine was coupled to (S)-3-tert-Butoxycarbonylamino-2-(9H-fluoren-9-ylmethoxycarbonylamino)-propionic acid as described in the method of Example 6. Purification by HPLC produced the title compound. 1H NMR (400 MHz, DMSO-d₆) δ 10.14 (s, 1H), 7.97 (br s, 3H), 7.90 (d, J=7.5 Hz, 2H), 7.83 (d, J=8.2 Hz, 1H), 7.73 (dd, J=1.9, 7.2 Hz, 2H), 7.50 (d, J=8.3 Hz, 2H), 7.42 (t, J=7.4 Hz, 2H), 7.33 (t, J=7.4 Hz, 2H), 7.14 (d, J=8.3 Hz, 2H), 4.47-4.33 (m, 3H), 4.26 (t, J=6.7 Hz, 1H), 3.24 (m, 1H), 3.05 (m, 1H), 2.26 (s, 3H); Low resolution mass spectrum (ES) m/e 416[(M+1)+, calcd for C₂₅H₂₆N₃O₃: 416]; 98.6% purity based on HPLC.

### Example 104

### (S)-(6-AMINO-1-P-TOLYLCARBAMOYL-HEXYL)-CARBAMIC ACID 9H-FLUOREN-9-YLMETHYL ESTER

p-Tolylamine was coupled to (S)-8-tert-Butoxycarbonylamino-2-(9H-fluoren-9-ylmethoxycarbonylamino)-Octanoic acid as described in the method of Example 6. Purification by HPLC produced the title compound. 1H NMR (400 MHz, DMSO-d₆) δ 9.94 (s, 1H), 7.89 (d, J=7.5 Hz, 2H), 7.76-7.65 (m, 6H), 7.49 (d, J=8.4 Hz, 2H), 7.42 (t, J=7.4 Hz, 2H), 7.32 (dt, J=3.1, 7.3 Hz, 2H), 7.11 (d, J=8.3 Hz, 2H), 4.29-4.2 (m, 3H), 4.12 (dd, J=8.2, 14.0 Hz, 1H), 2.79-2.74 (m, 2H), 2.25 (s, 3H), 1.71-1.61 (m, 2H), 1.55-1.48 (m, 2H), 1.39-1.31 (m, 4H); Low resolution mass spectrum (ES) m/e 473[(M+1)+, calcd for C₂₉H₃₄N₃O₃: 473]; 98.3% purity based on HPLC.

### Example 105

### (S)-[5-AMINO-1-(4-TERT-BUTYL-PHENYLCARBAMOYL)-PENTYL]-CARBAMIC ACID 9H-FLUOREN-9-YLMETHYL ESTER

p-t-Butylphenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d₆) δ 9.96 (s, 1H), 7.89 (d, J=7.5 Hz, 2H), 7.73 (t, J=7.6 Hz, 2H), 7.69-7.59 (m, 4H), 7.50 (d, J=8.7 Hz, 2H), 7.41 (t, J=6.8 Hz, 2H), 7.36-7.28 (m, 4H), 4.35-4.17 (m, 3H), 4.12 (ddd, J=5.7, 8.3, 8.3 Hz, 1H), 2.77 (dddd, J=6.4, 6.4, 6.6, 12.8 Hz, 2H), 1.77-1.28 (m, 6H), 1.24 (s, 9H); Low resolution mass spectrum (ES) m/e 500[(M+1)+, calcd for C₃₁H₃₈N₃O₃: 500]; 92.4% purity based on HPLC.

### Example 106

### (S)-[5-AMINO-1-(4-DIETHYLAMINO-PHENYLCARBAMOYL)-PENTYL]-CARBAMIC ACID 9H-FLUOREN-9-YLMETHYL ESTER

N,N-Diethyl-benzene-1,4-diamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d₆) δ 11.22 (br s, 1H), 10.36 (br s, 1H), 9.67 (br s, 1H), 7.89 (d, J=7.5 Hz, 2H), 7.72 (t, J=7.3 Hz, 4H), 7.65 (br s, 2H), 7.41 (ddd, J=2.3, 7.3, 7.4 Hz, 2H), 7.31 (ddd, J=5.3, 7.3, 7.3 Hz, 2H), 6.63 (br s, 1H), 4.34-4.26 (m, 2H), 4.22 (dd, J=6.8, 13.4 Hz, 1H), 4.1 (dd, J=8.2, 13.6 Hz, 2H), 3.42 (br s, 4H), 2.77 (dd, J=6.2, 12.3 Hz, 2H), 1.77-1.24 (m, 6H), 1.00 (t, J=5.9 Hz, 6H); Low resolution mass spectrum (ES) m/e 515[(M+1)+, calcd for C₃₁H₃₉N₄O₃: 515]; 92.4% purity based on HPLC.

### Example 107

### (S)-[5-AMINO-1-(4-CYCLOHEXYL-PHENYLCARBAMOYL)-PENTYL]-CARBAMIC ACID 9H-FLUOREN-9-YLMETHYL ESTER

p-Cyclohexylphenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 9.93 (br s, 1H), 7.89 (d, J=7.5 Hz, 2H), 7.72 (t, J=7.7 Hz, 2H), 7.69-7.57 (m, 4H), 7.49 (d, J=8.4 Hz, 2H), 7.41 (t, J=6.8 Hz, 2H), 7.31 (dd, J=4.7, 7.3, 7.3 Hz, 2H), 7.14 (d, J=8.4 Hz, 2H), 4.34-4.18 (m, 3H), 4.11 (ddd, J=6.2, 8.3, 8.3 Hz, 1H), 2.7 (dd, J=6.4, 12.7 Hz, 2H), 2.42 (br s, 1H), 1.86-1.59 (m, 7H), 1.59-1.47 (m, 2H), 1.45-1.26 (m, 6H), 1.26-1.14 (m, 1H); Low resolution mass spectrum (ES) m/e 526[(M+1)+, calcd for C₃₃H₄₀N₃O₃: 526]; 95.9% purity based on HPLC.

### Example 108

### (S)-(3-AMINO-1-P-TOLYLCARBAMOYL-PROPYL)-CARBAMIC ACID 9H-FLUOREN-9-YLMETHYL ESTER

p-Tolylamine was coupled to (S)-4-tert-Butoxycarbonylamino-2-(9H-fluoren-9-ylmethoxycarbonylamino)-butanoic acid as described in the method of Example 6. Purification by HPLC produced the title compound. 1H NMR (400 MHz, DMSO-d₆) δ 9.99 (s, 1H), 7.89 (d, J=7.5 Hz, 2H), 7.84 (d, J=7.9 Hz, 1H), 7.76-7.66 (m, 4H), 7.47 (d, J=8.3 Hz, 2H), 7.41 (t, J=7.4 Hz, 2H), 7.32 (ddd, J=3.8, 7.3, 7.3 Hz, 2H), 7.12 (d, J=8.2 Hz, 2H), 4.36-4.18 (m, 4H), 2.83 (dd, J=6.1, 11.8 Hz, 2H), 2.25 (s, 3H), 2.05-1.85 (m, 2H); Low resolution mass spectrum (ES) m/e 430[(M+1)+, calcd for C₂₆H₂₈N₃O₃: 430]; 98.2% purity based on HPLC.

### Example 109

### (3-P-TOLYLCARBAMOYL-PIPERIDIN-3-YL)-CARBAMIC ACID 9H-FLUOREN-9-YLMETHYL ESTER

p-Tolylamine was coupled to 3-(9H-Fluoren-9-ylmethoxycarbonylamino)-piperidine-1,3-dicarboxylic acid 1-tert-butyl ester as described in the method of Example 6. Purification by HPLC produced the title compound. 1H NMR (400 MHz, DMSO-d₆) δ 9.76 (s, 1H), 8.74 (br s, 1H), 8.26 (br s, 1H), 7.88 (d, J=7.3 Hz, 2H), 7.81 (br s, 1H), 7.73 (d, J=4.5 Hz, 2H), 7.46-7.22 (m, 6H), 7.11 (d, J=8.3 Hz, 2H), 4.37 (dd, J=7.1, 10.4 Hz, 1H), 4.29 (dd, J=6.7, 10.4 Hz, 2H), 4.2 (t, J=6.7 Hz, 1H), 3.19-2.92 (m, 4H), 2.24 (s, 2H), 1.89 (br s, 2H), 1.56 (br s, 2H); Low resolution mass spectrum (ES) m/e 456[(M+1)+, calcd for C₂₈H₃₀N₃O₃: 456]; 98.2% purity based on HPLC.

### Example 110

### (S)-[5-((9H-FLUOREN-9-YLMETHOXYCARBONYL)AMINO)-5-P-TOLYLCARBAMOYL-PENTYL]-TRIMETHYL-AMMONIUM

p-Tolylamine was coupled to (S)-[5-Carboxy-5-(9H-fluoren-9-ylmethoxycarbonylamino)-pentyl]-trimethyl-ammonium chloride as described in the method for Intermediate # 1 of Example 1. Purification by HPLC produced the title compound. 1H NMR (400 MHz, DMSO-d₆) δ 9.97 (s, 1H), 7.90 (d, J=7.5 Hz, 2H), 7.74 (t, J=8.1 Hz, 2H), 7.69 (d, J=8.0 Hz, 1H), 7.49 (d, J=8.3 Hz, 2H), 7.42 (t, J=7.3 Hz, 2H), 7.33 (dt, J=4.0, 7.4 Hz, 2H), 7.11 (d, J=8.3 Hz, 2H), 4.34-4.20 (m, 3H), 4.15 (dd, J=8.5, 13.9 Hz, 1H), 3.28-3.24 (m, 2H), 3.02 (s, 9H), 2.25 (s, 2H), 1.79-1.62 (m, 4H), 1.43-1.24 (m, 2H); Low resolution mass spectrum (ES) m/e 500[(M)+, calcd for C₃₁H₃₈N₃O₃: 500]; 96.4% purity based on HPLC.

### Example 111

### (S)-[4-(3,3-DIMETHYL-GUANIDINO)-1-P-TOLYLCARBAMOYL-BUTYL]-CARBAMIC ACID 9H-FLUOREN-9-YLMETHYL ESTER

p-Tolylamine was coupled to (S)-6-(3,3-Dimethyl-guanidino)-2-(9H-fluoren-9-ylmethoxycarbonylamino)-hexanoic acid as described in the method for Intermediate #1 of Example 1. Purification by HPLC produced the title compound. 1H NMR (400 MHz, DMSO-d₆) δ 9.96 (s, 1H), 7.90 (d, J=7.5 Hz, 2H), 7.73 (d, J=7.8 Hz, 2H), 7.67 (d, J=7.9 Hz, 1H), 7.49 (d, J=8.3 Hz, 2H), 7.43-7.30 (m, 7H), 7.11 (d, J=8.3 Hz, 2H), 4.34-4.21 (m, 3H), 4.15 (dd, J=8.0, 13.6 Hz, 1H), 3.17 (m, 2H), 2.93 (s, 6H), 2.25 (s, 3H), 1.76-1.46 (m, 4H); Low resolution mass spectrum (ES) m/e 514[(M+1)+, calcd for C₃₀H₃₆N₅O₃: 514]; 94.5% purity based on HPLC.

### Example 112

### (S)-(3-METHYLSULFANYL-1-P-TOLYLCARBAMOYL-PROPYL)-CARBAMIC ACID 9H-FLUOREN-9-YLMETHYL ESTER

p-Tolylamine was coupled to (S)-2-(9H-Fluoren-9-ylmethoxycarbonylamino)-4-methylsulfanyl-butyric acid as described in the method for Intermediate #1 of Example 1. Purification by HPLC produced the title compound. 1H NMR (400 MHz, DMSO-d₆) δ 9.95 (s, 1H), 7.89 (d, J=7.5 Hz, 2H), 7.73 (dd, J=7.2,13.3 Hz, 3H), 7.48 (d, J=8.3 Hz, 2H), 7.42 (t, J=7.4 Hz, 2H), 7.32 (dt, J=3.1, 7.4 Hz, 2H), 7.11 (d, J=8.3 Hz, 2H), 4.3-4.2 (m, 4H), 2.58-2.43 (m, 2H), 2.25 (s, 3H), 2.06 (s, 3H), 1.99-1.85 (m, 2H); Low resolution mass spectrum (ES) m/e 461[(M+1)+, calcd for C₂₇H₂₉N₂O₃S: 461]; 90.4% purity based on HPLC.

### Example 113

### (S)-(1-P-TOLYLCARBAMOYL-4-UREIDO-BUTYL)-CARBAMIC ACID 9H-FLUOREN-9-YLMETHYL ESTER

p-Tolylamine was coupled to (S)-2-(9H-Fluoren-9-ylmethoxycarbonylamino)-6-ureido-hexanoic acid as described in the method for Intermediate #1 of Example 1. Purification by HPLC produced the title compound. 1H 1H NMR (400 MHz, DMSO-d₆) δ 9.93 (s, 1H), 7.89 (d, J=7.5 Hz, 2H), 7.74 (dd, J=4.7, 7.2 Hz, 2H), 7.66 (d, J=8.0 Hz, 1H), 7.49 (d, J=8.4 Hz, 2H), 7.41 (d, J=7.4 Hz, 2H), 7.33 (dt, J=4.5, 7.1 Hz, 2H), 7.10 (d, J=8.3 Hz, 2H), 5.90 (t, J=5.5 Hz, 1H), 5.42 (s, 2H), 4.28-4.2 (m, 2H), 4.15 (dd, J=8.5, 13.6 Hz, 1H), 3.07-2.91 (m, 2H), 2.25 (s, 3H), 1.72-1.34 (m, 4H); Low resolution mass spectrum (ES) m/e 487[(M+1)+, calcd for C₂₈H₃₁N₄O₄: 487]; 97.5% purity based on HPLC.

### Example 114

### (S)-2,6-DIAMINO-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid as described in the method of Example 5 and further deprotected as described in the method of Example 2. Purification by HPLC produced the title compound. ¹H NMR (400 MHz, DMSO-d₆) δ 10.47 (s, 1H), 8.29 (br s, 3H), 7.78 (br s, 3H), 7.49 (d, J=8.4 Hz, 2H), 7.16 (d, J=8.3 Hz, 2H), 3.92 (br s, 1H), 2.76 (br s, 2H), 2.27 (s, 3H), 1.85-1.75 (m, 2H), 1.59-1.51 (m, 2H), 1.41-1.35 (m, 2H); Low resolution mass spectrum (ES) m/e 236[(M+1)+, calcd for C₁₃H₂₁N₃O: 236]; 100% purity based on HPLC.

### Example 115

### (S)-[2-(2-AMINO-ACETYLAMINO)-1-P-TOLYLCARBAMOYL-ETHYL]-CARBAMIC ACID 9H-FLUOREN-9-YLMETHYL ESTER

tert-Butoxycarbonylamino-acetic acid was coupled to the compound of Example 103 as described in the method of Example 6. Purification by HPLC produced the title compound. 1H NMR (400 MHz, DMSO-d₆) δ 10.01 (s, 1H), 8.43 (br s, 1H), 7.98 (br s, 3H), 7.90 (d, J=7.5 Hz, 2H), 7.73 (dd, J=3.6, 7.2 Hz, 2H), 7.64 (d, J=8.1 Hz, 1H), 7.49 (d, J=8.3 Hz, 2H), 7.42 (t, J=7.4 Hz, 2H), 7.33 (dd, J=6.9, 13.3 Hz, 2H), 7.12 (d, J=8.3 Hz, 2H), 4.38-4.22 (m, 4H), 3.53-3.46 (m, 4H), 2.25 (s, 3H); Low resolution mass spectrum (ES) m/e 473[(M+1)+, calcd for C₂₇H₂₉N₄O₄: 473]; 99.2% purity based on HPLC.

### Example 116

### (S)-[6-AMINO-1-(4-CYCLOHEXYL-PHENYLCARBAMOYL)-HEXYL]-CARBAMIC ACID 9H-FLUOREN-9-YLMETHYL ESTER

p-Cyclohexyphenylamine was coupled to (S)-8-tert-Butoxycarbonylamino-2-(9H-fluoren-9-ylmethoxycarbonylamino)-Octanoic acid as described in the method of Example 6. Purification by HPLC produced the title compound. 1H NMR (400 MHz, DMSO-d₆) δ 9.92 (s, 1H), 7.89 (d, J=7.5 Hz, 2H), 7.73 (t, J=7.8 Hz, 2H), 7.67-7.54 (m, 3H), 7.48 (d, J=8.5 Hz, 2H), 7.41 (t, J=7.3 Hz, 3H), 7.31 (ddd, J=3.1, 7.3, 7.4 Hz, 2H), 7.13 (d, J=8.5 Hz, 2H), 4.31-4.18 (m, 3H), 4.11 (dd, J=8.1, 14.1 Hz, 1H), 2.75 (dd, J=6.7, 13.4 Hz, 2H), 2.42 (br s, 1H), 1.82-1.44 (m, 9H), 1.44-1.12 (m, 9H); Low resolution mass spectrum (ES) m/e 540[(M+1)+, calcd for C₃₄H₄₂N₃O₃: 540]; 98% purity based on HPLC.

### Example 117

### (S)-6-AMINO-2-[3-(4-BENZYLOXY-PHENYL)-UREIDO]-HEXANOIC ACID (4-CYCLOHEXYL-PHENYL)-AMIDE

p-Cyclohexyphenylamine was coupled to (S)-6-tert-Butoxycarbonylamino-2-(9H-fluoren-9-ylmethoxycarbonylamino)-hexanoic acid, and then to 1-Benzyloxy-4-isocyanato-benzene as described in the method of Example 26. Purification by HPLC produced the title compound. 1H NMR (400 MHz, DMSO-d₆) δ 10.07 (s, 1H), 8.54 (s, 1H), 7.66 (br s, 3H), 7.51 (d, J=8.6 Hz, 2H), 7.44-7.36 (m, 4H), 7.32 (d, J=7.1 Hz, 1H), 7.28 (d, J=9.1 Hz, 2H), 7.15 (d, J=8.6 Hz, 2H), 6.89 (d, J=9.1 Hz, 2H), 6.43 (d, J=8.3 Hz, 1H), 5.03 (s, 2H), 4.38 (dd, J=7.9, 13.7 Hz, 1H), 2.8-2.75 (m, 2H), 2.43 (m, 1H), 1.78-1.68 (m, 6H), 1.64-1.19 (m, 10H); Low resolution mass spectrum (ES) m/e 529[(M+1)+, calcd for C₃₂H₄₁N₄O₃: 529]; 90.1% purity based on HPLC.

### Example 118

### (S)-6-AMINO-2-[3-(3-BROMO-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid,and then to 1-Bromo-3-isocyanato-benzene as described in the method of Example 26. Purification by HPLC produced the title compound. ¹H NMR (400 MHz, DMSO-d₆) δ 10.09 (s, 1H), 8.97 (m, 1H), 7.82 (s, 1H), 7.66 (br s, 3H), 7.49 (d, J=8.4 Hz, 2H), 7.19 (d, J=5.3 Hz, 2H), 7.12 (d, J=8.3 Hz, 2H), 7.07 (m, 1H), 6.65 (m, 1H), 4.38 (dd, J=7.9, 13.4 Hz, 1H), 2.78 (br s, 2H), 2.25 (s, 3H), 1.79-1.7 (m, 1H), 1.66-1.5 (m, 3H), 1.45-1.3 (m, 2H); Low resolution mass spectrum (ES) m/e 433[(M)+, calcd for C₂₀H₂₆BrN₄O₂: 433]; 100% purity based on HPLC.

### Example 119

### (S)-6-AMINO-2-(3-BIPHENYL-4-YL-UREIDO)-HEXANOIC ACID (4-CYCLOHEXYL-PHENYL)-AMIDE

p-Cyclohexylphenylamine was coupled to (S)-6-tert-Butoxycarbonylamino-2-(9H-fluoren-9-ylmethoxycarbonylamino)-hexanoic acid, and then to 4-Isocyanato-biphenyl as described in the method of Example 26. Purification by HPLC produced the title compound. 1H NMR (400 MHz, DMSO-d₆) δ 10.11 (s, 1H), 8.88 (m, 1H), 7.70 (br s, 3H), 7.61 (d, J=7.3 Hz, 2H), 7.57-7.47 (m, 6H), 7.42 (t, J=7.7 Hz, 2H), 7.29 (t, J=7.3 Hz, 1H), 7.16 (d, J=8.5 Hz, 2H), 6.63 (m, 1H), 4.41 (dd, J=7.8, 13.5 Hz, 1H), 2.83-2.75 (m, 2H), 2.44 (m, 1H), 1.76-1.5 (m, 9H), 1.43-1.30 (m, 6H), 1.26-1.19 (m, 1H); Low resolution mass spectrum (ES) m/e 499[(M+1)+, calcd for C₃₁H₃₉N₄O₂: 499]; 98.1% purity based on HPLC.

### Example 120

### (S)-7-AMINO-2-(3-BENZYL-UREIDO)-HEPTANOIC ACID (4-CYCLOHEXYL-PHENYL)-AMIDE

p-Cyclohexyphenylamine was coupled to (S)-8-tert-Butoxycarbonylamino-2-(9H-fluoren-9-ylmethoxycarbonylamino)-Octanoic acid, and then to benzyl isocyanate as described in the method of Example 26. Purification by HPLC produced the title compound. 1H NMR (400 MHz, DMSO-d₆) δ 9.97 (s, 1H), 7.61 (br s, 3H), 7.48 (d, J=8.5 Hz, 2H), 7.34-7.26 (m, 2H), 7.26-7.17 (m, 3H), 7.14 (d, J=8.5 Hz, 2H), 6.54 (t, J=6.0 Hz, 1H), 6.29 (d, J=8.5 Hz, 1H), 4.43 (dd, J=7.6, 14.1 Hz, 2H), 2.79-2.68 (m, 2H), 2.42 (br s, 1H), 1.86-1.58 (m, 6H), 1.57-1.43 (m, 3H), 1.42-1.12 (m, 9H); Low resolution mass spectrum (ES) m/e 451 [(M+1)+, calcd for C₂₇H₃₉N₄O₂: 450]; 100% purity based on HPLC.

### Example 121

### (S)-7-AMINO-2-[3-(4-BENZYLOXY-PHENYL)-UREIDO]-HEPTANOIC ACID (4-CYCLOHEXYL-PHENYL)-AMIDE

p-Cyclohexyphenylamine was coupled to (S)-8-tert-Butoxycarbonylamino-2-(9H-fluoren-9-ylmethoxycarbonylamino)-Octanoic acid, and then to 1-Benzyloxy-4-isocyanato-benzene as described in the method of Example 26. Purification by HPLC produced the title compound. 1H NMR (400 MHz, DMSO-d₆) δ 10.00 (s, 1H), 8.49 (s, 1H), 7.59 (br s, 3H), 7.49 (d, J=8.5 Hz, 2H), 7.45-7.21 (m, 7H), 7.14 (d, J=8.5 Hz, 2H), 6.88 (d, J=9.0 Hz, 2H), 6.40 (d, J=8.3 Hz, 1H), 5.01 (s, 2H), 4.37 (dd, J=7.4, 13.8 Hz, 1H), 2.79-2.69 (m, 2H), 2.43 (br s, 1H), 1.86-1.62 (m, 6H), 1.62-1.42 (m, 3H), 1.42-1.12 (m, 9H); Low resolution mass spectrum (ES) m/e 543[(M+1)+, calcd for C₃₃H₄₃N₄O₃: 543]; 97% purity based on HPLC.

### Example 122

### (S)-2-P-TOLYLCARBAMOYL-PIPERIDINE-1-CARBOXYLIC ACID 9H-FLUOREN-9-YLMETHYL ESTER

p-Tolylamine was coupled to (S)-Piperidine-1,2-dicarboxylic acid 1-(9H-fluoren-9-ylmethyl) ester as described in the method for Intermediate #1 of Example 1. Purification by HPLC produced the title compound. 1H NMR (400 MHz, DMSO-d₆) δ 7.92-7.75 (m, 2H), 7.68-7.25 (m, 7H), 7.16-6.96 (m, 3H), 4.79 (br s, 0.5H), 4.68 (br s, 0.5H), 4.41-4.11 (m, 3H), 3.92 (d, J=11.6 Hz, 0.5H), 3.77 (d, J=11.2 Hz, 0.5H), 3.37-3.16 (m, 1H), 2.28-2.00 (m, 4H), 1.78-1.50 (m, 3H), 1.40-1.16 (m, 2H); Low resolution mass spectrum (ES) m/e 441 [(M+1)+, calcd for C₂₈H₂₉N₂O₃: 441]; 95% purity based on HPLC.

### Example 123

### (R)-2-P-TOLYLCARBAMOYL-PIPERIDINE-1-CARBOXYLIC ACID 9H-FLUOREN-9-YLMETHYL ESTER

p-Tolylamine was coupled to (R)-Piperidine-1,2-dicarboxylic acid 1-(9H-fluoren-9-ylmethyl) ester as described in the method for Intermediate #1 of Example 1. Purification by HPLC produced the title compound. 1H NMR (400 MHz, DMSO-d₆) δ 7.92-7.75 (m, 2H), 7.68-7.25 (m, 7H), 7.16-6.96 (m, 3H), 4.79 (br s, 0.5H), 4.68 (br s, 0.5H), 4.41-4.11 (m, 3H), 3.92 (d, J=11.6 Hz, 0.5H), 3.77 (d, J=11.2 Hz, 0.5H), 3.37-3.16 (m, 1H), 2.28-2.00 (m, 4H), 1.78-1.50 (m, 3H), 1.40-1.16 (m, 2H); Low resolution mass spectrum (ES) m/e 441 [(M+1)+, calcd for C₂₈H₂₉N₂O₃: 441]; 95% purity based on HPLC.

### Example 124

### (S)-(5-DIMETHYLAMINO-1-P-TOLYLCARBAMOYL-PENTYL)-CARBAMIC ACID 9H-FLUOREN-9-YLMETHYL ESTER

p-Tolylamine was coupled to (S)-6-Dimethylamino-2-(9H-fluoren-9-ylmethoxycarbonylamino)-hexanoic acid as described in the method for Intermediate #1 of Example 1. Purification by HPLC produced the title compound. 1H NMR (400 MHz, DMSO-d₆) δ 7.86 (d, J=7.5 Hz, 2H), 7.70 (t, J=7.9 Hz, 2H), 7.47-7.36 (m, 4H), 7.32 (d, J=6.9 Hz, 1H), 7.29 (d, J=7.2 Hz, 1H), 7.10 (d, J=8.3 Hz, 2H), 4.34-4.03 (m, 4H), 2.99 (t, J=7.7 Hz, 2H), 2.72 (s, 6H), 2.22 (s, 3H), 1.75-1.52 (m, 4H), 1.44.1.18 (m, 2H); Low resolution mass spectrum (ES) m/e 486[(M+1)+, calcd for C₃₀H₃₆N₃O₃: 486]; 85% purity based on HPLC.

### Example 125

### (S)-6-AMINO-2-[3-(9H-FLUOREN-2-YL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 2-Isocyanato-9H-fluorene as described in the method of Example 26. Purification by HPLC produced the title compound. 1H NMR (400 MHz, DMSO-d₆) δ 10.10 (s, 1H), 8.86 (s, 1H), 7.74 (m, 3H), 7.69 (br s, 3H), 7.51 (m, 3H), 7.32 (m, 2H), 7.22 (dt, J=1.1, 7.4 Hz, 1H), 7.12 (dt, J=8.3 Hz, 2H), 6.61 (t, J=8.2 Hz, 1H), 4.41 (dt, J=5.5, 8.1 Hz, 1H), 3.85 (s, 2H), 2.83-2.76 (m, 2H), 2.25 (s, 3H), 1.79-1.72 (m, 1H), 1.66-1.53 (m, 3H), 1.45-1.33 (m, 2H); Low resolution mass spectrum (ES) m/e 443[(M+1)+, calcd for C₂₇H₃₁N₄O₂: 443]; 99.7% purity based on HPLC.

### Example 126

### (S)-6-AMINO-2-[3-(9H-FLUOREN-9-YL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid then to 9-Isocyanato-9H-fluorene as described in the method of Example 26. Purification by HPLC produced the title compound. 1H NMR (400 MHz, DMSO-d₆) δ 10.09 (s, 1H), 7.84 (dd, J=2.7, 7.5 Hz, 2H), 7.70 (br s, 3H), 7.52 (m, 4H), 7.41 (dd, J=6.9, 14.0 Hz, 2H), 7.31 (ddt, J=0.9, 7.5, 8.4 Hz, 2H), 7.13 (d, J=8.5 Hz, 2H), 6.64 (d, J=8.7 Hz, 1H), 6.57 (s, 0.4H), 6.25 (d, J=8.5 Hz, 1H), 5.82 (d, J=8.6 Hz, 1H), 4.47 (dt, J=5.9, 8.2 Hz, 1H), 2.82-2.78 (m, 2H), 2.26 (s, 3H), 1.77-1.7 (m, 1H), 1.63-1.55 (m, 3H), 1.45-1.34 (m, 2H); Low resolution mass spectrum (ES) m/e 443[(M+1)+, calcd for C₂₇H₃₁N₄O₂: 443]; 98% purity based on HPLC.

### Example 127

### (S)-6-AMINO-2-(3-BIPHENYL-3-YL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid then to 3-Isocyanato-biphenyl as described in the method of Example 26. Purification by HPLC produced the title compound. 1H NMR (400 MHz, DMSO-d₆) δ 10.09 (s, 1H), 8.87 (s, 1H), 7.73 (m, 1H), 7.68 (br s, 3H), 7.58 (dd, J=1.2, 8.3 Hz, 2H), 7.50 (d, J=8.5 Hz, 2H), 7.46 (t, J=7.7 Hz, 2H), 7.35 (m, 3H), 7.19 (m, 1H), 7.12 (dd, J=8.3 Hz, 2H), 6.62 (dd, J=8.2 Hz, 1H), 4.40 (dt, J=5.6, 8.1 Hz, 1H), 2.79 (dd, J=7.3, 13.2 Hz, 2H), 2.25 (s, 3H), 1.78-1.72 (m, 1H), 1.66-1.54 (m, 3H), 1.44-1.34 (m, 2H); Low resolution mass spectrum (ES) m/e 431 [(M+1)+, calcd for C₂₆H₃₁N₄O₂: 431]; 95.9% purity based on HPLC.

### Example 128

### (R)-[5-AMINO-1-(4-CYCLOHEXYL-PHENYLCARBAMOYL)-PENTYL]-CARBAMIC ACID 9H-FLUOREN-9-YLMETHYL ESTER

p-Cyclohexylphenylamine was coupled to (R)-6-tert-Butoxycarbonylamino-2-(9H-fluoren-9-ylmethoxycarbonylamino)-hexanoic acid as described in the method of Example 6 and purified by HPLC to produce the title compound. 1H NMR (400 MHz, DMSO-d₆) δ 9.95 (s, 1H), 7.89 (d, J=7.5 Hz, 2H), 7.72 (t, J=7.7 Hz, 2H), 7.68 (br s, 3H), 7.62 (d, J=8.0 Hz, 1H), 7.5 (d, J=8.4 Hz, 2H), 7.42 (t, J=6.9 Hz, 2H), 7.32 (dd, J=7.2, 12.0 Hz, 2H), 7.14 (d, J=8.4 Hz, 2H), 4.33-4.20 (m, 3H), 4.12 (dd, J=8.2, 13.8 Hz, 1H), 2.8-2.75 (m, 2H), 2.43 (br s, 1H), 1.78-1.19 (m, 16H); Low resolution mass spectrum (ES) m/e 526[(M+1)+, calcd for C₃₃H₄₀N₃O₃: 526]; 95.8% purity based on HPLC.

### Example 129

### (S)-[2-(4-AMINOMETHYL-PHENYL)-1-P-TOLYLCARBAMOYL-ETHYL]-CARBAMIC ACID 9H-FLUOREN-9-YLMETHYL ESTER

p-Tolylamine was coupled to (S)-3-[4-(tert-Butoxycarbonylamino-methyl)-phenyl]-2-(9H-fluoren-9-ylmethoxycarbonylamino)-propionic acid as described in the method of Example 6 and purified by HPLC to produce the title compound. 1H NMR (400 MHz, DMSO-d6) δ 7.85 (d, J=7.0 Hz, 2H), 7.67-7.60 (br, 2H), 7.48-7.24 (m, 10H), 7.11 (d, J=8.3 Hz, 2H), 4.39-4.09 (m, 4H), 3.02 (dd, J=4.2, 14.0 Hz, 2H), 2.87 (t, J=11.2, 13.2 Hz, 2H), 2.23 (s, 3H); Low resolution mass spectrum (ES) m/e 506.2 [(M+H)+, calcd for C₃₂H₃₂N₃O₃: 506.6]; 90% purity based on NMR.

### Example 130

### (S)-(4-(R)-(3-AMINO-PROPIONYLOXY)-2-P-TOLYLCARBAMOYL-PYRROLIDINE-1-CARBOXYLIC ACID 9H-FLUOREN-9-YLMETHYL ESTER)

(4R,S)-4-Hydroxy-2-p-tolylcarbamoyl-pyrrolidine-1-carboxylic acid 9H-fluoren-9-ylmethyl ester from Example 87 was coupled to 3-tert-Butoxycarbonylamino-propionic acid as described in the method of Example 6 and purified by HPLC to produce the title compound. 1H NMR (400 MHz, DMSO-d6) δ 7.88 (dd, J=3.1, 7.5 Hz, 1H), 7.80 (d, J=7.7 Hz, 1H), 7.66-7.48 (m, 3H), 7.47-7.38 (m, 2H), 7.36-7.28 (m, 2H), 7.14-7.03 (m, 3H), 5.31 (br s, 1H), 4.57 (t, J=7.7, 7.2 Hz, 1H), 4.38 (t, J=8.3, 7.7 Hz, 1H), 4.33-4.22 (m, 2H), 4.10-4.00 (m, 2H), 3.75 (dd, J=5.0, 12.1 Hz, 1H), 3.65 (d, J=12.5 Hz, 1H), 3.03 (t, J=6.6 Hz, 2H), 2.71-2.62 (m, 2H), 2.22 (s, 3H); Low resolution mass spectrum (ES) m/e 514.2 [(M+H)+, calcd for C₃₀H₃₂N₃O₅: 514.6]; 90% purity based on NMR.

### Example 131

### (S)-[4-(R)-(2-AMINO-ACETOXY)-2-P-TOLYLCARBAMOYL-PYRROLIDINE-1-CARBOXYLIC ACID 9H-FLUOREN-9-YLMETHYL ESTER]

(4R,S)-4-Hydroxy-2-p-tolylcarbamoyl-pyrrolidine-1-carboxylic acid 9H-fluoren-9-ylmethyl ester of Example 87 was coupled to tert-Butoxycarbonylamino-acetic acid as described in the method for M-2024 and purified by HPLC to produce the title compound. 1H NMR (400 MHz, DMSO-d6) δ 7.88 (d, J=7.7 Hz, 1H), 7.79 (d, J=7.7 Hz, 1H), 7.62 (t, J=7.0, 7.2 Hz, 1H), 7.59-7.47 (m, 2H), 7.47-7.37 (m, 2H), 7.37-7.27 (m, 2H), 7.14-7.03 (m, 3H), 5.40 (br s, 1H), 4.58 (t, J=7.7, 7.2 Hz, 1H), 4.40 (t, J=8.2, 7.7 Hz, 1H), 4.34-4.20 (m, 2H), 4.09-3.98 (m, 2H), 3.87-3.74 (m, 3H), 3.55 (d, J=11.8 Hz, 1H), 2.22 (s, 3H); Low resolution mass spectrum (ES) m/e 500.2 [(M+H)+, calcd for C₂₉H₃₀N₃O₅: 500.6]; 90% purity based on NMR.

### Example 132

### (R)-4-[2-(S)-(2-AMINO-ACETYLAMINO)-ACETOXY]-2-P-TOLYLCARBAMOYL-PYRROLIDINE-1-CARBOXYLIC ACID 9H-FLUOREN-9-YLMETHYL ESTER

(4R,S)-4-Hydroxy-2-p-tolylcarbamoyl-pyrrolidine-1-carboxylic acid 9H-fluoren-9-ylmethyl ester from Example 87 was coupled to (2-tert-Butoxycarbonylamino-acetylamino)-acetic acid as described in the method of Example 6 and purified by HPLC to produce the title compound. 1H NMR (400 MHz, DMSO-d6) δ 7.89 (d, J=7.7 Hz, 1H), 7.80 (d, J=8.1 Hz, 1H), 7.66-7.49 (m, 3H), 7.47-7.38 (m, 2H), 7.38-7.28 (m, 2H), 7.15-7.02 (m, 3H), 5.31 (br s, 1H), 4.59 (t, J=8.3, 7.7 Hz, 1H), 4.39 (t, J=7.7, 7.5 Hz, 1H), 4.33-4.21 (m, 2H), 4.12-4.01 (m, 4H), 3.99 (s, 2H), 3.76 (dd, J=5.3, 12.3 Hz, 1H), 3.66-3.59 (m, 3H), 2.22 (s, 3H); Low resolution mass spectrum (ES) m/e 557.2 [(M+H)+, calcd for C₃₁H₃₃N₄O₆: 557.7]; 90% purity based on NMR.

### Example 133

### (S)-6-AMINO-2-(3-BENZYL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid then to benzyl isocyanate as described in the method of Example 26. Purification by HPLC produced the title compound. 1H NMR (400 MHz, DMSO-d₆) δ 9.99 (s, 1H), 7.72 (br s, 3H), 7.49 (d, J=8.4 Hz, 2H), 7.32-7.29 (m, 2H), 7.25-7.20 (m, 3H), 7.11 (d, J=8.3 Hz, 2H), 6.60 (dt, J=2.5, 5.8 Hz, 1H), 6.33 (dd, J=2.1, 8.4 Hz, 1H), 4.32 (dd, J=8.1, 13.9 Hz, 1H), 4.22 (d, J=5.9 Hz, 2H), 2.77 (t, J=7.4 Hz, 2H), 2.25 (s, 3H), 1.72-1.63 (m, 1H), 1.59-1.50 (m, 3H), 1.43-1.27 (m, 2H); Low resolution mass spectrum (ES) m/e 369[(M+1)+, calcd for C₂₁H₂₉N₄O₂: 369]; 99.4% purity based on HPLC.

### Example 134

### (S)-6-AMINO-2-(3-PHENETHYL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (2-Isocyanato-ethyl)-benzene as described in the method of Example 26. Purification by HPLC produced the title compound. 1H NMR (400 MHz, DMSO-d₆) δ 9.97 (s, 1H), 7.68 (br s, 3H), 7.48 (d, J=8.4 Hz, 2H), 7.30-7.27 (m, 2H), 7.21-7.17 (m, 3H), 7.10 (d, J=8.3 Hz, 2H), 6.25 (d, J=8.4 Hz, 1H), 6.11 (t, J=5.4 Hz, 1H), 4.28 (dd, J=8.1, 13.8 Hz, 1H), 3.23 (ddd, J=2.4, 7.2, 12.6 Hz, 2H), 2.80-2.72 (m, 2H), 2.67 (t, J=7.2 Hz, 2H), 2.25 (s, 3H), 1.69-1.6 (m, 1H), 1.57-1.46 (m, 3H), 1.40-1.24 (m, 2H); Low resolution mass spectrum (ES) m/e 383[(M+1)+, calcd for C₂₂H₃₁N₄O₂: 383]; 100% purity based on HPLC.

### Example 135

### (S)-6-AMINO-2-(3-P-TOLYL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to p-tolyl isocyanate as described in the method of Example 26. Purification by HPLC produced the title compound. 1H NMR (400 MHz, DMSO-d₆) δ 10.07 (s, 1H), 8.65-8.62 (m, 1H), 7.71 (br s, 3H), 7.50 (d, J=8.3 Hz, 2H), 7.26 (d, J=8.1 Hz, 2H), 7.11 (d, J=8.3 Hz, 2H), 7.02 (d, J=8.3 Hz, 2H), 6.54-6.49 (m, 1H), 4.37 (dd, J=7.9, 13.5 Hz, 1H), 2.79 (m, 2H), 2.25 (s, 3H), 2.21 (s, 3H), 1.77-1.68 (m, 1H), 1.64-1.51 (m, 3H), 1.45-1.31 (m, 2H); Low resolution mass spectrum (ES) m/e 369[(M+1)+, calcd for C₂₁H₂₉N₄O₂: 369]; 100% purity based on HPLC.

### Example 136

### (S)-6-AMINO-2-[3-(4-METHYL-BENZYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanatomethyl-4-methyl-benzene as described in the method of Example 26. Purification by HPLC produced the title compound. 1H NMR (400 MHz, DMSO-d6) δ 9.98 (s, 1H), 7.69 (br s, 3H), 7.48 (d, J=8.4 Hz, 2H), 7.12 (m, 6H), 6.53 (t, J=5.8 Hz, 1H), 6.29 (d, J=8.4 Hz, 1H), 4.31 (dd, J=8.1, 13.9 Hz, 1H), 4.16 (d, J=5.7 Hz, 2H), 2.81-2.73 (m, 2H), 2.26 (s, 3H), 2.25 (s, 3H), 1.71-1.63 (m, 1H), 1.60-1.49 (m, 3H), 1.42-1.24 (m, 2H); Low resolution mass spectrum (ES) m/e 383[(M+1)+, calcd for C₂₂H₃₁N₄O₂: 383]; 99.6% purity based on HPLC.

### Example 137

### (R)-6-AMINO-2-[3-(9H-FLUOREN-2-YL)-UREIDO]-HEXANOIC ACID (4-CYCLOHEXYL-PHENYL)-AMIDE

p-Cyclohexylphenyl amine was coupled to (R)-6-tert-Butoxycarbonylamino-2-(9H-fluoren-9-ylmethoxycarbonylamino)-hexanoic acid, and then to 2-Isocyanato-9H-fluorene as described in the method of Example 26. Purification by HPLC produced the title compound. 1H NMR (400 MHz, DMSO-d₆) δ 10.11 (s, 1H), 8.84 (s, 1H), 7.74 (m, 3H), 7.66 (br s, 3H), 7.52 (m, 3H), 7.32 (m, 2H), 7.22 (dt, J=1.0, 7.5 Hz, 1H), 7.16 (d, J=8.6 Hz, 2H), 6.59 (d, J=8.2 Hz, 1H), 4.42 (dd, J=7.8, 13.5 Hz, 1H), 3.85 (s, 2H), 2.83-2.75 (m, 2H), 2.47-2.41 (m, 1H), 1.78-1.19 (m, 16H); Low resolution mass spectrum (ES) m/e 511 [(M+1)+, calcd for C₃₂H₃₉N₄O₂: 511]; 91.1% purity based on HPLC.

### Example 138

### (4-P-TOLYLCARBAMOYL-PIPERIDIN-4-YL)-CARBAMIC ACID 9H-FLUOREN-9-YLMETHYL ESTER

p-Tolylamine was coupled to 4-(9H-Fluoren-9-ylmethoxycarbonylamino)-piperidine-1,4-dicarboxylic acid mono-tert-butyl ester as described in the method of Example 6 and purified by HPLC to produce the title compound. 1H NMR (400 MHz, DMSO-d₆) δ 9.48 (s, 1H), 8.48 (br s, 1H), 8.30 (br s, 3H), 7.89 (d, J=7.5 Hz, 2H), 7.78-7.62 (m, 3H), 7.38 (t, J=7.5 Hz, 4H), 7.26 (t, J=7.0 Hz, 2H), 7.06 (d, J=8.4 Hz, 2H), 4.33 (d, J=6.8 Hz, 2H), 4.18 (t, J=5.9 Hz, 1H), 3.23-2.97 (m, 4H), 2.21 (s, 3H), 2.12 (br s, 4H); Low resolution mass spectrum (ES) m/e 456[(M+1)+, calcd for C₂₈H₃₀N₃O₃: 456]; 98% purity based on HPLC.

### Example 139

### (S)-6-AMINO-2-[3-(4-METHOXY-BENZYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanatomethyl-4-methoxy-benzene as described in the method of Example 26. Purification by HPLC produced the title compound. 1H NMR (400 MHz, DMSO-d₆) δ 9.48 (s, 1H), 9.98 (s, 1H), 7.70 (br s, 3H), 7.48 (d, J=8.4 Hz, 2H), 7.16 (d, J=8.6 Hz, 2H), 7.11 (d, J=8.3 Hz, 2H), 6.86 (d, J=8.6 Hz, 2H), 6.50 (t, J=5.9 Hz, 1H), 6.27 (d, J=8.5 Hz, 1H), 4.31 (dd, J=8.1, 13.8 Hz, 1H), 4.14 (dd, J=2.3, 5.5 Hz, 2H), 3.72 (s, 3H), 2.81-2.73 (m, 2H), 2.25 (s, 3H), 1.71-1.49 (m, 4H), 1.42-1.24 (m, 2H); Low resolution mass spectrum (ES) m/e 399[(M+1)+, calcd for C₂₂H₃₁N₄O₃: 399]; 99.3% purity based on HPLC.

### Example 140

### (S)-6-AMINO-2-[3-(3,4-DICHLORO-BENZYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1,2-Dichloro-4-isocyanatomethyl-benzene as described in the method of Example 26. Purification by HPLC produced the title compound. 1H NMR (400 MHz, DMSO-d₆) δ 9.98 (s, 1H), 7.71 (br s, 3H), 7.56 (d, J=8.3 Hz, 1H), 7.49 (m, 3H), 7.23 (dd, J=1.9, 8.3 Hz, 1H), 7.10 (d, J=8.3 Hz, 2H), 6.70 (m, 1H), 6.42 (m, 1H), 4.29 (dd, J=8.2, 13.9 Hz, 1H), 4.21 (d, J=6.1 Hz, 2H), 2.81-2.73 (m, 2H), 2.25 (s, 3H), 1.73-1.64 (m, 1H), 1.58-1.51 (m, 3H), 1.43-1.27 (m, 2H); Low resolution mass spectrum (ES) m/e 437[(M)+, calcd for C₂₁H₂₇Cl₂N₄O₂: 438]; 99.3% purity based on HPLC.

### Example 141

### (R)-6-AMINO-2-(3-BENZYL-UREIDO)-HEXANOIC ACID (4-CYCLOHEXYL-PHENYL)-AMIDE

p-Cyclohexylphenyl amine was coupled to (R)-6-tert-Butoxycarbonylamino-2-(9H-fluoren-9-ylmethoxycarbonylamino)-hexanoic acid then to benzyl isocyanate as described in the method of Example 26. Purification by HPLC produced the title compound. 1H NMR (400 MHz, DMSO-d₆) δ 10.00 (s, 1H), 7.68 (br s, 3H), 7.50 (d, J=8.5 Hz, 2H), 7.32-7.28 (m, 2H), 7.25-7.20 (m, 3H), 7.15 (d, J=8.5 Hz, 2H), 6.59 (t, J=6.0 Hz, 1H), 6.32 (d, J=8.5 Hz, 1H), 4.32 (dd, J=8.0, 13.9 Hz, 1H), 4.22 (dd, J=2.6, 5.7 Hz, 2H), 2.79-2.74 (m, 2H), 2.43 (br s, 1H), 1.78-1.51 (m, 9H), 1.42-1.17 (m, 7H); Low resolution mass spectrum (ES) m/e 437[(M+1)+, calcd for C₂₆H₃₇N₄O₂: 438]; 97.4% purity based on HPLC.

### Example 142

### (R)-6-AMINO-2-(3-BIPHENYL-4-YL-UREIDO)-HEXANOIC ACID (4-CYCLOHEXYL-PHENYL)-AMIDE

p-Cyclohexylphenyl amine was coupled to (R)-6-tert-Butoxycarbonylamino-2-(9H-fluoren-9-ylmethoxycarbonylamino)-hexanoic acid, and then to 4-Isocyanato-biphenyl as described in the method of Example 26. Purification by HPLC produced the title compound. 1H NMR (400 MHz, DMSO-d₆) δ 10.09 (s, 1H), 8.85 (s, 1H), 7.66 (br s, 3H), 7.61 (d, J=7.2 Hz, 2H), 7.57-7.47 (m, 6H), 7.42 (t, J=7.7 Hz, 2H), 7.30 (t, J=7.3 Hz, 1H), 7.16 (d, J=8.5 Hz, 2H), 6.59 (d, J=8.2 Hz, 1H), 4.42 (dd, J=7.8, 13.5 Hz, 1H), 2.83-2.75 (m, 2H), 2.44 (m, 1H), 1.76-1.50 (m, 9H), 1.41-1.12 (m, 7H); Low resolution mass spectrum (ES) m/e 499[(M+1)+, calcd for C₃₁H₃₉N₄O₂: 499]; 97% purity based on HPLC.

### Example 143

### (S)-[5-AMINO-1-(BIPHENYL-4-YLCARBAMOYL)-PENTYL]-CARBAMIC ACID 9H-FLUOREN-9-YLMETHYL ESTER

Biphenyl-4-ylamine was coupled to (S)-6-tert-Butoxycarbonylamino-2-(9H-fluoren-9-ylmethoxycarbonylamino)-hexanoic acid as described in the method of Example 6, and then purified by HPLC to produce the title compound. 1H NMR (400 MHz, DMSO-d₆) δ 10.15 (s, 1H), 7.90 (d, J=7.5 Hz, 2H), 7.76-7.63 (m, 12H), 7.46-7.40 (m, 4H), 7.35-7.31 (m, 3H), 4.36-4.22 (m, 3H), 4.17 (dd, J=8.3, 13.7 Hz, 1H), 2.82-2.77 (m, 2H), 1.78-1.30 (m, 6H); Low resolution mass spectrum (ES) m/e 520[(M+1)+, calcd for C₃₃H₃₄N₃O₃: 520]; 98% purity based on HPLC.

### Example 144

### (R)-[5-AMINO-1-(BIPHENYL-4-YLCARBAMOYL)-PENTYL]-CARBAMIC ACID 9H-FLUOREN-9-YLMETHYL ESTER

Biphenyl-4-ylamine was coupled to (R)-6-tert-Butoxycarbonylamino-2-(9H-fluoren-9-ylmethoxycarbonylamino)-hexanoic acid as described in the method of Example 6 and then purified by HPLC to produce the title compound. 1H NMR (400 MHz, DMSO-d₆) δ 10.16 (s, 1H), 7.90 (d, J=7.5 Hz, 2H), 7.76-7.63 (m, 12H), 7.46-7.40 (m, 4H), 7.35-7.31 (m, 3H), 4.35-4.22 (m, 3H), 4.17 (dd, J=8.4, 13.7 Hz, 1H), 2.82-2.77 (m, 2H), 1.78-1.32 (m, 6H); Low resolution mass spectrum (ES) m/e 520[(M+1)+, calcd for C₃₃H₃₄N₃O₃: 520]; 98.7% purity based on HPLC.

### Example 145

### (S)-3-(4-AMINOMETHYL-PHENYL)-2-(3-BIPHENYL-4-YL-UREIDO)-N-P-TOLYL-PROPIONAMIDE

p-Tolylamine was coupled to (S)-3-[4-(tert-Butoxycarbonylamino-methyl)-phenyl]-2-(9H-fluoren-9-ylmethoxycarbonylamino)-propionic acid, and then to 4-Isocyanato-biphenyl as described in the method of Example 26. Purification by HPLC produced the title compound. 1H NMR (400 MHz, DMSO-d₆) δ 10.15 (s, 1H), 8.84 (s, 1H), 8.13 (br s, 3H), 7.59 (d, J=7.3 Hz, 2H), 7.53 (d, J=8.7 Hz, 2H), 7.49-7.27 (m, 11H), 7.12 (d, J=8.4 Hz, 2H), 6.61 (d, J=8.3 Hz, 1H), 4.68 (dt, J=5.3, 8.4 Hz, 1H), 4.01-3.97 (m, 2H), 3.12 (dd, J=4.9, 13.8 Hz, 1H), 2.91 (dd, J=8.7, 13.8 Hz, 1H), 2.26 (s, 3H); Low resolution mass spectrum (ES) m/e 479[(M+1)+, calcd for C₃₀H₃₁N₄O₂: 479]; 94.5% purity based on HPLC.

### Example 146

### (S)-3-(4-AMINOMETHYL-PHENYL)-2-[3-(4-BENZYLOXY-PHENYL)-UREIDO]-N-P-TOLYL-PROPIONAMIDE

p-Tolylamine was coupled to (S)-3-[4-(tert-Butoxycarbonylamino-methyl)-phenyl]-2-(9H-fluoren-9-ylmethoxycarbonylamino)-propionic acid, and then to 1-Benzyloxy-4-isocyanato-benzene as described in the method of Example 26. Purification by HPLC produced the title compound. 1H NMR (400 MHz, DMSO-d₆) δ 10.11 (s, 1H), 8.53 (s, 1H), 8.14 (s, 3H), 7.47 (d, J=8.4 Hz, 2H), 7.42-7.30 (m, 9H), 7.24 (d, J=9.0 Hz, 2H), 7.12 (d, J=8.4 Hz, 2H), 6.87 (d, J=9.1 Hz, 2H), 6.46 (d, J=8.4 Hz, 1H), 5.02 (s, 2H), 4.64 (dt, J=5.3, 8.5 Hz, 1H), 4.01-3.97 (m, 2H), 3.09 (dd, J=5.0, 13.8 Hz, 1H), 2.88 (dd, J=8.7, 13.7 Hz, 1H), 2.25 (s, 3H); Low resolution mass spectrum (ES) m/e 509[(M+1)+, calcd for C₃₁H₃₃N₄O₃: 509]; 95.9% purity based on HPLC.

### Example 147

### (S)-3-(4-AMINOMETHYL-PHENYL)-2-[3-(9H-FLUOREN-2-YL)-UREIDO]-N-P-TOLYL-PROPIONAMIDE

p-Tolylamine was coupled to (S)-3-[4-(tert-Butoxycarbonylamino-methyl)-phenyl]-2-(9H-fluoren-9-ylmethoxycarbonylamino)-propionic acid, and then to 2-Isocyanato-9H-fluorene as described in the method of Example 26. Purification by HPLC produced the title compound. 1H NMR (400 MHz, DMSO-d₆) δ 10.15 (s, 1H), 8.82 (s, 1H), 8.12 (br s, 3H), 7.75-7.68 (m, 3H), 7.50 (m, 3H), 7.39-7.28 (m, 6H), 7.22 (t, J=7.4 Hz, 1H), 7.12 (d, J=8.3 Hz, 2H), 6.60 (d, J=8.3 Hz, 1H), 4.68 (dt, J=5.4, 8.4 Hz, 1H), 4.00 (m, 2H), 3.84 (s, 2H), 3.13 (dd, J=5.0, 13.8 Hz, 1H), 2.92 (dd, J=8.8, 13.9 Hz, 1H), 2.26 (s, 3H); Low resolution mass spectrum (ES) m/e 491 [(M+1)+, calcd for C₃₁H₃₁N₄O₂: 491]; 99.0% purity based on HPLC.

### Example 148

### (S)-3-(4-AMINOMETHYL-PHENYL)-2-(3-BENZYL-UREIDO)-N-(4-TERT-BUTYL-PHENYL)-PROPIONAMIDE

p-t-Butyl-phenylamine was coupled to (S)-3-[4-(tert-Butoxycarbonylamino-methyl)-phenyl]-2-(9H-fluoren-9-ylmethoxycarbonylamino)-propionic acid, and then to benzyl isocyanate as described in the method of Example 26. Purification by HPLC produced the title compound. 1H NMR (400 MHz, DMSO-d6) δ 10.08 (s, 1H), 8.15 (br s, 3H), 7.54 (d, J=8 Hz, 1H), 7.49 (d, J=9 Hz, 2H), 7.36-7.27 (m, 8H), 7.23-7.19 (m, 3H), 6.58 (t, J=6 Hz, 1H), 6.36 (d, J=8.5 Hz, 1H), 4.59 (dt, J=5.76, 8.39 Hz, 1H), 4.17 (dd, J=2.79, 5.53 Hz, 2H), 4.00-3.99 (m, 2H), 3.04 (dd, J=5.25, 13.64 Hz, 1H), 2.84 (dd, J=8.54, 13.60 Hz, 1H), 1.26 (s, 9H), Low resolution mass spectrum (ES) m/e 459 [(M+H)+, calcd for C₂₈H₃₅N₄O₂: 548]; 99.7% purity based on HPLC.

### Example 149

### (S)-3-(4-AMINOMETHYL-PHENYL)-N-(4-TERT-BUTYL-PHENYL)-2-[3-(3,4-DICHLORO-BENZYL)-UREIDO]-PROPIONAMIDE

p-t-Butyl-phenylamine was coupled to (S)-3-[4-(tert-Butoxycarbonylamino-methyl)-phenyl]-2-(9H-fluoren-9-ylmethoxycarbonylamino)-propionic acid, and then to 1,2-Dichloro-4-isocyanatomethyl-benzene as described in the method of Example 26. Purification by HPLC produced the title compound. 1H NMR (400 MHz, DMSO-d6) d 10.07 (s, 1H), 8.15 (br s, 3H), 7.54 (d, J=8 Hz, 1H), 7.49 (d, J=9 Hz, 2H),7.45 (d, J=2 Hz, 1H), 7.36-7.28 (m, 6H), 7.18 (dd, J=2 and 8 Hz, 1H), 6.69-6.66 (m, 1H), 6.48-6.45 (m, 1H), 4.56 (dt, J=6 and 8 Hz, 1H), 4.17 (dd, J=4 and 5 Hz, 2H), 3.99 (d, J=5 Hz, 2H), 3.04 (dd, J=5 and 14 Hz, 2H), 2.84 (dd, J=9 and 14 Hz, 1H), 1.26 (s, 9H), Low resolution mass spectrum (ES) m/e 527 [(M+H)+, calcd for C₂₈H₃₃C₁₂N₄O₂: 527]; 99.6% purity based on HPLC.

### Example 150

### (S)-3-(4-AMINOMETHYL-PHENYL)-2-(3-BIPHENYL-4-YL-UREIDO)-N-(4-TERT-BUTYL-PHENYL)-PROPIONAMIDE

p-t-Butyl-phenylamine was coupled to (S)-3-[4-(tert-Butoxycarbonylamino-methyl)-phenyl]-2-(9H-fluoren-9-ylmethoxycarbonylamino)-propionic acid, and then to 4-Isocyanatomethyl-biphenyl as described in the method of Example 26. Purification by HPLC produced the title compound. 1H NMR (400 MHz, DMSO-d6) d 10.20 (s, 1H), 8.86 (s, 1H), 8.13 (br s, 3H), 7.59 (d, J=7.45 Hz, 2H), 7.53 (t, J=9.00 Hz, 4H), 7.46-7.27(m, 11H), 6.62 (d, J=8.27 Hz, 1H), 4.69 (dt, J=5.71, 8.30 Hz, 1H), 3.99 (d, J=5.18 Hz, 2H), 3.12 (dd, J=4.98, 13.76 Hz, 1H), 2.91 (dd, J=8.56, 13.74 Hz, 1H), 1,26 (s, 9H); Low resolution mass spectrum (ES) m/e 521 [(M+H)+, calcd for C₃₃H₃₇N₄O₂: 521]; 100% purity based on HPLC.

### Example 151

### (S)-3-(4-AMINOMETHYL-PHENYL)-2-(3-BENZHYDRYL-UREIDO)-N-(4-TERT-BUTYL-PHENYL)-PROPIONAMIDE

p-t-Butyl-phenylamine was coupled to (S)-3-[4-(tert-Butoxycarbonylamino-methyl)-phenyl]-2-(9H-fluoren-9-ylmethoxycarbonylamino)-propionic acid, and then to (Phenyl-isocyanato-methyl)-benzene as described in the method of Example 26. Purification by HPLC produced the title compound. ¹H NMR (400 MHz, DMSO-d6) d 10.08 (s, 1H), 8.13 (br s, 3H), 7.47 (d, J=8.65 Hz, 1H), 7.35-7.18 (m, 17H), 6.35 (d, J=8.44 Hz, 1H), 5.85 (d, J=8.52 Hz, 1H), 4.58 (dd, J=7.95, 13.72 Hz, 1H), 3.98 (d, J=5.45 Hz, 2H), 3.05 (dd, J=5.28, 13.62 Hz, 1H), 2.84 (dd, J=7.94, 13.67 Hz, 1H), 1.25 (s,9H); Low resolution mass spectrum (ES) m/e 535 [(M+H)+, calcd for C₃₄H₃₉N₄O₂: 535]; 100% purity based on HPLC.

### Example 152

### (S)-3-(4-AMINOMETHYL-PHENYL)-N-(4-TERT-BUTYL-PHENYL)-2-[3-(9H-FLUOREN-2-YL)-UREIDO]-PROPIONAMIDE

p-t-Butyl-phenylamine was coupled to (S)-3-[4-(tert-Butoxycarbonylamino-methyl)-phenyl]-2-(9H-fluoren-9-ylmethoxycarbonylamino)-propionic acid then to 2-Isocyanato-9H-fluorene as described in the method of Example 26. Purification by HPLC produced the title compound. ¹H NMR (400 MHz, DMSO-d6) d 10.20 (s, 1H), 8.86 (s, 1H), 8.15 (br s, 3H), 7.73 (dd, J=8.00, 14.41 Hz, 2H), 7.69 (s, 1H), 7.53-7.50 (m, 3H), 7.39-7.28 (m, 8H), 7.22 (t, J=7.41, 7.41 Hz, 1H), 6.62 (d, J=8.29 Hz, 1H), 4.68 (dd, J=8.29, 13.52 Hz, 1H), 3.99 (d, J=5.52 Hz, 2H), 3.84 (s, 2H), 3.13 (dd, J=4.94, 13.75 Hz, 1H), 2.92 (dd, J=8.50, 13.74 Hz, 1H), 1.26 (s, 9H); Low resolution mass spectrum (ES) m/e 533 [(M+H)+, calcd for C₃₄H₃₇N₄O₂: 533]; 96.1% purity based on HPLC.

### Example 153

### (S)-FURAN-2-CARBOXYLIC ACID {4-[6-AMINO-2-(3-BENZYL-UREIDO)-HEXANOYLAMINO]-BENZYL}-CYCLOHEXYL-AMIDE

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid, and then to benzyl isocyante as described in the method of Example 26. Purification by HPLC produced the title compound. ¹H NMR (400 MHz, DMSO-d6) d 10.05 (s, 1H), 7.79 (br s, 1H), 7.66 (br s, 3H), 7.53 (d, J=8.40 Hz, 2H), 7.32-7.28 (m, 2H), 7.24-7.19 (m, 5H), 6.89 (br s, 1H), 6.58-6.56 (m, 2H), 6.30 (d, J=8.43 Hz, 1H), 4.64 (s, 2H), 4.32 (dd, J=7.79, 13.75 Hz, 1H), 4.22 (d, J=4.82 Hz, 2H), 4.11 (m, 1H), 2.80-2.74 (m, 2H), 1.71-1.18 (m, 15H), 1.08-0.99 (m, 1H); Low resolution mass spectrum (ES) m/e 560 [(M+H)+, calcd for C₃₂H₄₂N₅O₄: 560]; 100% purity based on HPLC.

### Example 154

### (S)-FURAN-2-CARBOXYLIC ACID (4-{6-AMINO-2-[3-(3,4-DICHLORO-BENZYL)-UREIDO]-HEXANOYLAMINO}-BENZYL)-CYCLOHEXYL-AMIDE

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid, and then to 1,2-Dichloro-4-isocyanatomethyl-benzene as described in the method of Example 26. Purification by HPLC produced the title compound. ¹H NMR (400 MHz, DMSO-d6) d 10.06 (s, 1H), 7.80 (br s, 1H), 7.67 (br s, 3H), 7.57-7.52 (m, 3H), 7.47 (s, 1H), 7.24-7.18 (m, 3H), 6.89 (br s, 1H), 6.68 (t, J=5.97, 5.97 Hz, 1H), 6.58 (br s, 1H), 6.42 (d, J=8.38 Hz, 1H), 4.64 (br s, 2H), 4.29 (dd, J=7.88, 13.82 Hz, 1H), 4.21 (d, J=5.91 Hz, 2H), 4.11 (br m, 1H), 2.79-2.74 (m, 2H), 1.71-1.17 (m, 15H), 1.08-0.98 (m, 1H); Low resolution mass spectrum (ES) m/z: 628 and 630 [(M+H)+, calcd for C32H40N504: 628]; 100% purity based on HPLC.

### Example 155

### 2-[(4-CYCLOHEXYL-PHENYLCARBAMOYL)-METHYL]-PIPERAZINE-1-CARBOXYLIC ACID 9H-FLUOREN-9-YLMETHYL ESTER

p-Cyclohexylaniline was coupled to (+/−)-2-Carboxymethyl-piperazine-1,4-dicarboxylic acid 4-tert-butyl ester 1-(9H-fluoren-9-ylmethyl) ester as described in the method of Example 6. Purification by HPLC produced the title compound. ¹H NMR (400 MHz, DMSO-d6) d 9.99 (s, 1H), 9.20 (d, J=8.11 Hz, 1H), 8.74 (d, J=8.78 Hz, 1H), 7.86 (d, J=7.45 Hz, 2H), 7.61 (d, J=7.43 Hz, 1H), 7.47 (d, J=7.78 Hz, 3H), 7.39 (t, J=7.35, 7.35 Hz, 2H), 7.30 (t, J=7.45, 7.45 Hz, 1H), 7.25 (br s, 1H), 7.12 (d, J=8.04 Hz, 2H), 4.71-4.69 (m, 1H), 4.22 (s, 2H), 4.14 (s, 1H), 3.99 (d, J=13.77 Hz, 1H), 3.41 (d, J=12.64 Hz, 1H), 3.28-3.19 (m, 3H), 2.93-2.85 (m, 2H), 2.60 (dd, J=6.79, 14.93 Hz, 1H), 2.41 (br m, 1H), 1.77-1.67 (m, 5H), 1.38-1.18 (m, 5H); Low resolution mass spectrum (ES) m/z 524 [(M+H)+, calcd for C₃₃H₃₈N₃O₃: 524]; 100% purity based on HPLC.

### Example 156

### (+/−)-2-(4-CYCLOHEXYL-PHENYLCARBAMOYL)-PIPERAZINE-1-CARBOXYLIC ACID 9H-FLUOREN-9-YLMETHYL ESTER

p-Cyclohexylaniline was coupled to (+/−)-Piperazine-1,2,4-tricarboxylic acid 4-tert-butyl ester 1-(9H-fluoren-9-ylmethyl) ester as described in the method of Example 6. Purification by HPLC produced the title compound. ¹H NMR (400 MHz, DMSO-d6) d 10.15 (d, J=28.52 Hz, 1H), 9.29 (br s, 1H), 8.65 (br m, 1H), 7.88 (d, J=18.64 Hz, 2H), 7.67-7.09 (m, 10H), 4.90 (s, 1H), 4.40-4.25 (m, 3H), 4.10-3.29 (m, under D20), 2.98 (s, 1H), 1.77-1.68 (m, 5H), 1.36-1.21 (m, 5H); Low resolution mass spectrum (ES) m/e 510 [(M+H)+, calcd for C₃₂H₃₆N₃O₃: 510]; 100% purity based on HPLC.

### Example 157

### 3-(4-CYCLOHEXYL-PHENYLCARBAMOYL)-PIPERAZINE-1-CARBOXYLIC ACID 9H-FLUOREN-9-YLMETHYL ESTER

p-Cyclohexylaniline was coupled to (+/−)-Piperazine-1,2,4-tricarboxylic acid 1-tert-butyl ester 4-(9H-fluoren-9-ylmethyl) ester as described in the method of Example 6. Purification by HPLC produced the title compound. 1H NMR (400 MHz, DMSO-d6) d 10.67 (s, 1H), 9.43 (br s, 2H), 7.89 (d, J=6.82 Hz, 2H), 7.63 (d, J=7.25 Hz, 2H), 7.50 (d, J=8.38 Hz, 2H), 7.44-7.40 (m, 2H), 7.33 (br, 2H), 7.22 (d, J=8.38 Hz, 2H), 4.43-4.29 (m, 4H), 4.10 (m, 1H), 3.88 (d, J=13.76 Hz, 1H), 3.37-3.25 (m, 3H under D2O), 3.09 (br s, 1H), 2.46 (m, 1H under DMSO-D6), 1.83-1.68 (m, 5H), 1.42-1.20 (m, 5H); Low resolution mass spectrum (ES) m/z 510 [(M+H)+, calcd for C₃₂H₃₆N₃O₃: 510]; 100% purity based on HPLC.

### Example 158

### 2-[(4-CYCLOHEXYL-PHENYLCARBAMOYL)-METHYL]-PIPERAZINE-1-CARBOXYLIC ACID BENZYLAMIDE

p-Cyclohexylaniline was coupled to (+/−)-2-Carboxymethyl-piperazine-1,4-dicarboxylic acid 4-tert-butyl ester 1-(9H-fluoren-9-ylmethyl) ester, and then to benyl isocyante as described in the method of Example 26. Purification by HPLC produced the title compound. 1H NMR (400 MHz, DMSO-d6) d 9.99 (s, 1H), 9.15 (d, J=9.59 Hz, 1H), 8.76-8.74 (m, 1H), 7.47 (d, J=8.48 Hz, 2H), 7.31 (t, J=5.72, 5.72 Hz, 1H), 7.26-7.17 (m, 5H), 7.14 (d, J=8.49 Hz, 2H), 4.70 (br s, 1H), 4.24 (d, J=5.59 Hz, 2H), 4.03 (d, J=13.88 Hz, 1H), 3.46 (d, J=12.58 Hz, 1H), 3.26 (d, J=11.80 Hz, 1H), 3.18-3.07 (m, 2H), 2.98-2.88 (m, 2H), 2.60 (dd, J=5.61, 15.44 Hz, 1H), 2.45-2.41 (m, 1H), 1.78-1.68 (m, 5H), 1.42-1.17 (m, 5H); Low resolution mass spectrum (ES) m/e 435 [(M+H)+, calcd for C₂₆H₃₅N₄O₂: 435]; 98.9% purity based on HPLC.

### Example 159

### 2-[(4-CYCLOHEXYL-PHENYLCARBAMOYL)-METHYL]-PIPERAZINE-1-CARBOXYLIC ACID 4-CHLORO-BENZYLAMIDE

p-Cyclohexylaniline was coupled to (+/−)-2-Carboxymethyl-piperazine-1,4-dicarboxylic acid 4-tert-butyl ester 1-(9H-fluoren-9-ylmethyl) ester, and then to 1-Chloro-4-isocyanatomethyl-benzene as described in the method of Example 26. Purification by HPLC produced the title compound. 1H NMR (400 MHz, DMSO-d6) d 9.99 (s, 1H), 9.21 (d, J=9.23 Hz, 1H), 8.80-8.77 (br m, 1H), 7.46 (d, J=8.48 Hz, 2H), 7.33 (t, J=5.65, 5.65 Hz, 1H), 7.24 (s, 4H), 7.14 (d, J=8.54 Hz, 2H), 4.72-4.71 (br m, 1H), 4.21 (d, J=5.57 Hz, 2H), 4.02 (d, J=13.60 Hz, 1H), 3.44 (d, J=12.58 Hz, 1H), 3.26 (d, J=11.80 Hz, 1H), 3.16-3.09 (m, 2H), 2.91 (dd, J=8.29, 15.36 Hz, 2H), 2.62 (dd, J=6.13, 15.29 Hz, 1H), 2.46-2.41 (m, 1H), 1.78-1.67 (m, 5H), 1.41-1.16 (m, 5H); Low resolution mass spectrum (ES) m/e 469, 471 [(M+H)+, calcd for C₂₆H₃₄ClN₄O₂: 469]; 99.7% purity based on HPLC.

### Example 160

### 2-[(4-CYCLOHEXYL-PHENYLCARBAMOYL)-METHYL]-PIPERAZINE-1-CARBOXYLIC ACID 3,4-DICHLORO-BENZYLAMIDE

p-Cyclohexylaniline was coupled to (+/−)-2-Carboxymethyl-piperazine-1,4-dicarboxylic acid 4-tert-butyl ester 1-(9H-fluoren-9-ylmethyl) ester, and then to 1,2-Dichloro-4-isocyanatomethyl-benzene as described in the method of Example 26. Purification by HPLC produced the title compound. 1H NMR (400 MHz, DMSO-d6) d 9.97 (s, 1H), 9.14 (br s, 1H), 8.74-8.73 (br m,1H), 7.50 (d, J=1.89 Hz, 1H), 7.44 (d, J=8.38 Hz, 3H), 7.38 (t, J=5.71, 5.71 Hz, 1H), 7.21 (dd, J=1.94, 8.29 Hz, 1H), 7.13 (d, J=8.54 Hz, 2H), 4.70-4.69 (br m, 1H), 4.21 (d, J=5.55 Hz, 2H), 4.01 (d, J=13.84 Hz, 1H), 3.44 (d, J=12.53 Hz, 1H), 3.26 (d, J=11.69 Hz, 1H), 3.18-3.07 (m, 2H), 2.94-2.88 (m, 2H), 2.61 (dd, J=6.12, 15.35 Hz, 1H), 2.45-2.40 (m, 1H), 1.78-1.68 (m, 5H), 1.41-1.16 (m, 5H); Low resolution mass spectrum (ES) m/e 503, 505 [(M+H)+, calcd for C₂₆H₃₃Cl₂N₄O₂: 503]; 99.3% purity based on HPLC.

### Example 161

### 2-[(4-CYCLOHEXYL-PHENYLCARBAMOYL)-METHYL]-PIPERAZINE-1-CARBOXYLIC ACID BIPHENYL-4-YLAMIDE

p-Cyclohexylaniline was coupled to (+/−)-2-Carboxymethyl-piperazine-1,4-dicarboxylic acid 4-tert-butyl ester 1-(9H-fluoren-9-ylmethyl) ester, and then to 4-Isocyanato-biphenyl as described in the method of Example 26. Purification by HPLC produced the title compound. 1H NMR (400 MHz, DMSO-d6) d 10.05 (s, 1H), 9.18 (br d, J=8.83 Hz, 1H), 8.95, (s, 1H), 8.81-8.78 (br m, 1H), 7.62 (d, J=7.25 Hz, 2H), 7.56 (d, J=8.77 Hz, 2H), 7.50-7.41 (m, 6H), 7.31 (t, J=7.34, 7.34 Hz, 1H), 7.12 (d, J=8.54 Hz, 1H), 4.87-4.86 (br m, 1H), 4.17 (d, J=13.84 Hz, 1H), 3.49 (d, J=12.60 Hz, 1H), 3.34-3.18 (m, 3H), 3.05-32.94 (m, 3H), 2.74 (dd, J=6.27, 15.47 Hz, 1H), 2.42 (br m, 1H), 1.77-1.67 (m, 5H), 1.39-1.15 (m, 5H); Low resolution mass spectrum (ES) m/e 497 [(M+H)+, calcd for C₃₁H₃₇N₄O₂: 497]; 99.1% purity based on HPLC.

### Example 162 2-[(4-CYCLOHEXYL-PHENYLCARBAMOYL)-METHYL]-PIPERAZINE-1-CARBOXYLIC ACID (9H-FLUOREN-2-YL)-AMIDE

p-Cyclohexylaniline was coupled to (+/−)-2-Carboxymethyl-piperazine-1,4-dicarboxylic acid 4-tert-butyl ester 1-(9H-fluoren-9-ylmethyl) ester, and then to 2-Isocyanato-9H-fluorene as described in the method of Example 26. Purification by HPLC produced the title compound. 1H NMR (400 MHz, DMSO-d6) d 10.05 (s, 1H), 9.18 (d, J=9.54 Hz, 1H), 8.94 (s, 1H), 8.81-8.79 (m, 1H), 7.76 (dd, J=7.91, 12.57 Hz, 2H), 7.67 (d, J=1.03 Hz, 1H), 7.52 (d, J=7.42 Hz, 1H), 7.46 (d, J=8.54 Hz, 2H), 7.37-7.32 (m, 2H), 7.24 (dt, J=0.99, 7.43, 7.44 Hz, 1H), 7.12 (d, J=8.55 Hz, 2H), 4.87-4.86 (m, 1H), 4.18 (d, J=13.89 Hz, 1H), 3.83 (s, 2H), 3.49 (d, J=12.43 Hz, 1H), 3.34-3.19 (m, 3H), 3.05-2.93 (m, 2H), 2.75 (dd, J=6.37, 15.42 Hz, 1H), 2.41 (br m, 1H), 1.77-1.66 (m, 5H), 1.39-1.14 (m, 5H); Low resolution mass spectrum (ES) m/e 509 [(M+H)+, calcd for C₃₂H₃₇N₄O₂: 509]; 99.2% purity based on HPLC.

### Example 163 2-[(4-CYCLOHEXYL-PHENYLCARBAMOYL)-METHYL]-PIPERAZINE-1-CARBOXYLIC ACID (4-BENZYLOXY-PHENYL)-AMIDE

p-Cyclohexylaniline was coupled to (+/−)-2-Carboxymethyl-piperazine-1,4-dicarboxylic acid 4-tert-butyl ester 1-(9H-fluoren-9-ylmethyl) ester, and then to 1-Benzyloxy-4-isocyanato-benzene as described in the method of Example 26. Purification by HPLC produced the title compound. 1H NMR (400 MHz, DMSO-d6) d 10.03 (s, 1H), 9.19 (d, J=8.78 Hz, 1H), 8.80-8.78 (br m, 1H), 8.68 (s, 1H), 7.47-7.36 (m, 6H), 7.32 (td, J=2.12, 2.12, 5.29 Hz, 1H), 7.27 (d, J=9.07 Hz, 2H), 7.13 (d, J=8.54 Hz, 2H), 6.90 (d, J=9.07 Hz, 2H), 5.04 (s, 2H), 4.81-4.80 (br m, 1H), 4.13 (d, J=13.82 Hz, 1H), 3.48 (d, J=12.45 Hz, 1H), 3.31-3.14 (m, 3H), 3.00-2.94 (m, 2H), 2.68 (dd, J=5.91, 15.47 Hz, 1H), 2.43 (br m, 1H), 1.78-1.67 (m, 5H), 1.41-1.16 (m, 5H); Low resolution mass spectrum (ES) m/e 527 [(M+H)+, calcd for C₃₂H₃₉N₄O₃: 527]; 99.2% purity based on HPLC.

### Example 164

### (R)-6-AMINO-2-[3-(4-CHLORO-BENZYL)-UREIDO]-HEXANOIC ACID (4-CYCLOHEXYL-PHENYL)-AMIDE

4-Cyclohexyl-phenylamine was coupled to (R)-6-tert-Butoxycarbonylamino-2-(9H-fluoren-9-ylmethoxycarbonylamino)-hexanoic acid, and then to 1-chloro-4-isocyanatomethyl-benzene as described in the method of Example 26. Purification by HPLC produced the title compound. 1H NMR (400 MHz, DMSO-d6) d 10.00 (s, 1H), 7.67 (br s, 3H), 7.49 (d, J=8.53 Hz, 2H), 7.36 (d, J=8.43 Hz, 2H), 7.25 (d, J=8.43 Hz, 2H), 7.15 (d, J=8.54 Hz, 2H), 6.63 (t, J=6.07, 6.07 Hz, 1H), 6.35 (d, J=8.46 Hz, 1H), 4.31 (dd, J=7.95, 13.92 Hz, 1H), 4.20 (d, J=6.08 Hz, 2H), 2.79-2.74 (m, 2H), 2.43 (br m, 1H), 1.78-1.50 (m, 9H), 1.41-1.19 (m, 7H); Low resolution mass spectrum (ES) m/e 471, 473 [(M+H)+, calcd for C₂₆H₃₆ClN₄O₂: 471]; 99.1% purity based on HPLC.

### Example 165

### (R)-6-AMINO-2-[3-(3,4-DICHLORO-BENZYL)-UREIDO]-HEXANOIC ACID (4-CYCLOHEXYL-PHENYL)-AMIDE

4-Cyclohexyl-phenylamine was coupled to (R)-6-tert-Butoxycarbonylamino-2-(9H-fluoren-9-ylmethoxycarbonylamino)-hexanoic acid, and then to 1,2-Dichloro-4-isocyanatomethyl-benzene as described in the method of Example 26. Purification by HPLC produced the title compound. 1H NMR (400 MHz, DMSO-d6) d 9.99 (s, 1H), 7.66 (br s, 3H), 7.56 (d, J=8.25 Hz, 1H), 7.51-7.47 (m, 3H), 7.23 (dd, J=1.97, 8.28 Hz, 1H), 7.14 (d, J=8.55 Hz, 2H), 6.68 (t, J=6.14, 6.14 Hz, 1H), 6.42 (d, J=8.46 Hz, 1H), 4.30 (dd, J=8.08, 13.97 Hz, 1H), 4.21 (d, J=6.12 Hz, 2H), 2.79-2.74 (m, 2H), 2.43 (br m, 1H), 1.78-1.50 (m, 9H), 1.41-1.16 (m, 7H); Low resolution mass spectrum (ES) m/e 505, 507 [(M+H)+, calcd for C₂₆H₃₅ClN₄O₂: 505]; 99.1% purity based on HPLC.

### Example 166

### (R)-6-AMINO-2-[3-(4-BENZYLOXY-PHENYL)-UREIDO]-HEXANOIC ACID (4-CYCLOHEXYL-PHENYL)-AMIDE

4-Cyclohexyl-phenylamine was coupled to (R)-6-tert-Butoxycarbonylamino-2-(9H-fluoren-9-ylmethoxycarbonylamino)-hexanoic acid, and then to 1-Benzyloxy-4-isocyanato-benzene as described in the method of Example 26. Purification by HPLC produced the title compound. 1H NMR (400 MHz, DMSO-d6) d 10.08 (s, 1H), 8.54 (s, 1H), 7.65 (br s, 3H), 7.51 (d, J=8.54 Hz, 2H), 7.44-7.36 (m, 5H), 7.32 (d, J=7.22 Hz, 1H), 7.28 (d, J=9.03 Hz, 2H), 7.15 (d, J=8.56 Hz, 2H), 6.89 (d, J=9.05 Hz, 2H), 6.43 (d, J=8.27 Hz, 1H), 5.03 (s, 2H), 4.38 (dd, J=7.86, 13.64 Hz, 1H), 2.82-2.74 (m, 2H), 2.43 (br m, 1H), 1.78-1.19 (m, 16H); Low resolution mass spectrum (ES) m/e 529 [(M+H)+, calcd for C₃₂H₄₁N₄O₃: 529]; 92.3% purity based on HPLC.

### Example 167

### (S)-6-AMINO-2-[3-(4-CHLORO-BENZYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-chloro-4-isocyanatomethyl-benzene as described in the method of Example 26. Purification by HPLC produced the title compound. 1H NMR (400 MHz, DMSO-d6) d 10.08 (s, 1H), 8.54 (s, 1H), 7.65 (br s, 3H), 7.51 (d, J=8.54 Hz, 2H), 7.44-7.36 (m, 5H), 7.32 (d, J=7.22 Hz, 1H), 7.28 (d, J=9.03 Hz, 2H), 7.15 (d, J=8.56 Hz, 2H), 6.89 (d, J=9.05 Hz, 2H), 6.43 (d, J=8.27 Hz, 1H), 5.03 (s, 2H), 4.38 (dd, J=7.86, 13.64 Hz, 1H), 2.82-2.74 (m, 2H), 2.43 (br m, 1H), 1.78-1.19 (m, 16H); Low resolution mass spectrum (ES) m/e 403 [(M+H)+, calcd for C₂₁H₂₈ClN₄O₂: 403]; 92.3% purity based on HPLC.

### Example 168

### (R)-6-AMINO-2-(3-BENZYL-UREIDO)-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to benzyl isocyanate as described in the method of Example 26. Purification by HPLC produced the title compound. 1H NMR (400 MHz, DMSO-d6) d 10.00 (s, 1H), 7.72 (br s, 3H), 7.49 (d, J=8.41 Hz, 2H), 7.32-7.20 (m, 5H), 7.11 (d, J=8.35 Hz, 2H), 6.60 (t, J=5.97, 5.97 Hz, 1H), 6.33 (d, J=8.46 Hz, 1H), 4.32 (dd, J=8.28, 13.97 Hz, 1H), 4.22 (d, J=5.80 Hz, 2H), 2.81-2.73 (m,2H), 2.25 (s, 3H), 1.72-1.50 (m, 4H), 1.43-1.24 (m, 2H); Low resolution mass spectrum (ES) m/e 369 [(M+H)+, calcd for C₂₁H₂₉N₄O₂: 369]; 96.8% purity based on HPLC.

### Example 169

### (R)-6-AMINO-2-[3-(4-CHLORO-BENZYL)-UREIDO]-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to 1-chloro-4-isocyanatomethyl-benzene as described in the method of Example 26. Purification by HPLC produced the title compound. 1H NMR (400 MHz, DMSO-d6) d 9.99 (s, 1H), 7.70 (br s, 3H), 7.48 (d, J=8.39 Hz, 2H), 7.36 (d, J=8.42 Hz, 2H), 7.25 (d, J=8.45 Hz, 2H), 7.11 (d, J=8.30 Hz, 2H), 6.64 (t, J=6.05, 6.05 Hz, 1H), 6.36 (d, J=8.45 Hz, 1H), 4.30 (dd, J=8.09, 13.82 Hz, 1H), 4.20 (d, J=5.97 Hz, 2H), 2.79-2.73 (m, 2H), 2.25 (s, 3H), 1.72-1.63 (m,1H), 1.58-1.51 (m, 3H), 1.42-1.24 (m,2H); Low resolution mass spectrum (ES) m/e 403, 405 [(M+H)+, calcd for C₂₁H₂₈ClN₄O₂: 403]; 95.7% purity based on HPLC.

### Example 170

### (R)-6-AMINO-2-[3-(3,4-DICHLORO-BENZYL)-UREIDO]-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to 1,2-dichloro-4-isocyanatomethyl-benzene as described in the method of Example 26. Purification by HPLC produced the title compound. 1H NMR (400 MHz, DMSO-d6) d 9.98 (s, 1H), 7.71 (br s,3H), 7.56 (d, J=8.25 Hz, 1H), 7.50-7.48 (m, 3H), 7.23 (dd, J=1.95, 8.27 Hz, 1H), 7.10 (d, J=8.35 Hz, 2H), 6.70 (t, J=5.44, 5.44 Hz, 1H), 6.43 (d, J=8.33 Hz, 1H), .29 (dd, J=8.21, 13.84 Hz, 1H), 4.21 (d, J=6.06 Hz, 2H), 2.81-2.73 (m, 2H), 2.25 (s, 3H), 1.73-1.51 (m, 4H), 1.43-1.24 (m, 2H); Low resolution mass spectrum (ES) m/e 437, 439 [(M+H)+, calcd for C₂₁H₂₇Cl₂N₄O₂: 437]; 99.8% purity based on HPLC.

### Example 171

### (R)-6-AMINO-2-(3-BIPHENYL-4-YL-UREIDO)-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to 4-Isocyanato-biphenyl as described in the method of Example 26. Purification by HPLC produced the title compound. 1H NMR (400 MHz, DMSO-d6) d 10.10 (s, 1H), 8.93 (s, 1H), 7.74 (br s, 3H), 7.61 (d, J=7.31 Hz, 2H), 7.55 (d, J=8.75 Hz, 2H), 7.50 (t, J=8.84, 8.84 Hz, 4H), 7.42 (t, J=7.72, 7.72 Hz, 2H), 7.29 (t, J=7.33, 7.33 Hz, 1H), 7.12 (d, J=8.39 Hz, 2H), 6.67 (d, J=7.87 Hz, 1H), 4.40 (dd, J=7.98, 13.42 Hz, 1H), 2.83-2.76 (m, 2H), 2.25 (s, 3H), 1.80-1.71 (m, 1H), 1.67-1.52 (m, 3H), 1.49-1.31 (m, 2H); Low resolution mass spectrum (ES) m/e 431 [(M+H)+, calcd for C₂₆H₃₁N₄O₂: 431]; 99.9% purity based on HPLC.

### Example 172

### (R)-6-AMINO-2-[3-(4-BENZYLOXY-PHENYL)-UREIDO]-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to 1-Benzyloxy-4-isocyanato-benzene as described in the method of Example 26. Purification by HPLC produced the title compound. 1H NMR (400 MHz, DMSO-d6) d 10.07 (s, 1H), 8.58 (s, 1H), 7.71 (br s, 3H), 7.50 (d, J=8.42 Hz, 2H), 7.43-7.27 (m, 7H), 7.11 (d, J=8.40 Hz, 2H), 6.89 (d, J=9.05 Hz, 2H), 6.47 (d, J=8.26 Hz, 1H), 5.03 (s, 2H), 4.37 (dd, J=8.02, 13.53 Hz, 1H), 2.82-2.74 (m, 2H), 2.25 (s, 3H), 1.77-1.68 (m, 1H), 1.64-1.52 ( m, 3H), 1.44-1.28 (m, 2H); Low resolution mass spectrum (ES) m/e 461 [(M+H)+, calcd for C₂₇H₃₃N₄O₃: 461]; 99.7% purity based on HPLC.

### Example 173

### (R)-6-AMINO-2-[3-(9H-FLUOREN-2-YL)-UREIDO]-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to 2-Isocyanato-9H-fluorene as described in the method of Example 26. Purification by HPLC produced the title compound. 1H NMR (400 MHz, DMSO-d6) d 10.11 (s, 1H), 8.86 (s, 1H), 7.76-7.69 (m, 6H), 7.52-7.50 (m,3H), 7.34-7.31 (m, 2H), 7.22 (dt, J=0.98, 7.45, 7.47 Hz, 1H), 7.12 (d, J=8.39 Hz, 2H), 6.61 (d, J=8.07 Hz, 1H), 4.41 (dd, J=7.98, 13.44 Hz, 1H), 3.85 (s, 2H), 2.82-2.76 (m, 2H), 2.25 (s, 3H), 1.80-1.72 (m, 1H), 1.67-1.52 (m, 3H), 1.47-1.33 (m, 2H); Low resolution mass spectrum (ES) m/e 443 [(M+H)+, calcd for C₂₇H₃₁N₄O₂: 443]; 99.5% purity based on HPLC.

### Example 174

### (S)-3-(4-AMINOMETHYL-PHENYL)-2-[3-(4-BENZYLOXY-PHENYL)-UREIDO]-N-(4-TERT-BUTYL-PHENYL)-PROPIONAMIDE

p-t-Butyl-phenylamine was coupled to (S)-3-[4-(tert-Butoxycarbonylamino-methyl)-phenyl]-2-(9H-fluoren-9-ylmethoxycarbonylamino)-propionic acid, and then to 1-Benzyloxy-4-isocyanato-benzene as described in the method of Example 26. Purification by HPLC produced the title compound. 1H NMR (400 MHz, DMSO-d6) δ 10.16 (s, 1H), 8.54 (s, 1H), 8.13 (br s, 3H), 7.50 (d, J=8.74 Hz, 2H), 7.43-7.29 (m, 11H), 7.25 (d, J=6.97 Hz, 2H), 6.88 (d, J=7.03 Hz, 2H), 6.46 (d, J=8.41 Hz, 1H), 5.02 (s, 2H), 4.64 (dt, J=5.35, 8.42, 8.46 Hz, 1H), 4.00-3.98 (m, 2H), 3.09 (dd, J=5.02, 13.74 Hz, 1H), 2.88 (dd, J=8.60, 13.74 Hz, 1H), 1.26 (s, 9H); Low resolution mass spectrum (ES) m/e 551 [(M+H)+, calcd for C₃₄H₃₉N₄O₃: 551]; 99.8% purity based on HPLC.

### Example 175

### (R)-[2-(4-AMINOMETHYL-PHENYL)-1-P-TOLYLCARBAMOYL-ETHYL]-CARBAMIC ACID 9H-FLUOREN-9-YLMETHYL ESTER

p-Tolylamine to (R)-3-[4-(tert-Butoxycarbonylamino-methyl)-phenyl]-2-(9H-fluoren-9-ylmethoxycarbonylamino)-propionic acid as described in the method of Example 6. Purification by HPLC produced the title compound. 1H NMR (400 MHz, DMSO-d6) δ 7.85 (d, J=7.5 Hz, 2H), 7.64 (br d, J=6.6 Hz, 2H), 7.44 (d, J=8.6 Hz, 2H), 7.42-7.24 (m, 8H), 7.11 (d, J=8.1 Hz, 2H), 4.35 (dd, J=4.4, 10.1 Hz, 1H), 4.27-4.09 (m, 3H), 3.94 (s, 2H), 3.02 (dd, J=4.4, 13.8 Hz, 1H), 2.86 (dd, J=10.5, 13.6 Hz, 1H), 2.23 (s, 3H), 1.42 (s, 1H); Low resolution mass spectrum (ES) m/e 506 [(M+H)+, calcd for C₃₂H₃₂N₃O₃: 506]; 95% purity based on HPLC.

### Example 176

### (S)-[5-(2-AMINO-ACETYLAMINO)-1-P-TOLYLCARBAMOYL-PENTYL]-CARBAMIC ACID 9H-FLUOREN-9-YLMETHYL ESTER

(S)-6-Amino-2-(2-9H-fluoren-9-yl-acetylamino)-hexanoic acid p-tolylamide of Example 53 was coupled to tert-Butoxycarbonylamino-acetic acid as described in the method of Example 6. Purification by HPLC produced the title compound. 1H NMR (400 MHz, DMSO-d6) δ 7.87 (d, J=7.5 Hz, 2H), 7.70 (t, J=7.7 Hz, 2H), 7.44 (d, J=8.4 Hz, 2H), 7.40 (ddd, J=2.1, 7.5, 8.1 Hz, 2H), 7.30 (dd, J=8.4, 12.7 Hz, 2H), 7.09 (d, J=8.3 Hz, 2H), 4.33-4.16 (m, 3H), 4.07 (dd, J=5.3, 9.0 Hz, 1H), 3.46 (s, 1H), 3.13-3.06 (m, 1H), 2.74 (t, J=7.7 Hz, 2H), 2.22 (s, 3H), 1.22-1.72 (m, 6H+A22); Low resolution mass spectrum (ES) m/e 515 [(M+H)+, calcd for C₃₀H₃₅N₄O₄: 515]; 85% purity based on HPLC.

### Example 177

### (S)-[4-(2-AMINO-ACETYLAMINO)-1-P-TOLYLCARBAMOYL-BUTYL]-CARBAMIC ACID 9H-FLUOREN-9-YLMETHYL ESTER

(S)-(4-Amino-1-p-tolylcarbamoyl-butyl)-carbamic acid 9H-fluoren-9-ylmethyl ester of Example 101 was coupled to tert-Butoxycarbonylamino-acetic acid as described in the method of Example 6. Purification by HPLC produced the title compound. 1H NMR (400 MHz, DMSO-d6) δ 7.86 (d, J=7.5 Hz, 2H), 7.69 (t, J=7.3 Hz, 2H), 7.44 (d, J=8.3 Hz, 2H). 7.40 (ddd, J=2.1, 7.2, 8.5 Hz, 2H), 7.30 (dd, J=7.0, 13.2 Hz, 2H), 7.09 (d, J=8.3 Hz, 2H), 4.32-4.16 (m, 3H), 4.08 (dd, J=5.3, 8.8 Hz, 1H), 3.47 (s, 2H). 3.18-3.07 (m, 2H), 2.22 (s, 3H), 1.77-1.33 (m, 4H); Low resolution mass spectrum (ES) m/e 501 [(M+H)+, calcd for C₂₉H₃₃N₄O₄: 501]; 85% purity based on HPLC.

### Example 178

### (S)-[2-(3-AMINO-PROPIONYLAMINO)-1-P-TOLYLCARBAMOYL-ETHYL]-CARBAMIC ACID 9H-FLUOREN-9-YLMETHYL ESTER

(S)-(2-Amino-1-p-tolylcarbamoyl-ethyl)-carbamic acid 9H-fluoren-9-ylmethyl ester of Example 103 was coupled to 3-tert-Butoxycarbonylamino-propionic acid as described in the method of Example 6. Purification by HPLC produced the title compound. 1H NMR (400 MHz, DMSO-d6) δ 7.87 (d, J=7.5 Hz, 2H), 7.69 (t, J=6.4, 5.9 Hz, 2H), 7.44 (d, J=8.4 Hz, 2H), 7.40 (t, J=7.5 Hz, 2H), 7.31 (dd, J=6.8, 14.1 Hz, 2H), 7.10 (d, J=8.3 Hz, 2H), 4.35-4.17 (m, 4H), 3.43 (dd, J=6.6, 13.5 Hz, 1H), 3.35 (dd, J=6.2, 13.4 Hz, 1H), 2.93 (t, J=6.9 Hz, 2H), 2.40 (t, J=6.8 Hz, 2H), 2.22 (s, 3H); Low resolution mass spectrum (ES) m/e 487 [(M+H)+, calcd for C₂₈H₃₁N₄O₄: 487]; 98% purity based on HPLC.

### Example 179

### (S)-[3-(3-AMINO-PROPIONYLAMINO)-1-P-TOLYLCARBAMOYL-PROPYL]-CARBAMIC ACID 9H-FLUOREN-9-YLMETHYL ESTER

(S)-(3-Amino-1-p-tolylcarbamoyl-propyl)-carbamic acid 9H-fluoren-9-ylmethyl ester of Example 108 was coupled to 3-tert-Butoxycarbonylamino-propionic acid as described in the method of Example 6. Purification by HPLC produced the title compound. 1H NMR (400 MHz, DMSO-d6) δ 7.87 (d, J=7.5 Hz, 2H), 7.71 (t, J=6.5 Hz, 2H), 7.44 (d, J=8.3 Hz, 2H), 7.40 (ddd, J=1.5, 7.7, 8.1 Hz, 2H), 7.31 (dd, J=7.2, 14.0 Hz, 2H), 7.10 (d, J=8.3 Hz, 2H), 4.31-4.17 (m, 3H), 4.12 (dd, J=5.9, 8.6 Hz, 1H), 3.17-3.08 (m, 2H), 2.96 (t, J=7.0, 6.6 Hz, 2H), 2.42 (t, J=6.8 Hz, 2H), 2.23 (s, 3H), 1.91-1.69 (m, 2H); Low resolution mass spectrum (ES) m/e 501 [(M+H)+, calcd for C₂₉H₃₃N₄O₄: 501]; 100% purity based on HPLC.

### Example 180

### (S)-[2-(4-AMINO-PHENYL)-1-P-TOLYLCARBAMOYL-ETHYL]-CARBAMIC ACID 9H-FLUOREN-9-YLMETHYL ESTER

p-Tolylamine was coupled to (S)-3-(4-tert-Butoxycarbonylamino-phenyl)-2-(9H-fluoren-9-ylmethoxycarbonylamino)-propionic acid as described in the method of Example 6. Purification by HPLC produced the title compound. 1H NMR (400 MHz, DMSO-d6) δ 7.86 (d, J=7.5 Hz, 2H), 7.64 (dd, J=4.4, 7.5 Hz, 2H), 7.43 (d, J=8.1 Hz, 2H), 7.39 (t, J=7.7 Hz, 2H), 7.32-7.21 (m, 4H), 7.10 (d, J=8.3 Hz, 2H), 6.98-6.87 (br, 2H), 4.30 (dd, J=5.3, 9.9 Hz, 1H), 4.21-4.07 (m, 3H), 2.95 (dd, J=4.6, 13.6 Hz, 1H), 2.80 (dd, J=10.3, 13.6 Hz, 1H), 2.23 (s, 3H); Low resolution mass spectrum (ES) m/e 492 [(M+H)+, calcd for C₃₁H₃₀N₃O₃: 492]; 91% purity based on HPLC.

### Example 181

### (S)-(5-ACETYLAMINO-1-P-TOLYLCARBAMOYL-PENTYL)-CARBAMIC ACID 9H-FLUOREN-9-YLMETHYL ESTER

(S)-6-Amino-2-(2-9H-fluoren-9-yl-acetylamino)-hexanoic acid p-tolylamide of Example 53 was coupled to acetyl chloride as described in the method for Intermediate #3 of Example 1. Purification by HPLC produced the title compound. 1H NMR (400 MHz, DMSO-d6) δ 9.90 (s, 1H), 7.86 (d, J=7.7 Hz, 2H), 7.70 (t, J=6.6 Hz, 2H), 7.43 (d, J=8.3 Hz, 2H), 7.40 (ddd, J=1.8, 7.2, 8.1 Hz, 2H), 7.30 (dd, J=7.2, 12.3 Hz, 2H), 7.08 (d, J=8.3 Hz, 2H), 4.29-4.16 (m, 3H), 4.05 (dd, J=5.5, 9.0, 1H), 3.04-2.93 (m, 2H), 2.22 (s, 3H), 1.75 (s, 3H), 1.68-1.52 (m, 3H), 1.46-1.22 (m, 3H); Low resolution mass spectrum (ES) m/e 500 [(M+H)+, calcd for C₃₀H₃₄N₃O₄: 500]; 97% purity based on HPLC.

### Example 182

### (+/−)-4-(2-AMINO-ACETYL)-2-(P-TOLYLCARBAMOYL-METHYL)-PIPERAZINE-1-CARBOXYLIC ACID 9H-FLUOREN-9-YLMETHYL ESTER

p-Tolylamine was coupled to (+/−)-2-Carboxymethyl-piperazine-1,4-dicarboxylic acid 4-tert-butyl ester 1-(9H-fluoren-9-ylmethyl) ester, and final coupling to tert-Butoxycarbonylamino-acetic acid was as described in the method of Example 6. Purification by HPLC produced the title compound. 1H NMR (400 MHz, DMSO-d6) δ 10.01 (s, 0.5H), 9.77 (br s, 0.5H), 8.07-8.02 (m, 3H), 7.88 (s, 2H), 7.63-7.29 (m, 8H), 7.09 (s, 2H), 4.56 (d, J=30.72 Hz, 1H), 4.32-4.01 (m, 5H), 3.89-3.78 (m, 3H) under D20, 3.32-3.30 (m, 1H) under D2O, 3.21-2.99 (m, 3H), 2.58-2.55 (m, 0.5H), 2.45-2.32 (m, 1.5H), 2.23 (d, J=5.36 Hz, 3H); Low resolution mass spectrum (ES) m/e 513 [(M+H)+, calcd for C₃₀H₃₃N₄O₄: 513]; 98% purity based on HPLC.

### Example 183

### 4-(3-AMINO-PROPIONYL)-2-(P-TOLYLCARBAMOYL-METHYL)-PIPERAZINE-1-CARBOXYLIC ACID 9H-FLUOREN-9-YLMETHYL ESTER

p-Tolylamine was coupled to (+/−)-2-Carboxymethyl-piperazine-1,4-dicarboxylic acid 4-tert-butyl ester 1-(9H-fluoren-9-ylmethyl) ester, and then to 3-tert-Butoxycarbonylamino-propionic acid as described in the method of Example 182. Purification by HPLC produced the title compound. 1H NMR (400 MHz, DMSO-d6) δ 9.96 (s, 0.5H), 9.78 (s, 0.5H), 7.87 (d, J=6.18 Hz, 2H), 7.64-7.29 (m, 11H), 7.08 (s, 2H), 4.56 (d, J=20.79 Hz, 1H), 4.31-4.22 (m, 5H), 3.31 (d, J=12.11 Hz, 1H), 3.12-2.92 (m, 4H), 2.82-2.64 (m, 3H), 2.45-2.32 (m, 1H), 2.23 (d, J=3.09 Hz, 3H); Low resolution mass spectrum (ES) m/e 527 [(M+H)+, calcd for C₃₁H₃₅N₄O₄: 527]; 96.6% purity based on HPLC.

### Example 184

### 4-((S)-2-AMINO-PROPIONYL)-2-(P-TOLYLCARBAMOYL-METHYL)-PIPERAZINE-1-CARBOXYLIC ACID 9H-FLUOREN-9-YLMETHYL ESTER

p-Tolylamine was coupled to (+/−)-2-Carboxymethyl-piperazine-1,4-dicarboxylic acid 4-tert-butyl ester 1-(9H-fluoren-9-ylmethyl) ester, and then to (S)-2-tert-Butoxycarbonylamino-propionic acid as described in the method of Example 182. Purification by HPLC produced the title compound. 1H NMR (400 MHz, DMSO-d6) δ 10.16-9.59 (m, 1H), 8.11 (s, 3H), 7.87 (d, J=6.73 Hz, 2H), 7.64-7.28 (m, 8H), 7.12-7.09 (m, 2H), 4.65-4.11 (m, 6.5H), 3.98-3.84 (m, 1.5H), 3.29-2.87 (m, 2.5H), 2.77-2.65 (m, 1H), 2.42-2.31 (m, 0.5H), 2.23 (s, 3H), 1.41 (d, J=6.82 Hz, 0.6H), 1.32-1.28 (m, 2.2H); Low resolution mass spectrum (ES) m/e 527 [(M+H)+, calcd for C₃₁H₃₅N₄O₄: 527]; 97.7% purity based on HPLC.

### Example 185

### 4-(R-2-AMINO-PROPIONYL)-2-(P-TOLYLCARBAMOYL-METHYL)-PIPERAZINE-1-CARBOXYLIC ACID 9H-FLUOREN-9-YLMETHYL ESTER

p-Tolylamine was coupled to (+/−)-2-Carboxymethyl-piperazine-1,4-dicarboxylic acid 4-tert-butyl ester 1-(9H-fluoren-9-ylmethyl) ester, and then to (R)-2-tert-Butoxycarbonylamino-propionic acid as described in the method of Example 182. Purification by HPLC produced the title compound. 1H NMR (400 MHz, DMSO-d6) δ 10.16-9.60 (m, 1H), 8.12 (s,3H), 7.87 (d, J=6.62 Hz, 2H), 7.73-7.28 (m, 8H), 7.12-7.09 (m, 2H), 4.65-4.11 (m, 5H), 3.98-3.34 (m, XH under D2O), 3.25-2.65 (m, 4H), 2.59-2.42 (m, nH under DMSO), 2.23 (s, 3H), 1.42-1.28 (m, 3H); Low resolution mass spectrum (ES) m/e 527 [(M+H)+, calcd for C₃₁H₃₅N₄O₄: 527]; 99.2% purity based on HPLC.

### Example 186

### 4-(2-ACETYLAMINO-ACETYL)-2-(P-TOLYLCARBAMOYL-METHYL)-PIPERAZINE-1-CARBOXYLIC ACID 9H-FLUOREN-9-YLMETHYL ESTER

(+/−)-4-(2-Amino-acetyl)-2-(p-tolylcarbamoyl-methyl)-piperazine-1-carboxylic acid 9H-fluoren-9-ylmethyl ester of example 182 was coupled to acetyl chloride as described in the method for Intermediate #3 of Example 1. Purification by HPLC produced the title compound. 1H NMR (400 MHz, DMSO-d6) δ 9.94 (s, 0.5H), 9.75 (s, 0.5H), 8.05-8.01 (m, 1H), 7.87 (d, J=5.91 Hz, 2H), 7.64-7.29 (m, 8H), 7.08 (m, 2H), 4.28-4.05 (m, 4H), 3.97-3.82 (m, 3H), 3.28-2.88 (m, 3H), 2.71 (br, 0.5H), 2.40-2.32 (m, 0.5), 2.23 (s, 3H), 1.87 (d, J=8.34 Hz, 3H); Low resolution mass spectrum (ES) m/e 555 [(M+H)+, calcd for C₃₂H₃₅N₄O₅: 555 ]; 97.7% purity based on HPLC.

### Example 187

### 4-(2-ETHYLAMINO-ACETYL)-2-(P-TOLYLCARBAMOYL-METHYL)-PIPERAZINE-1-CARBOXYLIC ACID 9H-FLUOREN-9-YLMETHYL ESTER

p-Tolylamine was coupled to (+/−)-2-Carboxymethyl-piperazine-1,4-dicarboxylic acid 4-tert-butyl ester 1-(9H-fluoren-9-ylmethyl) ester, and then to Ethylamino-acetic acid as described in the method of Example 182. Purification by HPLC produced the title compound. 1H NMR (400 MHz, DMSO-d6) δ 10.00 (s, 0.5H), 9.80 (s, 0.5H), 8.76 (br m, 2H), 8.87 (brm, 2H), 7.63-7.29 (m, 8H), 7.08 (m, 2H), 4.60-4.56 (m, 1H), 4.33-4.07 (m, 5H), 3.21-2.67 (m, 5H), 2.44-2.36 (m, 0.6), 2.23-2.22 (d, 3H), 1.45 (s, 2.6H), 1.21 (t, J=7.25, 1.8H), 1.16 (t, J=7.26, 1.2H); Low resolution mass spectrum (ES) m/e 541 [(M+H)+, calcd for C₃₂H₃₇N₄O₄: 541]; 93.9% purity based on HPLC.

### Example 188

### (S)-5-AMINO-2-(3-BIPHENYL-4-YL-UREIDO)-PENTANOIC ACID P-TOLYLAMIDE

p-Tolylamine was coupled to (S)-5-tert-Butoxycarbonylamino-2-(9H-fluoren-9-ylmethoxycarbonylamino)-pentanoic acid, and then to 4-Isocyanato-biphenyl as described in the method of Example 26. Purification by HPLC produced the title compound. 1H NMR (400 MHz, DMSO-d6) δ 10.16 (s, 1H), 8.93 (br s, 3H), 7.61 (d, J=7.23 Hz, 2H), 7.56 (d, J=8.77 Hz, 2H), 7.49 (t, J=8.80, 8.80 Hz, 4H), 7.41 (d, J=7.92 Hz, 2H), 7.30 (t, J=7.86, 7.86 Hz, 1H), 7.13 (d, J=8.37 Hz, 2H), 6.66 (d, J=8.28 Hz, 1H), 4.48 (dd, J=6.28, 13.97 Hz, 1H), 2.85-2.83 (m, 2H), 2.26 (s, 3H), 1.84-1.56 (m, 4H); Low resolution mass spectrum (ES) m/e 417 [(M+H)+, calcd for C₂₅H₂₉N₄O₂: 417]; 99.2% purity based on HPLC.

### Example 189

### (S)-5-AMINO-2-[3-(4-BENZYLOXY-PHENYL)-UREIDO]-PENTANOIC ACID P-TOLYLAMIDE

p-Tolylamine was coupled to (S)-5-tert-Butoxycarbonylamino-2-(9H-fluoren-9-ylmethoxycarbonylamino)-pentanoic acid, and then to 1-Benzyloxy-4-isocyanato-benzene as described in the method of Example 26. Purification by HPLC produced the title compound. 1H NMR (400 MHz, DMSO-d6) δ 10.13 (s, 1H),8.62 (s, 1H), 7.71 (br s, 3H), 7.50 (d, J=8.42 Hz, 2H), 7.44-7.36 (m, 4H), 7.33-7.27 (m, 3H), 7.12 (d, J=8.39 Hz, 2H), 6.90 (d, J=9.04 Hz, 2H), 6.50 (d, J=8.33 Hz, 1H), 5.03 (s, 2H), 4.43 (dd, J=6.09, 13.66 Hz, 1H), 2.83-2.82 (m, 2H), 2.25 (s, 3H), 1.79-1.71 (m, 1H), 1.67-1.56 (m, 3H);Low resolution mass spectrum (ES) m/e 447 [(M+H)+, calcd for C₂₆H₃₁N₄O₃: 447]; 98.2% purity based on HPLC.

### Example 190

### (S)-5-AMINO-2-[3-(9H-FLUOREN-2-YL)-UREIDO]-PENTANOIC ACID P-TOLYLAMIDE

p-Tolylamine was coupled to (S)-5-tert-Butoxycarbonylamino-2-(9H-fluoren-9-ylmethoxycarbonylamino)-pentanoic acid, and then to 2-Isocyanato-9H-fluorene as described in the method of Example 26. Purification by HPLC produced the title compound. ¹H NMR (400 MHz, DMSO-d6) δ (S)-5-Amino-2-[3-(9H-fluoren-2-yl)-ureido]-pentanoic acid p-tolylamide: 1H NMR (400 MHz, DMSO-d6) δ 10.16 (s, 1H), 8.93 (s, 1H), 7.77-7.72 (br m, 6H), 7.52-7.50 (m, 3H), 7.34-7.31 (m, 2H), 7.22 (t, J=7.40, 7.40 Hz, 1H), 7.13 (d, J=8.40 Hz, 2H), 6.67 (d, J=8.23 Hz, 1H), 4.48 (dd, J=6.07, 13.59 Hz, 1H), 3.86 (s, 2H), 2.85-2.84 (m, 2H), 2.26 (s, 3H), 1.84-1.62 (m, 4H); Low resolution mass spectrum (ES) m/e 429 [(M+H)+, calcd for C₂₆H₂₉N₄O₂: 429]; 99.6% purity based on HPLC.

### Example 191

### (R)-5-AMINO-2-(3-BIPHENYL-4-YL-UREIDO)-PENTANOIC ACID P-TOLYLAMIDE

p-Tolylamine was coupled to (R)-5-tert-Butoxycarbonylamino-2-(9H-fluoren-9-ylmethoxycarbonylamino)-pentanoic acid, and then to 4-Isocyanato-biphenyl as described in the method of Example 26. Purification by HPLC produced the title compound. ¹H NMR (400 MHz, DMSO-d6) δ 10.16 (s, 1H), 8.92 (s, 1H), 7.70 (br s, 3H), 7.62-7.60 (m, 2H), 7.56 (d, J=8.73 Hz, 2H), 7.49 (t, J=8.85, 8.85 Hz, 4H), 7.42 (t, J=7.73, 7.73 Hz, 2H), 7.30 (t, J=7.35, 7.35 Hz, 1H), 7.13 (d, J=8.37 Hz, 2H), 6.66 (d, J=8.28 Hz, 1H), 4.48 (dd, J=6.19, 13.83 Hz, 1H), 2.85-2.83 (m, 2H), 2.26 (s, 3H), 1.84-1.61 (m, 4H); Low resolution mass spectrum (ES) m/e 417 [(M+H)+, calcd for C₂₅H₂₉N₄O₂: 417]; 93.9% purity based on HPLC.

### Example 192

### (R)-5-AMINO-2-[3-(4-BENZYLOXY-PHENYL)-UREIDO]-PENTANOIC ACID P-TOLYLAMIDE

p-Tolylamine was coupled to (R)-5-tert-Butoxycarbonylamino-2-(9H-fluoren-9-ylmethoxycarbonylamino)-pentanoic acid, and then to 1-Benzyloxy-4-isocyanato-benzene as described in the method of Example 26. Purification by HPLC produced the title compound. ¹H NMR (400 MHz, DMSO-d6) δ 10.13 (s, 1H), 8.63 (s, 1H), 7.73 (br s,3H), 7.44-7.36 (m, 4H), 7.33-7.27 (m, 3H), 7.12 (d, J=8.40 Hz, 2H), 6.90 (d, J=9.07 Hz, 2H), 6.52 (d, J=8.31 Hz, 1H), 5.03 (s, 2H), 4.43 (dd, J=6.35, 13.88 Hz, 1H), 2.83-2.82 (m, 2H), 2.25 (s, 3H), 1.78-1.58 (m, 4H); Low resolution mass spectrum (ES) m/e 447 [(M+H)+, calcd for C₂₆H₃₁N₄O₃: 447]; 94.7% purity based on HPLC.

### Example 193

### (R)-5-AMINO-2-[3-(9H-FLUOREN-2-YL)-UREIDO]-PENTANOIC ACID P-TOLYLAMIDE

p-Tolylamine was coupled to (R)-5-tert-Butoxycarbonylamino-2-(9H-fluoren-9-ylmethoxycarbonylamino)-pentanoic acid then to 2-Isocyanato-9H-fluorene as described in the method of Example 26. Purification by HPLC produced the title compound. ¹H NMR (400 MHz, DMSO-d6) δ 10.16 (s, 1H), 8.92 (s, 1H), 7.81-7.72 (m, 6H), 7.52-7.50 (m, 3H), 7.34-7.31 (m, 2H), 7.24-7.12 (m, 3H), 6.66 (d, J=8.24 Hz, 1H), 4.48 (dd, J=6.11, 13.66 Hz, 1H), 3.86 (s, 2H), 2.85-2.83 (m, 2H), 2.26 (s, 3H), 1.84-1.62 (m, 4H); Low resolution mass spectrum (ES) m/e 429 [(M+H)+, calcd for C₂₆H₂₉N₄O₂: 429]; 93.6% purity based on HPLC.

### Example 194

### (S)-6-AMINO-2-[3-(4-HYDROXY-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Benzyloxy-4-isocyanato-benzene as described in the method of Example 26. Hydrogenation overnight using 10% palladium on carbon in ethanol under a hydrogen atmosphere followed by purification by HPLC produced the title compound. ¹H NMR (400 MHz, DMSO-d6) δ 10.05 (s, 1H), 8.97 (s, 1H), 8.39 (s, 1H), 7.68 (br s, 3H), 7.49 (d, J=8.42 Hz, 2H), 7.14 (d, J=8.87 Hz, 2H), 7.11 (d, J=8.48 Hz, 2H), 6.63 (d, J=8.86 Hz, 2H), 6.56 (br s,0.5H), 6.38 (d, J=8.28 Hz, 1H), 4.36 (dd, J=8.01, 13.54 Hz, 1H), 2.80-2.76 (m, 2H), 2.25 (s, 3H), 1.76-1.67 (m, 1H), 1.63-1.52 (m, 3H), 1.43-1.31 (m, 2H); Low resolution mass spectrum (ES) m/e 371 [(M+H)+, calcd for C₂₀H₂₇N₄O₃: 371]; 86.6% purity based on HPLC.

### Example 195

### (R)-6-AMINO-2-[3-(9H-FLUOREN-9-YL)-UREIDO]-HEXANOIC ACID (4-CYCLOHEXYL-PHENYL)-AMIDE

4-Cyclohexyl-phenylamine was coupled to (R)-6-tert-Butoxycarbonylamino-2-(9H-fluoren-9-ylmethoxycarbonylamino)-hexanoic acid, and then to 9-Isocyanato-9H-fluorene as described in the method of Example 26. Purification by HPLC produced the title compound. ¹H NMR (400 MHz, DMSO-d6) δ 10.09 (s, 1H), 7.84 (d, J=6.42 Hz, 2H), 7.67 (br, 3H), 7.55-7.48 (m, 3H), 7.44-7.37 (m, 2H), 7.31 (td, J=7.48, 7.48, 10.62 Hz, 2H), 7.17 (d, J=8.54 Hz, 2H), 6.63 (d, J=8.65 Hz, 1H), 6.26 (d, J=8.47 Hz, 1H), 5.82 (d, J=8.59 Hz, 1H), 4.47 (dd, J=7.90, 13.97 Hz, 1H), 2.82-2.77 (m, 2H), 2.44 (br m, 1H), 1.79-1.69 (m, 6H), 1.64-1.53 (m, 3H), 1.46-1.31 (m, 6H), 1.26-1.20 (m, 1H); Low resolution mass spectrum (ES) m/e 511 [(M+H)+, calcd for C₃₂H₃₉N₄O₂: 511]; 90.5% purity based on HPLC.

### Example 196

### (S)-6-AMINO-2-{3-[4-(4-NITRO-BENZYLOXY)-PHENYL]-UREIDO}-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-(4-nitrophenyl)methyloxy-4-isocyanato-benzene as described in the method of Example 26. Purification by HPLC produced the title compound. ¹H NMR (400 MHz, DMSO-d6) δ 10.06 (s, 1H), 8.56 (s, 1H), 8.25 (d, J=8.77 Hz, 2H), 7.71-7.65 (m, 5H), 7.49 (d, J=8.40 Hz, 2H), 7.29 (d, J=7.05 Hz, 2H), 7.11 (d, J=8.30 Hz, 2H), 6.92 (d, J=9.06 Hz, 2H), 6.44 (d, J=8.29 Hz, 1H), 5.21 (s, 2H), 4.37 (dd, J=8.02, 13.64 Hz, 1H), 2.80-2.75 (m, 2H), 2.25 (s, 3H), 1.77-1.68 (m, 1H), 1.63-1.50 (m, 3H), 1.43-1.30 (m, 2H); Low resolution mass spectrum (ES) m/e 506 [(M+H)+, calcd for C₂₇H₃₂N₅O₅: 506]; 97.5% purity based on HPLC.

### Example 197

### 2-(P-TOLYLCARBAMOYL-METHYL)-PIPERAZINE-1-CARBOXYLIC ACID 9H-FLUOREN-9-YLMETHYL ESTER

p-Tolylamine was coupled to (+/−)-2-Carboxymethyl-piperazine-1,4-dicarboxylic acid 4-tert-butyl ester 1-(9H-fluoren-9-ylmethyl) ester as described in the method of Example 6. Purification by HPLC produced the title compound. ¹H NMR (400 MHz, DMSO-d6) δ 9.99 (s, 1H), 9.23 (s, 1H), 8.75 (s, 1H), 7.86 (d, J=7.30 Hz, 2H), 7.62 (d, J=7.41 Hz, 1H), 7.48-7.46 (br m, 3H), 7.39 (t, J=7.40, 7.40 Hz, 2H), 1.32-1.26 (m, 2H), 7.10 (d, J=8.07 Hz, 2H), 4.68-4.67 (m, 1H), 4.25 (br s,2H), 4.17 (br s,1H), 3.98 (d, J=12.60 Hz, 1H), 3.41 (d, J=12.72 Hz, 1H), 3.28-3.17 (m, 3H), 2.90 (dd, J=8.30, 15.06 Hz, 2H), 2.56 (dd, J=6.27, 15.03 Hz, 1H), 2.23 (s, 3H); Low resolution mass spectrum (ES) m/e 456 [(M+H)+, calcd for C₂₈H₃₀N₃O₃: 456]; 99.7% purity based on HPLC.

### Example 198

### (R)-[4-AMINO-1-(4-CYCLOHEXYL-PHENYLCARBAMOYL)-BUTYL]-CARBAMIC ACID 9H-FLUOREN-9-YLMETHYL ESTER

4-Cyclohexyl-phenylamine was coupled to (R)-5-tert-Butoxycarbonylamino-2-(9H-fluoren-9-ylmethoxycarbonylamino)-pentanoic acid as described in the method of Example 6. Purification by HPLC produced the title compound. ¹H NMR (400 MHz, DMSO-d6) δ 10.02 (s, 1H), 7.89 (d, J=7.51 Hz, 2H), 7.76-7.71 (m, 6H), 7.50 (d, J=8.29 Hz, 2H), 7.42 (t, J=7.30, 7.30 Hz, 2H), 7.32 (dd, J=6.81, 12.89 Hz, 2H), 7.15 (d, J=8.46 Hz, 2H), 4.34-4.14 (m, 4H), 2.81-2.80 (m, 2H), 2.43 (br, 1H), 1.78-1.60 (m, 9H), 1.41-1.19 (m, 5H); Low resolution mass spectrum (ES) m/e 512 [(M+H)+, calcd for C₃₂H₃₈N₃O₃: 512]; 99.5% purity based on HPLC.

### Example 199

### (S)-4-{4-13-(5-AMINO-1-P-TOLYLCARBAMOYL-PENTYL)-UREIDO]-PHENOXYMETHYL}-BENZOIC ACID METHYL ESTER

p-Tolylamine was coupled to (S)-6-tert-Butoxycarbonylamino-2-(9H-fluoren-9-ylmethoxycarbonylamino)-hexanoic acid, and then to 4-(4-Isocyanato-phenoxymethyl)-benzoic acid methyl ester as described in the method of Example 26. Purification by HPLC produced the title compound. ¹H NMR (400 MHz, DMSO-d6) δ 10.07 (s, 1H), 8.54 (s, 1H), 7.97 (d, J=8.27 Hz, 2H), 7.64 (br s, 3H), 7.57 (d, J=8.29 Hz, 2H), 7.49 (d, J=8.39 Hz, 2H), 7.28 (d, J=9.04 Hz, 2H), 7.11 (d, J=8.35 Hz, 2H), 6.90 (d, J=9.05 Hz, 2H)6.43 (d, J=8.28 Hz, 1H), 5.14 (s, 2H), 4.37 (dd, J=7.97, 13.62 Hz, 1H), 3.85 (s, 3H), 2.82-2.74 (m, 2H), 2.25 (s, 3H), 1.77-1.68 (m, 1H), 1.63-1.52 (m, 3H), 1.44-1.29 (m, 2H0; Low resolution mass spectrum (ES) m/e 519 [(M+H)+, calcd for C₂₉H₃₅N₄O₅: 519]; 93.0% purity based on HPLC.

### Example 200

### (S)-[4-AMINO-1-(4-CYCLOHEXYL-PHENYLCARBAMOYL)-BUTYL]-CARBAMIC ACID 9H-FLUOREN-9-YLMETHYL ESTER

4-Cyclohexyl-phenylamine was coupled to (S)-5-tert-Butoxycarbonylamino-2-(9H-fluoren-9-ylmethoxycarbonylamino)-pentanoic acid as described in the method of Example 6. Purification by HPLC produced the title compound. ¹H NMR (400 MHz, DMSO-d6) δ 10.01 (s, 1H), 7.90 (d, J=7.51 Hz, 2H), 7.75-7.71 (m, 5H), 7.50 (d, J=8.42 Hz, 2H), 7.42 (t, J=7.34, 7.34 Hz, 2H), 7.32 (dd, J=6.91, 12.74 Hz, 2H), 7.15 (d, J=8.44 Hz, 2H), 4.34-4.13 (m, 4H), 2.81-2.79 (m, 2H), 2.43_br, 1H), 1.78-1.56 (m, 9H), 1.41-1.19 (m, 5H); Low resolution mass spectrum (ES) m/e 512 [(M+H)+, calcd for C₃₂H₃₈N₃O₃: 512]; 99.5% purity based on HPLC.

### Example 201

### (R)-5-AMINO-2-(3-BIPHENYL-4-YL-UREIDO)-PENTANOIC ACID (4-CYCLOHEXYL-PHENYL)-AMIDE

4-Cyclohexyl-phenylamine was coupled to (R)-5-tert-Butoxycarbonylamino-2-(9H-fluoren-9-ylmethoxycarbonylamino)-pentanoic acid, and then to 4-Isocyanato-biphenyl as described in the method of Example 26. Purification by HPLC produced the title compound. ¹H NMR (400 MHz, DMSO-d6) δ 10.17 (s, 1H), 8.91 (s, 1H), 7.67 (br s, 3H), 7.61 (d, J=7.28 Hz, 2H), 7.57-7.47 (m, 6H), 7.42 (t, J=7.69, 7.69 Hz, 2H), 7.30 (t, J=7.34, 7.34 Hz, 1H), 7.17 (d, J=8.53 Hz, 2H), 6.64 (d, J=8.25 Hz, 1H), 4.48 (dd, J=6.47,13.77 Hz, 1H), 2.84-2.82 (m, 2H), 2.44 (br m, 1H), 1.78-1.57 (m, 8H), 1.42-1.20 (m, 4H); Low resolution mass spectrum (ES) m/e 485 [(M+H)+, calcd for C₃₀H₃₇N₄O₂: 485]; 99.5% purity based on HPLC.

### Example 202

### (R)-5-AMINO-2-[3-(4-BENZYLOXY-PHENYL)-UREIDO]-PENTANOIC ACID (4-CYCLOHEXYL-PHENYL)-AMIDE

4-Cyclohexyl-phenylamine was coupled to (R)-5-tert-Butoxycarbonylamino-2-(9H-fluoren-9-ylmethoxycarbonylamino)-pentanoic acid, and then to 1-Benzyloxy-4-isocyanato-benzene as described in the method of Example 26. Purification by HPLC produced the title compound. ¹H NMR (400 MHz, DMSO-d6) δ 10.14 (s, 1H), 8.60 (s, 1H), 7.67 (br s, 3H), 7.51 (d, J=8.53 Hz, 2H), 7.43-7.36 (m, 4H), 7.32 (d, J=8.24 Hz, 1H), 7.28 (d, J=9.05 Hz, 2H), 7.16 (d, J=8.54 Hz, 2H), 6.90 (d, J=9.03 Hz, 2H), 6.48 (d, J=8.31 Hz, 1H), 5.03 (s, 2H), 4.44 (dd, J=6.22, 13.70 Hz, 1H), 2.83-2.81 (m, 2H), 2.44 (br m, 1H), 1.78-1.60 1.42-1.19 (m, 4H); Low resolution mass spectrum (ES) m/e 515[(M+H)+, calcd for C₃₁H₃₉N₄O₃: 515]; 99.0% purity based on HPLC.

### Example 203

### (R)-5-AMINO-2-[3-(9H-FLUOREN-2-YL)-UREIDO]-PENTANOIC ACID (4-CYCLOHEXYL-PHENYL)-AMIDE

4-Cyclohexyl-phenylamine was coupled to (R)-5-tert-Butoxycarbonylamino-2-(9H-fluoren-9-ylmethoxycarbonylamino)-pentanoic acid then to 2-Isocyanato-9H-fluorene as described in the method of Example 26. Purification by HPLC produced the title compound. ¹H NMR (400 MHz, DMSO-d6) δ 10.17 (s, 1H), 8.90 (s, 1H), 7.77-7.68 (m, 5H), 7.53-7.51 (m, 3H), 7.35-7.31 (m, 3H), 7.22 (dt, J=0.98, 7.47, 7.51 Hz, 1H), 7.17 (d, J=8.56 Hz, 2H), 6.64 (d, J=8.21 Hz, 1H), 4.48 (dd, J=6.47, 13.83 Hz, 1H), 3.86 (s, 2H), 2.84-2.83 (m, 2H), 2.44 (br m, 1H), 1.82-1.62 (m, 8H), 1.42-1.19 (m, 4H); Low resolution mass spectrum (ES) m/e 497[(M+H)+, calcd for C₃₁H₃₇N₄O₂: 497]; 99.0% purity based on HPLC.

### Example 204

### (S)-6-AMINO-2-[3-(3-BENZYLOXY-PHENYL)-UREIDO]-HEXANOIC ACID P-TOLYLAMIDE

(S)-(5-Amino-5-p-tolylcarbamoyl-pentyl)-carbamic acid tert-butyl ester from Intermediate #2 of Example 1 was coupled to 1-Benzyloxy-3-isocyanato-benzene as described in the method of Example 26. Purification by HPLC produced the title compound. ¹H NMR (400 MHz, DMSO-d6) δ 10.08 (s, 1H), 8.78 (s, 1H), 7.71 (br s, 3H), 7.50 (d, J=8.40 Hz, 2H), 7.44-7.36 (m, 4H), 7.34-7.30 (m, 1H), 7.21 (s, 1H), 7.14-7.10 (m, 3H), 6.86 (d, J=8.17 Hz, 1H), 6.61-6.55 (m, 2H), 5.04 (s, 2H), 4.37 (dd, J=7.90, 13.46 Hz, 1H), 2.82-2.74 (m, 2H), 2.25 (s, 3H), 1.78-1.69 (m, 1H), 1.65-1.53 (m, 3H), 1.45-1.30 (m, 2H); Low resolution mass spectrum (ES) m/e 461 [(M+H)+, calcd for C₂₇H₃₃N₄O₃: 461]; 99.6% purity based on HPLC.

### Example 205

### (R)-6-AMINO-2-[3-(3-BENZYLOXY-PHENYL)-UREIDO]-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to 1-Benzyloxy-3-isocyanato-benzene as described in the method of Example 26. Purification by HPLC produced the title compound. ¹H NMR (400 MHz, DMSO-d6) δ 10.08 (s, 1H), 8.78 (s, 1H), 7.72 (br s, 3H), 7.50 (d, J=8.42 Hz, 2H), 7.44-7.36 (m, 4H), 7.32 (t, J=7.11, 7.11 Hz, 1H), 7.21 (t, J=2.15, 2.15 Hz, 1H), 7.14-7.10 (m, 3H), 6.86 (dd, J=1.17, 8.09 Hz, 1H), 6.61-6.55 (m, 2H), 5.04 (s, 2H), 4.37 (dd, J=8.00, 13.47 Hz, 1H), 2.81-2.75 (m, 2H), 2.25 (s, 3H), 1.78-1.69 (m, 1H), 1.65-1.53 (m, 3H), 1.46-1.31 (m, 2H); Low resolution mass spectrum (ES) m/e 461 [(M+H)+, calcd for C₂₇H₃₃N₄O₃: 461; 99.6% purity based on HPLC.

### Example 206

### (S)-6-AMINO-2-[3-(4-BENZYLOXY-PHENYL)-UREIDO]-HEXANOIC ACID (2-METHYL-1H-INDOL-5-YL)-AMIDE

2-Methyl-1H-indol-5-ylamine was coupled to (S)-6-tert-Butoxycarbonylamino-2-(9H-fluoren-9-ylmethoxycarbonylamino)-hexanoic acid, and then to 1-Benzyloxy-4-isocyanato-benzene as described in the method of Example 26. Purification by HPLC produced the title compound. ¹H NMR (400 MHz, DMSO-d6) δ 9.42 (s, 1H), 8.55 (s, 1H), 8.43 (s, 4H), 7.77 (br s, 1H), 7.49 (d, J=8 Hz, 2H), 7.14 (d, J=9 Hz, 2H), 6.86 (br s, 1H), 6.56 (br s, 1H), 4.62 (br s, 2H), 4.10 (br s, 1H), 3.87 (s, 3H), 2.68 (s, 4H), 1.69-1.42 (m, 13H), 1.24-0.96 (m, 3H); Low resolution mass spectrum (ES) m/e 500 [(M+H)+, calcd for C₂₉H₃₄N₅O₃: 500]; 99.9% purity based on HPLC.

### Example 207

### (S)-6-AMINO-2-[3-(9H-FLUOREN-2-YL)-UREIDO]-HEXANOIC ACID (2-METHYL-1H-INDOL-5-YL)-AMIDE

2-Methyl-1H-indol-5-ylamine was coupled to (S)-6-tert-Butoxycarbonylamino-2-(9H-fluoren-9-ylmethoxycarbonylamino)-hexanoic acid then to 2-Isocyanato-9H-fluorene as described in the method of Example 26. Purification by HPLC produced the title compound. ¹H NMR (400 MHz, DMSO-d6) δ 10.84 (s, 1H), 9.92 (s, 1H), 8.89 (s, 1H), 7.76-7.72 (br m, 7H), 7.51 (d, J=7.43 Hz, 1H), 7.32 (t, J=7.23, 7.23 Hz, 2H), 7.22 (dt, J=1.00, 7.59, 7.73 Hz, 1H), 7.18 (d, J=8.64 Hz, 1H), 7.14 (dd, J=1.86, 8.65 Hz, 1H), 6.60 (d, J=8.20 Hz, 1H), 6.06 (s, 1H), 4.44 (dd, J=7.80, 13.51 Hz, 1H), 3.86 (s, 2H), 2.84-2.76 (m, 2H), 2.35 (s, 3H), 1.82-1.74 (m, 1H), 1.69-1.54 (m, 3H), 1.49-1.35 (m, 2H); Low resolution mass spectrum (ES) m/e 482 [(M+H)+, calcd for C₂₉H₃₂N₅O₂: 482]; 99.5% purity based on HPLC.

### Example 208

### (S)-[5-AMINO-1-(1H-INDOL-4-YLCARBAMOYL)-PENTYL]-CARBAMIC ACID 9H-FLUOREN-9-YLMETHYL ESTER

1H-Indol-4-ylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 11.15 (s, 1H); 9.60 (s, 1H); 7.75 (t, J=6.58 Hz, 2H); 7.71-7.61 (m, 4H); 7.57 (d, J=7.63 Hz, 1 H); 7.42 (t, J=7.45 Hz, 2 H); 7.36-7.26 (m, 3 H); 7.16 (d, 8.07 Hz, 1 H); 7.02 (t, J=7.88 Hz, 1 H); 6.65 (br s, 1 H); 4.42-4.19 (m, 4H); 2.79 (dddd, J=6.7; 6.7; 6.1; 11.6 Hz, 2H); 1.84-1.63 (m, 2H); 1.63-1.52 (m, 2H); 1.52-1.32 (m, 2H); Low resolution mass spectrum (ES) m/e 483 [(M+H)+, calcd for C₂₉H₃₁N₄O₃: 483]; 91.5% purity based on HPLC.

### Example 209

### (S)-[5-AMINO-1-(4-METHYLAMINO-PHENYLCARBAMOYL)-PENTYL]-CARBAMIC ACID 9H-FLUOREN-9-YLMETHYL ESTER

N-Methyl-benzene-1,4-diamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 10.15 (s, 1H); 8.1 (br s, 3 H); 7.90 (d, J=7.49 Hz, 2H); 7.77-7.60 (m, 8 H); 7.46-7.29 (m, 6 H); 4.30 (d, J=6.71, 2H); 4.27-4.19 (m, 1 H); 4.16-4.08 (m, 1 H); 3.98 (br s, 1 H); 2.78 (br s, 2H); 1.77-1.28 (m, 6 H) ; Low resolution mass spectrum (ES) m/e 473 [(M+H)+, calcd for C₂₈H₃₃N₄O₃: 473]; 99% purity based on HPLC.

### Example 210

### (S)-[5-AMINO-1-(4-BENZYLOXY-3-CHLORO-PHENYLCARBAMOYL)-PENTYL]-CARBAMIC ACID 9H-FLUOREN-9-YLMETHYL ESTER

4-Benzyloxy-3-chloro-phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 10.1 (s, 1H); 7.90 (d, J=7.54 Hz, 2H); 7.82 (d, J=2.38 Hz, 1H); 7.73 (t, J=7.18 Hz, 2H); 7.69-7.58 (m, 4H); 7.49-7.37 (m, 7H); 7.36-7.29 (m, 3H); 7.2 (d, J=9 Hz, 1H); 5.17 (s, 2H); 4.35-4.19 (m, 3H); 4.07 (dd, J=8.29; 13.9 Hz; 1H); 2.78 (dddd, J=6.04; 6.04; 6.27; 12.67 Hz; 2H); 1.76-1.59 (m, 2H); 1.59-1.47 (m, 2H); 1.47-1.25 (m, 2H); Low resolution mass spectrum (ES) m/e 584 [(M+H)+, calcd for C₃₄H₃₅ClN₃O₄: 584]; 98% purity based on HPLC.

### Example 211

### (S)-[5-AMINO-1-(3-BENZYLOXY-PHENYLCARBAMOYL)-PENTYL]-CARBAMIC ACID 9H-FLUOREN-9-YLMETHYL ESTER

3-Benzyloxy-phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ; 10.0 (s, 1H); 7.90 (d, J=7.56 Hz, 2H); 7.74 (t, J=7.41 Hz, 2H); 7.70-7.56 (m, 4H); 7.46-7.26 (m, 10H); 7.21 (d, J=8.1 Hz, 1H); 7.14 (d, J=8.39; 1H); 6.72 (dd, J=1.83; 8.09 Hz; 1 H); 5.07 (s, 2H); 4.35-4.19 (m, 3H); 4.12 (ddd, J=5.66; 8.42; 8.28 Hz; 1H); 2.78 (dddd, J=6.04; 6.04; 6.27; 12.67 Hz; 2H); 1.75-1.60 (m, 2H); 1.60-1.48 (m, 2H); 1.48-1.25 (m, 2H). Low resolution mass spectrum (ES) m/e 550 [(M+H)+, calcd for C₃₁H₃₇N₃O₆: 550]; 97% purity based on HPLC.

### Example 212

### (S)-6-AMINO-2-[3-(3-BENZYL-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Benzyl-3-isocyanato-benzene as described in the method of Example 26. Purification by HPLC produced the title compound. ¹H NMR (400 MHz, DMSO-d6) δ 10.06 (s, 1H), 8.69 (s, 1H), 7.70 (br s, 3H), 7.49 (d, J=8.42 Hz, 2H), 7.30-7.10 (m, 10H), 6.78 (d, J=7.51 Hz, 1H), 6.52 (d, J=8.19 Hz, 1H), 4.35 (dd, J=8.01, 13.48 Hz, 1H), 3.86 (s, 2H), 2.80-2.75 (m, 2H), 2.25 (s, 3H), 1.77-1.68 (m, 1H), 1.63-1.51 (m, 3H), 1.43-1.27 (m, 2H); Low resolution mass spectrum (ES) m/e 445 [(M+H)+, calcd for C₂₇H₃₃N₄O₂: 445]; 99.4% purity based on HPLC.

### Example 213

### (S)-6-AMINO-2-[3-(2,3-DIHYDRO-BENZO[1,4]DIOXIN-6-YL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 6-Isocyanato-2,3-dihydro-benzo[1,4]dioxine as described in the method of Example 26. Purification by HPLC produced the title compound. ¹H NMR (400 MHz, DMSO-d6) δ 10.07 (s, 1H), 8.53 (s, 1H), 7.67 (br s, 3H), 7.49 (d, J=8.42 Hz, 2H), 7.11 (d, J=8.37 Hz, 2H), 7.04 (d, J=1.07 Hz, 1H), 6.70-6.69 (m, 2H), 6.43 (d, J=8.28 Hz, 1H), 4.36 (dd, J=8.03, 13.59 Hz, 1H), 4.17 (dd, J=5.10, 11.93 Hz, 4H), 2.82-2.74 (m, 2H), 2.25 (s, 3H), 1.76-1.52 (m, 4H), 1.44-1.30 (m, 2H); Low resolution mass spectrum (ES) m/e 413 [(M+H)+, calcd for C₂₂H₂₉N₄O₄: 413]; 99.9% purity based on HPLC.

### Example 214

### (S)-6-AMINO-2-{3-[4-(6-METHYL-BENZOTHIAZOL-2-YL)-PHENYL]-UREIDO}-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 2-(4-Isocyanato-phenyl)-benzothiazole as described in the method of Example 26. Purification by HPLC produced the title compound. ¹H NMR (400 MHz, DMSO-d6) δ 10.12 (s, 1H), 9.18 (s, 1H), 7.94 (d, J=8.81 Hz, 2H), 7.88-7.86 (m, 2H), 7.69 (br m, 3H), 7.58 (d, J=8.80 Hz, 2H), 7.51 (d, J=8.42 Hz, 2H), 7.32 (dd, J=1.13, 8.35 Hz, 1H), 7.12 (d, J=8.40 Hz, 2H), 6.75 (d, J=8.12 Hz, 1H), 4.42 (dd, J=7.95, 13.37 Hz, 1H), 2.83-2.75 (m, 2H), 2.44 (s, 3H), 2.25 (s, 3H), 1.81-1.73 (m, 1H), 1.68-1.52 (m, 3H), 1.47-1.32 (m, 2H); Low resolution mass spectrum (ES) m/e 502 [(M+H)+, calcd for C₂₈H₃₂N₅O₂S: 502]; 99.8% purity based on HPLC.

### Example 215

### (S)-[2-(4-AMINOMETHYL-PHENYL)-1-(2-METHYL-1H-INDOL-5-YLCARBAMOYL)-ETHYL]-CARBAMIC ACID 9H-FLUOREN-9-YLMETHYL ESTER

2-Methyl-1H-indol-5-ylamine was coupled to (S)-3-[4-(tert-Butoxycarbonylamino-methyl)-phenyl]-2-(9H-fluoren-9-ylmethoxycarbonylamino)-propionic acid as described in the method of Example 6. Purification by HPLC produced the title compound. ¹H NMR (400 MHz, DMSO-d6) δ 10.84 (s, 1H), 9.89 (s, 1H), 8.14 (br s, 3H), 7.89 (d, J=7.54 Hz, 2H), 7.78 (d, J=8.55 Hz, 1H), 7.73-7.68 (m, 3H), 7.43-7.36 (m, 6H), 7.31 (t, J=7.39, 7.39 Hz, 2H), 7.19 (d, J=8.55 Hz, 1H), 7.11 (dd, J=1.74, 8.65 Hz, 1H), 6.07 (s, 1H), 4.42 (dt, J=4.65, 9.63, 9.68 Hz, 1H), 4.26-4.13 (m, 3H), 4.01-3.97 (m, 2H), 3.07 (dd, J=4.39, 13.59 Hz, 1H), 2.92 (dd, J=10.43, 13.57 Hz, 1H), 2.36 (s, 3H); Low resolution mass spectrum (ES) m/e 545 [(M+H)+, calcd for C₃₄H₃₃N₄O₃: 545]; 100% purity based on HPLC.

### Example 216

### (S)-[5-AMINO-1-(3,4-DIMETHYL-PHENYLCARBAMOYL)-PENTYL]-CARBAMIC ACID 9H-FLUOREN-9-YLMETHYL ESTER

3,4-Dimethyl-phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 9.85 (s, 1H); 7.90 (d, J=7.52 Hz, 1H); 7.74 (t, J=7.26 Hz, 2H); 7.69-7.55 (m, 4H); 7.42 (t, J=7.43 Hz, 2H); 7.38-7.28 (m, 4H); 7.05 (d, J=8.19 Hz, 1H); 4.36-4.19 (m, 3H); 4.11 (ddd, J=5.8; 8.4; 8.4 Hz, 1H); 2.78 (d, J=5.96 Hz, 2H); 2.18 (s, 3H); 2.16 (s, 3H); 1.75-1.46 (m, 4H); 1.44-1.25 (m, 2H). Low resolution mass spectrum (ES) m/e 472 [(M+H)+, calcd for C₂₉H₃₄N₃O₃: 472]; 99% purity based on HPLC.

### Example 217

### (S)-[1-(3,4-DICHLORO-PHENYLCARBAMOYL)-5-FORMYLAMINO-PENTYL]-CARBAMIC ACID 9H-FLUOREN-9-YLMETHYL ESTER

3,4-Dichloro-phenylamine was coupled to (S)-6-tert-Butoxycarbonylamino-2-(9H-fluoren-9-ylmethoxycarbonylamino)-hexanoic acid as described in the method of Example 6. Purification by HPLC produced unexpectedly the title compound. ¹H NMR (400 MHz, DMSO-d6) δ 7.95 (t, J=5.6 Hz, 1H); 7.89 (d, J=7.49 Hz, 2H); 7.72 (dd, J=4.56; 7.24 Hz, 2H); 7.64 (br s, 3H); 7.52-7.45 (m, 2H); 7.42 (t, J=7.50 Hz, 3H); 7.33 (t, J=7.44 Hz, 2H); 7.20 (dd, J=1.83; 8.24 Hz, 1H); 4.33-4.14 (m, 3H); 3.87 (ddd, J=5.5; 8.7; 8.7Hz, 1H); 2.7 (t, J=6.60 Hz, 1H); 1.59-1.39 (m, 4H); 1.36-1.14 (m, 2H). Low resolution mass spectrum (ES) m/e 540 [(M+H)+, calcd for C₂₈H₂₈N₃O₄: 540]; 99% purity based on HPLC.

### Example 218

### (S)-[5-AMINO-1-(3-TRIFLUOROMETHYL-PHENYLCARBAMOYL)-PENTYL]-CARBAMIC ACID 9H-FLUOREN-9-YLMETHYL ESTER

3-trifluoromethyl-phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 10.4 (s, 1H); 8.11 (s, 1H); 7.89 (d, J=7.52 Hz, 2H); 7.79 (d, J=8.37 Hz, 1H); 7.71 (dd, J=7.04; 13.1 Hz, 3H); 7.66-7.59 (m, 2H); 7.56 (d, J=7.99 Hz, 1H); 7.46-7.37 (m, 3H); 7.54-7.27 (m, 2H); 4.35-4.15 (m, 3); 4.11 (dd, J=8.30; 13.55 Hz, 1H); 2.78 (dt, J=6.14; 18.9 Hz, 2H); 1.81-1.45 (m, 4H); 1.43-1.25 (m, 2H). Low resolution mass spectrum (ES) m/e 512 [(M+H)+, calcd for C₂₈H₂₉N₃O₃: 512]; 95% purity based on HPLC.

### Example 219

### (S)-[1-(3-ACETYL-PHENYLCARBAMOYL)-5-AMINO-PENTYL]-CARBAMIC ACID 9H-FLUOREN-9-YLMETHYL ESTER

3-acetyl-phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 10.26 (s, 1H); 8.18 (s, 1H); 7.87 (dd, 1H, J=4.3; 11.2 Hz 3H); 7.73 (t, J=7.15 Hz, 2H); 7.69-7.58 (m, 4H); 7.47 (t, J=7.91 Hz, 1H); 7.41 (ddd, J=1.4; 7.4; 7.4 Hz; 7.32 (ddd, J=2.8; 6.7; 7.4 Hz, 2H); 4.35-4.18 (m, 3H); 4.12 (dd, J=8.4; 13.7, 1H); 2.78 (dt, J=6.13; 18.6, 2H); 2.55 (s, 3H); 1.79-1.47 (m, 4H); 1.47-1.26 (m, 2H). Low resolution mass spectrum (ES) m/e 486 [(M+H)+, calcd for C₂₉H₃₂N₃O₄: 486]; 94% purity based on HPLC.

### Example 220

### (S)-{4-[R-(PYRROLIDINE-2-CARBONYL)-AMINO]-1-P-TOLYLCARBAMOYL-BUTYL}-CARBAMIC ACID 9H-FLUOREN-9-YLMETHYL ESTER

(S)-(4-Amino-1-p-tolylcarbamoyl-butyl)-carbamic acid 9H-fluoren-9-ylmethyl ester from Example 101 was coupled to (R)-Pyrrolidine-1,2-dicarboxylic acid 1-tert-butyl ester as described in the method of Example 6. Purification by HPLC produced the title compound. ¹H NMR (400 MHz, DMSO-d6) 7.87 (d, J=8 Hz, 2H), 7.70 (t, J=9 and 8 Hz, 2H), 7.45 (d, 8 Hz, 2H), 7.40 (ddd, J=2, 7, 8 Hz, 2H), 7.35-7.26 (m, 2H), 7.10 (d, J=8 Hz, 2H), 4.36-4.07 (m, 5H), 3.15-3.05 (m, 3H), 2.22 (s, 3H), 1.81-1.44 (m, 5H), 1.38-1.08 (m, 3H); Low resolution mass spectrum (ES) m/e 542 [(M+H)+, calcd for C₃₂H₃₇N₄O₄: 542]; 89% purity based on HPLC.

### Example 221

### (S)-[3-(2-AMINO-ACETYLAMINO)-1-P-TOLYLCARBAMOYL-PROPYL]-CARBAMIC ACID 9H-FLUOREN-9-YLMETHYL ESTER

(S)-(3-Amino-1-p-tolylcarbamoyl-propyl)-carbamic acid 9H-fluoren-9-ylmethyl ester of Example 108 was coupled to tert-Butoxycarbonylamino-acetic acid as described in the method of Example 6. Purification by HPLC produced the title compound. ¹H NMR (400 MHz, DMSO-d6) 7.86 (d, J=8 Hz, 2H), 7.70 (t, J=7 Hz, 2H), 7.46-7.36 (m, 4H), 7.31 (dd, J=7, 14 Hz, 2H), 7.10 (d, J=8 Hz, 2H), 4.32-4.17 (m, 3H), 4.12 (dd, J=5, 9 Hz, 1H), 3.49 (s, 2H), 3.28-3.06 (m, 2H), 2.22 (s, 3H), 1.93-1.70 (m, 2H); Low resolution mass spectrum (ES) m/e 487 [(M+H)+, calcd for C₂₈H₃₁N₄O₄: 487]; 99% purity based on HPLC.

### Example 222

### (S)-{2-[2-(2-AMINO-ACETYLAMINO)-ACETYLAMINO]-1-P-TOLYLCARBAMOYL-ETHYL}-CARBAMIC ACID 9H-FLUOREN-9-YLMETHYL ESTER

(S)-(2-Amino-1-p-tolylcarbamoyl-ethyl)-carbamic acid 9H-fluoren-9-ylmethyl ester of Example 103 was coupled to (2-tert-Butoxycarbonylamino-acetylamino)-acetic acid as described in the method of Example 6. Purification by HPLC produced the title compound. ¹H NMR (400 MHz, DMSO-d6) 7.87 (d, J=8 Hz, 2H), 7.69 (dd, J=4, 7 Hz, 2H), 7.48-7.36 (m, 4H), 7.31 (dd, J=7, 15 Hz, 2H), 7.09 (d, J=8.0 Hz, 2H), 4.35-4.14 (m, 6H), 3.76 (s, 2H), 3.40 (d, J=6 Hz, 2H), 2.23 (s, 3H); Low resolution mass spectrum (ES) m/e 530 [(M+H)+, calcd for C₂₉H₃₂N₅O₅: 530]; 95% purity based on HPLC.

### Example 223

### (S)-[4-(2-METHYLAMINO-ACETYLAMINO)-1-P-TOLYLCARBAMOYL-BUTYL]-CARBAMIC ACID 9H-FLUOREN-9-YLMETHYL ESTER

(S)-(4-Amino-1-p-tolylcarbamoyl-butyl)-carbamic acid 9H-fluoren-9-ylmethyl ester from Example 101 was coupled to Methylamino-acetic acid as described in the method of Example 6. Purification by HPLC produced the title compound. ¹H NMR (400 MHz, DMSO-d6) 7.86 (d, J=7 Hz, 2H), 7.69 (t, J=7 Hz, 2H), 7.44 (d, J=8 Hz, 2H), 7.38 (ddd, J=2, 8, 8 Hz, 2H), 7.30 (dd, J=7, 13 Hz, 2H), 7.09 (d, J=8 Hz, 2H), 4.33-4.04 (m, 4H), 3.18-3.04 (m, 2H), 2.51 (s, 2H), 2.22 (s, 3H), 1.77-1.43 (m, 4H), 1.42 (s, 3H); Low resolution mass spectrum (ES) m/e 515 [(M+H)+, calcd for C₃₀H₃₅N₄O₄: 515]; 95% purity based on HPLC.

### Example 224

### (S)-6-AMINO-2-[3-(4-BENZYLAMINO-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanato-4-nitro-benzene as described in the method of Example 26. Hydrogenation for 4 hours using 10% pallidium on carbon in ethanol under a hydrogen atmosphere followed by mono-alkylation with benzyl bromide and sodium hydride in THF. Deprotection was as described in the method of Example 2 and purification by HPLC produced the title compound. ¹H NMR (400 MHz, DMSO-d6) δ 10.05 (s, 1H), 8.45 (s, 1H), 7.67 (br s, 3H), 7.49 (d, J=8.42 Hz, 2H), 7.39-7.16 (m, 9H), 7.11 (d, J=8.37 Hz, 2H), 6.72( br, 3H), 6.42 (d, J=7.09 Hz, 1H), 4.37-4.29 (m, 3.5H), 3.36 (t, J=5.23 1H ), 2.81-2.74 (m, 2H), 2.25 (s, 3H), 1.75-1.67 (m, 1H), 1.62-1.28 (m, 5H); Low resolution mass spectrum (ES) m/e 461 [(M+H)+, calcd for C₂₇H₃₄N₅O₂: 461]; 83% purity based on HPLC.

### Example 225

### (S)-6-AMINO-2-{3-[4-(4-FLUORO-BENZYLAMINO)-PHENYL]-UREIDO}-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanato-4-nitro-benzene as described in the method of Example 26. Hydrogenation for 4 hours using 10% pallidium on carbon in ethanol under a hydrogen atmosphere was followed by mono-alkylation with 4-fluorobenzyl bromide and sodium hydride in THF. Deprotection was as described in the method of Example 2 and purification by HPLC produced the title compound. ¹H NMR (400 MHz, DMSO-d6) δ 10.05 (s, 1H), 8.39 (s, 1H), 7.67 (br s, 3H), 7.49 (d, J=8.43 Hz, 2H), 7.40-7.37 (m, 3H), 7.18-7.10 (m, 7H0, 6.39 (d, J=7.86 Hz, 1H), 4.34 (dd, J=7.90, 13.55 Hz, 1H), 4.25 (s, 2H), 3.37-3.34 (m, 1H), 2.81-2.73 (m, 2H), 2.25 (s, 3H), 1.75-1.66 (m, 1H), 1.62-1.29 (m, 5H); Low resolution mass spectrum (ES) m/e 478 [(M+H)+, calcd for C₂₇H₃₃FN₅O₂: 478]; 85% purity based on HPLC.

### Example 226

### (S)-6-AMINO-2-[3-(9H-FLUOREN-2-YL)-UREIDO]-HEXANOIC ACID (4-CYCLOHEXYL-PHENYL)-AMIDE

4-Cyclohexyl-phenylamine was coupled to (S)-6-tert-Butoxycarbonylamino-2-(9H-fluoren-9-ylmethoxycarbonylamino)-hexanoic acid, and then to 2-Isocyanato-9H-fluorene as described in the method of Example 26. Purification by HPLC produced the title compound. ¹H NMR (400 MHz, DMSO-d6) δ 10.11 (s, 1H), 8.86 (s, 1H), 7.76-7.72 (m, 3H), 7.68 (br s, 3H), 7.53-7.51 (m, 3H), 7.34-7.31 (m, 2H), 7.22 (t, J=7.86, 7.86 Hz, 1H), 7.16 (d, J=8.53 Hz, 2H), 6.60 (d, J=8.16 Hz, 1H), 4.42 (dd, J=7.79, 13.48 Hz, 1H), 3.85 (s, 2H), 2.83-2.75 (m, 2H), 2.44 (m, 1H), 1.78-1.54 (m, 9H), 1.42-1.19 (m, 7H); Low resolution mass spectrum (ES) m/e 511 [(M+H)+, calcd for C₃₂H₃₉FN₄O₂: 511]; 99.0% purity based on HPLC.

### Example 227

### (S)-3-(4-AMINOMETHYL-PHENYL)-2-[3-(4-BENZYLOXY-PHENYL)-UREIDO]-N-(2-METHYL-1H-INDOL-5-YL)-PROPIONAMIDE

2-Methyl-1H-indol-5-ylamine was coupled to (S)-3-[4-(tert-Butoxycarbonylamino-methyl)-phenyl]-2-(9H-fluoren-9-ylmethoxycarbonylamino)-propionic acid, and then to 1-Benzyloxy-4-isocyanato-benzene as described in the method of Example 26. Purification by HPLC produced the title compound. ¹H NMR (400 MHz, DMSO-d6) δ 10.84 (s, 1H), 9.94 (s, 1H), 8.53 (s, 1H), 8.12 (br s, 3H), 7.69 (d, J=1.66 Hz, 1H), 7.43-7.29 (m, 9H), 7.25 (d, J=9.05 Hz, 2H), 7.18 (d, J=8.59 Hz, 1H), 7.10 (dd, J=1.91, 8.65 Hz, 1H), 6.88 (d, J=9.06 Hz, 2H), 6.43 (d, J=9.06 Hz, 1H), 6.06 (s, 1H), 5.02 (s, 2H), 4.66 (dd, J=8.35, 13.70 Hz, 1H), 3.99 (q, J=5.4 Hz, 2H), 3.11 (dd, J=5.15, 13.71 Hz, 1H), 2.90 (dd, J=8.46, 13.70 Hz, 1H), 2.35 (s, 3H); Low resolution mass spectrum (ES) m/e 548[(M+H)+, calcd for C₃₃H₃₄N₅O₃: 548]; 99.6% purity based on HPLC.

### Example 228

### (S)-[5-AMINO-1-(4-AMINOMETHYL-PHENYLCARBAMOYL)-PENTYL]-CARBAMIC ACID 9H-FLUOREN-9-YLMETHYL ESTER

(4-Amino-benzyl)-carbamic acid tert-butyl ester was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 10.2 (s, 1H); 8.11 (br s, 3H); 7.90 (d, J=7.54 Hz, 2H); 7.78-7.60 (m, 7H); 7.47-7.28 (m, 5H); 4.29 (d, J=6.71 Hz, 1H); 4.26-4.20 (m, 1H); 4.13 (ddd, J=5.49, 8.61, 8.59 Hz, 1H); 3.98 (d, J=4.78 Hz, 2H); 2.85-2.70 (m, 2H); 1.78-1.48 (m, 4H); 1.48-1.27 (m, 2H); Low resolution mass spectrum (ES) m/e 473 [(M+H)+, calcd for C₂₈H₃₃N₄O₃: 473]; 98% purity based on HPLC.

### Example 229

### (S)-[5-AMINO-1-(6-METHYL-PYRIDIN-3-YLCARBAMOYL)-PENTYL]-CARBAMIC ACID 9H-FLUOREN-9-YLMETHYL ESTER

6-Methyl-pyridin-3-ylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 10.4 (s, 1H); 8.16 (s, 1H); 7.97 (d, J=8.41 Hz, 1H); 7.90 (d, J=7.48 Hz, 2H); 7.23 (t, J=7.0 Hz, 2H); 7.69-7.55 (m, 4H); 7.42 (ddd, J=1.60, 7.35,7.28 Hz, 2H); 7.32 (dd, J=6.99, 13.43 Hz, 2H); 4.37-4.18 (m, 5H); 3.92 (td, J=4.72, 9.68 Hz, 1H); 2.78 (ddd. J=7.01, 12.47, 12.14 Hz, 2H); 2.25 (s, 3H); 1.76-1.46 (m, 4H); 1.47-1.28 (m, 2H); Low resolution mass spectrum (ES) m/e 459 [(M+H)+, calcd for C₂₇H₃₁N₄O₃: 459]; 94% purity based on HPLC.

### Example 230

### (S)-[1-(4-ACETYLAMINO-PHENYLCARBAMOYL)-5-AMINO-PENTYL]-CARBAMIC ACID 9H-FLUOREN-9-YLMETHYL ESTER

N-(4-Amino-phenyl)-acetamide was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 9.96 (s, 1H); 9.87 (s, 1H); 7.89 (d, J=7.57 Hz, 2H); 7.72 (t, J=7.57 Hz, 2H); 7.68-7.55 (m, 4H); 7.49 (s, 4H); 7.41 (t, J=7.44 Hz, 2H); 7.35-7.27 (m, 2H); 4.35-4.18 (m, 3H); 4.10 (ddd, J=5.65, 8.44, 8.39 Hz, 1H); 2.77 (dddd, J=6.49, 6.49, 6.15, 12.60 Hz, 2H); 2.00 (s, 3H); 1.77-1.46 (m, 4H); 1.46-1.24 (m, 2H); Low resolution mass spectrum (ES) m/e 501 [(M+H)+, calcd for C₂₉H₃₃N₄O₄: 501]; 99% purity based on HPLC.

### Example 231

### (S)-[1-(3-ACETYLAMINO-PHENYLCARBAMOYL)-5-AMINO-PENTYL]-CARBAMIC ACID 9H-FLUOREN-9-YLMETHYL ESTER

N-(3-Amino-phenyl)-acetamide was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) d 10.0 (s, 1H); 9.92 (s, 1H); 7.95 (s, 1H); 7.89 (J=7.57 Hz, 2H); 7.72 (t, J=8.18 Hz, 2H); 7.68-7.54 (m, 3H); 7.41 (ddd, J=2.27, 7.32, 7.35 Hz, 2H); 7.36-7.28 (m, 3H); 7.23-7.14 (m, 2H); 4.35-4.17 (m, 3H); 4.13 (ddd, J=5.61, 8.56, 8.61 Hz, 1H); 2.77 (dddd, J=5.53, 6.19, 6.13, 11.73 Hz, 2H); 1.74-1.46 (m, 2); 1.46-1.25 (m, 2H); Low resolution mass spectrum (ES) m/e 501 [(M+H)+, calcd for C₂₉H₃₃N₄O₄: 501]; 97% purity based on HPLC.

### Example 232

### (S)-[5-AMINO-1-(PYRIDIN-4-YLCARBAMOYL)-PENTYL]-CARBAMIC ACID 9H-FLUOREN-9-YLMETHYL ESTER

Pyridin-4-ylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 11.2 (s, 1H); 8.64 (d, J=6.34 Hz, 2H); 7.94 (d, J=6.54 Hz, 2H); 7.89 (d, J=7.54 Hz, 2H); 7.84 (d, J=7.04 Hz, 1H); 7.75-7.60 (m, 4H); 7.41 (t, J=7.46 Hz, 2H); 7.32 (t, J=7.43 Hz, 2H); 4.38-4.19 (m, 3H); 4.19-4.10 (m, 1H); 2.77 (td, J=5.71, 6.08, 6.06, 12.5 Hz, 2H); 1.81-1.27 (m, 6H); Low resolution mass spectrum (ES) m/e 445 [(M+H)+, calcd for C₂₆H₂₉N₄O₃: 445]; 100% purity based on HPLC.

### Example 233

### (S)-[5-(2-AMINO-ACETYLAMINO)-1-(2-METHYL-1H-INDOL-5-YLCARBAMOYL)-PENTYL]-CARBAMIC ACID 9H-FLUOREN-9-YLMETHYL ESTER

(S)-[5-Amino-1-(2-methyl-1H-indol-5-ylcarbamoyl)-pentyl]-carbamic acid 9H-fluoren-9-yl methyl ester of Example 38 was coupled to tert-Butoxycarbonylamino-acetic acid as described in the of Example 6. Purification by HPLC produced the title compound. ¹H NMR (400 MHz, DMSO-d6) 7.87 (d, J=7.9 Hz, 2H), 7.75-7.64 (m, 3H), 7.44-7.36 (m, 2H), 7.31 (dd, J=6.4 and 12.9 Hz, 2H), 7.16 (d, J=7.9 Hz, 1H), 7.08 (dd, J=1.8, 9.6 Hz, 1H), 6.04 (s, 1H), 4.33-4.15 (m, 3H), 4.10 (dd, J=5.3, 9.2 Hz, 1H), 3.47 (s, 2H), 3.15-3.05 (m, 2H), 2.33 (s, 3H), 1.77-1.54 (m, 3H), 1.40-1.22 (m, 3H); Low resolution mass spectrum (ES) m/e 554 [(M+H⁾⁺, calcd for C₃₂H₃₆N₅O₄: 554]; 78% purity based on HPLC.

### Example 234

### (S)-3-(4-AMINOMETHYL-PHENYL)-2-(3-BIPHENYL-4-YL-UREIDO)—NR-(2-METHYL-1H-INDOL-5-YL)-PROPIONAMIDE

2-Methyl-1H-indol-5-ylamine coupled to (S)-3-[4-(tert-Butoxycarbonylamino-methyl)-phenyl]-2-(9H-fluoren-9-ylmethoxycarbonylamino)-propionic acid, and then to 4-Isocyanato-biphenyl as described in the method of Example 26. Purification by HPLC produced the title compound. ¹H NMR (400 MHz, DMSO-d6) δ 10.84 (s, 1H), 9.98 (s, 1H), 8.85 (s, 1H), 8.12 (br s, 3H), 7.70 (d, J=1.48 Hz, 1H), 7.60 (d, J=8.39 Hz, 2H), 7.54 (d, J=8.73 Hz, 2H), 7.46-7.27 (m, 9H), 7.19 (d, J=8.61 Hz, 1H), 7.11 (dd, J=1.87, 8.65 Hz, 1H), 6.59 (d, J=8.20 Hz, 1H), 6.07 (s, 1H), 4.70 (dd, J=8.23, 13.60 Hz, 1H), 3.99 (q, J=4.83, 4.83, 4.90 Hz, 2H), 3.14 (dd, J=5.08, 13.74 Hz, 1H), 2.93 (dd, J=8.41, 13.74 Hz, 1H), 2.36 (s, 3H); Low resolution mass spectrum (ES) m/e 518 [(M+H)+, calcd for C₃₂H₃₂N₅O₂: 518]; 99.3% purity based on HPLC.

### Example 235

### (S)-3-(4-AMINOMETHYL-PHENYL)-2-[3-(9H-FLUOREN-2-YL)-UREIDO]-N-(2-METHYL-1H-INDOL-5-YL)-PROPIONAMIDE

2-Methyl-1H-indol-5-ylamine was coupled to (S)-3-[4-(tert-Butoxycarbonylamino-methyl)-phenyl]-2-(9H-fluoren-9-ylmethoxycarbonylamino)-propionic acid, and then to 2-Isocyanato-9H-fluorene as described in the method of Example 26. Purification by HPLC produced the title compound. ¹H NMR (400 MHz, DMSO-d6) δ 10.83 (s, 1H), 9.96 (s, 1H), 8.81 (s, 1H), 8.09 (br s, 3H), 7.75-7.69 (m, 4H), 7.50 (d, J=7.43 Hz, 1H), 7.38-7.27 (m, 6H), 7.22 (d, J=7.43 Hz, 1H), 7.18 (d, J=9.10 Hz, 1H), 7.10 (dd, J=1.85, 8.65 Hz, 1H), 6.56 (d, J=8.36 Hz, 1H), 6.06 (s, 1H), 4.69 (dd, J=8.19, 13.57 Hz, 1H), 3.98 (d, J=5.51 Hz, 2H), 3.83 (s, 2H), 3.14 (dd, J=5.09, 13.71 Hz, 1H), 2.93 (dd, J=8.33, 13.76 Hz, 1H), 2.34 (s, 3H); Low resolution mass spectrum (ES) m/e 530 [(M+H)+, calcd for C₃₃H₃₂N₅O₂: 530]; 95.3% purity based on HPLC.

### Example 236

### (S)-6-AMINO-2-[3-(4-STYRYL-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanato-4-Styryl-benzene as described in the method of Example 26. Purification by HPLC produced the title compound. ¹H NMR (400 MHz, DMSO-d6) δ 10.09 (s, 1H), 8.86 (s, 1H), 7.67 (br s, 3H), 7.55 (d, J=7.40 Hz, 2H), 7.51-7.47 (m, 4H), 7.40 (d, J=8.72 Hz, 2H), 7.35 (t, J=7.67 Hz, 2H), 7.23 (t, J=7.32 Hz, 1H), 7.14 (d, J=5.05 Hz, 2H), 7.11 (d, J=3.48 Hz, 2H), 6.60 (d, J=8.21 Hz, 1H), 4.40 (dd, J=7.99, 13.49 Hz, 1H), 2.79 (br m, 2H), 2.25 (s, 3H), 1.79-1.71 (m, 1H), 1.66-1.53 (m, 3H), 1.46-1.31 (m, 2H); Low resolution mass spectrum (ES) m/e 457 [(M+H)+, calcd for C₂₈H₃₃N₄O₂: 457]; 96.7% purity based on HPLC.

### Example 237

### (S)-[3-(2-AMINO-ACETYLAMINO)-1-(2-METHYL-1H-INDOL-5-YLCARBAMOYL)-PROPYL]-CARBAMIC ACID 9H-FLUOREN-9-YLMETHYL ESTER

2-Methyl-1H-indol-5-ylamine was coupled to (S)-4-tert-Butoxycarbonylamino-2-(9H-fluoren-9-ylmethoxycarbonylamino)-butyric acid, and then to tert-Butoxycarbonylamino-acetic acid as described in the method of Example 182. Purification by HPLC produced the title compound. ¹H NMR (400 MHz, DMSO-d6) δ 7.87 (d, J=7.7 Hz, 2H), 7.72 (t, J=7.0 Hz, 2H), 7.66 (d, J=1.3 Hz, 1H), 7.40 (ddd, J=2.9, 7.5, 7.2 Hz, 2H), 7.31 (dd, J=7.2, 14.3 Hz, 2H), 7.17 (d, J=8.6 Hz, 1H), 7.08 (dd, J=2.0, 8.8 Hz, 1H), 4.34-4.12 (m, 4H), 3.32-3.09 (m, 3H), 2.33 (s, 3H), 1.96-1.72 (m, 2H); Low resolution mass spectrum (ES) m/e 526 [(M+H)+, calcd for C₃₀H₃₂N₅O₄: 526]; 86% purity based on HPLC.

### Example 238

### (S)-[4-(2-AMINO-ACETYLAMINO)-1-(2-METHYL-1H-INDOL-5-YLCARBAMOYL)-BUTYL]-CARBAMIC ACID 9H-FLUOREN-9-YLMETHYL ESTER

2-Methyl-1H-indol-5-ylamine was coupled to (S)-5-tert-Butoxycarbonylamino-2-(9H-fluoren-9-ylmethoxycarbonylamino)-pentanoic acid, and then to tert-Butoxycarbonylamino-acetic acid as described in the method of Example 182. Purification by HPLC produced the title compound. ¹H NMR (400 MHz, DMSO-d6) δ 7.87 (d, J=7.7 Hz, 2H), 7.75-7.65 (m, 3H), 7.40 (ddd, J=3.5, 7.5, 7.0 Hz, 2H), 7.31 (dd, J=7.0, 12.9 Hz, 2H), 7.16 (d, J=8.8 Hz, 1H), 7.09 (dd, J=1.8, 9.4 Hz, 1H), 4.36-4.17 (m, 3H), 4.12 (dd, J=5.3, 9.0 Hz, 1H), 3.48 (s, 2H), 3.20-3.04 (m, 2H), 2.33 (s, 3H), 1.81-1.36 (m, 4H); Low resolution mass spectrum (ES) m/e 540 [(M+H)+, calcd for C₃1H₃₄N₅O₄: 540]; 84% purity based on HPLC.

### Example 239

### (S)-6-(2-AMINO-ACETYLAMINO)-2-[3-(4-BENZYLOXY-PHENYL)-UREIDO]-HEXANOIC ACID (2-METHYL-1H-INDOL-5-YL)-AMIDE

2-Methyl-1H-indol-5-ylamine was coupled to (S)-6-tert-Butoxycarbonylamino-2-(9H-fluoren-9-ylmethoxycarbonylamino)-hexanoic acid as described in the method of Example 6, and then to tert-Butoxycarbonylamino-acetic acid as described in the method for Intermediate #1 of Example 1. Final coupling to 1-Benzyloxy-4-isocyanato-benzene as described in the method of Example 26 and purification by HPLC produced the title compound. ¹H NMR (400 MHz, DMSO-d6) δ 7.67 (d, J=1.8 Hz, 1H), 7.43-7.22 (m, 8H), 7.16 (d, J=8.6 Hz, 1H), 7.09 (dd, J=2.0, 8.6 Hz, 1H), 6.91-6.84 (m, 2H), 5.00 (s, 2H), 4.33 (dd, J=5.5, 8.1 Hz, 1H), 3.46 (s, 2H), 3.10 (t, J=8.1, 6.4 Hz, 2H), 2.33 (s, 3H), 1.80-1.23 (m, 6H); low resolution mass spectrum (ES) m/e 557 [(M+H)⁺, calcd for C₃₁H₃₇N₆O₄: 557]; 97% purity based on HPLC.

### Example 240

### (S)-4-(2-AMINO-ACETYLAMINO)-2-[3-(9H-FLUOREN-2-YL)-UREIDO]-N-(2-METHYL-1H-INDOL-5-YL)-BUTYRAMIDE

2-Methyl-1H-indol-5-ylamine was coupled to (S)-4-tert-Butoxycarbonylamino-2-(9H-fluoren-9-ylmethoxycarbonylamino)-butyric acid as described in the method of Example 6, and then to tert-Butoxycarbonylamino-acetic acid as described in the method for Intermediate #1 of Example 1. Final coupling to 2-isocyanato-9H-fluorene as described in the method of Example 26 and purification by HPLC produced the title compound. ¹H NMR (400 MHz, DMSO-d6) δ 7.77-7.64 (m, 4H), 7.50 (d, J=7.0 Hz, 1H), 7.36-7.27 (m, 3H), 7.22 (d, J=7.7 Hz, 1H), 7.18 (d, J=8.6 Hz, 1H), 7.09 (dd, J=1.5, 8.6 Hz, 1H), 4.42 (dd, J=5.5, 7.5 Hz, 1H), 3.83 (s, 2H), 3.50 (s, 2H), 3.33-3.10 (m, 2H), 2.33 (s, 3H), 2.03-1.72 (m, 2H); low resolution mass spectrum (ES) m/e 511 [(M+H)⁺, calcd for C₂₉H₃₁N₆O₃: 511]; 81% purity based on HPLC.

### Example 241

### (S)-4-(2-AMINO-ACETYLAMINO)-2-[3-(4-BENZYLOXY-PHENYL)-UREIDO]-N-(2-METHYL-1H-INDOL-5-YL)-BUTYRAMIDE

2-Methyl-1H-indol-5-ylamine was coupled to (S)-4-tert-Butoxycarbonylamino-2-(9H-fluoren-9-ylmethoxycarbonylamino)-butyric acid as described in the method of Example 6, and then to tert-Butoxycarbonylamino-acetic acid as described in the method for Intermediate #1 of Example 1. Final coupling to 1-Benzyloxy-4-isocyanato-benzene as described in the method of Example 26 and purification by HPLC produced the title compound. ¹H NMR (400 MHz, DMSO-d6) δ 7.65 (d, J=1.8 Hz, 1H), 7.45-7.22 (m, 9H), 7.17 (d, J=8.8 Hz, 1H), 7.08 (dd, J=1.8, 8.9 Hz, 1H), 7.03 (d, J=9.2 Hz, 1H), 6.95 (d, J=9.2 Hz, 1H), 6.88 (d, J=9.2 Hz, 2H), 5.06 (s, 1H), 5.00 (s, 2H), 4.38 (dd, J=5.5, 8.3 Hz, 1H), 3.49 (s, 2H), 3.32-3.08 (m, 2H), 2.32 (s, 3H), 1.97-1.68 (m, 2H); low resolution mass spectrum (ES) m/e 529 [(M+H)⁺, calcd for C₂₉H₃₃N₆O₄: 529]; 77% purity based on HPLC.

### Example 242

### (S)-[4-(2-ETHYLAMINO-ACETYLAMINO)-1-P-TOLYLCARBAMOYL-BUTYL]-CARBAMIC ACID 9H-FLUOREN-9-YLMETHYL ESTER

2-Methyl-1H-indol-5-ylamine was coupled to (S)-5-tert-Butoxycarbonylamino-2-(9H-fluoren-9-ylmethoxycarbonylamino)-pentanoic acid as described in the method of Example 6, and then to Ethylamino-acetic acid as described in the method for Intermediate #1 of Example 1. Purification by HPLC produced the title compound. ¹H NMR (400 MHz, DMSO-d6) δ 7.87 (d, J=7.7 Hz, 2H), 7.71 (t, J=7.7 Hz, 2H), 7.45 (d, J=8.3 Hz, 2H), 7.40 (ddd, J=2.0, 7.7, 7.7 Hz, 2H), 7.35-7.27 (m, 2H), 7.09 (d, J=8.3 Hz, 2H), 4.34-4.16 (m, 3H), 4.10 (dd, J=5.3, 9.0 Hz, 1H), 3.63 (s, 2H), 3.20-3.07 (m, 2H), 2.91 (dd, J=7.9, 13.6 Hz, 2H), 2.23 (s, 3H), 1.77-1.37 (m, 4H), 1.13 (t, J=7.0 Hz, 3H); low resolution mass spectrum (ES) m/e 529 [(M+H)⁺, calcd for C₃₁H₃₇N₄O₄: 529]; 86% purity based on HPLC.

### Example 243

### (S)-6-AMINO-2-[3-(4-PHENETHYL-PHENYL)-UREIDO]-HEXANOIC ACID P-TOLYLAMIDE

The title compound was prepared as described in Example 2 with a final coupling to 1-Isocyanato-4-phenethyl-benzene. ¹H NMR (400 MHz, DMSO-d6) δ 10.07 (s, 1H), 8.64 (s, 1H), 7.68 (br s, 3H), 7.50 (d, J=8.39 Hz, 2H), 7.28-7.24 (m, 4H), 7.20-7.10 (m, 5H), 7.06 (d, J=8.43 Hz, 2H), 6.51 (d, J=8.21 Hz, 1H), 4.38 (dd, J=7.98, 13.52 Hz, 1H), 2.85-2.77 (m, 6H), 2.25 (s, 3H), 1.77-1.69 (m, 1H), 1.64-1.51 (m, 3H), 1.45-1.30 (m, 2H); low resolution mass spectrum (ES) m/e 459 [(M+H)⁺, calcd for C₂₈H₃₅N₄O₂: 459]; 96.6% purity based on HPLC.

### Example 244

### (S)-6-AMINO-2-[3-(4-BUTOXY-PHENYL)-UREIDO]-HEXANOIC ACID P-TOLYLAMIDE

The title compound was prepared as described in Example 2 with a final coupling to 1-Butoxy-4-isocyanato-benzene. ¹H NMR (400 MHz, DMSO-d6) δ 10.06 (s, 1H), 8.57-8.55 (m, 1H), 7.73 (br s, 3H), 7.50 (d, J=8.39 Hz, 2H), 7.26 (d, J=8.97 Hz, 2H), 7.11 (d, J=8.33 Hz, 2H), 6.80 (d, J=9.05 Hz, 2H), 6.48-6.46 (m, 1H), 4.36 (dd, J=7.94, 13.51 Hz, 1H), 3.88 (t, J=6.48 Hz, 2H), 2.79-2.78 (br m, 2H), 2.25 (s, 3H), 1.77-1.53 (m, 6H), 1.46-1.30 (m, 4H), 0.92 (t, J=7.39 Hz, 3H); low resolution mass spectrum (ES) m/e 427 [(M+H)⁺, calcd for C₂₄H₃₅N₄O₃: 427]; 99.5% purity based on HPLC.

### Example 245

### (S)-4-[3-(5-AMINO-1-P-TOLYLCARBAMOYL-PENTYL)-UREIDO]-BENZOIC ACID ETHYL ESTER

The title compound was prepared as described in Example 2 with a final coupling to 4-Isocyanato-benzoic acid ethyl ester. ¹H NMR (400 MHz, DMSO-d6) δ 10.10 (s, 1H), 9.24-9.23 (m, 1H), 7.84 (d, J=8.79 Hz, 2H), 7.71 (br s, 3H), 7.53-7.49 (m, 4H), 7.12 (d, J=8.36 Hz, 2H), 6.79 (t, J=6.27 Hz, 1H), 4.39 (dd, J=7.94, 13.33 Hz, 1H), 4.26 (q, J=7.09 Hz, 2H), 2.82-2.75 (m, 2H), 2.25 (s, 3H), 1.80-1.71 (m, 1H), 1.67-1.51 (m, 3H), 1.45-1.33 (m, 2H), 1.29 (t, J=7.09 Hz, 3H); low resolution mass spectrum (ES) m/e 427 [(M+H)⁺, calcd for C₂₃H₃₁N₄O₄: 427]; 99.6% purity based on HPLC.

### Example 246

### (S)-7-AMINO-3-[3-(4-BENZYLOXY-PHENYL)-UREIDO]-HEPTANOIC ACID P-TOLYLAMIDE

The title compound was prepared as described in Example 2 with an initial coupling with (S)-7-tert-Butoxycarbonylamino-3-(9H-fluoren-9-ylmethoxycarbonylamino)-heptanoic acid. ¹H NMR (500 MHz, DMSO-d6) δ 9.87 (s, 1H), 8.42 (d, J=3.84 Hz, 1H), 7.67 (br s, 3H), 7.47 (d, J=8.44 Hz, 2H), 7.42 (d, J=6.94 Hz, 2H), 7.38 (t, J=7.46, 7.46 Hz, 2H), 7.31 (t, J=7.20 Hz, 1H), 7.28 (d, J=9.07 Hz, 2H), 7.09 (d, J=8.47 Hz, 2H), 6.87 (d, J=9.03 Hz, 2H), 6.20 (dd, J=4.55, 8.47 Hz, 1H), 5.02 (s, 2H), 4.05-3.99 (m, 1H), 2.80-2.73 (m, 2H), 2.54-2.44 (m, 2H), 2.24 (s, 3H), 1.60-1.30 (m, 6H); low resolution mass spectrum (ES) m/e 475 [(M+H)⁺, calcd for C₂₈H₃₅N₄O₃: 475]; 98.7% purity based on HPLC.

### Example 247

### (S)-7-AMINO-3-[3-(9H-FLUOREN-2-YL)-UREIDO]-HEPTANOIC ACID P-TOLYLAMIDE

The title compound was prepared as described in Example 246 with a final coupling with 2-isocyanato-9H-fluorene. ¹H NMR (500 MHz, DMSO-d6) δ 9.90 (s, 1H), 8.72 (s, 1H), 7.75-7.74 (m, 2H), 7.71 (d, J=8.28 Hz, 1H), 7.67 (br s, 3H), 7.51 (d, J=7.47 Hz, 1H), 7.48 (d, J=8.44 Hz, 2H), 7.32 (t, J=7.96 Hz, 2H), 7.22 (dt, J=0.92, 7.47 Hz, 1H), 7.09 (d, J=8.44 Hz, 2H), 6.36 (d, J=8.71 Hz, 1H), 4.10-4.03 (m, 1H), 3.84 (s, 2H), 2.81-2.75 (m, 2H), 2.57-2.48 (m, 2H), 2.24 (s, 3H), 1.61-1.32 (m, 6H); low resolution mass spectrum (ES) m/e 457 [(M+H)⁺, calcd for C₂₈H₃₃N₄O₂: 457]; 99.8% purity based on HPLC.

### Example 248

### (S)-6-AMINO-2-{3-[4-(2-FLUORO-BENZYLOXY)-PHENYL]-UREIDO}-HEXANOIC ACID P-TOLYLAMIDE

Intermediate #3 (50 mg) of Example 1 was dissolved in 4 mL of ethanol and hydrogenated under atmospheric hydrogen at room temperature for 12 hours. Filtering off the insolubes and concentrating down in vacuo produced the crude phenol. Alkylation with 1-Bromomethyl-2-fluoro-benzene using standard alkylating conditions of sodium hydride (3 equivalents) in dimethylformaide (0.2M) for 4 hours and quenching with water produced crude material after isolation with ethyl acetate. Deprotection of the crude material was performed described in Example 2. Purification by HPLC produced the title compound. ¹H NMR (500 MHz, DMSO-d6) δ 10.07 (s, 1H), 8.59 (s, 1H), 7.70 (br s, 3H), 7.53 (dt, J=1.42, 7.64, 7.78 Hz, 1 H), 7.50 (d, J=8.42 Hz, 2H), 7.41 (dt, J=1.74, 7.51, 7.57 Hz, 1H), 7.29 (d, J=9.06 Hz, 2H), 7.26-7.21 (m, 2H), 7.11 (d, J=8.46 Hz, 2H), 6.91 (d, J=9.03 Hz, 2H), 6.47 (d, J=8.33 Hz, 1H), 5.06 (s, 2H),4.37 (dt, J=5.61, 8.11, 8.17 Hz, 1H), 2.79-2.78 (m, 2H), 2.25 (s, 3H), 1.76-1.69 (m, 1H), 1.63-1.52 (m, 3H), 1.44-1.31 (m, 2H); low resolution mass spectrum (ES) m/e 479 [(M+H)⁺, calcd for C₂₇H₃₂N₄O₃: 479]; 99.6% purity based on HPLC.

### Example 249

### (S)-6-AMINO-2-{3-[4-(3-FLUORO-BENZYLOXY)-PHENYL]-UREIDO}-HEXANOIC ACID P-TOLYLAMIDE

The title compound was prepared as described in Example 248 with a final coupling reaction with 1-Bromomethyl-3-fluoro-benzene. ¹H NMR (500 MHz, DMSO-d6) δ 10.07 (s, 1H), 8.57 (s, 1H), 7.69 (br s, 3H), 7.49 (d, J=8.43 Hz, 2H), 7.42 (dd, J=8.10, 14.04 Hz, 1H), 7.30-7.24 (m, 4H), 7.16-7.10 (m, 3H), 6.90 (d, J=8.97 Hz, 2H), 6.46 (d, J=8.24 Hz, 1H), 5.06 (s, 2H), 4.37 (dt, J=5.70, 8.08, Hz, 1H), 2.81-2.75 (m, 2H), 2.25 (s, 3H), 1.76-1.69 (m, 1H), 1.63-1.51 (m, 3H), 1.44-1.31 (m, 2H); low resolution mass spectrum (ES) m/e 479 [(M+H)⁺, calcd for C₂₇H₃₂N₄O₃: 479]; 99.0% purity based on HPLC.

### Example 250

### (S)-6-AMINO-2-{3-[4-(4-FLUORO-BENZYLOXY)-PHENYL]-UREIDO}-HEXANOIC ACID P-TOLYLAMIDE

The title compound was prepared as described in Example 248 with a final coupling reaction with 1-Bromomethyl-4-fluoro-benzene. ¹H NMR (500 MHz, DMSO-d6) δ 10.07 (s, 1H), 8.57 (s, 1H), 7.69 (br s, 3H), 7.50-7.46 (m, 4H), 7.28 (d, J=9.09 Hz, 2H), 7.20 (t, J=8.88 Hz, 2H), 7.11 (d, J=8.39 Hz, 2H), 6.89 (d, J=9.06 Hz, 2H), 6.46 (d, J=8.33 Hz, 1H), 5.01 (s, 2H), 4.37 (dt, J=5.67, 8.14 Hz, 1H), 2.81-2.75 (m, 2H), 2.25 (s, 3H), 1.75-1.69 (m, 1H), 1.63-1.51 (m, 3H), 1.44-1.31 (m, 2H); low resolution mass spectrum (ES) m/e 479 [(M+H)⁺, calcd for C₂₇H₃₂N₄O₃: 479]; 97.9% purity based on HPLC.

### Example 251

### (S)-6-AMINO-2-[3-(4-{[METHYL-(4-NITRO-PHENYL)-AMINO]-METHYL}-PHENYL)-UREIDO]-HEXANOIC ACID P-TOLYLAMIDE

The title compound was prepared as described in Example 2 with a final coupling reaction with (4-Isocyanato-benzyl)-methyl-(4-nitro-phenyl)-amine. (4-Isocyanato-benzyl)-methyl-(4-nitro-phenyl)-amine was prepared from the coupling of 1-Chloromethyl-4-isocyanato-benzene to Methyl-(4-nitro-phenyl)-amine in dimethylforamide at room temperature under an inert atmosphere and was used directly in the coupling reaction without further purification. ¹H NMR (500 MHz, DMSO-d6) δ 10.05 (s, 1H), 8.73 (s, 1H), 8.02 (d, J=9.45 Hz, 2H), 7.66 (br s, 3H), 7.49 (d, J=8.43 Hz, 2H), 7.34 (d, J=8.56 Hz, 2H), 7.09 (dd, J=8.50, 13.03 Hz, 4H), 6.81 (d, J=9.52 Hz, 2H), 6.52 (d, J=7.95 Hz, 1H), 4.66 (s, 2H), 4.40-4.34 (m, 1H), 3.16 (s, 3H), 2.81-2.73 (m, 2H), 2.25 (s, 3H), 1.77-1.68 (m, 1H), 1.64-1.52 (m, 3H), 1.43-1.31 (m, 2H); low resolution mass spectrum (ES) m/e 519 [(M+H)⁺, calcd for C₂₈H₃₅N₆O₄: 519]; 81.5% purity based on HPLC.

### Example 252

### (S)-6-AMINO-2-(3-{4-[2-(4-METHOXY-PHENYL)-VINYL]-PHENYL}-UREIDO)-HEXANOIC ACID P-TOLYLAMIDE

The title compound was prepared as described in Example 2 with a final coupling reaction with 1-Isocyanato-4-(p-methoxy)styryl-benzene. ¹H NMR (500 MHz, DMSO-d6) δ 10.09 (s, 1H), 8.64 (s, 1H), 8.83 (d, J=2.39 Hz, 1H), 7.68 (br s, 3H), 7.50 (t, J=8.20 Hz, 4H), 7.43 (d, J=8.70 Hz, 2H), 7.38 (d, J=8.67 Hz, 2H), 7.12 (d, J=8.45 Hz, 2H), 7.01 (d, J=5.72 Hz, 2H), 6.93 (d, J=8.61 Hz, 2H), 6.59 (dd, J=2.34, 8.23 Hz, 1H), 4.40 (dd, J=8.10, 13.54 Hz, 1H), 3.76 (s, 3H), 2.82-2.76 (m, 2H), 2.25 (s, 3H), 1.78-1.71 (m, 1H), 1.65-1.53 (m, 3H), 1.45-1.32 (m, 2H); low resolution mass spectrum (ES) m/e 487 [(M+H)⁺, calcd for C₂₉H₃₅N₄O₃: 487]; 96.7% purity based on HPLC.

### Example 253

### (S)-6-AMINO-2-[3-(4-BENZYLOXY-3-CHLORO-PHENYL)-UREIDO]-HEXANOIC ACID P-TOLYLAMIDE

The title compound was prepared as described in Example 2 with a final coupling reaction with 1-Benzyloxy-2-chloro-4-isocyanato-benzene. ¹H NMR (500 MHz, DMSO-d6) δ 10.07 (s, 1H), 8.77 (d, J=1.16 Hz, 1H), 7.68 (br s, 3H), 7.65 (s, 1H), 7.49 (d, J=8.43 Hz, 2H), 7.45 (d, J=7.05 Hz, 2H), 7.39 (t, J=7.59 Hz, 2H), 7.33 (t, J=7.25 Hz, 1H), 7.11 (dd, J=3.60, 4.85 Hz, 4H), 6.56 (d, J=8.26 Hz, 1H), 5.12 (s, 2H), 4.36 (dd, J=8.06, 13.48 Hz, 1H), 2.81-2.75 (m, 2H), 2.25 (s, 3H), 1.76-1.70 (m, 1H), 1.64-1.51 (m, 3H), 1.43-1.31 (m, 2H); low resolution mass spectrum (ES) m/e 495 [(M+H)⁺, calcd for C₂₉H₃₂ClN₄O₃: 495]; 94.9% purity based on HPLC.

### Example 254

### (S)-6-AMINO-2-[3-(4-BENZYLOXY-PHENYL)-UREIDO]-HEXANOIC ACID (3,4-DIMETHYL-PHENYL)-AMIDE

The title compound was prepared as described in Example 2 with a final coupling reaction with 3,4-Dimethyl-phenylamine. ¹H NMR (400 MHz, DMSO-d6) δ 7.43-7.20 (m, 9H), 7.03 (d, J=8.3 Hz, 1H), 6.87 (d, J=9.0 Hz, 2H), 5.00 (s, 2H), 4.31 (dd, J=5.5, 8.3 Hz, 1H), 2.75 (t, J=7.5 Hz, 2H), 2.16 (s, 3H), 2.14 (s, 3H), 1.77-1.63 (m, 1H), 1.63-1.46 (m, 3H), 1.43-1.24 (m, 2H); low resolution mass spectrum (ES) m/e 475 [(M+H)⁺, calcd for C₂₈H₃₅N₄O₃: 475]; 99% purity based on HPLC.

### Example 255

### (S)-6-AMINO-2-[3-(4-BENZYLOXY-PHENYL)-UREIDO]-HEXANOIC ACID CYCLOPENTYLAMIDE

The title compound was prepared as described in Example 2 with an initial coupling reaction to cyclopentylamine. ¹H NMR (400 MHz, DMSO-d6) δ 7.43-7.26 (m, 5H), 7.22 (d, J=8.8 Hz, 2H), 6.87 (d, J=9.4 Hz, 2H), 5.00 (s, 2H), 4.12 (dd, J=5.7, 7.7 Hz, 1H), 3.96 (qu, J=6.4, 5.9. 13.4 Hz, 1H), 2.73 (t, J=7.7 Hz, 2H), 1.84-1.70 (m, 2H), 1.67-1.16 (m, 12H); low resolution mass spectrum (ES) m/e 439 [(M+H)⁺, calcd for C₂₅H₃₅N₄O₃: 439 ]; 100% purity based on HPLC.

### Example 256

### (S)-6-AMINO-2-{3-[4-(3,5-DIFLUORO-BENZYLOXY)-PHENYL]-UREIDO}-HEXANOIC ACID P-TOLYLAMIDE

The title compound was prepared as described in Example 248 with a final coupling reaction with 1-Bromomethyl-3,5-difluoro-benzene. ¹H NMR (500 MHz, DMSO-d6) δ 10.07 (s, 1H), 8.57 (s, 1H), 7.67 (br s, 3H), 7.49 (d, J=8.43 Hz, 2H), 7.29 (d, J=8.94 Hz, 2H), 7.21-7.14 (m, 3H), 7.11 (d, J=8.47 Hz, 2H), 7.06-7.02 (m, 1H), 6.90 (d, J=9.00 Hz, 2H), 6.46 (dd, J=1.83, 8.36 Hz, 1H), 5.07 (s, 2H), 4.37 (dt, J=5.81, 8.03 Hz, 1H), 2.81-2.75 (m, 2H), 2.25 (s, 3H), 1.76-1.69 (m, 1H), 1.63-1.51 (m, 3H), 1.44-1.29 (m, 2H); low resolution mass spectrum (ES) m/e 497 [(M+H)⁺, calcd for C₂₇H₃₁F₂N₄O₃: 497]; 98.8% purity based on HPLC.

### Example 257

### (S)-6-AMINO-2-[3-(3-BENZOOXAZOL-2-YL-PHENYL)-UREIDO]-HEXANOIC ACID P-TOLYLAMIDE

The title compound was prepared as described in Example 251 with a final coupling reaction with (4-Isocyanato-benzyl)-methyl-p-tolyl-amine. (4-Isocyanato-benzyl)-methyl-p-tolyl-amine was prepared from the coupling of 1-Chloromethyl-4-isocyanato-benzene to Methyl-(4-methylphenyl)-amine in dimethylformaide at room temperature under an inert atmosphere and was used directly in the coupling reaction without further purification. ¹H NMR (500 MHz, DMSO-d6) δ 10.49 (s, 1H), 9.30(br, 2H), 8.13 (s, 1H), 7.71 (br s, 3H), 7.46 (dd, J=2.68, 8.57 Hz, 4H), 7.29 (d, J=8.56 Hz, 2H), 7.22-7.15 (m, 6H), 4.06 (br s, 2H), 3.83 (br s, 1H), 3.22 (s, 3H), 2.77-2.73 (m, 2H), 2.32 (s, 3H), 2.27 (s, 3H), 1.95-1.24 (m, 6H); low resolution mass spectrum (ES) m/e 488 [(M+H)⁺, calcd for C₂₉H₃₈N₅O₂: 488]; 91.7% purity based on HPLC.

### Example 258

### (S)-6-AMINO-2-[3-(4-BENZYLOXY-PHENYL)-UREIDO]-HEXANOIC ACID BUTYLAMIDE

The title compound was prepared as described in Example 2 with an initial coupling reaction with butylamine. ¹H NMR (400 MHz, DMSO-d6) δ 7.44-7.27 (m, 5H), 7.24 (d, J=8.9 Hz, 2H), 6.87 (d, J=8.9 Hz, 2H), 5.01 (s, 2H), 4.13 (t, J=8.1 Hz, 1H), 3.10-2.98 (m, 2H), 2.73 (t, J=7.7 Hz, 2H), 1.68-1.18 (m, 10H), 0.84 (t, J=7.3 Hz, 3H); low resolution mass spectrum (ES) m/e 427 [(M+H)⁺, calcd for C₂₄H₃₅N₄O₃: 427]; 100% purity based on HPLC.

### Example 259

### (S)-6-AMINO-2-[3-(4-BENZYLOXY-PHENYL)-UREIDO]-HEXANOIC ACID (2-AMINO-ETHYL)-ESTER

The title compound was prepared as described in Example 2 with an initial coupling reaction with 2-Amino-ethanol. Isolation and purification produced the title compound of this Example and of Example 260. ¹H NMR (400 MHz, DMSO-d6) δ 8.48 (s, 1H), 8.06 (t, J=5.6 Hz, 1H), 7.61 (br s, 3H), 7.45-7.28 (m, 5H), 7.25 (d, J=9.1 Hz, 2H), 6.88 (d, J=8.7 Hz, 2H), 6.27 (d, J=8.1 Hz, 1H), 5.02 (s, 2H), 4.67 (br, 1H), 4.19 (dd, J=7.5, 13.3 Hz, 1H), 3.43-3.35 (m, 2H), 3.13 (qu, 2H), 2.75 (t, J=7.7 Hz, 2H), 1.69-1.19 (m, 6H); low resolution mass spectrum (ES) m/e 415.1 [(M+H)⁺, calcd for C₂₂H₃₁N₄O₄: 414.5]; XX% purity based on HPLC.

### Example 260

### (S)-6-AMINO-2-[3-(4-BENZYLOXY-PHENYL)-UREIDO]-HEXANOIC ACID (2-HYDROXY-ETHYL)-AMIDE

The title compound was prepared as described in Example 2 with an initial coupling reaction with 2-Amino-ethanol. Isolation and purification produced the title compound of this Example and of Example 259. ¹H NMR (400 MHz, DMSO-d6) δ 8.49 (s, 1H), 8.06 (t, J=5.6 Hz, 1H), 7.64 (br s, 3H), 7.45-7.28 (m, 5H), 7.25 (d, J=9.1 Hz, 2H), 6.88 (d, J=8.7 Hz, 2H), 6.28 (d, J=8.3 Hz, 1H), 5.01 (s, 2H), 4.67 (br, 1H), 4.18 (dd, J=7.5, 13.3 Hz, 1H), 3.43-3.35 (m, 2H), 3.13 (qu, 2H), 2.75 (t, J=7.7 Hz, 2H), 1.69-1.19 (m, 6H); low resolution mass spectrum (ES) m/e 415 [(M+H)⁺, calcd for C₂₂H₃₁N₄O₄: 415]; 100% purity based on HPLC.

### Example 261

### (S)-6-AMINO-2-{3-[4-(PYRIDIN-3-YLMETHOXY)-PHENYL]-UREIDO}-HEXANOIC ACID P-TOLYLAMIDE

The title compound was prepared as described in Example 262 with a final coupling reaction with 3-Bromomethyl-pyridine. ¹H NMR (400 MHz, DMSO-d6) δ 10.06 (s, 1H), 7.69 (br s, 3H), 7.59 (dd, J=4.96, 7.71 Hz, 1H), 7.49 (d, J=8.41 Hz, 2H), 7.30 (d, J=9.16 Hz, 2H), 7.11 (d, J=8.31 Hz, 2H), 6.93 (d, J=9.05 Hz, 2H), 5.13 (s, 2H), 4.37 (dd, J=7.81, 13.39 Hz, 1H), 2.81-2.76 (m, 2H), 2.25 (s, 3H), 1.77-1.68 (m, 1H), 1.61-1.34 (m, 5H); low resolution mass spectrum (ES) m/e 462 [(M+H)⁺, calcd for C₂₈H₃₂N₅O₃: 462]; 80.1% purity based on HPLC.

### Example 262

### (S)-6-AMINO-2-[4-(3-PYRIDIN-4-YL-UREIDO)-BENZYLAMINO]-HEXANOIC ACID P-TOLYLAMIDE

The title compound was prepared as described in Example 2 with a final coupling reaction with 1-Chloromethyl-4-isocyanato-benzene and quenching with Pyridin-4-ylamine. Purification by HPLC produced the title compound. ¹H NMR (400 MHz, DMSO-d6) δ 10.07 (s, 1H), 8.94 (s, 1H), 8.23 (d, J=7.38 Hz, 2H), 8.13 (s, 2H), 7.73 (br s, 3H), 7.49 (d, J=8.43 Hz, 2H), 7.41 (d, J=8.62 Hz, 2H), 7.26 (d, J=8.63 Hz, 2H), 7.11 (d, J=8.38 Hz, 2H), 6.81 (d, J=7.47 Hz, 2H), 6.67 (d, J=8.08 Hz, 1H), 5.23 (s, 2H), 4.35 (dd, J=8.00, 13.54 Hz, 1H), 2.81-2.74 (m, 2H), 2.25 (s, 3H), 1.77-1.68 (m, 1H), 1.65-1.21 (m, 5H), ; low resolution mass spectrum (ES) m/e 461 [(M+H)⁺, calcd for C₂₆H₃₃N₆O₂: 461]; 93.8% purity based on HPLC.

### Example 263

### (S)—NR-(6-AMINO-HEXYL)-2-13-(4-BENZYLOXY-PHENYL)-UREIDO]-3-PHENYL-PROPIONAMIDE

((S)-2-(9H-Fluoren-9-ylmethoxycarbonylamino)-3-phenyl-propionic acid was coupled to (6-Amino-hexyl)-carbamic acid tert-butyl ester as described in Example 5. Coupling to 1-Benzyloxy-4-isocyanato-benzene as described in the preparation of Example 2 produced the titled compound. ¹H NMR (400 MHz, DMSO-d6) δ 7.42-7.15 (m, 12H), 6.85 (d, J=9.0 Hz, 2H), 4.99 (s, 2H), 4.36 (dd, J=5.7, 8.3 Hz, 1H), 3.09-2.86 (m, 3H), 2.82-2.69 (m, 3H), 1.47 (quintet, J=7.0, 8.1, 15.4 Hz, 2H), 1.37-1.10 (m, 6H); low resolution mass spectrum (ES) m/e 489 [(M+H)⁺, calcd for C₂₉H₃₇N₄O₃: 489]; 100% purity based on HPLC.

### Example 264

### (S)-6-AMINO-2-[3-(4-BENZYLOXY-PHENYL)-UREIDO]-HEXANOIC ACID (4-METHYL-CYCLOHEXYL)-AMIDE

The title compound was prepared as described in Example 2 with an initial coupling reaction with 4-Methyl-cyclohexylamine. ¹H NMR (400 MHz, DMSO-d6) δ 7.90 (d, J=7.9 Hz, 0.5H), 7.86 (d, J=7.5 Hz, 0.5H), 7.43-7.26 (m, 5H), 7.22 (d, J=9.0 Hz, 1H), 7.21 (d, J=9.2 Hz, 1H), 6.87 (d, J=8.8 Hz, 2H), 5.00 (s, 2H), 4.22 (dd, J=5.5, 7.5 Hz, 0.5H), 4.10 (dd, J=5.5, 7.6 Hz, 0.5H), 3.78-3.69 (br, 0.5H), 3.50-3.37 (br, 0.5H), 2.72 (t, J=7.5 Hz, 2H), 1.76-1.36 (m, 9H), 1.34-1.05 (m, 5H), 0.99-0.88 (m, 1H), 0.85 (d, J=8.8 Hz, 1.5H), 0.83 (d, J=8.6 Hz, 1.5H); low resolution mass spectrum (ES) m/e 467 [(M+H)⁺, calcd for C₂₇H₃₉N₄O₃: 467]; 99% purity based on HPLC.

### Example 265

### (s)-6-AMINO-2-[3-(4-BENZYLOXY-PHENYL)-UREIDO]-HEXANOIC ACID CYCLOHEXYLMETHYL-AMIDE

The title compound was prepared as described in Example 2 with an initial coupling reaction with Cyclohexyl-methylamine. ¹H NMR (400 MHz, DMSO-d6) 7.43-7.26 (m, 5H), 7.23 (d, J=9.0 Hz, 2H), 6.87 (d, J=9.0 Hz, 2H), 5.00 (s, 2H), 4.14 (dd, J=5.5, 7.7 Hz, 1H), 2.92 (dd, J=6.8, 13.4 Hz, 1H), 2.86 (dd, J=6.8, 13.2 Hz, 1H), 2.72 (t, J=7.9, 7.5 Hz, 2H), 1.68-1.01 (m, 15H), 0.83 (br, 2H); low resolution mass spectrum (ES) m/e 467 [(M+H)⁺, calcd for C₂₇H₃₉N₄O₃: 467]; 96% purity based on HPLC.

### Example 266

### (S)-6-AMINO-2-[3-(4-BENZYLOXY-PHENYL)-UREIDO]-HEXANOIC ACID CYCLOPROPYLAMIDE

The title compound was prepared as described in Example 2 with an initial coupling reaction with Cyclopropylamine. ¹H NMR (400 MHz, DMSO-d6) δ 7.43-7.26 (m, 5H), 7.22 (d, J=9.2 Hz, 2H), 6.87 (d, J=9.2 Hz, 2H), 5.00 (s, 2H), 4.06 (dd, J=5.7, 7.7 Hz, 1H), 2.73 (t, J=7.5, 7.7 Hz, 2H), 2.59 (septet, 1H), 1.64-1.37 (m, 4H), 1.35-1.16 (m, 2H), 0.65-0.57 (m, 2H), 0.42-0.33 (m, 2H); low resolution mass spectrum (ES) m/e 411 [(M+H)⁺, calcd for C₂₃H₃₁N₄O₃: 411]; 100% purity based on HPLC.

### Example 267

### (S)-[5-AMINO-1-(5-PHENYL-2H-PYRAZOL-3-YLCARBAMOYL)-PENTYL]-CARBAMIC ACID 9H-FLUOREN-9-YLMETHYL ESTER

The title compound was prepared as described in Example 6 with an initial coupling reaction with 5-Phenyl-2H-pyrazol-3-ylamine. ¹H NMR (400 MHz, DMSO-d6) δ H-NMR (400 MHz) 10.49 (s, 1H); 7.89 (d, 2H, J=7.5 Hz); 7.72 (dd, 4H, J=7.5 Hz, J=14.7 Hz); 7.57 (m, 3H); 7.42 (m, 4H); 7.33 (m, 3H); 6.85 (s, 1H); 4.34-4.14 (m, 4H); 2.78 (dddd, 2H, J=7.1 Hz, J=7.1 Hz, J=6.7 Hz, J=12.3 Hz); 1.75-1.28 (m, 6H); low resolution mass spectrum (ES) m/e 510 [(M+H)⁺, calcd for C₃₀H₃₂N₅O₃: 510]; 100% purity based on HPLC.

### Example 268

### (S)-3-(2-AMINO-ACETYLAMINO)-2-[3-(4-BENZYLOXY-PHENYL)-UREIDO]-N-P-TOLYL-PROPIONAMIDE

4-Methyl-phenylamine was coupled to (S)-3-tert-Butoxycarbonylamino-2-(9H-fluoren-9-ylmethoxycarbonylamino)-propionic acid, and then to tert-Butoxycarbonylamino-acetic acid as described in the method of Example 182. Coupling to 1-Benzyloxy-4-isocyanato-benzene as described in the method of Example 2. Purification by HPLC produced the title compound. ¹H NMR (400 MHz, DMSO-d6) δ 7.46 (d, J=8.6 Hz, 2H), 7.43-7.23 (m, 7H), 7.10 (d, J=8.1 Hz, 2H), 6.88 (d, J=9.2 Hz, 2H), 5.01 (s, 2H), 4.45 (t, J=6.4, 5.9 Hz, 1H), 3.51-3.44 (m, 4H), 2.23 (s, 3H); low resolution mass spectrum (ES) m/e 476 [(M+H)⁺, calcd for C₂₆H₃₀N₅O₄: 476]; 94% purity based on HPLC.

### Example 269

### (S)-6-AMINO-2-[3-(4-PIPERIDIN-1-YL-PHENYL)-UREIDO]-HEXANOIC ACID P-TOLYLAMIDE

The title compound was prepared as described in the method of Example 2 with a final coupling to 1-(4-Isocyanato-phenyl)-piperidine. ¹H NMR (500 MHz, DMSO-d6) δ 10.08 (s, 1H), 8.99 (br s, 1H), 7.70 (br s, 3H), 7.50-7.46 (br m, 6H), 7.11 (d, J=8.45 Hz, 2H), 6.68 (br s, 1H),4.36 (dd, J=7.98, 13.51 Hz, 1H), 3.38 (br s, 3H), 2.81-2.75 (m, 2H), 2.25 (s, 3H), 1.82-1.31 (m, 13H); low resolution mass spectrum (ES) m/e 438 [(M+H)⁺, calcd for C₂₅H₃₆N₅O₂: 438]; 95.1% purity based on HPLC.

### Example 270

### (S)-6-AMINO-2-[3-(4-BENZOOXAZOL-2-YL-PHENYL)-UREIDO]-HEXANOIC ACID P-TOLYLAMIDE

The title compound was prepared as described in the method of Example 2 with a final coupling to 2-(4-Isocyanato-phenyl)-benzooxazole. ¹H NMR (500 MHz, DMSO-d6) δ 10.12 (s, 1H), 9.27 (s, 1H), 8.08 (d, J=8.62 Hz, 2H), 7.74-7.71 (br m, 4H), 7.63 (d, J=8.60 Hz, 2H), 7.51 (d, J=8.08 Hz, 2H), 7.39-7.37 (m, 2H), 7.12 (d, J=7.97 Hz, 2H), 6.81 (d, J=7.79 Hz, 1H), 4.42 (dd, J=6.88, 13.04 Hz, 1H), 2.80-2.79 (m, 2H), 2.25 (s, 3H), 1.79-1.76 (m, 1H), 1.65-1.57 (m, 3H), 1.45-1.39 (m, 2H); low resolution mass spectrum (ES) m/e 472 [(M+H)⁺, calcd for C₂₇H₃₀N₅O₃: 472]; 97.3% purity based on HPLC.

### Example 271

### (S)-6-AMINO-2-[3-(3-BENZOOXAZOL-2-YL-PHENYL)-UREIDO]-HEXANOIC ACID P-TOLYLAMIDE

The title compound was prepared as described in the method of Example 2 with a final coupling to 2-(3-Isocyanato-phenyl)-benzooxazole. ¹H NMR (500 MHz, DMSO-d6) δ 10.12(s, 1H), 9.10 (s, 1H), 8.49 (s, 1H), 7.82-7.79 (m, 2H), 7.77-7.75 (m, 1H), 7.67 (br m, 3H), 7.51 (d, J=8.42 Hz, 2H), 7.48-7.40 (m, 4H), 7.13 (d, J=8.35 Hz, 2H), 6.67 (d, J=8.02 Hz, 1H), 4.42 (dt, J=5.95, 8.09 Hz, 1H), 2.83-2.77 (m, 2H), 2.25 (s, 3H), 1.82-1.75 (m, 1H), 1.69-1.55 (m, 3H), 1.46-1.34 (m, 2H); low resolution mass spectrum (ES) m/e 472 [(M+H)⁺, calcd for C₂₇H₃₀N₅O3: 472]; 98.5% purity based on HPLC.

### Example 272

### (S)-6-AMINO-2-[3-(5-P-TOLYLOXYMETHYL-[1,3,4] THIADIAZOL-2-YL)-UREIDO]-HEXANOIC ACID P-TOLYLAMIDE

The title compound was prepared as described in the method of Example 2 with a final coupling to 2-Isocyanato-5-p-tolyloxymethyl-[1,3,4]thiadiazole. ¹H NMR (500 MHz, DMSO-d6) δ 10.95 (br s, 1H), 10.14 (s, 1H), 7.65 (br s, 3H), 7.48 (d, J=8.41 Hz, 2H), 7.13-7.09 (m, 5H), 6.93 (d, J=8.55 Hz, 2H), 5.38 (s, 2H), 4.42 (dt, J=5.48, 7.90 Hz, 1H), 2.80-2.74 (m, 2H), 2.25 (s, 3H), 2.23 (s, 3H), 1.81-1.74 (m, 1H), 1.68-1.51 (m, 3H), 1.40-1.29 (m, 2H); low resolution mass spectrum (ES) m/e 483 [(M+H)⁺, calcd for C₂₄H₃₁N₆O₃S: 483]; 90.4% purity based on HPLC.

### Example 273

### (S)-6-AMINO-2-{3-[4-(4-CHLORO-BENZYLOXY)-PHENYL]-UREIDO}-HEXANOIC ACID P-TOLYLAMIDE

The title compound was prepared as described in the method of Example 248 with a final coupling to 1-Bromomethyl-4-chloro-benzene. ¹H NMR (400MHz, DMSO-d6) δ 10.05 (s, 1H), 8.56 (s, 1H), 7.68 (br s, 3H), 7.49 (d, J=8.42 Hz, 2H), 7.44 (s, 4H), 7.28 (d, J=9.04 Hz, 2H), 7.11 (d, J=8.33 Hz, 2H), 6.89 (d, J=9.06 Hz, 2H), 6.45 (d, J=8.24 Hz, 1H), 5.03 (s, 2H), 4.37 (dd, J=7.99, 13.57 Hz, 1H), 2.79-2.76 (m, 2H), 2.25 (s, 3H), 1.77-1.67 (m, 1H), 1.64-1.52 (m, 3H), 1.45-1.30 (m, 2H); low resolution mass spectrum (ES) m/e 495 [(M+H)⁺, calcd for C₂₇H₃₂ClN₄Q₃: 495]; 98.9% purity based on HPLC.

### Example 274

### (S)-6-AMINO-2-{3-[4-(2,4-DICHLORO-BENZYLOXY)-PHENYL]-UREIDO}-HEXANOIC ACID P-TOLYLAMIDE

The title compound was prepared as described in the method of Example 248 with a final coupling to 1-Bromomethyl-2,4-dichloro-benzene. ¹H NMR (400MHz, DMSO-d6) d 10.05 (s, 1H), 8.59 (s, 1H), 7.69 (br s, 3H), 7.67 (d, J=2.06 Hz, 1H), 7.59 (d, J=8.33 Hz, 1H), 7.49 (d, J=8.43 Hz, 2H), 7.47 (dd, J=2.11, 8.34 Hz, 1H), 7.30 (d, J=9.06 Hz, 2H),7.11 (d, J=8.39 Hz, 2H), 6.91 (d, J=9.05 Hz, 2H), 6.47 (d, J=7.32 Hz, 1H), 5.07 (s, 2H), 4.37 (dd, J=7.94, 13.54 Hz, 1H), 2.81-2.76 (m, 2H), 2.25 (s, 3H), 1.77-1.69 (m, 1H), 1.64-1.53 (m, 3H), 1.45-1.32 (m, 2H); low resolution mass spectrum (ES) m/e 529, 531 [(M+H)⁺, calcd for C₂₇H₃IC₁₂N₄O₃: 529, 531]; 99.4% purity based on HPLC.

### Example 275

### (S)-6-AMINO-2-{3-[4-(3-NITRO-BENZYLOXY)-PHENYL]-UREIDO}-HEXANOIC ACID P-TOLYLAMIDE

The title compound was prepared as described in the method of Example 248 with a final coupling to 1-Bromomethyl-3-nitro-benzene. ¹H NMR (400 MHz, DMSO-d6) δ 10.04 (s, 1H), 8.57 (s, 1H), 8.27 (s, 1H), 8.17 (d, J=8.32 Hz, 1H), 7.88 (d, J=7.68 Hz, 1H), 7.68 (t, J=7.91, Hz, 1H), 7.69 (br s, 3H), 7.48 (d, J=8.43 Hz, 2H),7.29 (d, J=9.06 Hz, 2H), 7.10 (d, J=8.34 Hz, 2H), 6.92 (d, J=9.07 Hz, 2H), 6.46 (br m, 1H), 5.19 (s, 2H), 4.36 (q, J=7.77 Hz, 1H), 2.81-2.73 (m, 2H), 2.25 (s, 3H), 1.77-1.68 (m, 1H), 1.64-1.51 (m, 3H), 1.45-1.30 (m, 2H); low resolution mass spectrum (ES) m/e 506 [(M+H)⁺, calcd for C₂₇H₃₂N₅O₅: 506]; 95.1% purity based on HPLC.

### Example 276

### (S)-3-{4-[3-(5-AMINO-1-P-TOLYLCARBAMOYL-PENTYL)-UREIDO]-PHENOXYMETHYL}-BENZOIC ACID METHYL ESTER

The title compound was prepared as described in the method of Example 248 with a final coupling of 3-Bromomethyl-benzoic acid methyl ester. ¹H NMR (400 MHz, DMSO-d6) δ 10.07 (s, 1H), 8.57 (s, 1H), 8.02 (s, 1H), 7.91 (d, J=7.77 Hz, 1H), 7.71 (d, J=7.69 Hz, 1H), 7.69 (br, 3H), 7.54 (t, J=7.70 Hz, 1H), 7.49 (d, J=8.43 Hz, 2H), 7.29 (d, J=9.05 Hz, 2H), 7.11 (d, J=8.40 Hz, 2H), 6.91 (d, J=9.06 Hz, 2H), 6.46 (d, J=6.12 Hz, 1H), 5.12 (s, 2H), 4.37 (dd, J=7.88, 13.55 Hz, 1H), 3.86 (s, 3H), 2.82-2.73 (m, 2H), 2.25 (s, 3H), 1.77-1.68 (m, 1H), 1.64-1.51 (m, 3H), 1.45-1.30 (m, 2H); low resolution mass spectrum (ES) m/e 519 [(M+H)⁺, calcd for C₂₉H₃₅N₄O₅: 519]; 94.8% purity based on HPLC.

### Example 277

### (2S)-6-AMINO-2-{3-[4-(1-PHENYL-ETHOXY)-PHENYL]-UREIDO}-HEXANOIC ACID P-TOLYLAMIDE

The title compound was prepared as described in the method of Example 248 with a final coupling of racemic (1-Bromo-ethyl)-benzene. ¹H NMR (400 MHz, DMSO-d6) δ 10.03 (s, 1H), 8.47 (s, 1H), 7.65 (br s, 3H), 7.48 (d, J=8.35 Hz, 2H), 7.37 (d, J=7.02 Hz, 2H), 7.32 (t, J=7.53 Hz, 2H), 7.23 (t, J=7.17 Hz, 1H), 7.18 (d, J=8.99 Hz, 2H), 7.10 (d, J=8.38 Hz, 2H), 6.77 (d, J=9.04 Hz, 2H), 6.40 (d, J=8.21 Hz, 1H), 5.38 (q, J=6.36 Hz, 1H), 4.35 (dd, J=8.00, 13.60 Hz, 1H), 2.79-2.74 (m, 2H), 2.24 (s, 3H), 1.75-1.66 (m, 1H), 1.61-1.51 (m, 3H), 1.51 (d, J=6.37 Hz, 3H), 1.42-1.28 (m, 2H); low resolution mass spectrum (ES) m/e 475 [(M+H)⁺, calcd for C₂₈H₃₅N₄O₃: 475]; 100% purity based on HPLC.

### Example 278

### 2-[1-(4-AMINO-BUTYL)-3-(4-BENZYLOXY-PHENYL)-UREIDO]-N-P-TOLYL-ACETAMIDE

[(4-tert-Butoxycarbonylamino-butyl)-(9H-fluoren-9-ylmethoxycarbonyl)-amino]-acetic acid was coupled to 4-methylphenylamine, and then to 1-Benzyloxy-4-isocyanato-benzene as described in the method of Example 2. Purification by HPLC produced the title compound. ¹H NMR (400 MHz, DMSO-d6) δ 9.91 (s, 1H), 8.26 (s, 1H), 7.65 (br s, 3H), 7.48 (d, J=8.36 Hz, 2H), 7.43 (d, J=6.94 Hz, 2H), 7.38 (t, J=7.22, 7.22 Hz, 2H), 7.34-7.30 (m, 3H), 7.11 (d, J=8.37 Hz, 2H), 6.90 (d, J=9.00 Hz, 2H), 5.04 (s, 2H), 4.11 (s, 2H), 3.38 (br, 2H), 2.81-2.80 (br m, 2H), 2.25 (s, 3H), 1.56 (br, 4H); low resolution mass spectrum (ES) m/e 461 [(M+H)⁺, calcd for C₂₇H₃₃N₄O₃: 461]; 99% purity based on HPLC.

### Example 279

### (S)-6-AMINO-2-{3-[4-(PYRIDIN-4-YLMETHOXY)-PHENYL]-UREIDO}-HEXANOIC ACID P-TOLYLAMIDE

The title compound was prepared as described in the method of Example 262 with a final coupling with 4-Bromomethyl-pyridine. ¹H NMR (400 MHz, DMSO-d6) δ 10.06 (s, 1H), 8.64 (m, 2H), 8.57-8.55 (m, 1H), 7.64 (br s, 3H), 7.56-7.53 (m, 2H), 7.49 (d, J=8.42 Hz, 2H), 7.29 (d, J=8.99 Hz, 2H), 7.11 (d, J=8.27 Hz, 2H), 6.91 (d, J=12.42 Hz, 2H), 6.44 (t, J=7.96 Hz, 1H), 5.18 (m, 2H), 4.37 (dd, J=7.53, 13.84 Hz, 1H), 2.80-2.75 (m, 2H), 2.25 (s, 3H), 1.75-1.68 (m, 1H), 1.63-1.52 (m, 3H), 1.44-1.29 (m, 2H); low resolution mass spectrum (ES) m/e 462 [(M+H)⁺, calcd for C₂₈H₃₂N₅O₃: 462]; 95.4% purity based on HPLC.

### Example 280

### (S)-{4-[(5-AMINO-1-P-TOLYLCARBAMOYL-PENTYLAMINO)-METHYL]-PHENYL}-CARBAMIC ACID

Intermediate #2 of Example 1 was coupled to 1-Chloromethyl-4-isocyanato-benzene as described for Intermediate #3 of Example 1 and quenched with water. Purification by HPLC produced the title compound. ¹H NMR (500 MHz, DMSO-d6) δ 10.08 (s, 1H), 8.69 (s, 1H), 7.68 (br s, 3H), 7.50 (d, J=8.45 Hz, 2H), 7.33 (d, J=8.52 Hz, 2H), 7.16 (d, J=8.47 Hz, 2H), 7.11 (d, J=8.46 Hz, 2H), 6.53 (d, J=8.26 Hz, 1H), 5.03 (br s,1H), 4.40-4.36 (m, 3H), 2.82-2.75 (m, 2H), 2.25 (s, 3H), 1.77-1.70 (m, 1H), 1.64-1.52 (m, 3H), 1.44-1.30 (m, 2H); low resolution mass spectrum (ES) m/e 385 [(M+H)⁺, calcd for C₂₁H₂₉N₄O₃: 385]; 98.9% purity based on HPLC.

### Example 281

### 2-[1-(4-AMINO-BUTYL)-3-(4-BENZYLOXY-PHENYL)-UREIDO]-NR-(1H-INDOL-4-YL)-ACETAMIDE

[(4-tert-Butoxycarbonylamino-butyl)-(9H-fluoren-9-ylmethoxycarbonyl)-amino]-acetic acid was coupled to 1H-Indol-4-ylamine, and then to 1-Benzyloxy-4-isocyanato-benzene as described in the method of Example 2. Purification by HPLC produced the title compound. ¹H NMR (400 MHz, DMSO-d6) δ 9.66 (s, 1H); 8.32 (s, 1H); 7.62 (m, 4H); 7.48-7.24 (m, 9H); 7.14 (d, 1H, J=8.1Hz); 7.00 (t, 1H, J=7.9Hz); 6.90 (d, 2H, J=9.1 Hz); 6.68 (s, 1H); 5.03 (s, 2H); 4.25 (s, 2H); 3.42 (t, 2H, J=6.2Hz); 2.81 (dd, 2H, J=6.1 Hz, J=11.9Hz); 1.58 (br s, 4H);

low resolution mass spectrum (ES) m/e 486 [(M+H)⁺, calcd for C₂₈H₃₂N₅O₃: 486]; 90% purity based on HPLC.

### Example 282

### 2-[1-(4-AMINO-BUTYL)-3-(4-BENZYLOXY-PHENYL)-UREIDO]-N-(4-TERT-BUTYL-PHENYL)-ACETAMIDE

[(4-tert-Butoxycarbonylamino-butyl)-(9H-fluoren-9-ylmethoxycarbonyl)-amino]-acetic acid was coupled to 4-t-butyl phenylamine, and then to 1-Benzyloxy-4-isocyanato-benzene as described in the method of Example 2. Purification by HPLC produced the title compound. ¹H NMR (400 MHz, DMSO-d6) δ 9.90 (s, 1H); 8.25 (s, 1H); 7.63 (s, 3H); 7.50 (d, 2H, J=8.7Hz); 7.39 (m, 4H); 7.31 (ddd, 5H, J=2.4Hz, J=4.1 Hz, J=8.7 Hz); 6.89 (d, 2H, J=9.1 Hz); 5.03 (s, 2H); 4.10 (s, 2H); 2.80 (dd, 2H, J=6.0Hz, J=11.3 Hz); 1.56 (s, 4H); 1.25 (s, 9H); low resolution mass spectrum (ES) m/e 503 [(M+H)⁺, calcd for C₃₀H₃₉N₄O₃: 503]; 98% purity based on HPLC.

### Example 283

### (S)-6-AMINO-2-[3-(4-BENZYLOXY-PHENYL)-UREIDO]-HEXANOIC ACID INDAN-5-YLAMIDE

The title compound was prepared as described in the method of Example 2 with an initial coupling to Indan-5-ylamine. ¹H NMR (400 MHz, DMSO-d6) δ 7.47 (br, 1H),7.42-7.21 (m, 8H), 7.12 (d, J=8.1 Hz, 1H), 6.87 (d, J=9.2 Hz, 2H), 5.00 (s, 2H), 4.32 (dd, J=5.7, 7.9 Hz, 1H), 2.77 (quintet, J=7.2, 13.8, 12.8 Hz, 6H), 1.97 (quintet, J=7.2, 11.0, 11.4 Hz, 2H), 1.76-1.48 (m, 4H), 1.41-1.25 (m, 2H); low resolution mass spectrum (ES) m/e 487 [(M+H)⁺, calcd for C₂₉H₃₅N₄O₃: 487]; 96% purity based on HPLC.

### Example 284

### (S)-6-AMINO-2-{3-[4-(2,3-DIFLUORO-BENZYLOXY)-PHENYL]-UREIDO}-HEXANOIC ACID P-TOLYLAMIDE

The title compound was prepared as described in the method of Example 248 with a final coupling to 1-Bromomethyl-2,3-difluoro-benzene. ¹H NMR (500 MHz, DMSO-d6) δ 10.07 (s, 1H), 8.56 (s, 1H), 7.65 (br s, 3H), 7.49 (d, J=8.42 Hz, 2H),7.43 (dt, J=1.30, 9.82 Hz, 1H), 7.35 (dd, J=6.45, 7.30 Hz, 1H), 7.30 (d, J=8.88 Hz, 2H), 7.23 (dt, J=0.99, 8.92 Hz, 1H), 7.11 (d, J=8.54 Hz, 2H), 6.92 (d, J=8.92 Hz, 2H), 6.44 (d, J=8.30 Hz, 1H), 5.12 (s, 2H), 4.38 (dt, J=5.83, 8.09 Hz, 1H), 2.81-2.75 (m, 2H), 2.25 (s, 3H), 1.76-1.69 (m, 1H), 1.63-1.51 (m, 3H), 1.44-1.31 (m, 2H); low resolution mass spectrum (ES) m/e 497 [(M+H)⁺, calcd for C₂₇H₃₁F₂N₄O₃: 497]; 85.4% purity based on HPLC.

### Example 285

### (S)-6-AMINO-2-{3-[4-(2,4-DIFLUORO-BENZYLOXY)-PHENYL]-UREIDO}-HEXANOIC ACID P-TOLYLAMIDE

The title compound was prepared as described in the method of Example 248 with a final coupling to 1-Bromomethyl-2,4-difluoro-benzene. ¹H NMR (500 MHz, DMSO-d6) δ 10.07 (s, 1H), 8.57 (s, 1H), 7.67 (br s, 3H), 7.59 (dd, J=8.56, 15.32 Hz, 1H), 7.49 (d, J=8.42 Hz, 2H), 7.32-7.27 (m, 3H), 7.13-7.10 (m, 3H), 6.91 (d, J=8.96 Hz, 2H), 6.45 (d, J=8.35 Hz, 1H), 5.03 (s, 2H), 4.37 (dt, J=5.66, 8.16 Hz, 1H), 2.81-2.75 (m, 2H), 2.25 (s, 3H), 1.76-1.69 (m, 1H), 1.63-1.51 (m, 3H), 1.44-1.31 (m, 2H); low resolution mass spectrum (ES) m/e 497 [(M+H)⁺, calcd for C₂₇H₃₁F₂N₄O₃: 497]; 97.3% purity based on HPLC.

### Example 286

### (S)-6-AMINO-2-{3-[4-(2,5-DIFLUORO-BENZYLOXY)-PHENYL]-UREIDO}-HEXANOIC ACID P-TOLYLAMIDE

The title compound was prepared as described in the method of Example 248 with a final coupling to 1-Bromomethyl-2,5-difluoro-benzene. ¹H NMR (500 MHz, DMSO-d6) δ 10.07 (s, 1H), 8.58 (s, 1H), 7.66 (br s, 3H), 7.49 (d, J=8.41 Hz, 2H), 7.38 (ddd, J=3.20, 5.62, 8.85 Hz, 1H), 7.33-7.29 (m, 3H), 7.27-7.22 (m, 1H), 7.11 (d, J=8.46 Hz, 2H), 6.92 (d, J=8.95 Hz, 2H), 6.45 (d, J=8.28 Hz, 1H), 5.06 (s, 2H), 4.38 (dt, J=5.79, 8.11 Hz, 1H), 2.81-2.75 (m, 2H), 2.25 (s, 3H), 1.76-1.69 (m, 1H), 1.63-1.52 (m, 3H), 1.44-1.31 (m, 2H); low resolution mass spectrum (ES) m/e 497 [(M+H)⁺, calcd for C₂₇H₃₁F₂N₄O₃: 497]; 97.3% purity based on HPLC.

### Example 287

### (S)-6-AMINO-2-{3-[4-(2,6-DIFLUORO-BENZYLOXY)-PHENYL]-UREIDO}-HEXANOIC ACID P-TOLYLAMIDE

The title compound was prepared as described in the method of Example 248 with a final coupling to 1-Bromomethyl-2,6-difluoro-benzene: ¹H NMR (500 MHz, DMSO-d6) δ 10.07 (s, 1H), 8.57 (s, 1H), 7.65 (br s, 3H), 7.54-7.48 (m, 3H), 7.30 (d, J=8.91 Hz, 2H), 7.21-7.15 (m, 3H), 7.12 (d, J=8.50 Hz, 2H), 6.91 (d, J=8.94 Hz, 2H), 6.44 (d, J=8.26 Hz, 1H), 5.03 (s, 2H), 4.38 (dt, J=5.59, 8.03 Hz, 1H), 2.81-2.75 (m, 2H), 2.25 (s, 3H), 1.76-1.69 (m, 1H), 1.63-1.51 (m, 3H), 1.43-1.32 (m, 2H); low resolution mass spectrum (ES) m/e 497 [(M+H)⁺, calcd for C₂₇H₃₁F₂N₄O₃: 497]; 81.7% purity based on HPLC.

### Example 288

### (S)-6-AMINO-2-{3-[4-(3,4-DIFLUORO-BENZYLOXY)-PHENYL]-UREIDO}-HEXANOIC ACID P-TOLYLAMIDE

The title compound was prepared as described in Example 248 with the final coupling completed with 1-Bromomethyl-3,4-difluoro-benzene: ¹H NMR (500 MHz, DMSO-d6) δ 10.07 (s, 1H), 8.56 (s, 1H), 7.67 (br s, 3H), 7.52-7.41 (m, 4H), 7.28 (d, J=9.03 Hz, 3H), 7.11 (d, J=8.39 Hz, 2H), 6.89 (d, J=9.05 Hz, 2H), 6.44 (d, J=8.30 Hz, 1H), 5.02 (s, 2H), 4.37 (dt, J=5.86, 8.17 Hz, 1H), 2.81-2.75 (m, 2H), 2.25 (s, 3H), 1.77-1.69 (m, 1H), 1.63-1.51 (m, 3H), 1.42-1.30 (m, 2H); low resolution mass spectrum (ES) m/e 497 [(M+H)⁺, calcd for C₂₇H₃₁F₂N₄O₃: 497]; 93.1% purity based on HPLC.

### Example 289

### (S)-1-[5-AMINO-1-(4-METHYL-PIPERAZINE-1-CARBONYL)-PENTYL]-3-(4-BENZYLOXY-PHENYL)-UREA

The title compound was prepared as described in the method of Example 2 with an initial coupling to 1-Methyl-piperazine. ¹H NMR (400 MHz, DMSO-d6) δ 7.42-7.26 (m, 5H), 7.22 (d, J=9.4 Hz, 2H), 6.87 (d, J=9.4 Hz, 2H), 5.00 (s, 3H), 4.63 (dd, J=5.2, 7.8 Hz, 1H), 3.38-2.83 (br, 4H), 2.80-2.69 (m, 5H), 1.68-1.21 (m, 6H), CH₂ obscured by water peak; low resolution mass spectrum (ES) m/e 454 [(M+H)⁺, calcd for C₂₅H₃₆N₅O₃: 453]; 99% purity based on HPLC.

### Example 290

### (S)-1-[5-AMINO-1-(PIPERIDINE-1-CARBONYL)-PENTYL]-3-(4-BENZYLOXY-PHENYL)-UREA

The title compound was prepared as described in the method of Example 2 with an initial coupling to Piperidine. ¹H NMR (400 MHz, DMSO-d6) δ 7.41-7.25 (m, 5H), 7.21 (d, J=9.4 Hz, 2H), 6.86 (d, J=8.9 Hz, 2H), 4.99 (s, 2H), 4.63 (dd, J=4.6, 8.3 Hz, 1H), 3.52-3.29 (m, 4H), 2.73 (t, J=7.4 Hz, 2H), 1.63-1.22 (m, 12H); low resolution mass spectrum (ES) m/e 439 [(M+H)⁺, calcd for C₂₅H₃₅N₄O₃: 438.6]; 99% purity based on HPLC.

### Example 291

### (S)-6-AMINO-2-[3-(4-BENZYLOXY-PHENYL)-UREIDO]-HEXANOIC ACID ISOPROPYLAMIDE

The title compound was prepared as described in the method of Example 2 with an initial coupling to Isopropylamine. ¹H NMR (400 MHz, DMSO-d6) δ 7.39-7.22 (m, 5H), 7.19 (d, J=8.9 Hz, 2H), 6.84 (d, J=8.9 Hz, 2H), 4.97 (s, 2H), 4.06 (dd, J=5.9, 7.4 Hz, 1H), 3.77 (m, 1H), 2.70 (t, J=7.6 Hz, 2H), 1.61-1.12 (m, 6H), 1.00 (t, J=6.3 Hz, 6H); low resolution mass spectrum (ES) m/e 413 [(M+H)⁺, calcd for C₂₃H₃₃N₄O₃: 412]; 97% purity based on HPLC.

### Example 292

### (S)-6-AMINO-2-(3-{4-[(METHYL-P-TOLYL-AMINO)-METHYL]-PHENYL}-UREIDO)-HEXANOIC ACID P-TOLYLAMIDE

The title compound was prepared as described in the method of Example 251. ¹H NMR (500 MHz, DMSO-d6) δ 10.49 (s, 1H), 9.30(br, 2H), 8.13 (s, 1H), 7.71 (br s, 3H), 7.46 (dd, J=2.68, 8.57 Hz, 4H), 7.29 (d, J=8.56 Hz, 2H), 7.22-7.15 (m, 6H), 4.06 (br s, 2H), 3.83 (br s, 1H), 3.22 (s, 3H), 2.77-2.73 (m, 2H), 2.32 (s, 3H), 2.27 (s, 3H), 1.95-1.24 (m, 6H); low resolution mass spectrum (ES) m/e 488 [(M+H)⁺, calcd for C₂₉H₃₈N₅O₂: 488]; 91.7% purity based on HPLC.

### Example 293

### (4-AMINO-BUTYL)-(P-TOLYLCARBAMOYL-METHYL)-CARBAMIC ACID 9H-FLUOREN-9-YLMETHYL ESTER

[(4-tert-Butoxycarbonylamino-butyl)-(9H-fluoren-9-ylmethoxycarbonyl)-amino]-acetic acid was coupled to 4-t-butyl phenylamine as described in the method of Example 6. Purification by HPLC produced the title compound. ¹H NMR (400 MHz, DMSO-d6) δ 10.04 (s, 0.54H); 9.88 (s, 0.43H); 7.91 (d, J=7.44 Hz, 0.85H); 7.84 (d, J=7.59 Hz, 1.05H); 7.71-7.61 (m, 2.75H); 7.59 (d, J=7.52Hz, 1H); 7.53 (d, J=8.34 Hz, 1H); 7.47-7.40 (m, 1.62H); 7.35 (dd, J=7.28, 14.6 Hz, 1.85H); 7.18-7.05 (m, 3H); 4.45 (d, J=5.60 Hz, 1H); 4.31 (t, J=5.53Hz, 0.63H); 4.24-4.07 (m, 3H); 3.93 (br s, 1H); 3.03 (t, J=6.98Hz, 1.23H); 2.89-2.72 (m, 1.2H); 2.71-2.59 (m, 0.98H); 2.27 (s, 1.68H); 1.37 (s, 1.37H); 1.63-1.41 (m, 2H); 1.40-1.16 (m, 2H). Low resolution mass spectrum (ES) m/e 458 [(M+H)⁺, calcd for C₂₈H₃₂N₃O₃: 458]; 100% purity based on HPLC.

### Example 294

### [5-AMINO-1-(6-METHYL-PYRIDIN-3-YLCARBAMOYL)-PENTYL]-CARBAMIC ACID 9H-FLUOREN-9-YLMETHYL ESTER

The title compound was prepared as described in the method of Example 6 with an initial coupling to 6-Methyl-pyridin-3-ylamine. 1H NMR (400 MHz, DMSO-d6) δ 10.04 (s, 0.59H); 9.87 (s, 0.41H); 7.90 (d, J=7.46 Hz, 0.83H); 7.83 (d, J=7.57 Hz, 1H); 7.73-7.59 (m, 3H); 7.57 (d, J=7.51 Hz, 1H); 7.53 (d, J=8.38 Hz, 1H); 7.42 (t, J=7.58, 1.42H); 7.39-7.27 (m, 1.63H); 7.23-6.99 (m, 2.52H); 4.44 (d, J=5.59 Hz, 0.83H); 4.30 (t, J=5.28 Hz, 0.54H); 4.23-4.06 (m, 2.46H); 3.92 (br s, 0.86H); 3.02 (t, J=6.81 Hz, 1H); 2.87-2.74 (m, 1 H); 2.70-2.57 (m, 1H); 1.86-1.62 (m, 4 H); 1.62-1.47 (m, 2H); 1.47-1.1 (m, 6H); Low resolution mass spectrum (ES) m/e 526.25 [(M+H)⁺, calcd for C₃₃H₃₉N₃O₃: 526.30]; 99.5% purity based on HPLC.

### Example 295

### (4-AMINO-BUTYL)-[(2-METHYL-1H-INDOL-5-YLCARBAMOYL)-METHYL]-CARBAMIC ACID 9H-FLUOREN-9-YLMETHYL ESTER

[(4-tert-Butoxycarbonylamino-butyl)-(9H-fluoren-9-ylmethoxycarbonyl)-amino]-acetic acid was coupled to 4-t-butyl phenylamine as described in the method of Example 6. Purification by HPLC produced the title compound. ¹H NMR (400 MHz, DMSO-d6) δ 10.82 and 10.79 (two singlets, 1H); 9.86 (s, 0.58H); 9.67 (s, 0.45H); 7.91 (d, J=7.47Hz, 1H); 7.83 (d, J=7.53Hz, 1H); 7.71-7.52 (m, 5H); 7.43 (t, J=7.36Hz, 1H); 7.39-7.28 (m, 2H); 7.23-7.01 (m, 3H); 6.04 (d, J=15.1Hz, 1H); 4.44 (d, J=5.59Hz, 1H); 4.30 (t, J=5.33 Hz, 0.68H); 4.18 (br s, 1.64H); 4.13 (br s, 1.36H); 3.93 (br s, 1H); 3.041 (t, J=6.85Hz, 1H); 2.88-2.76 (m, 1H);2.73-2.58 (m, 1H); 2.35 (s, 1.74H); 2.33 (s, 1.30H); 1.57 (br s, 2H); 2.12 (m, 2H). Low resolution mass spectrum (ES) m/e 497 [(M+H)⁺, calcd for C₃₀H₃₃N40₃: 497]; 97.4% purity based on HPLC.

### Example 296

### (S)-5-(2-AMINO-ACETYLAMINO)-2-[3-(4-BENZYLOXY-PHENYL)-UREIDO]-PENTANOIC ACID (2-METHYL-1H-INDOL-5-YL)-AMIDE

1H-Indol-5-ylamine was coupled to (S)-5-tert-Butoxycarbonylamino-2-(9H-fluoren-9-ylmethoxycarbonylamino)-pentanoic acid as described in the method of Example 6. Coupling to tert-Butoxycarbonylamino-acetic acid, and then to 1-Benzyloxy-4-isocyanato-benzene as described in the method of Example 2 provided the title compound. ¹H NMR (400 MHz, DMSO-d6) δ 7.67 (d, J=2.2 Hz, 1H), 7.43-7.27 (m, 6H), 7.24 (d, J=8.8 Hz, 2H), 7.17 (d, J=8.8 Hz, 1H), 7.09 (dd, J=2.2, 8.3 Hz, 1H), 6.87 (d, J=8.8 Hz, 2H), 5.00 (s, 2H), 4.35 (dd, J=5.9, 7.5 Hz, 1H), 3.47 (s, 2H), 3.13 (t, J=7.2, 6.4 Hz, 2H), 2.32 (s, 3H), 1.79-1.39 (m, 4H); low resolution mass spectrum (ES) m/e 543 [(M+H)⁺, calcd for C₃₀H₃₅N₆O₄: 543]; 93% purity based on HPLC.

### Example 297

### (S)-5-(2-AMINO-ACETYLAMINO)-2-[3-(9H-FLUOREN-2-YL)-UREIDO]-PENTANOIC ACID (2-METHYL-1H-INDOL-5-YL)-AMIDE

1H-Indol-5-ylamine was coupled to (S)-5-tert-Butoxycarbonylamino-2-(9H-fluoren-9-ylmethoxycarbonylamino)-pentanoic acid as described in the method of Example 6. Coupling to tert-Butoxycarbonylamino-acetic acid, and then to 2-Isocyanato-9H-fluorene as described in the method of Example 2 provided the title compound. ¹H NMR (400 MHz, DMSO-d6) δ 7.77-7.66 (m, 4H), 7.50 (d, J=7.5 Hz, 1H), 7.35-7.27 (m, 2H), 7.22 (d, J=7.5 Hz, 1H), 7.17 (d, J=8.8 Hz, 1H), 7.11 (dd, J=2.0, 8.3 Hz, 1H), 4.41 (dd, J=5.9, 7.5 Hz, 1H), 3.83 (s, 2H), 3.48 (s, 2H), 3.16 (t, J=7.5, 7.0 Hz, 2H), 2.33 (s, 3H), 1.81-1.43 (m, 4H); low resolution mass spectrum (ES) m/e 525 [(M+H)⁺, calcd for C₃₀H₃₃N₆O₃: 525]; 91% purity based on HPLC.

### Example 298

### (S)-6-(2-AMINO-ACETYLAMINO)-2-[3-(9H-FLUOREN-2-YL)-UREIDO]-HEXANOIC ACID (2-METHYL-1H-INDOL-5-YL)-AMIDE

1H-Indol-5-ylamine was coupled to (S)-6-tert-Butoxycarbonylamino-2-(9H-fluoren-9-ylmethoxycarbonylamino)-hexanoic acid as described in the method of Example 6. Coupling to tert-Butoxycarbonylamino-acetic acid, and then to 2-Isocyanato-9H-fluorene as described in the method of Example 2 provided the title compound. ¹H NMR (400 MHz, DMSO-d6) δ 7.77-7.66 (m, 4H), 7.50 (d, J=7.2 Hz, 1H), 7.35-7.27 (m, 3H), 7.24-7.15 (m, 2H), 7.11 (dd, J=2.2, 8.6 Hz, 1H), 4.39 (dd, J=5.5, 7.9 Hz, 1H), 3.84 (s, 2H), 3.47 (s, 2H), 3.11 (t, J=6.8 Hz, 2H), 2.33 (s, 3H), 1.82-1.27 (m, 6H); low resolution mass spectrum (ES) m/e 539 [(M+H)⁺, calcd for C₃₁H₃₄N₆O₃: 539]; 90% purity based on HPLC.

### Example 299

### (S)-6-AMINO-2-{3-[4-(PYRIDIN-2-YLMETHOXY)-PHENYL]-UREIDO}-HEXANOIC ACID P-TOLYLAMIDE

The title compound was prepared as described in the method of Example 262 with a final coupling to 2-Bromomethyl-pyridine. ¹H NMR (400 MHz, DMSO-d6) δ 10.07 (s, 1H), 8.60-8.57 (m, 2H), 7.90-7.86 (m, 1H), 7.66 (br s, 3H), 7.57-7.53 (m, 1H), 7.49 (d, J=8.41 Hz, 2H), 7.40-7.37 (m, 1H), 7.29 (d, J=9.00 Hz, 2H), 7.11 (d, J=8.45 Hz, 2H), 6.91 (d, J=9.05 Hz, 2H), 6.45 (br, 1H), 5.13 (s, 2H), 4.37 (dd, J=7.90, 13.50 Hz, 1H), 2.82-2.74 (m, 2H), 2.25 (s, 3H), 1.77-1.68 (m, 1H), 1.64-1.30 (m, 5H); low resolution mass spectrum (ES) m/e 462 [(M+H)⁺, calcd for C₂₆H₃₂N₅O₃: 461]; 88.7% purity based on HPLC.

### Example 300

### (S)-6-AMINO-2-[3-(4-METHOXY-PHENYL)-UREIDO]-HEXANOIC ACID P-TOLYLAMIDE

The title compound was prepared as described in the method of Example 2 with a final coupling to 1-Isocyanato-4-methoxy-benzene. ¹H NMR (400 MHz, DMSO-d6) δ 10.06 (s, 1H), 8.57 (s, 1H), 7.72 (br s, 3H), 7.50 (d, J=8.43 Hz, 2H), 7.28 (d, J=9.05 Hz, 2H), 7.11 (d, J=8.37 Hz, 2H), 6.81 (d, J=9.06 Hz, 2H), 6.46 (d, J=8.26 Hz, 1H), 4.37 (dd, J=8.04, 13.53 Hz, 1H)2.82-2.74 (m, 2H), 2.25 (s, 3H), 1.77-1.68 (m, 1H), 1.64-1.51 (m, 3H), 1.46-1.30 (m, 2H); low resolution mass spectrum (ES) m/e 385 [(M+H)⁺, calcd for C₂₁H₂₉N₄O₃: 385]; 95.3% purity based on HPLC.

### Example 301

### (S)-6-AMINO-2-{3-[4-(4-METHYL-BENZYLOXY)-PHENYL]-UREIDO}-HEXANOIC ACID P-TOLYLAMIDE

The title compound was prepared as described in the method of Example 248 with a final coupling to 1-Bromomethyl-4-methyl-benzene. ¹H NMR (500 MHz, DMSO-d6) δ 10.03 (s, 1H), 8.98 (s, 1H), 8.29 (s, 1H), 7.64 (br s, 3H), 7.48 (d, J=8.42 Hz, 2H), 7.11 (d, J=8.49 Hz, 2H), 7.08-7.03 (m, 5H), 6.90 (d, J=2.61 Hz, 1H), 6.66 (d, J=8.61 Hz, 1H), 6.29 (d, J=8.33 Hz, 1H)4.33 (dd, J=8.04, 13.67 Hz, 1H), 3.75 (s, 2H), 2.80-2.73 (m, 2H), 2.25 (s, 3H), 2.24 (s, 3H), 1.73-1.66 (m, 1H), 1.59-1.50 (m, 3H), 1.40-1.29 (m, 2H); low resolution mass spectrum (ES) m/e 475 [(M+H)⁺, calcd for C₂₈H₃₅N₄O₃: 475]; 98.1% purity based on HPLC.

### Example 302

### (S)-6-AMINO-2-[3-(4-BENZYLOXY-PHENYL)-UREIDO]-HEXANOIC ACID (FURAN-2-YLMETHYL)-AMIDE

The title compound was prepared as described in the method of Example 2 with an initial coupling to C-Furan-2-yl-methylamine. ¹H NMR (400 MHz, DMSO-d6) δ 7.54 (br s, 1H), 7.43-7.27 (m, 5H), 7.23 (d, J=8.6 Hz, 2H), 6.88 (d, J=9.0 Hz, 2H), 5.01 (s, 2H), 4.29-4.24 (br, 2H), 4.19 (dd, J=5.7, 7.7 Hz, 1H), 2.73 (t, J=7.7 Hz, 2H), 1.69-1.41 (m, 4H), 1.36-1.19 (m, 2H), low resolution mass spectrum (ES) m/e 451 [(M+H)⁺, calcd for C₂₅H₃₁N₄O₄: 450]; 100% purity based on HPLC.

### Example 303

### (S)-6-AMINO-2-[3-(4-BENZYLOXY-PHENYL)-UREIDO]-HEXANOIC ACID BENZOTHIAZOL-2-YLAMIDE

The title compound was prepared as described in the method of Example 2 with an initial coupling to Benzothiazol-2-ylamine. ¹H NMR (400 MHz, DMSO-d6) δ 7.96 (d, J=7.2 Hz, 1H), 7.74 (d, J=7.2 Hz, 1H), 7.48-7.21 (m, 10H), 6.88 (d, J=9.0 Hz, 2H), 5.01 (s, 2H), 4.49 (dd, J=5.9, 7.9 Hz, 1H), 2.77 (t, J=7.7 Hz, 2H), 1.87-1.49 (m, 4H), 1.48-1.29 (m, 2H); low resolution mass spectrum (ES) m/e 504 [(M+H)⁺, calcd for C₂₇H₃₀N₅O₃S: 503]; 92% purity based on HPLC.

### Example 304

### (S)-N-(4-AMINOMETHYL-BENZYL)-2-[3-(4-BENZYLOXY-PHENYL)-UREIDO]-2-PHENYL-ACETAMIDE

(S)-[(9H-Fluoren-9-ylmethoxycarbonylamino)]-phenyl-acetic acid was coupled to (4-Aminomethyl-benzyl)-carbamic acid tert-butyl ester, and then to 1-Benzyloxy-4-isocyanato-benzene as described in the method of Example 2. Purification by HPLC produced the title compound. ¹H NMR (400 MHz, DMSO-d6) δ 8.91 (t, J=5.7 Hz, 1H), 7.44-7.26 (m, 12H), 7.23 (d, J=9.4 Hz, 2H), 7.16 (d, J=8.3 Hz, 2H), 6.87 (d, J=8.8 Hz, 2H), 5.35 (s, 1H), 5.00 (s, 2H), 4.28 (d, J=5.5 Hz, 2H), 3.95 (s, 2H); low resolution mass spectrum (ES) m/e 495 [(M+H)⁺, calcd for C₃₀H₃₁N₄O₃: 495]; 90% purity based on HPLC.

### Example 305

### (S)-NR-(4-AMINOMETHYL-PHENYL)-2-[3-(4-BENZYLOXY-PHENYL)-UREIDO]-2-PHENYL-ACETAMIDE

(S)-[(9H-Fluoren-9-ylmethoxycarbonylamino)]-phenyl-acetic acid was coupled to (4-Amino-benzyl)-carbamic acid tert-butyl ester, and then to 1-Benzyloxy-4-isocyanato-benzene as described in the method of Example 2. Purification by HPLC produced the title compound. ¹H NMR (400 MHz, DMSO-d6) δ 10.51 (s, 1H), 7.59 (d, J=6.4 Hz, 2H), 7.46 (d, J=7.5 Hz, 2H), 7.42-7.26 (m, 10H), 7.23 (d, J=9.2 Hz, 2H), 6.87 (d, J=9.2 Hz, 2H), 5.48 (s, 1H), 5.00 (s, 2H), 3.93 (s, 2H); low resolution mass spectrum (ES) m/e 481 [(M+H)⁺, calcd for C₂₉H₂₉N₄O₃: 481]; 99% purity based on HPLC.

### Example 306

### (S)-3-(2-AMINO-ACETYLAMINO)-2-[3-(4-BENZYLOXY-PHENYL)-UREIDO]-N-(4-METHYL-CYCLOHEXYL)-PROPIONAMIDE

4-Methyl-cyclohexylamine was coupled to 3-tert-Butoxycarbonylamino-2-(9H-fluoren-9-ylmethoxycarbonylamino)-propionic acid as described in the method of Example 6. Coupling to tert-Butoxycarbonylamino-acetic acid, and then to 1-Benzyloxy-4-isocyanato-benzene as described in the method of Example 2 produced the title compound. ¹H NMR (400 MHz, DMSO-d6) δ 8.59 (s, 1H); 8.23 (t, J=5.60 Hz, 1H); 7.93 (br s, 3H); 7.45-7.21 (m, 6H); 6.66 (d, J=9 Hz, 2H); 6.34 (d, J=8Hz, 1H); 5.00 (s, 2H); 4.37 (dd, J=6.03; 13.87 Hz; 1H); 3.8 (br s, 1H); 3.47 (br s, 2H); 3.38 (dddd, J=7.56, 13.62; 13.38, 2H); 1.79-1.11 (m, 9H); 0.87 (d, J=6.6 Hz, 3H); low resolution mass spectrum (ES) m/e 492 [(M+H)⁺, calcd for C₂₆H₃₆N₅O₄: 482]; 95% purity based on HPLC.

### Example 307

### (S)-6-AMINO-2-[3-(4-BENZYLOXY-PHENYL)-UREIDO]-HEXANOIC ACID (1H-INDOL-7-YL)-AMIDE

The title compound was prepared as described in the method of Example 2 with an initial coupling reaction to 1H-Indol-7-ylamine. ¹H NMR (400 MHz, DMSO-d6) δ 10.69 (s, 1H); 9.90 (s, 1H); 8.57 (s, 1H); 7.65 (br s, 3H); 7.56-7.10 (m, 12H); 6.94 (t, 2H, J=7.8Hz); 6.89 (d, 2H, J=8.9Hz); 6.50 (d, 1H, J=7.9Hz); 6.44 (br s, 1H); 5.02 (s, 2H); 4.52 (dd, 1H, J=7.3; 13.3Hz); 2.79 (br s, 2H); 1.91-1.28 (m, 6H); low resolution mass spectrum (ES) m/e 486 [(M+H)⁺, calcd for C₂₈H₃₂N₅O₃: 485]; 87% purity based on HPLC.

### Example 308

### (±)-4-(2-AMINO-ACETYL)-PIPERAZINE-1,2-DICARBOXYLIC ACID 1-[(4-BENZYLOXY-PHENYL)-AMIDE]2-P-TOLYLAMIDE

Piperazine-1,2-dicarboxylic acid 1-(9H-fluoren-9-ylmethyl) ester was coupled to 4-methyl phenylamine, and then to tert-Butoxycarbonylamino-acetic acid as described in the method of Example 26. Purification by HPLC produced the title compound. ¹H NMR (400 MHz, DMSO-d6) δ 9.95 (d, J=7.5 Hz, 1H), 7.51-7.26 (m, 9H), 7.10 (dd, J=8.8, 11.4 Hz, 2H), 6.91 (dd, J=6.3, 9.0 Hz, 2H ), 5.04 (s, 2H), 4.96-4.91 (br, 0.5 H), 4.86-4.80 (br, 0.5H), 4.72 (d, J=13.1 Hz, 0.5H), 4.25-3.60 (m, 5H), 3.32-3.20 (m, 2H), 3.05-2.92 (m, 1H), 2.25 (s, 1.5H), 2.23 (s, 1.5H); low resolution mass spectrum (ES) m/e 502 [(M+H)⁺, calcd for C₂₈H₃₂N₅O₄: 502]; 100% purity based on HPLC.

### Example 309

### (S)-2-[3-(4-BENZYLOXY-PHENYL)-UREIDO]-6-HYDROXY-HEXANOIC ACID P-TOLYLAMIDE

The title compound was prepared as described in the method of Example 2 with an initial coupling to (S)-2-(9H-Fluoren-9-ylmethoxycarbonylamino)-6-hydroxy-hexanoic acid. ¹H NMR (400 MHz, DMSO-d6) 10.00 (s, 1H), 8.46 (s, 1H), 7.48 (d, J=8.2 Hz, 2H), 7.44-7.23 (m, 7H), 7.09 (d, J=8.6 Hz, 2H), 6.88 (d, J=9.2 Hz, 2H), 6.33 (d, J=8.2 Hz, 1H), 5.02 (s, 2H), 4.35 (m, 1H), 3.37 (dd, J=6.3, 11.4 Hz, 2H), 2.24 (s, 3H), 1.75-1.25 (m, 6H); low resolution mass spectrum (ES) m/e 462 [(M+H)⁺, calcd for C₂₇H₃₂N₃O₄: 462]; 100% purity based on HPLC.

### Example 310

### [(2-AMINO-PHENYLCARBAMOYL)-METHYL]-CARBAMIC ACID 9H-FLUOREN-9-YLMETHYL ESTER

(9H-Fluoren-9-ylmethoxycarbonylamino)-acetic acid was coupled to (2-Amino-phenyl)-carbamic acid tert-butyl ester as described in the method of Example 6. Purification by HPLC provided the title compound. ¹H NMR (400MHz, DMSO-d₆) δ 9.19 (s, 1H), 7.90 (d, J=7.2 Hz, 2H), 7.73 (d, J=7.2Hz, 2H), 7.59 (t, J=5.8Hz, 1H), 7.43 (t, J=7.4Hz, 2H), 7.35(t, J=7.6Hz, 2H), 7.12 (d, J=8.0, 1H), 6.94 (t, J=7.7Hz, 1H), 6.73 (d, J=7.6Hz, 1H), 6.57 (t, J=7.0Hz, 1H), 4.33 (d, J=6.8Hz, 2H), 4.25 (t, J=6.8Hz, 1H); low resolution mass spectrum (ES) m/e 388 [(M+H)⁺, calcd for C₂₃H₂₂N₃O₃: 388]; 94% purity based on HPLC.

### Example 311

### {[2-(2-AMINO-ACETYLAMINO)-PHENYLCARBAMOYL]-METHYL}-CARBAMIC ACID 9H-FLUOREN-9-YLMETHYL ESTER

The compound of Example 310 was coupled to tert-Butoxycarbonylamino-acetic acid as described in the method of Example 6 to produce the title compound. ¹H NMR (400MHz, DMSO-d₆) δ 9.95 (s, 1H), 9.61 (s, 1H), 8.23 (br s, 3H), 7.91 (d, J=7.6Hz, 2H), 7.68-7.77 (m, 3H), 7.58-7.65 (m, 2H), 4.35 (d, J=7.2Hz, 2H), 4.27 (t, J=6.6 Hz, 1H), 3.81-3.93 (m, 4H); low resolution mass spectrum (ES) m/e 445 [(M+H)⁺, calcd for C₂₅H₂₅N₄O₄: 445]; 96% purity based on HPLC.

### Example 312

### {[2-(3-AMINO-PROPIONYLAMINO)-PHENYLCARBAMOYL]-METHYL}-CARBAMIC ACID 9H-FLUOREN-9-YLMETHYL ESTER

The compound of Example 310 was coupled to 3-tert-Butoxycarbonylamino-propionic acid as described in the method of Example 6 to produce the title compound. ¹H NMR (400MHz, DMSO-d₆) δ 9.78 (s, 1H), 9.52 (s, 1H), 7.88-7.98 (m, 5H), 7.71-7.77 (m, 3H), 7.63 (d, J=8.0Hz, 1H), 7.55 (d, J=8.0Hz, 1H), 7.44 (t, J=7.6Hz, 2H), 7.35 (t, J=7.4Hz, 2H), 7.15-7.21 (m, 2H), 4.35 (d, J=6.8Hz, 2H), 4.27 (t, J=6.6Hz, 1H), 3.88 (d, J=6.0 Hz, 2H), 3.07-3.14 (m, 2H), 2.80 (t, J=6.6Hz, 2H); low resolution mass spectrum (ES) m/e 459 [(M+H)⁺, calcd for C₂₆H₂₇N₄O₄: 459]; 94% purity based on HPLC.

### Example 313

### (S)-[(6-AMINO-HEXYLCARBAMOYL)-PHENYL-METHYL]-CARBAMIC ACID 9H-FLUOREN-9-YLMETHYL ESTER

### (S)-[(9H-Fluoren-9-ylmethoxycarbonylamino)]-phenyl-acetic acid was coupled to

(6-Amino-hexyl)-carbamic acid tert-butyl ester as described in the method of Example 6. Purification by HPLC produced the title compound. 1H NMR (400 MHz, DMSO-d6) δ 7.86 (d, J=7.5 Hz, 2H), 7.72 (d, J=7.5 Hz, 2H), 7.44-7.24 (m, 9H), 5.15 (s, 1H), 4.29-4.12 (m, 3H), 3.02 (m, 2H), 2.69 (t, J=7.9 Hz, 2H), 1.51-1.28 (m, 4H), 1.27-1.08 (m, 4H); Low resolution mass spectrum (ES) m/e 472.2 [(M+H)⁺, calcd for C₂₉H33N303: 472.6]; 98% purity based on HPLC.

### Example 314

### (S)-[(4-AMINOMETHYL-BENZYLCARBAMOYL)-PHENYL-METHYL]-CARBAMIC ACID 9H-FLUOREN-9-YLMETHYL ESTER

(S)-[(9H-Fluoren-9-ylmethoxycarbonylamino)]-phenyl-acetic acid was coupled to (4-Aminomethyl-benzyl)-carbamic acid tert-butyl ester as described in the method of Example 6. Purification by HPLC produced the title compound. 1H NMR (400 MHz, DMSO-d6) 7.87 (d, J=7.9 Hz, 2H), 7.74 (d, J=7.9 Hz, 2H), 7.48-7.24 (m, 11H), 7.15 (d, J=8.5 Hz, 2H), 5.24 (s, 1H), 4.33-4.13 (m, 5H), 3.95 (s, 2H); Low resolution mass spectrum (ES) m/e 492.2 [(M+H)⁺, calcd for C₃₁H29N303: 492.6]; 100% purity based on HPLC.

### Example 315

### (S)-[(4-AMINOMETHYL-PHENYLCARBAMOYL)-PHENYL-METHYL]-CARBAMIC ACID 9H-FLUOREN-9-YLMETHYL ESTER

(S)-[(9H-Fluoren-9-ylmethoxycarbonylamino)]-phenyl-acetic acid was coupled to (4-Amino-benzyl)-carbamic acid tert-butyl ester as described in the method of Example 6. Purification produced the title compound. 1H NMR (400 MHz, DMSO-d6) 10.41 (s, 1H), 7.87 (d, J=8.1 Hz, 2H), 7.74 (d, J=7.5 Hz, 2H), 7.59 (d, J=7.9 Hz, 2H), 7.50 (d, J=7.5 Hz, 2H), 7.43-7.25 (m, 9H), 5.38 (s, 1H), 4.29-4.16 (m, 3H), 3.94 (s, 2H); Low resolution mass spectrum (ES) m/e 478.3 [(M+H)⁺, calcd for C₃₀H27N303: 478.6]; 100% purity based on HPLC.

### Example 316

(S)-6-AMINO-2-[3-(4-BENZYLOXY-PHENYL)-UREIDO]-HEXANOIC ACID (5-METHYL-2-PHENYL-2H-PYRAZOL-3-YL)-AMIDE

The title compound was prepared as described in the method of Example 2 with an initial coupling to 5-Methyl-2-phenyl-2H-pyrazol-3-ylamine. ¹H NMR (400 MHz, DMSO-d6) 7.45-7.26 (m, 10H), 7.24 (d, J=9.0 Hz, 2H), 6.89 (d, J=9.0 Hz, 2H), 6.19 (s, 1H), 5.01 (s, 2H), 4.25 (dd, J=3.9, 8.1 Hz, 1H), 2.75-2.63 (m, 2H), 2.18 (s, 3H), 1.67-1.39 (m, 4H), 1.28-1.13 (m, 2H); Low resolution mass spectrum (ES) m/e 527.2 [(M+H)⁺, calcd for C₃₀H34N6O3: 527.6]; 100% purity based on HPLC.

### Example 317

### (S)-6-AMINO-2-[3-(4-BENZYLOXY-PHENYL)-UREIDO]-HEXANOIC ACID (4-IMIDAZOL-1-YL-PHENYL)-AMIDE

The title compound was prepared as described in the method of Example 2 with an initial coupling to 4-Imidazol-1-yl-phenylamine. 1H NMR (400 MHz, DMSO-d6) 9.34 (s, 1H), 8.09 (s, 1H), 7.81 (d, J=8.8 Hz, 2H), 7.75-7.67 (m, 3H), 7.42-7.22 (m, 7H), 6.88 (d, J=9.0 Hz, 2H), 5.01 (s, 2H), 4.34 (dd, J=5.7, 8.3 Hz, 1H), 2.76 (t, J=7.0 Hz, 2H), 1.82-1.25 (m, 6H); Low resolution mass spectrum (ES) m/e 513.2 [(M+H)⁺, calcd for C₂₉H32N603: 513.6]; 100% purity based on HPLC.

### Example 318

### (S)-l -[5-AMINO-1-(MORPHOLINE-4-CARBONYL)-PENTYL]-3-(4-BENZYLOXY-PHENYL)-UREA

The title compound was prepared as described in the method of Example 2 with an initial coupling to Morpholine. 1H NMR (400 MHz, DMSO-d6) 7.43-7.18 (m, 7H), 6.87 (d, J=8.8 Hz, 2H), 5.00 (s, 2H), 4.62 (dd, J=4.6, 8.6 Hz, 0.8H), 4.13 (dd, J=5.0, 8.3 Hz, 0.4H), 3.58-3.36 (m, 8H), 2.79-2.69 (m, 2H), 1.68-1.19 (m, 6H); Low resolution mass spectrum (ES) m/e 441.2 [(M+H)⁺, calcd for C₂₄H32N404: 441.5]; 75% purity based on HPLC.

### Example 319

### (S)-NR-(6-AMINO-HEXYL)-2-[3-(4-BENZYLOXY-PHENYL)-UREIDO]-2-PHENYL-ACETAMIDE

(S)-[(9H-Fluoren-9-ylmethoxycarbonylamino)]-phenyl-acetic acid was coupled to (6-Amino-hexyl)-carbamic acid tert-butyl ester then coupled to 1-Benzyloxy-4-isocyanato-benzene as described in the method of Example 2. Purification produced the title compound. 1H NMR (400 MHz, DMSO-d6) 7.46-7.17 (m, 12H), 6.86 (d, J=8.8 Hz, 2H), 5.26 (s, 1H), 5.00 (s, 2H), 3.02 (dd, J=7.0, 12.1 Hz, 2H), 2.69 (t, J=7.7 Hz, 2H), 1.49-1.10 (m, 8H); Low resolution mass spectrum (ES) m/e 475.1 [(M+H)⁺, calcd for C₂₈H34N403: 474.6]; 97% purity based on HPLC.

### Example 320

### (R)-NR-(6-AMINO-HEXYL)-2-[3-(4-BENZYLOXY-PHENYL)-UREIDO]-2-PHENYL-ACETAMIDE

(R)-[(9H-Fluoren-9-ylmethoxycarbonylamino)]-phenyl-acetic acid was coupled to (6-Amino-hexyl)-carbamic acid tert-butyl ester then coupled to 1-Benzyloxy-4-isocyanato-benzene as described in the method of Example 2. Purification produced the title compound. 1H NMR (400 MHz, DMSO-d6) 7.42-7.19 (m, 12H), 6.87 (d, J=8.8 Hz, 2H), 5.27 (s, 1H), 5.00 (s, 2H), 3.09-2.96 (m, 2H), 2.75-2.63 (m, 2H), 1.51-1.28 (m, 4H), 1.28-1.08 (m, 4H); Low resolution mass spectrum (ES) m/e 475.1 [(M+H)⁺, calcd for C₂₈H34N403: 474.6]; 99% purity based on HPLC.

### Example 321

### (S)-6-AMINO-2-[3-(4-BENZYLOXY-PHENYL)-UREIDO]-HEXANOIC ACID INDAN-4-YLAMIDE

The title compound was prepared as described in the method of Example 2 with an initial coupling to Indan-4-ylamine. 1H NMR (400 MHz, DMSO-d6) 7.96 (d, J=7.2 Hz, 1H), 7.74 (d, J=7.2 Hz, 1H), 7.48-7.21 (m, 10H), 6.88 (d, J=9.0 Hz, 2H), 5.01 (s, 2H), 4.49 (dd, J=5.9, 7.9 Hz, 1H), 2.77 (t, J=7.7 Hz, 2H), 1.87-1.49 (m, 4H), 1.48-1.29 (m, 2H); Low resolution mass spectrum (ES) m/e 487.1 [(M+H)⁺, calcd for C₂₉H34N403: 487.6]; 92% purity based on HPLC.

### Example 322

### (S)-5-(2-AMINO-ACETYLAMINO)-2-[3-(4-BENZYLOXY-PHENYL)-UREIDO]-PENTANOIC ACID P-TOLYLAMIDE

4-Methyl-phenylamine was coupled to 5-tert-Butoxycarbonylamino-2-(9H-fluoren-9-ylmethoxycarbonylamino)-pentanoic acid as described in the method of Example 6. Coupling to tert-Butoxycarbonylamino-acetic acid, and then to 1-Benzyloxy-4-isocyanato-benzene as described in the method of Example 2 produced the title compound. 1H NMR (400 MHz, DMSO-d6) δ 7.45 (d, J=8.3 Hz, 2H), 7.42-7.26 (m, 5H), 7.24 (d, J=9.3 Hz, 2H), 7.10 (d, J=8.1 Hz, 2H), 6.87 (d, J=9.3 Hz, 2H), 5.00 (s, 2H), 4.33 (t, J=7.5 Hz, 1H), 3.47 (s, 2H), 3.13 (t, J=6.8 Hz, 2H), 2.23 (s, 3H), 1.78-1.35 (m, 4H); Low resolution mass spectrum (ES) m/e 504.1 [(M+H)⁺, calcd for C₂₈H₃₃N₅O₄: 504.6]; 97% purity based on HPLC.

### Example 323

### 2-[1-(4-AMINO-BUTYL)-3-(4-BENZYLOXY-PHENYL)-UREIDO]-N-(4-CYCLOHEXYL-PHENYL)-ACETAMIDE

[(4-tert-Butoxycarbonylamino-butyl)-(9H-fluoren-9-ylmethoxycarbonyl)-amino]-acetic acid was coupled to 4-cyclohexyl phenylamine then to 1-Benzyloxy-4-isocyanato-benzene as described in the method of Example 2. Purification by HPLC produced the title compound. Low resolution mass spectrum (ES) m/e 529 [(M+H)⁺, calcd for C₃₂H₄₁N₄O₃: 529]; 98% purity based on HPLC.

### Example 324

### 2-[1-(4-AMINO-BUTYL)-3-(4-STYRYL-PHENYL)-UREIDO]-N-(4-CYCLOHEXYL-PHENYL)-ACETAMIDE

[(4-tert-Butoxycarbonylamino-butyl)-(9H-fluoren-9-ylmethoxycarbonyl)-amino]-acetic acid was coupled to 4-cyclohexyl phenylamine then to 1-Isocyanato-4-Styryl-benzene as described in the method of Example 2. Purification by HPLC produced the title compound. Low resolution mass spectrum (ES) m/e 525 [(M+H)⁺, calcd for C₃₃H₄₁N₄O₂: 525]; 99% purity based on HPLC.

### Example 325

### ({2-[2-(9H-FLUOREN-9-YLMETHOXYCARBONYLAMINO)-ACETYLAMINO]-BENZYLCARBAMOYL}-METHYL)-CARBAMIC ACID TERT-BUTYL ESTER

(9H-Fluoren-9-ylmethoxycarbonylamino)-acetic acid was coupled to (2-Amino-benzyl)-carbamic acid tert-butyl ester as described in the method of Example 6, and then coupled to tert-Butoxycarbonylamino-acetic acid as described for Intermediate #1 of Example 1. Purification by HPLC produced the title compound. Low resolution mass spectrum (ES) m/e 560 [(M+H)⁺, calcd for C₃₁H₃₅N₄O₆: 560]; 79% purity based on HPLC. SCHEME (II) COMPOUNDS

### Example 326

### (S)-6-AMINO-2-[2-(BIPHENYL-4-YLOXY)-ACETYLAMINO]-HEXANOIC ACID P-TOLYLAMIDE

Prepared by solid-phase as described in Scheme 2: Low resolution mass spectrum (ES) m/e 446 [(M+H)⁺, calcd for C₂₇H₃₂N₃O₃: 446]; 90% purity based on HPLC.

### Example 327

### (S)-6-AMINO-2-[2-(4-IODO-PHENOXY)-ACETYLAMINO]-HEXANOIC ACID P-TOLYLAMIDE

Prepared by solid-phase as described in Scheme 2: Low resolution mass spectrum (ES) m/e 496 [(M+H)⁺, calcd for C₂₁H₂₇IN₃O₃: 496]; 93% purity based on HPLC.

### Example 328

### (S)-⁶-AMINO-²-(²-PHENOXY-ACETYLAMINO)-HEXANOIC ACID P-TOLYLAMIDE

Prepared by solid-phase as described in Scheme 2. Low resolution mass spectrum (ES) m/e 370 [(M+H)⁺, calcd for C₂₁H₂₈N₃O₃: 470]; 97% purity based on HPLC.

### Example 329

### (S)-6-AMINO-2-[²-(⁴-FLUORO-PHENOXY)-ACETYLAMINO]-HEXANOIC ACID P-TOLYLAMIDE

Prepared by solid-phase as described in Scheme 2. Low resolution mass spectrum (ES) m/e 388 [(M+H)⁺, calcd for C₂₁H₂₇FN₃O₃: 388]; 98% purity based on HPLC.

### Example 330

### (S)-⁶-AMINO-2-[2-(³-CHLORO-PHENOXY)-ACETYLAMINO]-HEXANOIC ACID P-TOLYLAMIDE

Prepared by solid-phase as described in Scheme 2. Low resolution mass spectrum (ES) m/e 404 [(M+H)⁺, calcd for C₂₁H₂₇ClN₃O₃: 404]; 95% purity based on HPLC.

### Example 331

### (S)-6-AMINO-2-[2-(4-CHLORO-PHENOXY)-ACETYLAMINO]-HEXANOIC ACID P-TOLYLAMIDE

Prepared by solid-phase as described in Scheme 2. Low resolution mass spectrum (ES) m/e 404 [(M+H)⁺, calcd for C₂₁H₂₇ClN₃O₃: 404]; 98% purity based on HPLC.

### Example 332

### (S)-6-AMINO-2-[2-(3-TRIFLUOROMETHYL-PHENOXY)-ACETYLAMINO]-HEXANOIC ACID P-TOLYLAMIDE

Prepared by solid-phase as described in Scheme 2. Low resolution mass spectrum (ES) m/e 438 [(M+H)⁺, calcd for C₂₂H₂₇F₃N₃O₃: 438]; 94% purity based on HPLC.

### Example 333

### (S)-6-AMINO-2-[2-(4-PHENOXY-PHENOXY)-ACETYLAMINO]-HEXANOIC ACID P-TOLYLAMIDE

Prepared by solid-phase as described in Scheme 2. Low resolution mass spectrum (ES) m/e 462 [(M+H)⁺, calcd for C₂₇H₃₁N₃O₄: 462]; 88% purity based on HPLC.

### Example 334

### (S)-6-AMINO-2-(2-PHENETHYLAMINO-ACETYLAMINO)-HEXANOIC ACID P-TOLYLAMIDE

Prepared by solid-phase as described in Scheme 2. Low resolution mass spectrum (ES) m/e 397 [(M+H)⁺, calcd for C₂₃H₃₃N₄O₂: 397]; 85% purity based on HPLC.

### Example 335

### (S)-6-AMINO-2-{2-[2-(1H-INDOL-3-YL)-ETHYLAMINO]-ACETYLAMINO}-HEXANOIC ACID P-TOLYLAMIDE

Prepared by solid-phase as described in Scheme 2. Low resolution mass spectrum (ES) m/e 436 [(M+H)⁺, calcd for C₂₅H₃₄N₅O₂: 436]; 78% purity based on HPLC.

### Example 336

### (S)-6-AMINO-2-[2-(INDAN-2-YLAMINO)-ACETYLAMINO]-HEXANOIC ACID P-TOLYLAMIDE

Prepared by solid-phase as described in Scheme 2. Low resolution mass spectrum (ES) m/e 409 [(M+H)⁺, calcd for C₂₄H₃₃N₄O₂: 409]; 93% purity based on HPLC.

### Example 337

### (S)-6-AMINO-2-(2-BENZYLAMINO-ACETYLAMINO)-HEXANOIC ACID P-TOLYLAMIDE

Prepared by solid-phase as described in Scheme 2. Low resolution mass spectrum (ES) m/e 383 [(M+H)⁺, calcd for C₂₂H₃₁N₄O₂: 383]; 81% purity based on HPLC.

### Example 338

### (S)-6-AMINO-2-[2-(2-METHYL-BENZYLAMINO)-ACETYLAMINO]-HEXANOIC ACID P-TOLYLAMIDE

Prepared by solid-phase as described in Scheme 2. Low resolution mass spectrum (ES) m/e 397 [(M+H)⁺, calcd for C₂₃H₃₃N₄O₂: 397]; 91% purity based on HPLC.

### Example 339

### (S)-6-AMINO-2-[2-(3-METHYL-BENZYLAMINO)-ACETYLAMINO]-HEXANOIC ACID P-TOLYLAMIDE

Prepared by solid-phase as described in Scheme 2. Low resolution mass spectrum (ES) m/e 397 [(M+H)⁺, calcd for C₂₃H₃₃N₄O₂: 397]; 86% purity based on HPLC.

### Example 340

### (S)-6-AMINO-2-[2-(4-METHYL-BENZYLAMINO)-ACETYLAMINO]-HEXANOIC ACID P-TOLYLAMIDE

Prepared by solid-phase as described in Scheme 2. Low resolution mass spectrum (ES) m/e 397 [(M+H)⁺, calcd for C₂₃H₃₃N₄O₂: 397]; 88% purity based on HPLC.

### Example 341

### (S)-6-AMINO-2-[2-(2-METHOXY-BENZYLAMINO)-ACETYLAMINO]-HEXANOIC ACID P-TOLYLAMIDE

Prepared by solid-phase as described in Scheme 2. Low resolution mass spectrum (ES) m/e 413 [(M+H)⁺, calcd for C₂₃H₃₃N₄O₃: 413]; 83% purity based on HPLC.

### Example 342

### (S)-6-AMINO-2-[2-(3-METHOXY-BENZYLAMINO)-ACETYLAMINO]-HEXANOIC ACID P-TOLYLAMIDE

Prepared by solid-phase as described in Scheme 2. Low resolution mass spectrum (ES) m/e 413 [(M+H)⁺, calcd for C₂₃H₃₃N₄O₃: 413]; 82% purity based on HPLC.

### Example 343

### (S)-6-AMINO-2-[2-(4-METHOXY-BENZYLAMINO)-ACETYLAMINO]-HEXANOIC ACID P-TOLYLAMIDE

Prepared by solid-phase as described in Scheme 2. Low resolution mass spectrum (ES) m/e 413 [(M+H)⁺, calcd for C₂₃H₃₃N₄O₃: 413]; 86% purity based on HPLC.

### Example 344

### (S)-6-AMINO-2-[2-(2-FLUORO-BENZYLAMINO)-ACETYLAMINO]-HEXANOIC ACID P-TOLYLAMIDE

Prepared by solid-phase as described in Scheme 2. Low resolution mass spectrum (ES) m/e 401 [(M+H)⁺, calcd for C₂₂H₃₀FN₄O₂: 401]; 90% purity based on HPLC.

### Example 345

### (S)-6-AMINO-2-[2-(3-FLUORO-BENZYLAMINO)-ACETYLAMINO]-HEXANOIC ACID P-TOLYLAMIDE

Prepared by solid-phase as described in Scheme 2. Low resolution mass spectrum (ES) m/e 401 [(M+H)⁺, calcd for C₂₂H₃₀FN₄O₂: 401]; 85% purity based on HPLC.

### Example 346

### (S)-6-AMINO-2-[2-(4-FLUORO-BENZYLAMINO)-ACETYLAMINO]-HEXANOIC ACID P-TOLYLAMIDE

Prepared by solid-phase as described in Scheme 2. Low resolution mass spectrum (ES) m/e 401 [(M+H)⁺, calcd for C₂₂H₃₀FN₄O₂: 401]; 87% purity based on HPLC.

### Example 347

### (S)-6-AMINO-2-[2-(2-CHLORO-BENZYLAMINO)-ACETYLAMINO]-HEXANOIC ACID P-TOLYLAMIDE

Prepared by solid-phase as described in Scheme 2. Low resolution mass spectrum (ES) m/e 417 [(M+H)⁺, calcd for C₂₂H₃₀ClN₄O₂: 417]; 87% purity based on HPLC.

### Example 348

### (S)-6-AMINO-2-[2-(3-CHLORO-BENZYLAMINO)-ACETYLAMINO]-HEXANOIC ACID P-TOLYLAMIDE

Prepared by solid-phase as described in Scheme 2. Low resolution mass spectrum (ES) m/e 417 [(M+H)⁺, calcd for C₂₂H₃₀ClN₄O₂: 417]; 90% purity based on HPLC.

### Example 349

### (S)-6-AMINO-2-[2-(4-CHLORO-BENZYLAMINO)-ACETYLAMINO]-HEXANOIC ACID P-TOLYLAMIDE

Prepared by solid-phase as described in Scheme 2. Low resolution mass spectrum (ES) m/e 417 [(M+H)⁺, calcd for C₂₂H₃₀ClN₄O₂: 417]; 85% purity based on HPLC.

### Example 350

### (S)-6-AMINO-2-[2-(2-TRIFLUOROMETHYL-BENZYLAMINO)-ACETYLAMINO]-HEXANOIC ACID P-TOLYLAMIDE

Prepared by solid-phase as described in Scheme 2: Low resolution mass spectrum (ES) m/e 451 [(M+H)⁺, calcd for C₂₃H₃₀F₃N₄O₂: 451]; 87% purity based on HPLC.

### Example 351

### (S)-6-AMINO-2-[2-(3-TRIFLUOROMETHYL-BENZYLAMINO)-ACETYLAMINO]-HEXANOIC ACID P-TOLYLAMIDE

Prepared by solid-phase as described in Scheme 2: Low resolution mass spectrum (ES) m/e 451 [(M+H)⁺, calcd for C₂₃H₃₀F₃N₄O₂: 451]; 84% purity based on HPLC.

### Example 352

### (S)-6-AMINO-2-[2-(4-TRIFLUOROMETHYL-BENZYLAMINO)-ACETYLAMINO]-HEXANOIC ACID P-TOLYLAMIDE

Prepared by solid-phase as described in Scheme 2: Low resolution mass spectrum (ES) m/e 451 [(M+H)⁺, calcd for C₂₃H₃₀F₃N₄O₂: 451]; 86% purity based on HPLC.

### Example 353

### (S)-6-AMINO-2-[2-(BENZYL-METHYL-AMINO)-ACETYLAMINO]-HEXANOIC ACID P-TOLYLAMIDE

Prepared by solid-phase as described in Scheme 2: Low resolution mass spectrum (ES) m/e 397 [(M+H)⁺, calcd for C₂₃H₃₃N₄O₂: 397]; 95% purity based on HPLC.

### Example 354

### (S)-6-AMINO-2-[2-(CYCLOHEXYL-PHENYL-AMINO)-ACETYLAMINO]-HEXANOIC ACID P-TOLYLAMIDE

Prepared by solid-phase as described in Scheme 2: Low resolution mass spectrum (ES) m/e 451 [(M+H)⁺, calcd for C₂₇H₃₉N₄O₂: 451]; 90% purity based on HPLC.

### Example 355

### (S)-6-AMINO-2-(2-M-TOLYLAMINO-ACETYLAMINO)-HEXANOIC ACID P-TOLYLAMIDE

Prepared by solid-phase as described in Scheme 2: Low resolution mass spectrum (ES) m/e 383 [(M+H)⁺, calcd for C₂₂H₃₁N₄O₂: 383]; 95% purity based on HPLC.

### Example 356

### (S)-6-AMINO-2-(2-P-TOLYLAMINO-ACETYLAMINO)-HEXANOIC ACID P-TOLYLAMIDE

Prepared by solid-phase as described in Scheme 2: Low resolution mass spectrum (ES) m/e 383 [(M+H)⁺, calcd for C₂₂H₃₁N₄O₂: 383]; 89% purity based on HPLC.

### Example 357

### (S)-6-AMINO-2-[2-(2-METHOXY-PHENYLAMINO)-ACETYLAMINO]-HEXANOIC ACID P-TOLYLAMIDE

Prepared by solid-phase as described in Scheme 2: Low resolution mass spectrum (ES) m/e 399 [(M+H)⁺, calcd for C₂₂H₃₁N₄O₃: 399]; 84% purity based on HPLC.

### Example 358

### (S)-6-AMINO-2-[2-(3-METHOXY-PHENYLAMINO)-ACETYLAMINO]-HEXANOIC ACID P-TOLYLAMIDE

Prepared by solid-phase as described in Scheme 2: Low resolution mass spectrum (ES) m/e 399 [(M+H)⁺, calcd for C₂₂H₃₁N₄O₃: 399]; 87% purity based on HPLC.

### Example 359

### (S)-6-AMINO-2-[2-(2-FLUORO-PHENYLAMINO)-ACETYLAMINO]-HEXANOIC ACID P-TOLYLAMIDE

Prepared by solid-phase as described in Scheme 2: Low resolution mass spectrum (ES) m/e 387 [(M+H)⁺, calcd for C₂₁H₂₈FN₄O₂: 387]; 88% purity based on HPLC.

### Example 360

### (S)-6-AMINO-2-[2-(3-FLUORO-PHENYLAMINO)-ACETYLAMINO]-HEXANOIC ACID P-TOLYLAMIDE

Prepared by solid-phase as described in Scheme 2: Low resolution mass spectrum (ES) m/e 387 [(M+H)⁺, calcd for C₂₁H₂₈FN₄O₂: 387]; 92% purity based on HPLC.

### Example 361

### (S)-6-AMINO-2-[2-(4-FLUORO-PHENYLAMINO)-ACETYLAMINO]-HEXANOIC ACID P-TOLYLAMIDE

Prepared by solid-phase as described in Scheme 2: Low resolution mass spectrum (ES) m/e 387 [(M+H)⁺, calcd for C₂₁H₂₈FN₄O₂: 387]; 89% purity based on HPLC.

### Example 362

### (S)-6-AMINO-2-[2-(3-CHLORO-PHENYLAMINO)-ACETYLAMINO]-HEXANOIC ACID P-TOLYLAMIDE

Prepared by solid-phase as described in Scheme 2: Low resolution mass spectrum (ES) m/e 403 [(M+H)⁺, calcd for C₂₁H₂₈ClN₄O₂: 403]; 92% purity based on HPLC.

### Example 363

### (S)-6-AMINO-2-[2-(4-CHLORO-PHENYLAMINO)-ACETYLAMINO]-HEXANOIC ACID P-TOLYLAMIDE

Prepared by solid-phase as described in Scheme 2: Low resolution mass spectrum (ES) m/e 403 [(M+H)⁺, calcd for C₂₁H₂₈C₁N₄O₂: 403]; 90% purity based on HPLC.

### Example 364

### (S)-6-AMINO-2-[2-(3-TRIFLUOROMETHYL-PHENYLAMINO)-ACETYLAMINO]-HEXANOIC ACID P-TOLYLAMIDE

Prepared by solid-phase as described in Scheme 2: Low resolution mass spectrum (ES) m/e 437 [(M+H)⁺, calcd for C₂₂H₂₈F₃N₄O₂: 438]; 96% purity based on HPLC.

### Example 365

### (S)-6-AMINO-2-[2-(4-TRIFLUOROMETHYL-PHENYLAMINO)-ACETYLAMINO]-HEXANOIC ACID P-TOLYLAMIDE

Prepared by solid-phase as described in Scheme 2: Low resolution mass spectrum (ES) m/e 437 [(M+H)⁺, calcd for C₂₂H₂₈F₃N₄O₂: 438]; 82% purity based on HPLC.

### Example 366

### (S)-6-AMINO-2-[2-(2-PHENOXY-PHENYLAMINO)-ACETYLAMINO]-HEXANOIC ACID P-TOLYLAMIDE

Prepared by solid-phase as described in Scheme 2: Low resolution mass spectrum (ES) m/e 461 [(M+H)⁺, calcd for C₂₇H₃₃N₄O₃: 461]; 85% purity based on HPLC.

### Example 367

### (S)-6-AMINO-2-[2-(3-PHENOXY-PHENYLAMINO)-ACETYLAMINO]-HEXANOIC ACID P-TOLYLAMIDE

Prepared by solid-phase as described in Scheme 2: Low resolution mass spectrum (ES) m/e 461 [(M+H)⁺, calcd for C₂₇H₃₃N₄O₃: 461]; 91% purity based on HPLC.

### Example 368

### (S)-6-AMINO-2-[2-(4-PHENOXY-PHENYLAMINO)-ACETYLAMINO]-HEXANOIC ACID P-TOLYLAMIDE

Prepared by solid-phase as described in Scheme 2: Low resolution mass spectrum (ES) m/e 461 [(M+H)⁺, calcd for C₂₇H₃₃N₄O₃: 461]; 83% purity based on HPLC.

### Example 369

### (S)-6-AMINO-2-[2-(INDAN-5-YLAMINO)-ACETYLAMINO]-HEXANOIC ACID P-TOLYLAMIDE

Prepared by solid-phase as described in Scheme 2: Low resolution mass spectrum (ES) m/e 409 [(M+H)⁺, calcd for C₂₄H₃₃N₄O₂: 409]; 85% purity based on HPLC.

### Example 370

### (S)-3-(9H-FLUOREN-9-YLMETHOXYCARBONYLAMINO)-NR-P-TOLYL-SUCCINAMIC ACID

Prepared by solid-phase as described in Scheme 2: Low resolution mass spectrum (ES) m/e 445 [(M+H)⁺, calcd for C₂₆H₂₅N₂O₅: 445]; 95.2% purity based on HPLC.

### Example 371

### (R)-3-(9H-FLUOREN-9-YLMETHOXYCARBONYLAMINO)-NR-P-TOLYL-SUCCINAMIC ACID

Prepared by solid-phase as described in Scheme 2: Low resolution mass spectrum (ES) m/e 445 [(M+H)⁺, calcd for C₂₆H₂₅N₂O₅: 445]; 94.8% purity based on HPLC.

### Example 372

### (S)-4-(9H-FLUOREN-9-YLMETHOXYCARBONYLAMINO)-4-P-TOLYLCARBAMOYL-BUTYRIC ACID

Prepared by solid-phase as described in Scheme 2: Low resolution mass spectrum (ES) m/e 459 [(M+H)⁺, calcd for C₂₇H₂₇N₂O₅: 459]; 99% purity based on HPLC.

### Example 373

### (R)-4-(9H-FLUOREN-9-YLMETHOXYCARBONYLAMINO)-4-P-TOLYLCARBAMOYL-BUTYRIC ACID

Prepared by solid-phase as described in Scheme 2: Low resolution mass spectrum (ES) m/e 459 [(M+H)⁺, calcd for C₂₇H₂₇N₂O₅: 459]; 99.2% purity based on HPLC.

### Example 374

### (S)-(3-HYDROXY-1-P-TOLYLCARBAMOYL-PROPYL)-CARBAMIC ACID 9H-FLUOREN-9-YLMETHYL ESTER

Prepared by solid-phase as described in Scheme 2: Low resolution mass spectrum (ES) m/e 431 [(M+H)⁺, calcd for C₂₆H₂₇N₂O₄: 431]; 86.6% purity based on HPLC.

### Example 375

### (R)-(2-HYDROXY-1-P-TOLYLCARBAMOYL-ETHYL)-CARBAMIC ACID 9H-FLUOREN-9-YLMETHYL ESTER

Prepared by solid-phase as described in Scheme 2: Low resolution mass spectrum (ES) m/e 417 [(M+H)⁺, calcd for C₂₅H₂₅N₂O₄: 417]; 98.2% purity based on HPLC.

### Example 376

### (2+/−,3S)-(2-HYDROXY-1-P-TOLYLCARBAMOYL-PROPYL)-CARBAMIC ACID 9H-FLUOREN-9-YLMETHYL ESTER

Prepared by solid-phase as described in Scheme 2: Low resolution mass spectrum (ES) m/e 431 [(M+H)⁺, calcd for C₂₆H₂₇N₂O₄: 431]; 96.1% purity based on HPLC.

### Example 377

### (2+/−,3R)-(2-HYDROXY-1-P-TOLYLCARBAMOYL-PROPYL)-CARBAMIC ACID 9H-FLUOREN-9-YLMETHYL ESTER

Prepared by solid-phase as described in Scheme 2: Low resolution mass spectrum (ES) m/e 431 [(M+H)⁺, calcd for C₂₆H₂₇N₂O₄: 43 1 ]; 99.3% purity based on HPLC.

### Example 378

### (S)-[2-(1H-INDOL-3-YL)-1-P-TOLYLCARBAMOYL-ETHYL]-CARBAMIC ACID 9H-FLUOREN-9-YLMETHYL ESTER

Prepared by solid-phase as described in Scheme 2: Low resolution mass spectrum (ES) m/e 516 [(M+H)⁺, calcd for C₃₃H₃₀N₃O₃: 516]; 88.2% purity based on HPLC.

### Example 379

### (R)-[2-(1H-INDOL-3-YL)-1-P-TOLYLCARBAMOYL-ETHYL]-CARBAMIC ACID 9H-FLUOREN-9-YLMETHYL ESTER

Prepared by solid-phase as described in Scheme 2: Low resolution mass spectrum (ES) m/e 516 [(M+H)⁺, calcd for C₃₃H₃₀N₃O₃: 516]; 96.7% purity based on HPLC.

### Example 380

### (R)-[2-(4-HYDROXY-PHENYL)-1-P-TOLYLCARBAMOYL-ETHYL]-CARBAMIC ACID 9H-FLUOREN-9-YLMETHYL ESTER

Prepared by solid-phase as described in Scheme 2: Low resolution mass spectrum (ES) m/e 493 [(M+H)⁺, calcd for C₃₁H₂₉N₂O₄: 493]; 98.5% purity based on HPLC.

### Example 381

### (S)-(2-THIOPHEN-2-YL-1-P-TOLYLCARBAMOYL-ETHYL)-CARBAMIC ACID 9H-FLUOREN-9-YLMETHYL ESTER

Prepared by solid-phase as described in Scheme 2: Low resolution mass spectrum (ES) m/e 483 [(M+H)⁺, calcd for C₂₉H₂₇N₂O₄S: 483]; 88.2% purity based on HPLC.

### Example 382

### (R)-(3-CARBAMOYL-1-P-TOLYLCARBAMOYL-PROPYL)-CARBAMIC ACID 9H-FLUOREN-9-YLMETHYL ESTER

Prepared by solid-phase as described in Scheme 2: Low resolution mass spectrum (ES) m/e 458 [(M+H)⁺, calcd for C₂₇H₂₈N₃O₄: 458]; 98.5% purity based on HPLC.

### Example 383

### (R)-(3-METHYLSULFANYL-1-P-TOLYLCARBAMOYL-PROPYL)-CARBAMIC ACID 9H-FLUOREN-9-YLMETHYL ESTER

Prepared by solid-phase as described in Scheme 2: Low resolution mass spectrum (ES) m/e 461 [(M+H)⁺, calcd for C₂₇H₂₉N₂O₃S: 461]; 86.1% purity based on HPLC.

### Example 384

### (R)-[2-(3H-IMIDAZOL-4-YL)-1-P-TOLYLCARBAMOYL-ETHYL]-CARBAMIC ACID 9H-FLUOREN-9-YLMETHYL ESTER

Prepared by solid-phase as described in Scheme 2: Low resolution mass spectrum (ES) m/e 467 [(M+H)⁺, calcd for C₂₈H₂₇F₃N₄O₃: 467]; 96.2% purity based on HPLC.

### Example 385

### (S)-(2-PYRIDIN-3-YL-1-P-TOLYLCARBAMOYL-ETHYL)-CARBAMIC ACID 9H-FLUOREN-9-YLMETHYL ESTER

Prepared by solid-phase as described in Scheme 2: Low resolution mass spectrum (ES) m/e 478 [(M+H)⁺, calcd for C₃₀H₂₈N₃O₃: 478]; 98.9% purity based on HPLC.

### Example 386

### (S)-6-AMINO-2-(3,4-DIMETHOXY-BENZENESULFONYLAMINO)-HEXANOIC ACID P-TOLYLAMIDE

Prepared by solid-phase as described in Scheme 2: Low resolution mass spectrum (ES) m/e 436 [(M+H)⁺, calcd for C₂₁H₃₀N₃O₅S: 436]; 100% purity based on HPLC.

### Example 387

### (S)-6-AMINO-2-BENZENESULFONYLAMINO-HEXANOIC ACID P-TOLYLAMIDE

Prepared by solid-phase as described in Scheme 2: Low resolution mass spectrum (ES) m/e 376 [(M+H)⁺, calcd for C₁₉H₂₅N₃O₃S: 376]; 100% purity based on HPLC.

### Example 388

### (S)-6-AMINO-2-(4-BENZENESULFONYL-THIOPHENE-2-SULFONYLAMINO)-HEXANOIC ACID P-TOLYLAMIDE

Prepared by solid-phase as described in Scheme 2: Low resolution mass spectrum (ES) m/e 522 [(M+H)⁺, calcd for C₂₃H₂₈N₃O₅S₃: 522]; 85% purity based on HPLC.

### Example 389

### (S)-6-AMINO-2-(5-BENZENESULFONYL-THIOPHENE-2-SULFONYLAMINO)-HEXANOIC ACID P-TOLYLAMIDE

Prepared by solid-phase as described in Scheme 2: Low resolution mass spectrum (ES) m/e 522 [(M+H)⁺, calcd for C₂₃H₂₈N₃O₅S₃: 522]; 88% purity based on HPLC.

### Example 390

### (S)-6-AMINO-2-(4-BUTOXY-BENZENESULFONYLAMINO)-HEXANOIC ACID P-TOLYLAMIDE

Prepared by solid-phase as described in Scheme 2; Low resolution mass spectrum (ES) m/e 448 [(M+H)⁺, calcd for C₂₃H₃₄N₃O₄S: 448]; 92% purity based on HPLC.

### Example 391

### (S)-6-AMINO-2-(4-CHLORO-BENZENESULFONYLAMINO)-HEXANOIC ACID P-TOLYLAMIDE

Prepared by solid-phase as described in Scheme 2: Low resolution mass spectrum (ES) m/e 410 [(M+H)⁺, calcd for C₁₉H₂₅N₃O₃S: 410]; 100% purity based on HPLC.

### Example 392

### (S)-6-AMINO-2-(3,4-DICHLORO-BENZENESULFONYLAMINO)-HEXANOIC ACID P-TOLYLAMIDIE

Prepared by solid-phase as described in Scheme 2: Low resolution mass spectrum (ES) m/e 444 [(M+H)⁺, calcd for C₁₉H₂₄ C₂N₃O₃S: 444]; 94% purity based on HPLC.

### Example 393

### (S)-6-AMINO-2-(2,4-DICHLORO-BENZENESULFONYLAMINO)-HEXANOIC ACID P-TOLYLAMIDE

Prepared by solid-phase as described in Scheme 2: Low resolution mass spectrum (ES) m/e 444 [(M+H)⁺, calcd for C₁₉H₂₄ C₁₂N₃O₃S: 444]; 86% purity based on HPLC.

### Example 394

### (S)-6-AMINO-2-(3,5-DICHLORO-BENZENESULFONYLAMINO)-HEXANOIC ACID P-TOLYLAMIDE

Prepared by solid-phase as described in Scheme 2: Low resolution mass spectrum (ES) m/e 444 [(M+H)⁺, calcd for C₁₉H₂₄ C₁₂N₃O₃S: 444]; 93% purity based on HPLC.

### Example 395

### (S)-6-AMINO-2-(2,5-DICHLORO-THIOPHENE-3-SULFONYLAMINO)-HEXANOIC ACID P-TOLYLAMIDE

Prepared by solid-phase as described in Scheme 2; Low resolution mass spectrum (ES) m/e 450 [(M+H)⁺, calcd for C₁₇H₂₂ C₁₂N₃O₃S: 450]; 88% purity based on HPLC.

### Example 396

### (S)-6-AMINO-2-(TOLUENE-4-SULFONYLAMINO)-HEXANOIC ACID P-TOLYLAMIDE

Prepared by solid-phase as described in Scheme 2: Low resolution mass spectrum (ES) m/e 390 [(M+H)⁺, calcd for C₂₀H₂₈N₃O₃S: 390]; 100% purity based on HPLC.

### Example 397

### (S)-6-AMINO-2-(4-TRIFLUOROMETHYL-BENZENESULFONYLAMINO)-HEXANOIC ACID P-TOLYLAMIDE

Prepared by solid-phase as described in Scheme 2: Low resolution mass spectrum (ES) m/e 444 [(M+H)⁺, calcd for C₂₀H₂₅F₃N₃O₃S: 444]; 91% purity based on HPLC.

### Example 398

### (S)-6-AMINO-2-(BENZO[1,2,5]OXADIAZOLE-4-SULFONYLAMINO)-HEXANOIC ACID P-TOLYLAMIDE

Prepared by solid-phase as described in Scheme 2: Low resolution mass spectrum (ES) m/e 418 [(M+H)⁺, calcd for C₁₉H₂₄N₅O₄S: 418]; 92% purity based on HPLC.

### Example 399

### (S)-6-AMINO-2-(THIOPHENE-3-SULFONYLAMINO)-HEXANOIC ACID P-TOLYLAMIDE

Prepared by solid-phase as described in Scheme 2: Low resolution mass spectrum (ES) m/e 382 [(M+H)⁺, calcd for C₁₇H₂₄N₃O₃S₂: 382]; 98% purity based on HPLC.

### Example 400

### (R)-(3-AMINO-1-P-TOLYLCARBAMOYL-PROPYL)-CARBAMIC ACID 9H-FLUOREN-9-YLMETHYL ESTER

Prepared by solid-phase as described in Scheme 2: Low resolution mass spectrum (ES) m/e 430 [(M+H)⁺, calcd for C₂₆H₂₈N₃O₃: 430]; 100% purity based on HPLC.

### Example 401

### (R)-(2-AMINO-1-P-TOLYLCARBAMOYL-ETHYL)-CARBAMIC ACID 9H-FLUOREN-9-YLMETHYL ESTER

Prepared by solid-phase as described in Scheme 2: Low resolution mass spectrum (ES) m/e 416 [(M+H)⁺, calcd for C₂₅H₂₆N₃O₃: 416]; 100% purity based on HPLC.

### Example 402

### (S)-5-(9H-FLUOREN-9-YLMETHOXYCARBONYLAMINO)-5-P-TOLYLCARBAMOYL-PENTANOIC ACID

Prepared by solid-phase as described in Scheme 2: Low resolution mass spectrum (ES) m/e 473 [(M+H)⁺, calcd for C₂₈H₂₈N₂O₅: 473]; 91% purity based on HPLC.

### Example 403

### (S)-(5-AMINO-1-P-TOLYLCARBAMOYL-PENTYL)-CARBAMIC ACID BENZYL ESTER

Prepared by solid-phase as described in Scheme 2: Low resolution mass spectrum (ES) m/e 370 [(M+H)⁺, calcd for C₂₁H₂₈N₃O₃: 370]; 100% purity based on HPLC.

### Example 404

### IN VITRO ANTIVIRAL ACTIVITY—RDRP ASSAY WITH ELISA

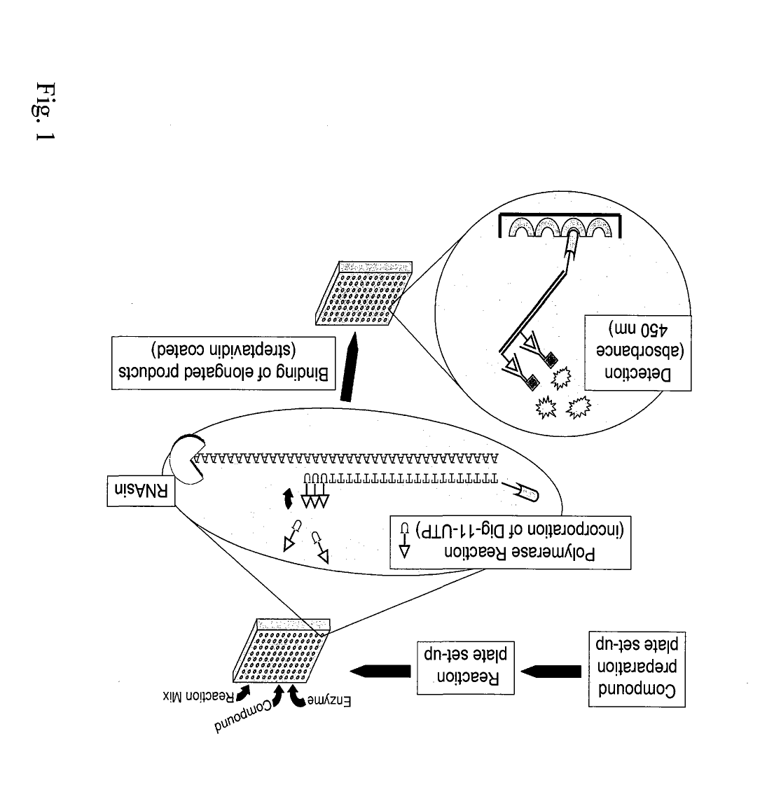

An RNA-dependant RNA polymerase assay was performed in a 96-well round bottom polypropylene plates (Costar) with a 50 μl reaction volume containing 20 mM HEPES pH 7.2-7.5 (Gibco), 1 mM DTT (Sigma), 0.4 mM MnCl2 (Sigma), 70 nM RNA template-primer PolyA (Amersham)-Oligo dT(20) biotin labelled (Roche), 20 μM UTP (Roche), 5U RNasin (Promega), 50 mM NaCl (Ambion), 4 μM DIG-11-UTP (Roche), test compound at desired concentration, 5% DMSO and 50-80 ng NS5B (Replizyme(V, UK) or 200-500 ng of recombinant NS5BA21 HCV RdRp enzyme. After 1 hour incubation at 30° C, the reaction is stopped by the addition of 5 μl 0.5 M EDTA and the solution is transferred to a streptavidin coated 96 well plate (Roche). Standard ELISA detection using Anti DIG-POD antibody (Roche) and BM Blue substrate is applied before measuring Abs at 450 nm. An illustration of the assay can be found in FIG. 1.

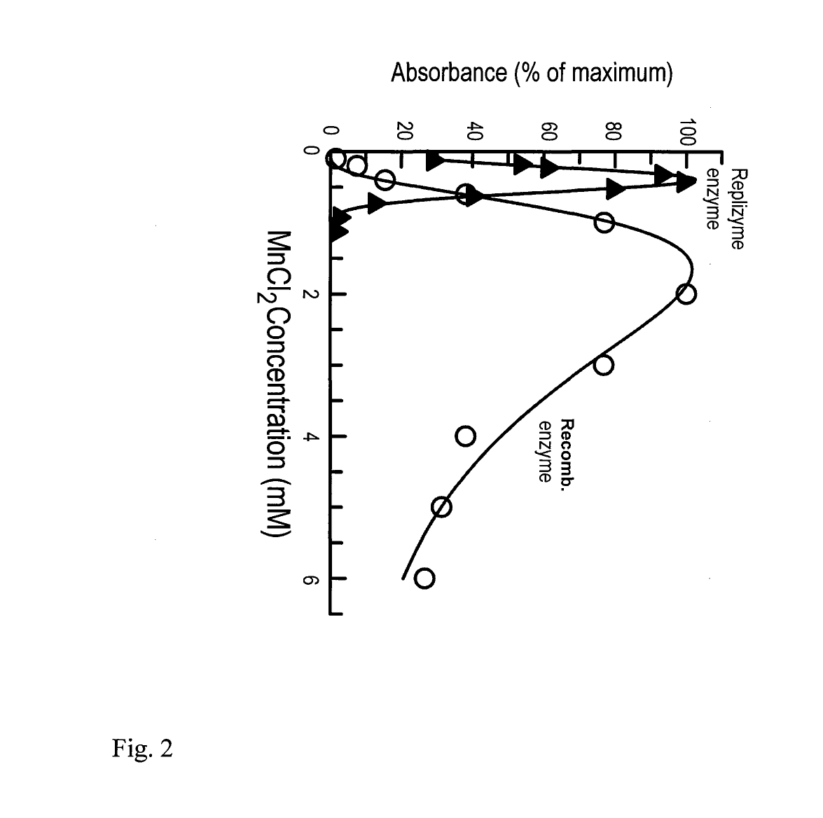

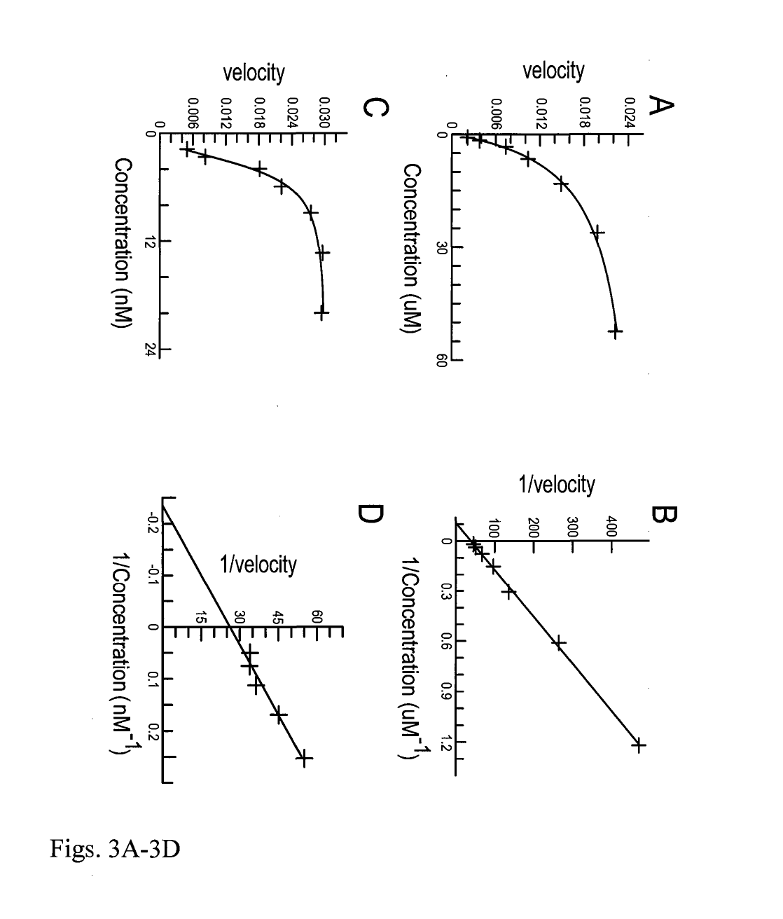

In this assay system, the polymerase reaction was very efficient in the presence of Mn²⁺ ; however, it did not take place in the presence of Mg²+(see FIG. 2). Most previous reports indicated that both cofactors were suitable. The two enzymes used in these experiments are of the same genotype; nevertheless, they showed different optimal Mn²⁺ concentrations (0.4 mM for the Replizyme®D enzyme vs. 2 mM for the in-house enzyme). It is interesting to note that the physiological Mn²⁺ concentration in vivo is in the low micromolar range. Reaction velocity was also measured as ΔAbs/min and plotted against the concentration of UTP (EM) used (see FIG. 3A). Both enzyme constructs showed a lower Kₘ (5 nM) for the template-primer than the Kₘ reported in literature (25-214 nM) (see FIG. 3B).

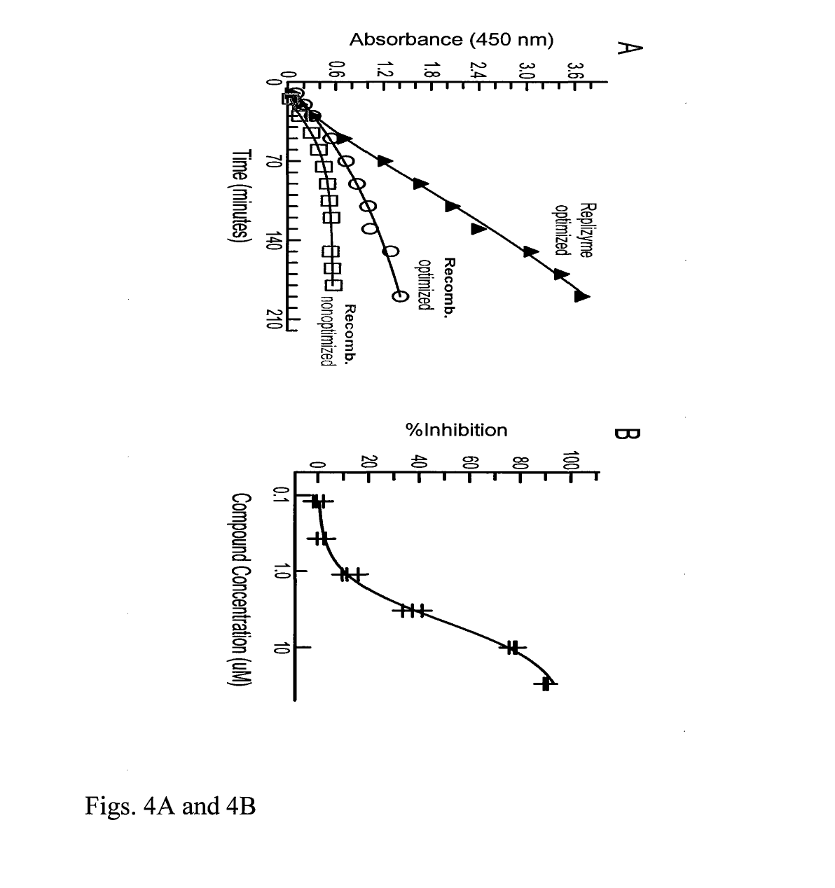

Optimized conditions lead to sustained linear response and lower enzyme quantity is needed due to the presence of stabilizers (see FIG. 4A). A dose-response inhibitory curve for compound E-HCV-5 is shown in FIG. 4B. One difference between the two enzyme sequences is the amino acid in position 499 (T→V). This amino acid difference is potentially relevant (see WO2004/99241) because of its proximity to the binding site for inhibitors 4 and 5.

The two HCV genotype1b Δ21 RdRp enzymes (produced in-house and from Replizyme(®) were used to evaluate several published RdRp inhibitors. Known anti-HCV compounds I, II and III showed similar levels of inhibition with both enzymes (see Table 2). However, the chemically related compounds IV and V showed about 20 times less inhibition with the Replizyme® enzyme than with the prepared enzyme (see Table 2). The antiviral compounds of the instant disclosure were tested in this assay as well and results are provided in Table 1.

TABLE 1
IC₅₀ of Test Compounds in RdRp Assay

| Compound # | RdRp IC₅₀ (μM) |
|---|---|
| 2 | 10 to 50 |
| 3 | >50 |
| 4 | >50 |
| 4 | >50 |
| 5 | >50 |
| 6 | <10 |
| 7 | >50 |
| 8 | >50 |
| 9 | >50 |
| 10 | >50 |
| 11 | >50 |
| 12 | >50 |
| 13 | >50 |
| 14 | 10 to 50 |
| 15 | 10 to 50 |
| 16 | <10 |
| 17 | 10 to 50 |
| 18 | >50 |
| 19 | >50 |
| 20 | 10 to 50 |
| 21 | >50 |
| 22 | 10 to 50 |
| 23 | 10 to 50 |
| 24 | 10 to 50 |
| 25 | 10 to 50 |
| 26 | <10 |
| 27 | >50 |
| 28 | >50 |
| 29 | >50 |
| 30 | >50 |
| 31 | 10 to 50 |
| 32 | >50 |
| 33 | 10 to 50 |
| 34 | 10 to 50 |
| 35 | <10 |
| 36 | 10 to 50 |
| 37 | >50 |
| 38 | 10 to 50 |
| 39 | >50 |
| 40 | 10 to 50 |
| 41 | >50 |
| 42 | 10 to 50 |
| 43 | >50 |
| 44 | >50 |
| 45 | >50 |
| 46 | 10 to 50 |
| 47 | >50 |
| 48 | >50 |
| 49 | >50 |
| 50 | >50 |
| 51 | >50 |
| 52 | >50 |
| 53 | >50 |
| 54 | >50 |
| 55 | >50 |
| 56 | >50 |
| 57 | >50 |
| 58 | >50 |
| 59 | 10 to 50 |
| 60 | >50 |
| 61 | >50 |
| 62 | >50 |
| 63 | >50 |
| 64 | >50 |
| 65 | >50 |
| 66 | >50 |
| 67 | >50 |
| 68 | >50 |
| 69 | >50 |
| 70 | >50 |
| 71 | >50 |
| 72 | 10 to 50 |
| 73 | >50 |
| 74 | 10 to 50 |
| 75 | >50 |
| 76 | 10 to 50 |
| 77 | >50 |
| 78 | >50 |
| 79 | >50 |
| 80 | >50 |
| 81 | 10 to 50 |
| 82 | >50 |
| 83 | 10 to 50 |
| 84 | >50 |
| 85 | >50 |
| 86 | >50 |
| 87 | >50 |
| 88 | >50 |
| 89 | >50 |
| 90 | >50 |
| 91 | >50 |
| 92 | >50 |
| 93 | >50 |
| 94 | <10 |
| 95 | 10 to 50 |
| 96 | >50 |
| 97 | >50 |
| 98 | >50 |
| 99 | >50 |
| 100 | >50 |
| 101 | >50 |
| 102 | >50 |
| 103 | >50 |
| 104 | <10 |
| 105 | 10 to 50 |
| 106 | >50 |
| 107 | <10 |
| 108 | >50 |
| 109 | >50 |
| 110 | >50 |
| 111 | >50 |
| 112 | >50 |
| 113 | >50 |
| 114 | >50 |
| 115 | 10 to 50 |
| 116 | <10 |
| 117 | <10 |
| 118 | >50 |
| 119 | <10 |
| 120 | <10 |
| 121 | <10 |
| 122 | >50 |
| 123 | >50 |
| 124 | >50 |
| 125 | <10 |
| 126 | <10 |
| 127 | 10 to 50 |
| 128 | <10 |
| 129 | <10 |
| 130 | >50 |
| 131 | >50 |
| 132 | >50 |
| 133 | >50 |
| 134 | >50 |
| 135 | >50 |
| 136 | >50 |
| 137 | <10 |
| 138 | >50 |
| 139 | >50 |
| 140 | 10 to 50 |
| 141 | <10 |
| 142 | <10 |
| 143 | <10 |
| 144 | <10 |
| 145 | <10 |
| 146 | <10 |
| 147 | <10 |
| 148 | >50 |
| 149 | <10 |
| 150 | <10 |
| 151 | <10 |
| 152 | <10 |
| 153 | >50 |
| 154 | 10 to 50 |
| 155 | 10 to 50 |
| 156 | >50 |
| 157 | >50 |
| 158 | 10 to 50 |
| 159 | 10 to 50 |
| 160 | 10 to 50 |
| 161 | 10 to 50 |
| 162 | 10 to 50 |
| 163 | 10 to 50 |
| 164 | <10 |
| 165 | 10 to 50 |
| 166 | <10 |
| 167 | >50 |
| 168 | >50 |
| 169 | >50 |
| 170 | >50 |
| 171 | 10 to 50 |
| 172 | 10 to 50 |
| 173 | <10 |
| 174 | <10 |
| 175 | <10 |
| 176 | <10 |
| 177 | <10 |
| 178 | 10 to 50 |
| 179 | 10 to 50 |
| 180 | >50 |
| 181 | >50 |
| 182 | >50 |
| 183 | >50 |
| 184 | >50 |
| 185 | >50 |
| 186 | >50 |
| 187 | 10 to 50 |
| 188 | >50 |
| 189 | >50 |
| 190 | <10 |
| 191 | >50 |
| 192 | >50 |
| 193 | <10 |
| 194 | >50 |
| 195 | <10 |
| 196 | 10 to 50 |
| 197 | >50 |
| 198 | <10 |
| 199 | 10 to 50 |
| 200 | <10 |
| 201 | <10 |
| 202 | <10 |
| 203 | <10 |
| 204 | >50 |
| 205 | >50 |
| 206 | 10 to 50 |
| 207 | <10 |
| 208 | <10 |
| 209 | 10 to 50 |
| 210 | <10 |
| 211 | 10 to 50 |
| 212 | 10 to 50 |
| 213 | >50 |
| 214 | <10 |
| 215 | 10 to 50 |
| 216 | 10 to 50 |
| 217 | 10 to 50 |
| 218 | >50 |
| 219 | >50 |
| 310 | N/A |
| 311 | >50 |
| 312 | >50 |
| 326 | >50 |
| 327 | >50 |
| 328 | >50 |
| 329 | >50 |
| 330 | >50 |
| 331 | >50 |
| 332 | >50 |
| 333 | >50 |
| 334 | >50 |
| 335 | >50 |
| 336 | >50 |
| 337 | >50 |
| 338 | >50 |
| 339 | >50 |
| 340 | >50 |
| 341 | >50 |
| 342 | >50 |
| 343 | >50 |
| 344 | >50 |
| 345 | >50 |
| 346 | >50 |
| 347 | >50 |
| 348 | >50 |
| 349 | >50 |
| 350 | >50 |
| 351 | >50 |
| 352 | >50 |
| 370 | >50 |
| 371 | >50 |
| 372 | >50 |
| 373 | >50 |
| 374 | >50 |
| 375 | >50 |
| 376 | >50 |
| 377 | >50 |
| 378 | >50 |
| 379 | >50 |
| 380 | >50 |
| 381 | >50 |
| 382 | >50 |
| 383 | >50 |
| 384 | >50 |
| 385 | >50 |
| 386 | >50 |
| 387 | >50 |
| 388 | >50 |
| 220 | >50 |
| 221 | >50 |
| 222 | 10 to 50 |
| 223 | 10 to 50 |
| 224 | 10 to 50 |
| 225 | >50 |
| 226 | >50 |
| 227 | <10 |
| 228 | <10 |
| 229 | >50 |
| 230 | >50 |
| 231 | >50 |
| 232 | >50 |
| 233 | <10 |
| 234 | 10 to 50 |
| 235 | <10 |
| 236 | <10 |
| 237 | <10 |
| 238 | <10 |
| 239 | <10 |
| 240 | 10 to 50 |
| 241 | <10 |
| 242 | <10 |
| 243 | >50 |
| 244 | >50 |
| 245 | >50 |
| 246 | 10 to 50 |
| 247 | 10 to 50 |
| 248 | 10 to 50 |
| 249 | 10 to 50 |
| 250 | 10 to 50 |
| 251 | <10 |
| 252 | <10 |
| 253 | 10 to 50 |
| 254 | 10 to 50 |
| 255 | 10 to 50 |
| 256 | >50 |
| 257 | >50 |
| 258 | >50 |
| 259 | >50 |
| 260 | >50 |
| 261 | >50 |
| 262 | <10 |
| 263 | 10 to 50 |
| 264 | 10 to 50 |
| 265 | 10 to 50 |
| 266 | >50 |
| 267 | 10 to 50 |
| 268 | 10 to 50 |
| 269 | >50 |
| 270 | 10 to 50 |
| 271 | <10 |
| 272 | 10 to 50 |
| 273 | <10 |
| 274 | <10 |
| 275 | 10 to 50 |
| 276 | 10 to 50 |
| 277 | >50 |
| 278 | 10 to 50 |
| 279 | >50 |
| 280 | >50 |
| 281 | >50 |
| 282 | >50 |
| 283 | 10 to 50 |
| 284 | 10 to 50 |
| 285 | 10 to 50 |
| 286 | <10 |
| 287 | 10 to 50 |
| 288 | 10 to 50 |
| 289 | >50 |
| 290 | >50 |
| 291 | >50 |
| 292 | >50 |
| 293 | <10 |
| 294 | >50 |
| 295 | <10 |
| 296 | 10 to 50 |
| 297 | <10 |
| 298 | >50 |
| 299 | 10 to 50 |
| 300 | >50 |
| 301 | >50 |
| 302 | >50 |
| 303 | >50 |
| 304 | 10 to 50 |
| 305 | <10 |
| 306 | 10 to 50 |
| 307 | 10 to 50 |
| 308 | >50 |
| 309 | >50 |
| 313 | 10 to 50 |
| 314 | 10 to 50 |
| 315 | 10 to 50 |
| 316 | 10 to 50 |
| 317 | >50 |
| 318 | 10 to 50 |
| 319 | <10 |
| 320 | <10 |
| 321 | <10 |
| 322 | 10 to 50 |
| 323 | >50 |
| 324 | 10 to 50 |
| 325 | >50 |
| 353 | >50 |
| 354 | >50 |
| 355 | >50 |
| 356 | >50 |
| 357 | >50 |
| 358 | 10 to 50 |
| 359 | >50 |
| 360 | 10 to 50 |
| 361 | >50 |
| 362 | >50 |
| 363 | >50 |
| 364 | >50 |
| 365 | >50 |
| 366 | <10 |
| 367 | <10 |
| 368 | <10 |
| 369 | >50 |
| 389 | >50 |
| 390 | >50 |
| 391 | >50 |
| 392 | >50 |
| 393 | >50 |
| 394 | >50 |
| 395 | >50 |
| 396 | >50 |
| 397 | >50 |
| 398 | >50 |
| 399 | >50 |
| 400 | >50 |
| 401 | >50 |
| 402 | >50 |
| 403 | >50 |

TABLE 2
Comparative Results of different RdRp Enzymes

| | Experimental IC₅₀ (μM) | | |
|---|---|---|---|
| Compound | Replizyme ® | Recombinant | Published IC₅₀ (μM) |
| 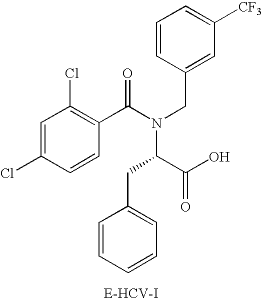 | 1.9 | 1.9 | 1.83 |
| 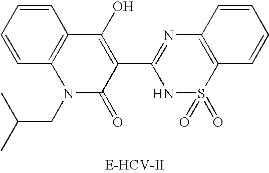 | 2.3 | 2.8 | 0.34 |
| 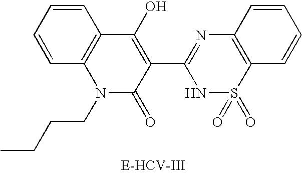 | 1.0 | 1.8 | 0.24 |
| 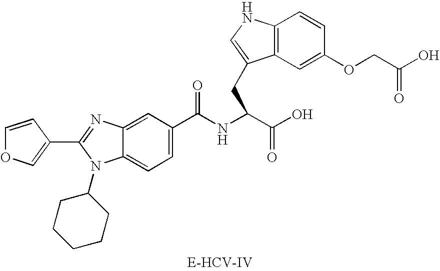 | 1.3 | 0.06 | 1.15 |
| 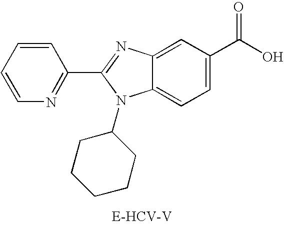 | 69 | 3.5 | 4.36 |
| 3′-dUTP | 0.13 | NA | NA |

In sum, a non-radioactive HCV RdRp assay is described for medium or high throughput evaluation of nucleoside and non-nucleoside inhibitors. The assay is robust and reproducible. Additionally, a differential behavior of two genotype 1b A21 enzymes was seen in their response to Mn²⁺ and their sensitivity to non-nucleoside inhibitors. Furthermore, we found that at 2 mM Mn²⁺, non-nucleoside compounds lost their ability to inhibit the recombinantly-produced enzyme.

### Example 405

### INHIBITION OF HCV REPLICATION IN CELL CULTURE

Antiviral activity of the test compounds was assessed in Huh-7 cells stably transfected with a sub-genomic, genotype 1a HCV replicon: H/SG-Neo (J. Virol. 77:3181, 2003) or genotype 1b. Culture conditions and antiviral treatments were performed as previously described (Blight et al., Science 290:1972, 2000; Okuse et al. Antivir. Res. 65:23, 2005). Test compounds were solubilized at 30 mM in 100% tissue culture grade DMSO (Sigma, Inc.). Aliquots of test compounds sufficient for one daily treatment were made in individual tubes and all material was stored at −20° C. On each day of treatment, daily aliquots of the test compounds were suspended in culture medium at room temperature and immediately added to the cell cultures. Compounds were added to dividing cultures once daily for three days at concentrations ranging from about 30 μM to about 0.0003 μM. Media was changed with each addition of compound. The assays were started when cell cultures were at about 30-50% confluence, and the cells reached confluence during the last day of treatment. The assays included untreated control cultures (six total), and triplicate cultures treated with α-interferon and ribavirin as positive antiviral and toxicity controls.

Intracellular HCV RNA levels and cytotoxicity were assessed in triplicate 24 hours after the last compound dose. Intracellular HCV RNA levels were measured using a blot hybridization method, in which HCV RNA levels were normalized to the levels of B-actin RNA in each individual culture (Okuse et al., Antivir. Res. 65:23, 2005). Cytotoxicity was measured using a neutral red dye uptake assay (Korba and Gerin, Antivir. Res. 19:55, 1992; Okuse et al. Antivir. Res. 65:23, 2005). The EC₅₀ was calculated, which represents the concentration of compound that inhibits 50% of HCV RNA produced in cells as compared to an untreated control. The CC₅₀ is a measure of cytotoxicity caused by the test compound and equals the concentration that affects the viability of 50% of the treated cells as compared to untreated cells. All of the test compounds showed anti-HCV activity, with some compounds having activity at an EC₅₀ below 0.5 μM (see Table 3). Additionally, the compounds showed a favorable Selectivity Index (i.e., values greater than about 10 means the detected activity is likely due to an antiviral effect rather than a cytotoxic effect).

TABLE 3
Anti-HCV Activity in Cell Culture

| Compound No. | EC₅₀ (μM) | Selectivity Index (CC₅₀/EC₅₀) |
|---|---|---|
| 2 | <0.5 | >100 |
| 2* | <0.5 | 20 to 100 |
| 26 | >2 | <20 |
| 94 | >2 | <20 |
| 117 | >2 | <20 |
| 119 | >2 | <20 |
| 120 | >2 | <20 |
| 121 | <0.5 | <20 |
| 125 | >2 | <20 |
| 127 | >2 | <20 |
| 137 | <0.5 | 20 to 100 |
| 142 | <0.5 | <20 |
| 145 | >2 | <20 |
| 146 | <0.5 | 20 to 100 |
| 163 | <0.5 | <20 |
| 166 | >2 | <20 |
| 172 | 2 to 0.5 | <20 |
| 172* | <0.5 | 20 to 100 |
| 173 | 2 to 0.5 | <20 |
| 199 | 2 to 0.5 | 20 to 100 |
| 206 | >2 | <20 |
| 206* | >2 | <20 |
| 207 | >2 | <20 |
| 214 | >2 | <20 |
| 228 | 0.5 | 20 to 100 |
| 237 | >2 | <20 |
| 240 | >2 | <20 |
| 246 | >2 | <20 |
| 262 | 2 to 0.5 | 20 to 100 |
| 262* | <0.5 | >100 |
| 264 | <0.5 | >100 |
| 268 | >2 | <20 |
| 270 | >2 | <20 |
| 272 | 2 to 0.5 | 20 to 100 |
| 297 | <0.5 | >100 |
| 297* | <0.5 | >100 |
| 306 | >2 | <20 |
| 324 | >2 | <20 |
| 366 | >2 | <20 |
| 367 | >2 | <20 |
| 368 | 2 to 0.5 | <20 |

*These compounds were tested in Huh-7 cells genotype 1a HCV replicon, while the unmarked compounds were tested in genotype 1b.

### Example 406

### Protection of MDBK Cells from BVDV-Induced Cytotoxicity

Cell proliferation assays were performed using a non-radioactive cell proliferation MTS/PMS assay (MTS: 3-(4,5-dimethylthiazol-2-yl)-5-(3-carboxymethoxy phenyl)-2-(4-sulfophenyl)-2H-tetrazolium (Promega Cat# PRG1112, Promega Corporation, Madison, Wis.); PMS: phenazine methosulfate (Sigma Cat# P9625, Sigma Aldrich, St. Louis, Mo.)). MDBK cells were seeded into 96-well plates at a density of approximately 2×10⁴ cells per well. The cultures were incubated for about 3 to about 24 hours to permit attachment of the cells to the plates prior to infection and addition of test compounds. The appropriate number of plaque forming units (PFU) of BVDV-NADL were added to each well to achieve the desired multiplicity of infection (MOI, <1 or >0.001); the cells were exposed to the virus diluted at the appropriate concentration in phosphate buffered saline (PBS) containing 1% horse serum (HS) for about 1 to about 2 hours. The virus inoculum was then removed and the cells were washed with PBS containing 1% HS. The test compounds were diluted in cell growth media with 2% HS and added to the cells at varying concentrations. The plates were incubated at 37° C in the presence of 5% CO₂ for 3-4 days. Uninfected cells and infected, untreated cells (without a test compound) were used as additional controls. The final volume was 100 Al per well. After about 3 to about 4 days of incubation, a volume of 20 μL of the combined MTS/PMS solution was added into each well of the 96 well assay plate containing 100 μL of cells in culture medium to obtain final concentrations of 333 μg/ml MTS and 25 μM PMS. A 96-well microtiter spectrophotometer plate reader was used to measure the absorbance at 490 nm after incubation of the 96-well plate for about 1 to about 4 hours at 37° C. in a humidified, 5% CO₂ atmosphere incubator. The mean absorbance in each set of triplicate wells was determined. Antiviral activity was measured as MTS conversion relative to the differential between the conversion for cell (non-infected) and viral (non-drug-treated) controls. The cytopathic effect (CPE) reduction for each concentration of the tested compound, which is a measurement that is correlated to antiviral activity, was calculated as follows: % CPE reduction=[(D−ND)/(NI−ND)]×100, where D (drug-treated)=the absorbance of drug-treated cells; ND (non drug-treated)=the absorbance of untreated infected cells; and NI (non-infected) =the absorbance of non-infected cells. EC₅₀ represents the concentration of test compound that protects 50% of the cells from BVDV induced cytotoxicity (50% CPE reduction). CC₅₀ equals the concentration that affects the viability of 50% of the MDBK cells. The EC₅₀ is the concentration of compound that inhibits 50% of viral release in the media of infected cells compared to the untreated control. The EC₅₀s of the test compounds varied from about 2.9 μM to >100 μM.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

All literature and patent references cited throughout the application are incorporated by reference into the application for all purposes.

1. A compound having a structure of formula (IV): 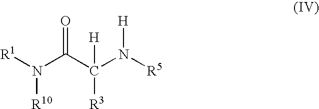
or a stereoisomer, prodrug or pharmaceutically acceptable salt thereof, wherein:
R¹, R³ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;
R⁵ is selected from —C(═O)NR⁹, —C(═O)(NR¹⁰)SO₂R⁹, —C(═S)NR¹⁰R⁹, or —C(═NR¹⁰)NR¹⁰R⁹; and
R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl.

2. The compound of claim 1, wherein R³ is not hydrogen.

3. The compound of claim 1, wherein R³ has an ionizable nitrogen.

4. The compound of claim 1, wherein the compound is compound 2, 297, 137, 146, 172, 199, 228, 272, 121, 142, 26, 94, 117, 119, 120, 125, 127, 145, 166, 173, 206, 207, 214, 237, 240, 268, 270, or 306 as shown in FIG. 5.

5. A compound having a structure of formula (III): 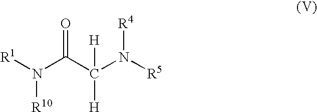
or a stereoisomer, prodrug or pharmaceutically acceptable salt thereof, wherein:
R¹, R⁴ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ and R⁴ are not hydrogen;
R⁵ is selected from —C(═O)NR⁹, —C(═O)(NR¹⁰)SO₂R⁹, —C(═S)NR¹⁰R⁹, or —C(═NR¹⁰)NR¹⁰R⁹; and
R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl.

6. The compound of claim 1, wherein R³ has an ionizable nitrogen.

7. The compound of claim 1, wherein the compound is compound 234, 262, 279, 281, 282, 294, 295, or 324 as shown in FIG. 5.

8. A compound having a structure of formula (VI): 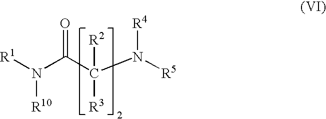
or a stereoisomer, prodrug or pharmaceutically acceptable salt thereof, wherein:
R¹ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;
(i) R², R³ and R⁴ are each independently the same or different substituent as defined for R⁹; or (ii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iii) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;
R⁵ is selected from H, —C(═O)R⁹, —C(═S)R⁹, —C(═NR¹⁰)R⁹, —CO₂R⁹, —C(═O)NR⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, or —SO₂R⁹; and
R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl;
and wherein at least one but not more than three of R², R³, R⁴ and R⁵ is hydrogen, provided that R¹ is not an amino acid when R⁴ and R⁵ are both H.

9. The compound of claim 8, wherein the compound has a structure of formula (VII): 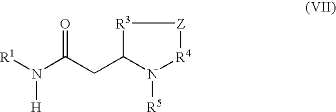
wherein:
R³ and R⁴ are each independently selected from —CH₂— or —(CH₂)₂—;
Z is —N(R⁹)—; and
R¹, R⁵, R⁹, and R¹⁰ are as defined in claim 8.

10. The compound of claim 8 or claim 9, wherein R⁹ has an ionizable nitrogen.

11. The compound of claim 8 or 9, wherein R³ is —CH₂— and R⁴ is —(CH₂)₂—.

12. The compound of claim 8, wherein the compound is compound 155, 158, 159, 160, 161, 162, 163, 183, 184, 186, 187, or 197 as shown in FIG. 5.

13. A compound having a structure of formula (VIII): 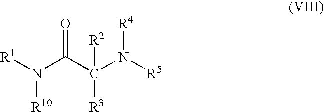
or a stereoisomer, prodrug or pharmaceutically acceptable salt thereof, wherein:
R¹ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkyl optionally substituted with one or more of the same or different R¹⁰ groups; provided that R¹ is not hydrogen;
(i) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (ii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iii) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;
R⁵ is selected from H, —C(═O)R⁹, —C(═S)R⁹, —C(═NR¹⁰)R⁹, —CO₂R⁹, —C(═O)NR⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, or —SO₂R⁹; and
R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl;
and wherein at least one but not more than three of R², R³, R⁴ and R⁵ is hydrogen, provided that R¹ is not an amino acid when R⁴ and R⁵ are both H.

14. The compound of claim 13, wherein at least one of R², R³ or R⁴ have an ionizable nitrogen.

15. The compound of claim 13 or 14, wherein R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent; and
R⁴ is selected from R⁹; and
R¹, R⁵, R⁹ and R¹⁰ are as defined in claim 13.

16. The compound of claim 15, wherein the compound is compound 85, 86, 87, 122, 123, 130, 131, 132, or 156 as shown in FIG. 5.

17. The compound of claim 13 or 14, wherein R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent; and
R² is selected from R⁹; and
R¹, R⁵, R⁹ and R¹⁰ are as defined in claim 13.

18. The compound of claim 17, wherein the compound is compound 109 or 138 as shown in FIG. 5.

19. A compound having a structure of formula (IX): 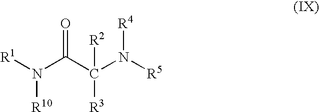
or a stereoisomer, prodrug or pharmaceutically acceptable salt thereof, wherein:
R¹ is the same as R⁹ provided an ionizable nitrogen is present;
(i) R², R³ and R⁴ are each independently the same or different substituent as defined for R⁹; or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;
R⁵ is selected from H, —C(═O)R⁹, —C(═S)R⁹, —C(═NR¹⁰)R⁹, —CO₂R⁹, —C(═O)NR⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, —SO₂R⁹;
R⁹ is selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;
R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl;
and wherein at least one but not more than three of R², R³, R⁴ and R⁵ is hydrogen, provided that R¹ is not an amino acid when R⁴ and R⁵ are both H.

20. The compound of claim 19, wherein the compound is compound 314, 315, 316, 319, or 320 as shown in FIG. 5.

21. A compound having a structure of formula (X): 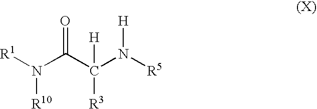
or a stereoisomer, prodrug or pharmaceutically acceptable salt thereof, wherein:
R¹, R³ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;
R⁵ is selected from H, —C(═O)R⁹, —C(═S)R⁹, —C(═NR¹⁰)R⁹, or —CO₂R⁹; R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl; and
R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl.

22. The compound of claim 21, wherein R³ is not hydrogen.

23. The compound of claim 21, wherein R³ has an ionizable nitrogen.

24. The compound of claim 21, wherein the compound is compound 358, 360, 366, 367 or 368 as shown in FIG. 5.
